(19) United States
(12) Patent Application Publication
(10) Pub. No.: US 2024/0316022 A1
(43) Pub. Date: Sep. 26, 2024

(54) INDAZOLE COMPOUNDS

(71) Applicant: TYRA BIOSCIENCES, INC., Carlsbad, CA (US)

(72) Inventors: Robert L. HUDKINS, Carlsbad, CA (US); Daniel C. BENSEN, Carlsbad, CA (US)

(21) Appl. No.: 18/573,731

(22) PCT Filed: Jun. 30, 2022
(86) PCT No.: PCT/US2022/073284

§ 371 Date: Dec. 22, 2023

Related U.S. Application Data
(60) Provisional application No. 63/216,893, filed on Jun. 30, 2021.

(51) Int. Cl. A61K 31/444; A61K 31/4545; A61K 31/4709; A61K 31/496; A61K 31/501; A61K 31/5377; A61K 31/5383; C07D 401/14; C07D 403/12; C07D 405/14; C07D 487/10; C07D 491/107; C07D 491/113; C07D 498/04

(52) U.S. Cl. CPC A61K 31/444; A61K 31/4545; A61K 31/4709; A61K 31/496; A61K 31/501; A61K 31/5377; A61K 31/5383; C07D 401/14; C07D 403/12; C07D 405/14; C07D 487/10; C07D 491/107; C07D 491/113; C07D 498/04

(57) ABSTRACT

Disclosed herein are compounds and methods of treating diseases and/or conditions associated with FGFR inhibition.

### CROSS-REFERENCE TO RELATED APPLICATION

This application claims the benefit of U.S. Provisional Application No. 63/216,893, filed Jun. 30, 2021, the entirety of which is incorporated by reference herein.

### TECHNICAL FIELD

The disclosure pertains to indazole compounds that are useful in treating cancer, pharmaceutical compositions that include one or more such indazole compounds, and methods of using such indazole compounds in treating cancer.

### BACKGROUND

Kinase inhibitors have been used to block the activity of kinases and thereby treat cancer (e.g., by inhibiting mitotic processes). These kinase inhibitors are often small molecules that target kinases to block the development, growth or spread of cancer.

However, although various inhibitors of kinases are known, there remains a need for selective inhibitors to be used for the treatment of diseases such as hyper-proliferative diseases, which offer one or more advantages over current compounds. Those advantages include: improved activity and/or efficacy; beneficial kinase selectivity profile according to the respective therapeutic need; improved side effect profile, such as fewer undesired side effects, lower intensity of side effects, or reduced (cyto)toxicity; improved targeting of mutant receptors in diseased cells; improved physicochemical properties, such as solubility/stability in water, body fluids, and/or pharmaceutical formulations; improved pharmacokinetic properties, allowing e.g. for dose reduction or an easier dosing scheme; easier drug substance manufacturing e.g. by shorter synthetic routes or easier purification.

### SUMMARY

The compounds disclosed herein provide small molecule kinase inhibitors that are both efficacious and selective.

In some aspects, the disclosure is directed to compounds of formula (I)

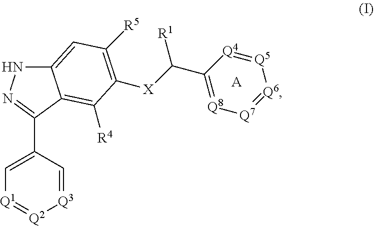

or a pharmaceutically acceptable salt thereof,
R¹ is C₁-C₆alkyl;
X=—O—, —S—, —NH—, or —N(C₁-C₃alkyl)-;
Q¹, Q², and Q³, are each independently N or C—R², wherein at least one of Q¹, Q², and Q³ is C—R²;
   or, when Q¹ and Q² are both C—R², said R² groups together with the atoms to which they are attached, form a substituted or unsubstituted 5-7 membered cycloalkyl ring or a substituted or unsubstituted 5-7 membered heterocycloalkyl ring;
each R² is independently H, halogen, substituted or unsubstituted —C₁-C₆alkyl, —OR^{2a}; —NR^{2b}R^{2c}, —C(O)NR^{2b}R^{2c}, —SO₂C₁-C₆alkyl, —CN, —CF₃, —CF₂, —NR^{2b}COR^{2a}, or —NR^{2b}CONR^{2a}R^{2b};
   or, when Q¹, Q², and Q³ are each C—R², each R² is independently H, halogen, substituted or unsubstituted —C₁-C₆alkyl, —OR^{2a}; —NR^{2b}R^{2c}, —C(O)NR^{2b}R^{2c}, —SO₂C₁-C₆alkyl, —CN, —CF₃, —CF₂, —NR^{2b}COR^{2a}, —NR^{2b}CONR^{2a}R^{2b}, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, or a substituted or unsubstituted spiroheterocycloalkyl;
each R^{2a} is independently H, substituted or unsubstituted —C₁-C₆alkyl, —C₁-C₆haloalkyl, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, substituted or unsubstituted spiroheterocycloalkyl, substituted or unsubstituted —C₁-C₆alk-C₃-C₆cycloalkyl, or substituted or unsubstituted —C₁-C₆alk-O—C₁-C₆alkyl,
each R^{2b} is independently H, —C(O)-(substituted or unsubstituted 5- to 10-membered heteroaryl), —C(O)-(substituted or unsubstituted 5- to 6-membered heterocycloalkyl), substituted or unsubstituted —C₁-C₆alkyl, substituted or unsubstituted —C₁-C₆alk-O—C₁-C₆alkyl, —C(O)-(substituted or unsubstituted —C₃-C₆cycloalkyl), —C(O)N (substituted or unsubstituted C₁-C₆alkyl)₂, —C(O)C₁-C₃alkN (substituted or unsubstituted C₁-C₆alkyl)₂, substituted or unsubstituted —C(O)—OC₁-C₆alkyl, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, substituted or unsubstituted spiroheterocycloalkyl, or —C(O)CH═CH₂;
each R^{2c} is independently H, —C(O)-(substituted or unsubstituted 5- to 10-membered heteroaryl), —C(O)-(substituted or unsubstituted 5- to 6-membered heterocycloalkyl), substituted or unsubstituted —C₁-C₆alkyl, substituted or unsubstituted —C₁-C₆alk-O—C₁-C₆alkyl, —C(O)-(substituted or unsubstituted —C₃-C₆cycloalkyl), —C(O)N (substituted or unsubstituted C₁-C₆alkyl)₂, —C(O)C₁-C₃alkN (substituted or unsubstituted C₁-C₆alkyl)₂, substituted or unsubstituted —C(O)—OC₁-C₆alkyl, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, substituted or unsubstituted spiroheterocycloalkyl, or —C(O)CH═CH₂;
Q⁴, Q⁵, Q⁶, Q⁷, and Q⁸, are each independently N or C—R³, wherein one or two of Q⁴, Q⁵, Q⁶, Q⁷, and Q⁸ is N and the remainder are C—R³; and each R³ is independently H, halogen, C₁-C₆alkyl, C₁-C₆alkoxyl, or cycloalkyl;
R⁴ is H, halogen, —C₁-C₆alkyl, —C₁-C₆alkoxyl, or -cycloalkyl;
R⁵ is H, halogen, —C₁-C₆alkyl, —C₁-C₆alkoxyl, or -cycloalkyl;
wherein when ring A is 3,5-dichloropyridin-4-yl, and two of Q¹, Q′, or Q³ are C—H, then the other of Q¹, Q², or Q³ is C—R² wherein R² is halogen, substituted or unsubstituted —C₁-C₆alkyl, —OR^{2a}, —NR^{2b}R^{2c}, —SO₂C₁-C₆alkyl, or —CN.

Stereoisomers of the compounds of formula (I), and the pharmaceutical salts and solvates thereof, are also described. Methods of using compounds of formula (I) are described, as well as pharmaceutical compositions including the compounds of formula (I).

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

The term “substituted” or “optionally substituted,” as used herein to describe a substituent defined herein, means that the substituent may, but is not required to be, substituted with one or more of: halo (i.e., —F, —Cl, —Br, —I), cyano, —OH, —C₁-C₆alkyl, C₃-C₆cycloalkyl, 3-7 membered heterocycloalkyl, —C₃-C₆spirocycloalkyl, 3-7 membered spiroheterocycloalkyl, bridged heterocycloalkyl, bridged cycloalkyl, C₂-C₆alkenyl, C₂-C₆ alkynyl, C₁-C₆haloalkyl (e.g., —CF₃; —CHF₂, —CH₂CF₃, and the like), —C₁-C₆alkoxy, —C₁-C₆ haloalkoxy (e.g., —OCF₃; —OCHF₂, —OCH₂CF₃, and the like), C₁-C₆alkylthio (e.g., —SCH₃; —SCH₂CH₃, and the like), C₁-C₆ alkylamino (e.g., —CH₂NH₂; —CH₂CH₂NH₂, and the like), —NH₂, —NH(C₁-C₆ alkyl), —N(C₁-C₆ alkyl)₂, —NH(C₁-C₆ alkoxy), —C(O)NHC₁-C₆alkyl, —C(O)N(C₁-C₆ alkyl)₂, —COOH, —C₁-C₆alkylCOOH, —C₃-C₆cycloalkylCOOH, —C(O)NH₂, —C₁-C₆alkylCONH₂, —C₃-C₆cycloalkylCONH₂, C₁-C₆alkylCONHC₁-C₆alkyl, C₁-C₆alkylCON(C₁-C₆alkyl)₂, —C(O)C₁-C₆ alkyl, —C(O)OC₁-C₆ alkyl, —NHCO(C₁-C₆ alkyl), —N(C₁-C₆ alkyl)C(O)(C₁-C₆ alkyl), —S(O)C₁-C₆ alkyl, —S(O)₂C₁-C₆ alkyl, oxo (i.e., ═O), 6-12 membered aryl, or 5 to 12 membered heteroaryl groups. In some embodiments, each of the above optional substituents are themselves optionally substituted by one or two of these groups.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃. Thus, for example, a “C₁ to C₄ alkyl” group refers to all alkyl groups having from 1 to 4 carbons (e.g., 1, 2, 3, or 4), that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. A “C₁ to C₆ alkyl” group refers to all alkyl groups having from 1 to 6 carbons (e.g., 1, 2, 3, 4, 5, or 6).

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. An alkyl group may be substituted or unsubstituted. By way of example only, “C₁-C₈ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl. In several embodiments, “Me” is methyl (e.g., CH₃).

As used herein, “alkenyl” refers to an alkyl group that contains in the straight or branched hydrocarbon chain one or more double bonds. An alkenyl group may be unsubstituted or substituted.

As used herein, “alkynyl” refers to an alkyl group that contains in the straight or branched hydrocarbon chain one or more triple bonds. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups may contain between 3 and 12 carbon atoms. For example, a C₃-C₆cycloalkyl group indicates that there three to six carbon atoms in the ring, that is, the ring is a cyclopropyl, cyclobutyl, cyclopentyl, or cyclohexyl group. A cycloalkyl group may be unsubstituted or substituted.

As used herein, the term “spirocycloalkyl ring” refers to a cycloalkyl ring that shares one carbon atom with another cyclic ring. For example, a 3-7 membered spirocycloalkyl ring indicates that there are 3, 4, 5, 6, or 7 carbon atoms in the cycloalkyl ring that shares a single carbon atom in common with another cyclic ring. By way of example, shown below are exemplary 3-7 membered spirocycloalkyl groups attached to a piperiding ring:

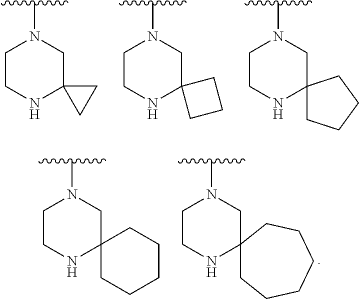

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. Heteroaryl rings may also include bridge head nitrogen atoms. For example but not limited to: pyrazolo[1,5-a]pyridine, imidazo[1,2-a]pyridine, and pyrazolo[1,5-a]pyrimidine. A heteroaryl group may be substituted or unsubstituted.

As used herein, “heterocycloalkyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycloalkyl may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycloalkyl may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocycloalkyl may be quaternized. Heterocycloalkyl groups may be unsubstituted or substituted. Examples of such “heterocycloalkyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl).

As used herein, the term “spiroheterocycloalkyl ring” refers to a heterocycloalkyl ring that shares one carbon atom with another cyclic ring. For example, a 3-7 membered spiroheterocycloalkyl ring indicates that there are 3, 4, 5, 6, or 7 atoms in the heterocycloalkyl ring, and only one of the carbon atoms in that heterocycloalkyl ring is also a member of another cyclic ring. By way of example, shown below are exemplary 3-7 membered spiroheterocycloalkyl groups attached to a piperiding ring:

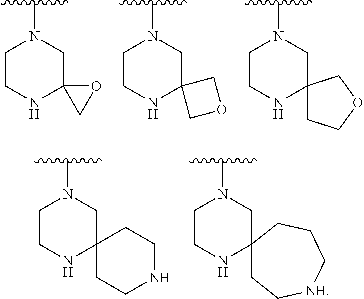

As used herein, the term “bridged bicyclic ring”, refers to a ring system comprising two joined cycloalkyl or heterocycloalkyl rings that share at least three at least three atoms For example, a 6-9 membered bridged bicyclic ring indicates that there are 6, 7, 8, or 9 atoms in the bridged bicyclic ring. By way of example, shown below are exemplary 6-9 membered bridged bicyclic rings:

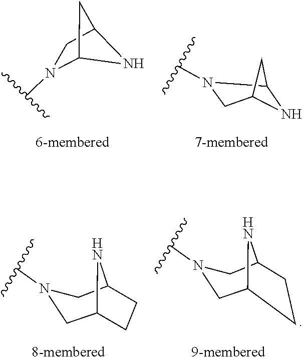 [SMILES: *N1CC2CC1N2 ; *N1CC2CC1N2 ; *N1CC2CCC(C1)N2 ; *N1CC2CCC(C1)N2]

As used herein, the term “amino” refers to a —NH₂ group.

As used herein, the term “hydroxy” refers to a —OH group.

As used herein, the term “halogen atom” or “halogen” refers to fluorine, chlorine, bromine and iodine.

The term “pharmaceutically acceptable salt” refers to a salt of a compound that does not cause significant irritation to an organism to which it is administered and does not abrogate the biological activity and properties of the compound. In several embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxy methyl)methylamine, C₁-C₇ alkylamine, cyclohexylamine, triethanolamine, ethylenediamine, and salts with amino acids such as arginine and lysine.

In some embodiments, the term “pharmaceutically acceptable salt” includes trifluoroacetic acid.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. It is understood that, in any compound described herein having one or more chiral centers, all possible diastereomers are also envisioned. It is understood that, in any compound described herein all tautomers are envisioned. It is also understood that, in any compound described herein, all isotopes of the included atoms are envisioned. For example, any instance of hydrogen, may include hydrogen-1 (protium), hydrogen-2 (deuterium), hydrogen-3 (tritium) or other isotopes; any instance of carbon may include carbon-12, carbon-13, carbon-14, or other isotopes; any instance of oxygen may include oxygen-16, oxygen-17, oxygen-18, or other isotopes; any instance of fluorine may include one or more of fluorine-18, fluorine-19, or other isotopes; any instance of sulfur may include one or more of sulfur-32, sulfur-34, sulfur-35, sulfur-36, or other isotopes.

As used herein, the term “kinase inhibitor” means any compound, molecule or composition that inhibits or reduces the activity of a kinase. The inhibition can be achieved by, for example, blocking phosphorylation of the kinase (e.g., competing with adenosine triphosphate (ATP), a phosphorylating entity), by binding to a site outside the active site, affecting its activity by a conformational change, or by depriving kinases of access to the molecular chaperoning systems on which they depend for their cellular stability, leading to their ubiquitylation and degradation.

As used herein, “subject,” “host,” “patient,” and “individual” are used interchangeably and shall be given its ordinary meaning and shall also refer to an organism that has FGFR proteins. This includes mammals, e.g., a human, a non-human primate, ungulates, canines, felines, equines, mice, rats, and the like. The term “mammal” includes both human and non-human mammals.

The term “sample” or “biological sample” shall be given its ordinary meaning and also encompasses a variety of sample types obtained from an organism and can be used in an imaging, a diagnostic, a prognostic, or a monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment,” “treating,” “treat” and the like shall be given its ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The terms “cancer,” “neoplasm,” and “tumor” are used interchangeably herein, shall be given its ordinary meaning and shall also refer to cells which exhibit relatively autonomous growth, so that they exhibit an aberrant growth phenotype characterized by a significant loss of control of cell proliferation. In general, cells of interest for detection or treatment in the present application include precursors, precancerous (e.g., benign), malignant, pre-metastatic, metastatic, and non-metastatic cells. As used herein, “FGFR related cancer” denotes those cancers that involve an increased activity in a mutant FGFR kinase, for example, the continued activation of FGFR.

The term “control” refers shall be given its ordinary meaning and shall also include a sample or standard used for comparison with a sample which is being examined, processed, characterized, analyzed, etc. In several embodiments, the control is a sample obtained from a healthy patient or a non-tumor tissue sample obtained from a patient diagnosed with a tumor. In several embodiments, the control is a historical control or standard reference value or range of values. In several embodiments, the control is a comparison to a wild-type FGFR arrangement or scenario.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

In some aspects, the disclosure is directed to compounds of formula (I)

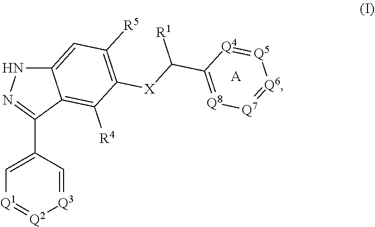

or a pharmaceutically acceptable salt thereof.

In some aspects of the disclosure, R¹ in the compounds of formula (I) is C₁-C₆alkyl, such as, for example, C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments of the compounds of the disclosure, R¹ is —CH₃.

In some aspects of the disclosure, X in the compounds of formula (I) is —O—, —S—, —NH—, or —N(C₁-C₃alkyl)-.

In some embodiments of the compounds of formula (I), X is O.

In some embodiments of the compounds of formula (I), X is S.

In some embodiments of the compounds of formula (I), X is —NH—.

In some embodiments of the compounds of formula (I), X is —N(C₁-C₃alkyl)-such as, for example, —N(C₁-C₃alkyl)-, —N(C₁-C₂alkyl)-, —N(C₁alkyl)-, —N(C₂alkyl)-, —N(C₃alkyl)-, —N(CH₃)—, —N(CH₂CH₃)—, —N(CH₂CH₂CH₃)—, and the like.

In some aspects, Q¹, Q², and Q³ in the compounds of formula (I), are each independently N or C—R², wherein at least one of Q¹, Q′, and Q³ is C—R²; or, when Q¹ and Q′ are both C—R², said R² groups together with the atoms to which they are attached form a substituted or unsubstituted 5-7 membered cycloalkyl ring or a substituted or unsubstituted 5-7 membered heterocycloalkyl ring.

In some embodiments, Q¹ in the compounds of formula (I) is N.

In some embodiments, Q² in the compounds of formula (I) is N.

In some embodiments, Q³ in the compounds of formula (I) is N.

In some embodiments, Q¹ in the compounds of formula (I) is C—R².

In some embodiments, Q² in the compounds of formula (I) is C—R².

In some embodiments, Q² in the compounds of formula (I) is C—H.

In some embodiments, Q³ in the compounds of formula (I) is C—R².

In some embodiments, Q¹, Q², and Q³ are each independently C—R².

In some embodiments, Q¹ in the compounds of formula (I) is N, and Q² and Q³ in the compounds of formula (I) are each independently C—R².

In some embodiments, Q² in the compounds of formula (I) is N, and Q¹ and Q³ in the compounds of formula (I) are each independently C—R².

In some embodiments, Q³ in the compounds of formula (I) is N, and Q¹ and Q² in the compounds of formula (I) are each independently C—R².

In some embodiments, Q¹ in the compounds of formula (I) is C—R², and Q′ and Q³ in the compounds of formula (I) is N.

In some embodiments, Q² in the compounds of formula (I) is C—R², and Q¹ and Q³ in the compounds of formula (I) is N.

In some embodiments, Q³ in the compounds of formula (I) is C—R², and Q¹ and Q² in the compounds of formula (I) is N.

In some aspects, each R² in the compounds of formula (I) is independently H, halogen, substituted or unsubstituted —C₁-C₆alkyl, —OR^{2a}, —NR^{2b}R^{2c}, —C(O)NR^{2b}R^{2c}, —SO₂C₁-C₆alkyl, —CN, —CF₃, —CF₂, —NR²⁶COR^{2a}, —NR^{2b}CONR^{2a}R^{2b}, or, when Q¹, Q², and Q³ are each C—R², each R² is independently H, halogen, substituted or unsubstituted —C₁-C₆alkyl, —OR^{2a}; —NR^{2b}R^{2c}, —C(O)NR^{2b}R^{2c}, —SOC₁-C₆alkyl, —CN, —CF₃, —CF₂, —NR^{2b}COR^{2a}, —NR^{2b}CONR^{2a}R^{2b}, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, or substituted or unsubstituted spiroheterocycloalkyl.

In some embodiments, at least one R² in the compounds of formula (I) is H.

In some embodiments, at least one R² in the compounds of formula (I) is halogen, such as, —F, —Cl, —Br, or —I.

In some embodiments, at least one R² in the compounds of formula (I) is F.

In some embodiments, at least one R² in the compounds of formula (I) is substituted or unsubstituted —C₁-C₆alkyl, such as, for example, substituted or unsubstituted C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, at least one R² in the compounds of formula (I) is —SO₂C₁-C₆alkyl, such as, for example, —SO₂C₁-C₆alkyl, —SO₂C₁-C₅alkyl, —SO₂C₁-C₄alkyl, —SO₂C₁-C₃alkyl, —SO₂C₁-C₂alkyl, —SO₂C₁alkyl, —SO₂C₂alkyl, —SO₂C₃alkyl, —SO₂C₄alkyl, —SO₂C₅alkyl, —SO₂C₆alkyl, —SO₂CH₃, —SO₂CH₂CH₃, and the like.

In some embodiments, at least one R² in the compounds of formula (I) is —CN.

In some embodiments, at least one R² in the compounds of formula (I) is —CF₃.

In some embodiments, at least one R² in the compounds of formula (I) is —CF₂.

In some embodiments, at least one R² in the compounds of formula (I) is —OR^{2a}.

In some embodiments, at least one R² in the compounds of formula (I) is —NR^{2b}COR^{2a}.

In some embodiments, at least one R² in the compounds of formula (I) is —NR^{2b}R^{2c}.

In some embodiments, at least one R² in the compounds of formula (I) is —C(O)NR^{2b}R^{2c}.

In some embodiments, at least one R² in the compounds of formula (I) is —NR^{2b}CONR^{2a}R^{2b}.

In some aspects, when Q¹, Q², and Q³ are each C—R², R² may also be substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, or a substituted or unsubstituted spiroheterocycloalkyl.

In some embodiments wherein Q¹, Q², and Q³ are each C—R², R² is substituted or unsubstituted —C₃-C₆cycloalkyl, such as, for example, substituted or unsubstituted —C₆cycloalkyl, —C₅cycloalkyl, —C₄cycloalkyl, —C₃cycloalkyl, cyclopropyl, cyclobutyl, cyclopentyl, cyclohexyl, or cycloheptyl, and the like.

In some embodiments wherein Q¹, Q², and Q³ are each C—R², R² is substituted or unsubstituted 3-6-membered heterocycloalkyl, such as, for example, substituted or unsubstituted: 3-membered heterocycloalkyl, 4-membered heterocycloalkyl, 5-membered heterocycloalkyl, 6-membered heterocycloalkyl, and the like.

In some embodiments wherein Q¹, Q², and Q³ are each C—R², R² is substituted or unsubstituted bridged heterocycloalkyl.

In some embodiments wherein Q¹, Q², and Q³ are each C—R², R² is substituted or unsubstituted bridged cycloalkyl.

In some embodiments wherein Q¹, Q², and Q³ are each C—R², R² is substituted or unsubstituted spiroheterocycloalkyl.

In some embodiments of the compounds of formula (I), when Q¹ and Q² are both C—R², the R² groups together with the atoms to which they are attached, form a substituted or unsubstituted 5-7 membered cycloalkyl ring, such as, for example, substituted or unsubstituted: 5-6 membered cycloalkyl ring, 5-membered cycloalkyl, 6-membered cycloalkyl, 7-membered cycloalkyl group, and the like.

In some embodiments of the compounds of formula (I), when Q¹ and Q² are both C—R², the R² groups together with the atoms to which they are attached, form a substituted or unsubstituted 5-7 membered heterocycloalkyl ring, such as, for example, substituted or unsubstituted: 5-membered heterocycloalkyl, 6-membered heterocycloalkyl, 7-membered heterocycloalkyl group, and the like.

In some embodiments, the R² groups, together with the atoms to which they are attached, form substituted or unsubstituted 1,3-dioxanyl or tetrahydropyranyl group.

In some embodiments, the R² groups, together with the atoms to which they are attached, form substituted or unsubstituted morpholinyl group.

In some embodiments of the compounds of formula (I) in which Q¹ and Q² are both C—R² and the R² groups together with the atoms to which they are attached, form a substituted or unsubstituted 5-7 membered cycloalkyl ring or a substituted or unsubstituted 5-7 membered heterocycloalkyl ring, the substituted 5-7 membered cycloalkyl ring or the substituted 5-7 membered heterocycloalkyl ring is substituted with a substituted or unsubstituted 5-7 membered spiroheterocycloalkyl ring.

In some embodiments, the 5-7 membered spiroheterocycloalkyl ring is a 5-membered spiroheterocycloalkyl ring (e.g., a spiropyrrolidinyl ring) or a 6-membered spiroheterocycloalkyl ring (e.g., a spiropiperidinyl ring).

In some embodiments, the 5-7 membered spiroheterocycloalkyl is substituted with —C₁-C₆alkyl, —C₃-C₆cycloalkyl, 3-7-membered heterocycloalkyl, —C(O)C₁-C₆alkyl, —C(O)C₃-C₆cycloalkyl, —C(O)N(C₁-C₆alkyl)₂, —C(O)NH(C₁-C₆alkyl), —C(O)OC₁-C₆alkyl, —C(O)OC₃-C₆cycloalkyl, or —SO₂C₁-C₆alkyl.

In some embodiments the 6-membered spiroheterocycloalkyl ring is

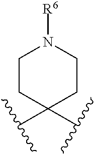

wherein R⁶ is C₁-C₆alkyl, —C₃-C₆cycloalkyl, 3-7-membered heterocycloalkyl, —C(O)C₁-C₆alkyl, —C(O)C₃-C₆cycloalkyl, —C(O)N(C₁-C₆alkyl)₂, —C(O)NH(C₁-C₆alkyl), —C(O)OC₁-C₆alkyl, —C(O)OC₃-C₆cycloalkyl, or —SO₂C₁-C₆alkyl.

In some embodiments of the compounds of formula (I), when Q¹ and Q² are both C—R², the R² groups together with the atoms to which they are attached, form a substituted 5-7 membered heterocycloalkyl ring that is substituted with a substituted or unsubstituted 5-7 membered spiroheterocycloalkyl ring, such as, for example, substituted or unsubstituted: 5-membered spiroheterocycloalkyl ring, 6-membered spiroheterocycloalkyl ring, or 7-membered spiroheterocycloalkyl ring, and the like.

In some embodiments of the compounds of formula (I), when Q¹ and Q² are both C—R², the R² groups together with the atoms to which they are attached is

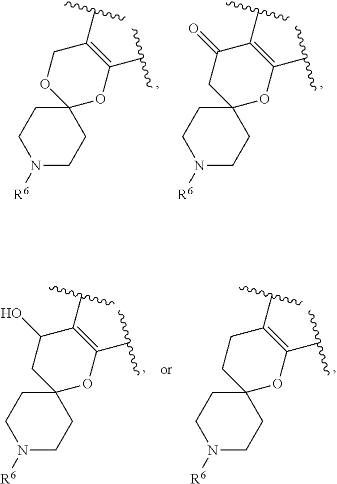

wherein the R⁶ is H, C₁-C₆alkyl, C₁-C₆haloalkyl, —C₃-C₆cycloalkyl, 3-7-membered heterocycloalkyl, —C(O)C₁-C₆alkyl, —C(O)C₃-C₆cycloalkyl, —C(O)N(C₁-C₆alkyl)₂, —C(O)NH(C₁-C₆alkyl), —C(O)OC₁-C₆alkyl, —C(O)OC₃-C₆cycloalkyl, or —SO₂C₁-C₆alkyl.

In some embodiments of the compounds of formula (I), when Q¹ and Q² are both C—R², the R² groups together with the atoms to which they are attached is

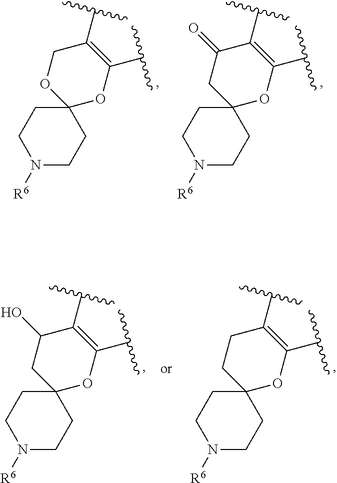

wherein the R⁶ is C₁-C₆alkyl, —C₃-C₆cycloalkyl, 3-7-membered heterocycloalkyl, —C(O)C₁-C₆alkyl, —C(O)C₃-C₆cycloalkyl, —C(O)N(C₁-C₆alkyl)₂, —C(O)NH(C₁-C₆alkyl), —C(O)OC₁-C₆alkyl, —C(O)OC₃-C₆cycloalkyl, or —SO₂C₁-C₆alkyl.

In some embodiments of the compounds of formula (I), when Q¹ and Q² are both C—R², the R² groups together with the atoms to which they are attached is

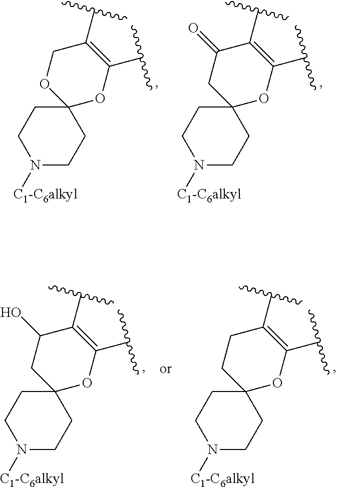

wherein the C₁-C₆alkyl is, for example, C₆alkyl, C₅alkyl, C₄alkyl, C₃alkyl, C₂alkyl, C₁alkyl, methyl, ethyl, or isopropyl.

In some embodiments of the compounds of formula (I), when Q¹ and Q² are both C—R², the R² groups together with the atoms to which they are attached is

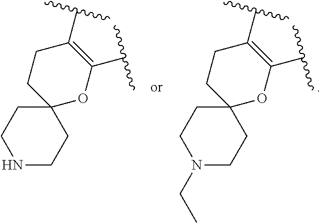 [SMILES: *C1=C(*)OC2(CCNCC2)CC1 ; *C1=C(*)OC2(CC1)CCN(CC)CC2]

In some embodiments of the compounds of formula (I), when Q¹ and Q² are both C—R², the R² groups together with the atoms to which they are attached is

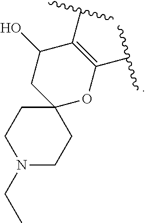 [SMILES: *C1=C(*)C(O)CC2(CCN(CC)CC2)O1]

In some embodiments of the compounds of formula (I), when Q¹ and Q² are both C—R², the R² groups together with the atoms to which they are attached is

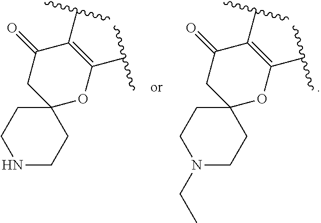 [SMILES: *C1=C(*)C(=O)CC2(CCNCC2)O1 ; *C1=C(*)C(=O)CC2(CCN(CC)CC2)O1]

In some embodiments of the compounds of formula (I), when Q¹ and Q² are both C—R², the R² groups together with the atoms to which they are attached is

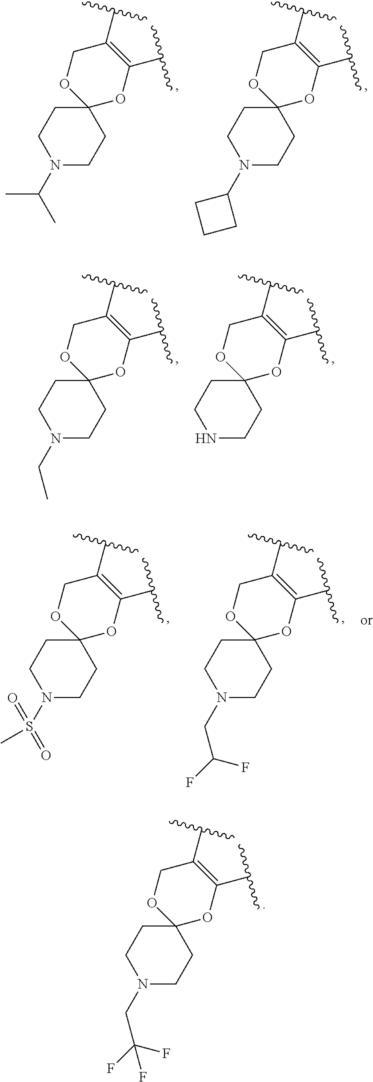

In some aspects, each R^{2a} in the compounds of formula (I) is independently H, substituted or unsubstituted —C₁-C₆alkyl, —C₁-C₆haloalkyl, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, substituted or unsubstituted spiroheterocycloalkyl, substituted or unsubstituted —C₁-C₆alk-C₃-C₆cycloalkyl, or substituted or unsubstituted —C₁-C₆alk-O—C₁-C₆alkyl.

In some embodiments, at least one R^{2a} is H.

In some embodiments, at least one R^{2a} is substituted or unsubstituted —C₁-C₆alkyl, such as, for example, substituted or unsubstituted: C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R^{2a} is methyl.

In some embodiments, R^{2a} is

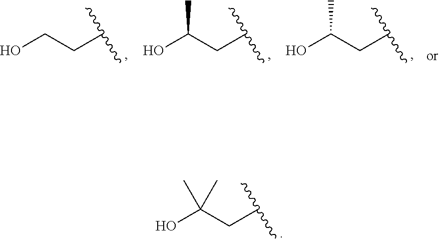

In some embodiments, at least one R^{2a} is —C₁-C₆haloalkyl, such as, for example, C₁-C₆haloalkyl, C₁-C₅haloalkyl, C₁-C₄haloalkyl, C₁-C₃haloalkyl, C₁-C₂haloalkyl, C₁haloalkyl, C₂haloalkyl, C₃haloalkyl, C₄haloalkyl, C₅haloalkyl, C₆haloalkyl, halomethyl, haloethyl, and the like.

In some embodiments, R^{2a} is CF₃.

In some embodiments, R^{2a} is substituted or unsubstituted —C₃-C₆cycloalkyl, such as, for example, substituted or unsubstituted —C₆cycloalkyl, —C₅cycloalkyl, —C₄cycloalkyl, —C₃cycloalkyl, cyclopropyl, cyclobutyl, cyclopentyl, cyclohexyl, and the like.

In some embodiments, R^{2a} is substituted or unsubstituted 3-6-membered heterocycloalkyl, such as, for example, substituted or unsubstituted: 3-membered heterocycloalkyl, 4-membered heterocycloalkyl, 5-membered heterocycloalkyl, 6-membered heterocycloalkyl, piperidinyl, tetrahydropyranyl, tetrahydrofuranyl, morpholinyl, and the like.

In other embodiments, R^{2a} is substituted or unsubstituted 3-6-membered heterocycloalkyl, such as, for example, morpholinyl, oxiranyl, aziridinyl, oxetanyl, azetidinyl, tetrahydrofuranyl, pyrrolidinyl, tetrahydropyranyl, piperidinyl, and the like and the like.

In some embodiments, R^{2a} is tetrahydrofuranyl, such as, for example,

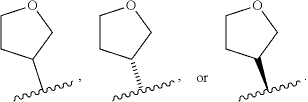

In some embodiments, R^{2a} is

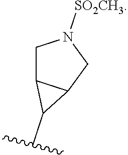 [SMILES: *C1C2CN(S(C)(=O)=O)CC12]

In some embodiments, R^{2a} is substituted or unsubstituted bridged heterocycloalkyl.

In some embodiments, R^{2a} is substituted or unsubstituted bridged cycloalkyl.

In some embodiments, R^{2a} is

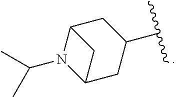 [SMILES: *C1CC2CC(C1)N2C(C)C]

In some embodiments, R^{2a} is substituted or unsubstituted spiroheterocycloalkyl.

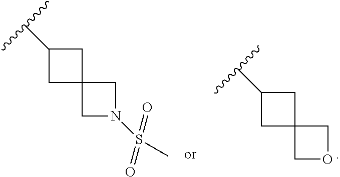 [SMILES: *C1CC2(C1)CN(S(C)(=O)=O)C2 ; *C1CC2(COC2)C1]

In some embodiments, R^{2a} is

In some embodiments, R^{2a} is unsubstituted piperidinyl, such as, for example,

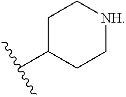 [SMILES: *C1CCNCC1]

In some embodiments, R^{2a} is N-substituted piperidinyl, wherein the N-substituent is, for example, C₁-C₆alkyl, such as methyl, ethyl, isopropyl and the like; C₃-C₆cycloalkyl, such as cyclopropyl, cyclobutyl, cyclopentyl, and the like; or C₂-C₅heterocycloalkyl, such as tetrahydrofuranyl, oxetanyl, oxiranyl, and the like.

In some embodiments, R^{2a} is N-substituted piperidinyl, wherein the N-substituent is, for example, C₁-C₆alkyl, such as methyl, ethyl, isopropyl and the like; C₃-C₆cycloalkyl, such as cyclopropyl, cyclobutyl, cyclopentyl, and the like; or C₂-C₅heterocycloalkyl, such as tetrahydrofuranyl, oxetanyl, oxiranyl, and the like.

In some embodiments, R^{2a} is:

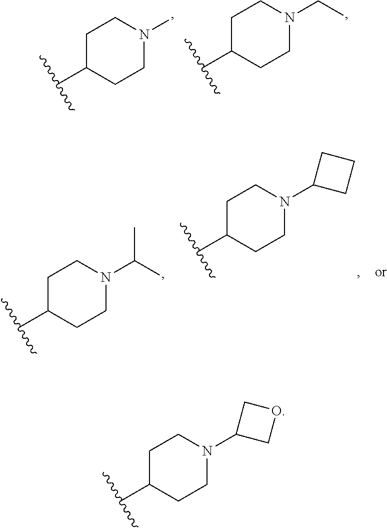

In some embodiments, R^{2a} is substituted or unsubstituted —C₁-C₆alk-C₃-C₆cycloalkyl, such as, for example, substituted or unsubstituted; —C₆alk-C₃-C₆cycloalkyl, —C₅alk-C₃-C₆cycloalkyl, —C₄alk-C₃-C₆cycloalkyl, —C₃alk-C₃-C₆cycloalkyl, —C₂alk-C₃-C₆cycloalkyl, C₁alk-C₃-C₆cycloalkyl, —C₁-C₆alk-C₆cycloalkyl, —C₁-C₆alk-C₅cycloalkyl, —C₁-C₆alk-C₄cycloalkyl, —C₁-C₆alk-C₃cycloalkyl, and the like.

In some embodiments, R^{2a} is substituted or unsubstituted —C₁-C₆alk-O—C₁-C₆alkyl, such as, for example, substituted or unsubstituted; —C₁alk-O—C₁-C₆alkyl, —C₂alk-O—C₁-C₆alkyl, —C₃alk-O—C₁-C₆alkyl, —C₄alk-O—C₁-C₆alkyl, —C₅alk-O—C₁-C₆alkyl, —C₆alk-O—C₁-C₆alkyl, —C₁-C₆alk-O—C₆alkyl, —C₁-C₆alk-O—C₅alkyl, —C₁-C₆alk-O—C₄alkyl, —C₁-C₆alk-O—C₃alkyl, —C₁-C₆alk-O—C₂alkyl, —C₁-C₆alk-O—C₁alkyl, and the like.

In some aspects, each R^{2b} in the compounds of formula (I) is independently H, substituted or unsubstituted —C(O)-(5- to 10-membered heteroaryl), substituted or unsubstituted —C(O)-(5- to 6-membered heterocycloalkyl), substituted or unsubstituted —C₁-C₆alkyl, substituted or unsubstituted —C₁-C₆alk-O—C₁-C₆alkyl, substituted or unsubstituted —C(O)—(C₃-C₆cycloalkyl), substituted or unsubstituted —C(O)N(C₁-C₆alkyl)₂, —C(O)C₁-C₃alkN (substituted or unsubstituted C₁-C₆alkyl)₂, substituted or unsubstituted —C(O)—OC₁-C₆alkyl, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, substituted or unsubstituted spiroheterocycloalkyl, or —C(O)CH═CH₂.

In some embodiments, R^{2b} is H.

In some embodiments, R^{2b} is substituted or unsubstituted —C(O)-(5- to 10-membered heteroaryl), such as, for example, substituted or unsubstituted; —C(O)-(5-membered heteroaryl), —C(O)-(6-membered heteroaryl), —C(O)-(7-membered heteroaryl), —C(O)-(8-membered heteroaryl), —C(O)-(9-membered heteroaryl), C(O)-(10-membered heteroaryl), and the like.

In some embodiments, R^{2b} is substituted or unsubstituted —C(O)-(5- to 6-membered heterocycloalkyl), such as, for example, substituted or unsubstituted; —C(O)-(5-membered heterocycloalkyl), or —C(O)-(6-membered heterocycloalkyl), and the like.

In some embodiments, R^{2b} is substituted or unsubstituted —C₁-C₆alkyl, such as, for example, substituted or unsubstituted: C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments. R^{2b} is substituted or unsubstituted —C₁-C₆alk-O—C₁-C₆alkyl, such as, for example, substituted or unsubstituted; —C₁alk-O—C₁-C₆alkyl, —C₂alk-O—C₁-C₆alkyl, —C₃alk-O—C₁-C₆alkyl, —C₄alk-O—C₁-C₆alkyl, —C₅alk-O—C₁-C₆alkyl, —C₆alk-O—C₁-C₆alkyl, —C₁-C₆alk-O—C₆alkyl, —C₁-C₆alk-O—C₅alkyl, —C₁-C₆alk-O—C₄alkyl, —C₁-C₆alk-O—C₆alkyl, —C₁-C₆alk-O—C₂alkyl, —C₁-C₆alk-O—C₁alkyl, and the like.

In some embodiments, R^{2b} is substituted or unsubstituted —C(O)—(C₃-C₆cycloalkyl), such as, for example, substituted or unsubstituted; —C(O)—(C₆cycloalkyl), —C(O)—(C₅cycloalkyl), —C(O)—(C₄cycloalkyl), —C(O)—(C₃cycloalkyl), —C(O)-cyclopropyl, —C(O)-cyclobutyl, —C(O)-cyclopentyl, —C(O)-cyclohexyl, and the like.

In some embodiments, R^{2b} is substituted or unsubstituted —C(O)N(C₁-C₆alkyl)₂, such as, for example, substituted or unsubstituted; —C(O)N(C₁alkyl)₂, —C(O)N(C₂alkyl)₂, —C(O)N(C₃alkyl)₂, —C(O)N(C₄alkyl)₂, —C(O)N(C₅alkyl)₂, —C(O)N(C₆alkyl)₂, —C(O)N(C₁alkyl)(C₁-C₆alkyl), —C(O)N(C₂alkyl)(C₁-C₆alkyl), —C(O)N(C₃alkyl)(C₁-C₆alkyl), —C(O)N(C₄alkyl)(C₁-C₆alkyl), —C(O)N(C₅alkyl)(C₁-C₆alkyl), —C(O)N(C₆alkyl)(C₁-C₆alkyl), and the like.

In some embodiments, R²⁰ is —C(O)C₁-C₃alkN (substituted or unsubstituted C₁-C₆alkyl)₂, such as, for example, —C(O)C₁alkN (substituted or unsubstituted C₁-C₆alkyl)₂, —C(O)C₂alkN (substituted or unsubstituted C₁-C₆alkyl)₂, —C(O)C₃alkN (substituted or unsubstituted C₁-C₆alkyl)₂, —C(O)C₁-C₃alkN (substituted or unsubstituted C₆alkyl)₂, —C(O)C₁-C₃alkN (substituted or unsubstituted C₅alkyl)₂, —C(O)C₁-C₃alkN (substituted or unsubstituted C₄alkyl)₂, —C(O)C₁-C₃alkN (substituted or unsubstituted C₃alkyl)₂, —C(O)C₁-C₃alkN (substituted or unsubstituted C₂alkyl)₂, —C(O)C₁-C₃alkN (substituted or unsubstituted C₁alkyl)₂, and the like.

In some embodiments, R^{2b} is substituted or unsubstituted —C(O)—OC₁-C₆alkyl, such as, for example, substituted or unsubstituted; —C(O)—OC₆alkyl, —C(O)—OC₅alkyl, —C(O)—OC₄alkyl, —C(O)—OC₃alkyl, —C(O)—OC₂alkyl, —C(O)—OC₁alkyl, and the like.

In some embodiments, R^{2b} is substituted or unsubstituted —C₃-C₆cycloalkyl, such as, for example, substituted or unsubstituted —C₆cycloalkyl, —C₅cycloalkyl, —C₄cycloalkyl, —C₃cycloalkyl, cyclopropyl, cyclobutyl, cyclopentyl, cyclohexyl, and the like.

In some embodiments, R^{2b} is substituted or unsubstituted 3-6-membered heterocycloalkyl, such as, for example, substituted or unsubstituted: 3-membered heterocycloalkyl, 4-membered heterocycloalkyl, 5-membered heterocycloalkyl, or 6-membered heterocycloalkyl.

In some embodiments, R^{2b} is substituted or unsubstituted 3-6-membered heterocycloalkyl, such as, for example, substituted or unsubstituted: 3-membered heterocycloalkyl, 4-membered heterocycloalkyl, 5-membered heterocycloalkyl, or 6-membered heterocycloalkyl.

In some embodiments, R^{2b} is

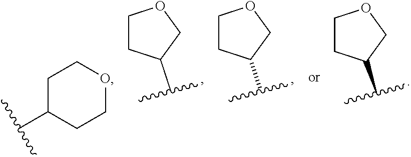

In some embodiments, R^{2b} is substituted or unsubstituted membered bridged heterocycloalkyl.

In some embodiments, R^{2b} is substituted or unsubstituted bridged cycloalkyl.

In some embodiments, R^{2b} is substituted or unsubstituted spiroheterocycloalkyl.

In some embodiments, R^{2b} is

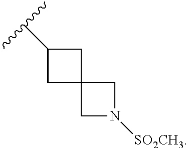 [SMILES: *C1CC2(C1)CN(S(C)(=O)=O)C2]

In some embodiments, R^{2b} is —C(O)CH═CH₂.

In some aspects, each R^{2c} in the compounds of formula (I) is independently H, substituted or unsubstituted —C(O)-(5- to 10-membered heteroaryl), substituted or unsubstituted —C(O)-(5- to 6-membered heterocycloalkyl), substituted or unsubstituted —C₁-C₆alkyl, substituted or unsubstituted —C₁-C₆alk-O—C₁-C₆alkyl, substituted or unsubstituted —C(O)—(C₃-C₆cycloalkyl), substituted or unsubstituted —C(O)N(C₁-C₆alkyl)₂, —C(O)C₁-C₃alkN (substituted or unsubstituted C₁-C₆alkyl)₂, substituted or unsubstituted —C(O)—OC₁-C₆alkyl, substituted or unsubstituted —C₃-C₆cycloalkyl, or —C(O)CH═CH₂.

In some embodiments, R^{2c} is H.

In some embodiments, R^{2c} is substituted or unsubstituted —C(O)-(5- to 10-membered heteroaryl), such as, for example, substituted or unsubstituted; —C(O)-(5-membered heteroaryl), —C(O)-(6-membered heteroaryl), —C(O)-(7-membered heteroaryl), —C(O)-(8-membered heteroaryl), —C(O)-(9-membered heteroaryl), C(O)-(10-membered heteroaryl), and the like.

In some embodiments, R^{2c} is substituted or unsubstituted —C(O)-(5- to 6-membered heterocycloalkyl), such as, for example, substituted or unsubstituted; —C(O)-(5-membered heterocycloalkyl), or —C(O)-(6-membered heterocycloalkyl), and the like.

In some embodiments, R^{2c} is substituted or unsubstituted —C₁-C₆alkyl, such as, for example, substituted or unsubstituted: C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R^{2c} is substituted or unsubstituted —C₁-C₆alk-O—C₁-C₆alkyl, such as, for example, substituted or unsubstituted; —C₁alk-O—C₁-C₆alkyl, —C₂alk-O—C₁-C₆alkyl, —C₃alk-O—C₁-C₆alkyl, —C₄alk-O—C₁-C₆alkyl, —C₅alk-O—C₁-C₆alkyl, —C₆alk-O—C₁-C₆alkyl, —C₁-C₆alk-O—C₆alkyl, —C₁-C₆alk-O—C₅alkyl, —C₁-C₆alk-O—C₄alkyl, —C₁-C₆alk-O—C₃alkyl, —C₁-C₆alk-O—C₂alkyl, —C₁-C₆alk-O—C₁alkyl, and the like.

In some embodiments, R^{2c} is substituted or unsubstituted —C(O)—(C₃-C₆cycloalkyl), such as, for example, substituted or unsubstituted; —C(O)—(C₆cycloalkyl), —C(O)—(C₅cycloalkyl), —C(O)—(C₄cycloalkyl), —C(O)—(C₃cycloalkyl), —C(O)-cyclopropyl, —C(O)-cyclobutyl, —C(O)-cyclopentyl, —C(O)-cyclohexyl, and the like.

In some embodiments, R^{2c} is substituted or unsubstituted —C(O)N(C₁-C₆alkyl)₂, such as, for example, substituted or unsubstituted; —C(O)N(C₁alkyl)₂. —C(O)N(C₂alkyl)₂, —C(O)N(C₃alkyl)₂, —C(O)N(C₄alkyl)₂, —C(O)N(C₅alkyl)₂, —C(O)N(C₆alkyl)₂, —C(O)N(C₁alkyl)(C₁-C₆alkyl), —C(O)N(C₂alkyl)(C₁-C₆alkyl), —C(O)N(C₃alkyl)(C₁-C₆alkyl), —C(O)N(C₄alkyl)(C₁-C₆alkyl), —C(O)N(C₅alkyl)(C₁-C₆alkyl), —C(O)N(C₆alkyl)(C₁-C₆alkyl), and the like.

In some embodiments, R^{2c} is —C(O)C₁-C₃alkN (substituted or unsubstituted C₁-C₆alkyl)₂, such as, for example, —C(O)C₁alkN (substituted or unsubstituted C₁-C₆alkyl)₂, —C(O)C₂alkN (substituted or unsubstituted C₁-C₆alkyl)₂, —C(O)C₃alkN (substituted or unsubstituted C₁-C₆alkyl)₂, —C(O)C₁-C₃alkN (substituted or unsubstituted C₆alkyl)₂, —C(O)C₁-C₃alkN (substituted or unsubstituted C₅alkyl)₂, —C(O)C₁-C₃alkN (substituted or unsubstituted C₄alkyl)₂, —C(O)C₁-C₃alkN (substituted or unsubstituted C₃alkyl)₂, —C(O)C₁-C₃alkN (substituted or unsubstituted C₂alkyl)₂, —C(O)C₁-C₃alkN (substituted or unsubstituted C₁alkyl)₂, and the like.

In some embodiments, R^{2c} is substituted or unsubstituted —C(O)—OC₁-C₆alkyl, such as, for example, substituted or unsubstituted; —C(O)—OC₆alkyl, —C(O)—OC₅alkyl, —C(O)—OC₄alkyl, —C(O)—OC₃alkyl, —C(O)—OC₂alkyl, —C(O)—OC₁alkyl, and the like.

In some embodiments, R^{2c} is substituted or unsubstituted —C₃-C₆cycloalkyl, such as, for example, substituted or unsubstituted —C₆cycloalkyl, —C₅cycloalkyl, —C₄cycloalkyl, —C₃cycloalkyl, cyclopropyl, cyclobutyl, cyclopentyl, cyclohexyl, and the like.

In some embodiments, R^{2c} is substituted or unsubstituted 3-6-membered heterocycloalkyl, such as, for example, substituted or unsubstituted: 3-membered heterocycloalkyl, 4-membered heterocycloalkyl, 5-membered heterocycloalkyl, or 6-membered heterocycloalkyl.

In some embodiments, R^{2c} is substituted or unsubstituted bridged heterocycloalkyl.

In some embodiments, R^{2b} is substituted or unsubstituted bridged cycloalkyl.

In some embodiments, R^{2c} is substituted or unsubstituted spiroheterocycloalkyl.

In some embodiments, R^{2c} is —C(O)CH═CH₂.

In some aspects of the disclosure, Q¹, Q⁵, Q⁶, Q⁷, and Q⁸ in the compounds of formula (I) are each independently N or CR³, wherein one or two of Q⁴, Q⁵, Q⁶, Q⁷, and Q⁸ is N and the remainder are each independently CR³.

In some embodiments, one of Q⁴, Q⁵, Q⁶, Q⁷, and Q⁸ is N, and the remainder are each independently CR³.

In some embodiments, Q⁴ is N and Q⁵, Q⁶, Q⁷, and Q⁸ are each independently CR³.

In some embodiments, Q⁵ is N and Q⁴, Q⁶, Q⁷, and Q⁸ are each independently CR³.

In some embodiments, Q⁶ is N and Q⁴, Q⁵, Q⁷, and Q⁸ are each independently CR³.

In some embodiments, Q⁷ is N and Q⁴, Q⁵, Q⁶, and Q⁸ are each independently CR³.

In some embodiments, Q⁸ is N and Q⁴, Q⁵, Q⁶, and Q⁷ are each independently CR³.

In other embodiments, two of Q⁴, Q⁵, Q⁶, Q⁷, or Q⁸ is N, and the remainder are each independently CR³.

In some embodiments, Q⁴ and Q⁵ are N, and Q⁶, Q⁷, and Q⁸ are each independently CR³.

In some embodiments, Q⁴ and Q⁶ are N, and Q⁵, Q⁷, and Q⁸ are each independently CR³.

In some embodiments, Q⁴ and Q⁷ are N, and Q⁵, Q⁶, and Q⁸ are each independently CR³.

In some embodiments, Q⁴ and Q⁸ are N, and Q⁵, Q⁶, and Q⁷ are each independently CR³.

In some embodiments, Q⁵ and Q⁶ are N, and Q⁴, Q⁷, and Q⁸ are each independently CR³.

In some embodiments, Q⁵ and Q⁷ are N, and Q⁴, Q⁶, and Q⁸ are each independently CR³.

In some embodiments, Q⁵ and Q⁸ are N, and Q⁴, Q⁶, and Q⁷ are each independently CR³.

In some embodiments, Q⁶ and Q⁷ are N, and Q⁴, Q⁵, and Q⁸ are each independently CR³.

In some embodiments, Q⁶ and Q⁸ are N, and Q¹, Q⁵, and Q⁷ are each independently CR³.

In some embodiments, Q⁷ and Q⁸ are N, and Q¹, Q⁵, and Q⁶ are each independently CR³.

In some aspects of the disclosure, each R³ in the compounds of formula (I), is independently H, halogen, C₁-C₃alkyl, C₁-C₃alkoxyl, or cycloalkyl. (each R³ is independently H, halogen, C₁-C₆alkyl; C₁-C₆ alkoxyl, or cycloalkyl)

In some embodiments of the compounds of formula (I), R³ is H.

In some embodiments of the compounds of formula (I), R³ is halogen, such as, —F, —Cl, —Br, or —I.

In some embodiments, at least one R³ is —Cl.

In some embodiments, at least one R³ is —F.

In some embodiments of the compounds of formula (I), R³ is C₁-C₃alkyl, such as, for example, C₁-C₃alkyl, C₁-C₂alkyl, C₁alkyl, C₂alkyl, C₃alkyl, —CH₃, —CH₂CH₃, -propyl, and the like.

In some embodiments, R³ is —CH₃.

In some embodiments of the compounds of formula (I), R³ is C₁-C₃alkoxyl, such as, for example, C₁-C₃alkoxyl, C₁-C₂alkoxyl, C₁alkoxyl, C₂alkoxyl, C₃alkoxyl, —OCH₃, —OCH₂CH₃, -propoxyl, and the like. In some embodiments, R³ is —OCH₃.

In some embodiments of the compounds of formula (I), R³ is cycloalkyl, such as, for example, cyclopropyl, cyclobutyl, cyclopentyl, cyclohexyl, or cycloheptyl, and the like.

In some embodiments of the compounds of formula (I), two R³ are halogen, and the remaining R³ are H.

In other embodiments of the compounds of formula (I), two R³ are —C₁, and the remaining R³ are H.

In some embodiments of the compounds of formula (I), Q⁴ and Q⁸ are each independently CR³ wherein R³ is halogen; Q⁵ and Q⁷ are CR³ wherein R³ is H; and Q⁶ is N.

In some embodiments of the compounds of formula (I), Q⁴ and Q⁸ are CR³ wherein R³ is —Cl; Q⁵ and Q⁷ are CR³ wherein R³ is H; and Q⁶ is N.

In some embodiments of the compounds of formula (I), Q⁴ and Q⁸ are each independently CR³ wherein R³ is halogen; Q⁵ is CR³ wherein R³ is H and Q⁷ is CR³ wherein R³ is C₁-C₃alkyl; and Q⁶ is N.

In some embodiments of the compounds of formula (I), Q⁴ and Q⁸ are CR³ wherein R³ is —Cl; Q⁵ is CR³ wherein R³ is H and Q⁷ is CR³ wherein R³ is —CH₃; and Q⁶ is N.

In some embodiments of the compounds of formula (I), Q⁴, Q⁷, and Q⁸ are each independently CR³ wherein each R³ is halogen; Q⁵ is CR³ wherein R³ is H; and Q⁶ is N.

In some embodiments of the compounds of formula (I), Q⁴ and Q⁸ are each independently CR³ wherein each R³ is —Cl; Q⁵ is CR³ wherein R³ is H; Q⁷ is CR³ wherein R³ is-F; and Q⁶ is N.

In some embodiments of the compounds of formula (I), Q⁴ and Q⁸ are each independently CR³ wherein each R³ is halogen; Q⁵ is CR³ wherein R³ is H and Q⁷ is N; and Q⁶ is CR³ wherein R³ is H.

In some embodiments of the compounds of formula (I), Q⁴ and Q⁸ are each independently CR³ wherein each R³ is —Cl; Q⁵ is CR³ wherein R³ is H and Q⁷ is N; and Q⁶ is CR³ wherein R³ is H.

In some embodiments of the compounds of formula (I), Q⁴ and Q⁸ are each independently CR³ wherein each R³ is halogen; Q⁵ is CR³ wherein R³ is H and Q⁷ is N; and Q⁶ is N.

In some embodiments of the compounds of formula (I), Q⁴ and Q⁸ are each independently CR³ wherein each R³ is —Cl; Q⁵ is CR³ wherein R³ is H and Q⁷ is N; and Q⁶ is N.

In some embodiments of the compounds of formula (I), Q⁴ and Q⁸ are each independently CR³ wherein each R³ is C₁-C₃alkyl; Q⁵ is CR³ wherein R³ is H and Q⁷ is N; and Q⁶ is N.

In some embodiments of the compounds of formula (I), Q⁴ and Q⁸ are each independently CR³ wherein each R³ is —CH₃; Q⁵ is CR³ wherein R³ is H and Q⁷ is N; and Q⁶ is N.

In some aspects, R⁴ in the compounds of formula (I) is H, halogen, —C₁-C₆alkyl; —C₁-C₆ alkoxyl, or -cycloalkyl.

In some embodiments, R⁴ in the compounds of formula (I) is H.

In some embodiments, R⁴ in the compounds of formula (I) is halogen, such as, for example, —F, —Cl, —Br, or —I.

In some embodiments, R⁴ in the compounds of formula (I) is —F.

In some embodiments, R⁴ in the compounds of formula (I) is —C₁-C₆alkyl, such as, for example, substituted or unsubstituted: C₁-C₆alkyl, C₁-C₄alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like. In some embodiments, R⁴ is —CH₃.

In some embodiments, R⁴ in the compounds of formula (I) is —C₁-C₆ alkoxyl, such as, for example, —C₁-C₆alkoxyl, —C₁-C₅alkoxyl, —C₁-C₄alkoxyl, —C₁-C₃alkoxyl, —C₁-C₂alkoxyl, —C₁alkoxyl, —C₂alkoxyl, —C₃alkoxyl, —C₄alkoxyl, —C₅alkoxyl, —C₆alkoxyl, —OCH₃, —OCH₂CH₃, -propoxyl, and the like. In some embodiments, R⁴ is —OCH₃.

In some embodiments, R⁴ in the compounds of formula (I) is -cycloalkyl, such as, for example, cyclopropyl, cyclobutyl, cyclopentyl, cyclohexyl, or cycloheptyl, and the like.

In some aspects, R⁵ is H, halogen, —C₁-C₆alkyl; —C₁-C₆ alkoxyl, or -cycloalkyl.

In some embodiments, R⁵ in the compounds of formula (I) is H.

In some embodiments, R⁵ in the compounds of formula (I) is halogen, such as, for example, —F, —Cl, —Br, or —I.

In some embodiments, R⁵ in the compounds of formula (I) is —F.

In some embodiments, R⁵ in the compounds of formula (I) is —C₁-C₆alkyl, such as, for example, substituted or unsubstituted: C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like. In some embodiments, R⁵ is —CH₃.

In some embodiments, R⁵ in the compounds of formula (I) is —C₁-C₆ alkoxyl, such as, for example, —C₁-C₆alkoxyl, —C₁-C₅alkoxyl, —C₁-C₄alkoxyl, —C₁-C₃alkoxyl, —C₁-C₂alkoxyl, —C₁alkoxyl, —C₂alkoxyl, —C₃alkoxyl, —C₄alkoxyl, —C₅alkoxyl, —C₆alkoxyl, —OCH₃, —OCH₂CH₃, -propoxyl, and the like. In some embodiments, R⁵ is —OCH₃.

In some embodiments, R⁵ in the compounds of formula (I) is -cycloalkyl, such as, for example, cyclopropyl, cyclobutyl, cyclopentyl, cyclohexyl, or cycloheptyl, and the like.

In the compounds of formula (I) of the disclosure, when ring A is 3,5-dichloropyridin-4-yl, and two of Q¹, Q², or Q³ are C—H, then the other of Q¹, Q², or Q³ is C—R² wherein R² is halogen, substituted or unsubstituted —C₁-C₆alkyl, —OR^{2a}, —NR^{2b}R^{2c}, —SO₂C₁-C₆alkyl, or —CN, wherein R^{2a}, R^{2b}, and R^{2c} are as defined above.

In some aspects, the compounds of formula (I) are compounds of formula (IA):

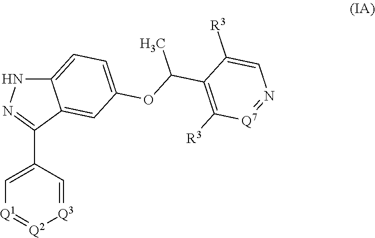

wherein Q¹, Q², and Q³ are as described above for formula (I), Q⁷ is N or C—R³; and each R³ is independently H, halogen, or C₁-C₃alkyl.

In some embodiments of the compounds of formula (IA), Q⁷ is N.

In other embodiments of the compounds of formula (IA), Q⁷ is C—R³.

In some embodiments of the compounds of formula (IA), R³ is H.

In some embodiments of the compounds of formula (IA), R³ is halogen, such as, for example, —F, —Cl, —Br, or —I. In some embodiments, R³ is —Cl. In some embodiments, R³ is —F.

In some embodiments of the compounds of formula (IA), R³ is C₁-C₃alkyl.

In some embodiments of the compounds of formula (IA), R³ is CH₃.

In some embodiments of the compound of formula (IA), Q⁷ is C—H and each R³ is —C₁, and the compounds of formula (IA) have the formula (IA-1):

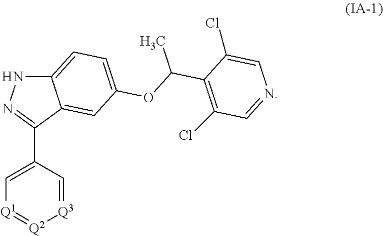

In some embodiments, the compounds of formula (IA-1) are compound of formula (IA-1-1):

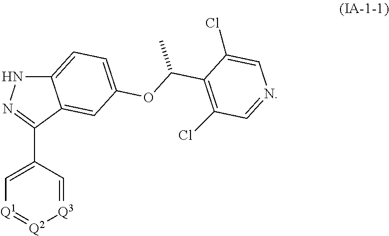

In some embodiments, the compounds of formula (IA-1) are compound of formula (LA-1-2):

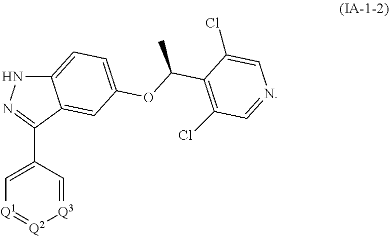

In some embodiments of the compounds of formula (LA) is a compound of formula (LA-2):

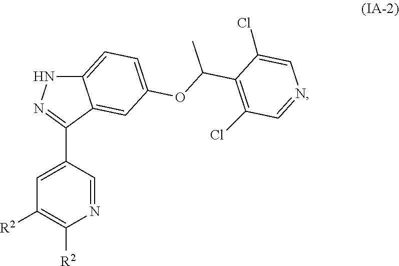

wherein each R² is independently as described above for formula (I).

In some embodiments, the compounds of formula (LA-2) are compounds of formula (IA-2-1):

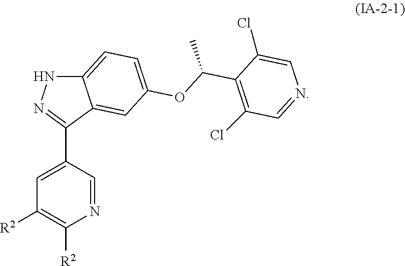

In other embodiments, the compounds of formula (IA-2) are compounds of formula (IA-2-2):

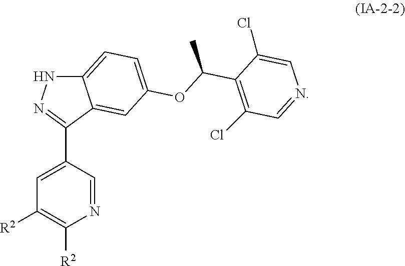

In some embodiments of the compounds of formula (IA-2), each R² is independently H, halogen, substituted or unsubstituted —C₁-C₆alkyl, —OR^{2a}; —NR^{2b}R^{2c}, —C(O)NR^{2d}R^{2e}, —SO₂C₁-C₆alkyl, or —CN;
wherein each R^{2a} is independently-C₁-C₆alkyl, —C₁-C₆haloalkyl, —C₃-C₆cycloalkyl, —C₂-C₅heterocycloalkyl, —CH₂—C₃-C₆cycloalkyl, —CH₂CH₂O—C₁-C₆alkyl;
each R^{2b} is independently H, —C(O)-(substituted or unsubstituted 5- to 10-membered heteroaryl), —C(O)-(substituted or unsubstituted 5- to 6-membered heterocycloalkyl), substituted or unsubstituted —C₁-C₆alkyl, —C₁-C₆alk-O—C₁-C₆alkyl, —C(O)—(C₃-C₆cycloalkyl), —C(O)N(C₁-C₆alkyl)₂, —C(O)C₁-C₃alkN(C₁-C₆alkyl)₂, —C(O)—OC₁-C₆alkyl, —C₃-C₆cycloalkyl, —C(O)CH═CH₂; and
each R^{2c} is independently H, —C(O)-(substituted or unsubstituted 5- to 10-membered heteroaryl), —C(O)-(substituted or unsubstituted 5- to 6-membered heterocycloalkyl), substituted or unsubstituted —C₁-C₆alkyl, —C₁-C₆alk-O—C₁-C₆alkyl, —C(O)—(C₃-C₆cycloalkyl), —C(O)N(C₁-C₆alkyl)₂, —C(O)C₁-C₃alkN(C₁-C₆alkyl)₂, —C(O)—OC₁-C₆alkyl, —C₃-C₆cycloalkyl, —C(O)CH═CH₂.

In some embodiments, each R² is independently H, —CH₂CH₂N(CH₃)₂, —NH₂, —NHCH₂ (substituted or unsubstituted heteroaryl), —OCH₃, —OCH₂CH₃, —OCH₂CF₃, —OCF₃, —F, substituted or unsubstituted-NH—C(O)-(5- to 10-membered heteroaryl), substituted or unsubstituted-NH—C(O)-(5- to 6-membered heterocycloalkyl), —NHC(O)N(CH₃)₂, —NHC(O)CH₂N(CH₃)₂, —NHC(O)CH₂CH₂N(CH₃)₂, —C(O)NH—C₁-C₆alkyl, —NHC(O)—OC₁-C₆alkyl, —C(O)NH₂, —C(O)—(C₃-C₆cycloalkyl), —SO₂CH₃, or —CN.

In some embodiments, each R² is independently H, —CH₂CH₂N(CH₃)₂, —NH₂, —OCH₃, —OCH₂CH₃, —OCH₂CF₃, —OCF₃, —F, —NHC(O)N(CH₃)₂, —NHC(O)CH₂N(CH₃)₂, —NHC(O)CH₂CH₂N(CH₃)₂, —C(O)NHCH₃, —C(O)NH₂, —SO₂CH₃, —CN,

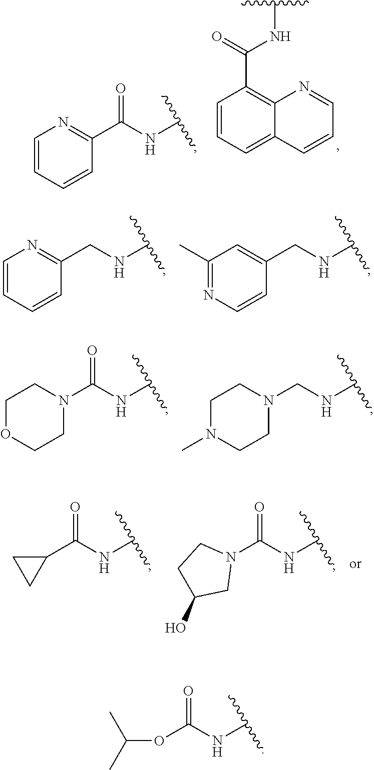

In some embodiments of the compounds of formula IA-2, the two R² groups together with the atoms to which they are attached, form a substituted or unsubstituted 5-7 membered cycloalkyl ring or a substituted or unsubstituted 5-7 membered heterocycloalkyl ring.

In some embodiments, the two R² groups together with the atoms to which they are attached, form a substituted or unsubstituted morpholino ring.

In some embodiments, the compounds of formula IA-2 are compounds of formula (IA-2-3):

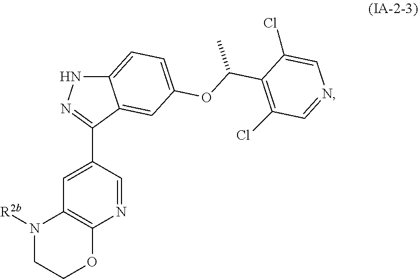

wherein R^{2b} is H, —C(O)-(substituted or unsubstituted 5- to 10-membered heteroaryl), —C(O)— (5- to 6-membered heterocycloalkyl), substituted or unsubstituted —C₁-C₆alkyl, —CH₂CH₂O— C₁-C₆alkyl, —C(O)—(C₃-C₆cycloalkyl), —C(O)N(C₁-C₆alkyl)₂, —C(O)C₁-C₃alkN(C₁-C₆alkyl)₂, substituted or unsubstituted —C(O)—OC₁-C₆alkyl, —C₃-C₆cycloalkyl, or —C(O)CH—CH₂.

In some embodiments of the compounds of formula IA-2, R^{2b} is H,

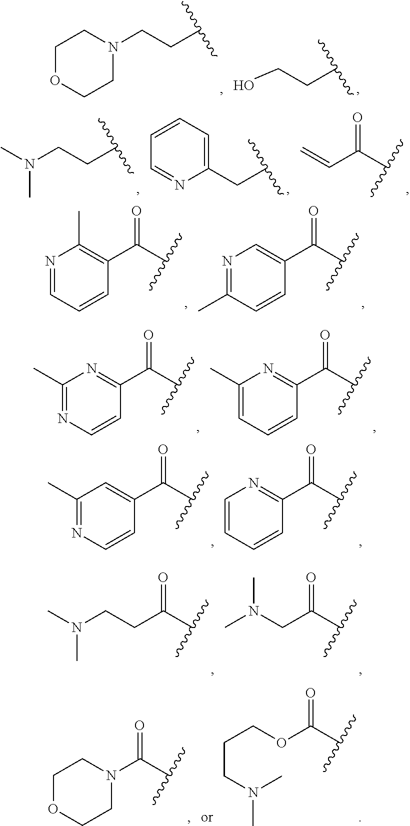

In some embodiments of the compounds of formula IA-2, R^{2b} is H, —CH(CH₃)₂, —SO₂(C₁-C₆alkyl), —SO₂(CH₃), —SO₂(CH₂CH₃), —CH₂CHF₂, or —CHCF₃.

In some embodiments, the compounds of formula IA-2 are compounds of formula (IA-2-4):

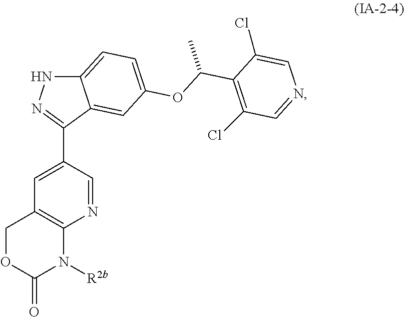

wherein R^{2b} is H, —C(O)-(substituted or unsubstituted 5- to 10-membered heteroaryl), —C(O)-(5- to 6-membered heterocycloalkyl), substituted or unsubstituted —C₁-C₆alkyl, —C₁-C₆alk-O—C₁-C₆alkyl, —C(O)—(C₃-C₆cycloalkyl), —C(O)N(C₁-C₆alkyl)₂, —C(O)C₁-C₃alkN(C₁-C₆alkyl)₂, substituted or unsubstituted —C(O)—OC₁-C₆alkyl, —C₃-C₆cycloalkyl, or —C(O)CH═CH₂.

In some embodiments of the compounds of formula IA-2-4, R^{2b} is H.

In some embodiments, the compounds of formula (IA) are compounds of formula (IA-3):

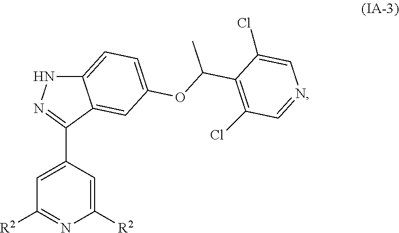

wherein each R² is independently as described above for formula (I).

In some embodiments, the compounds of formula (IA-3) are compounds of formula (IA-3-1):

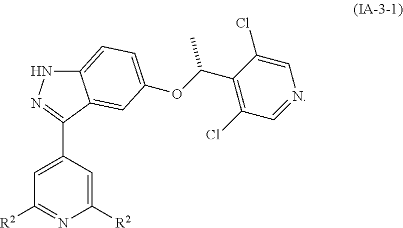

In other embodiments, the compounds of formula (IA-3) are compounds of formula (IA-3-2):

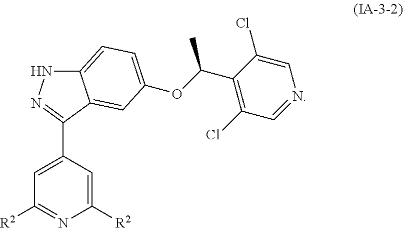

In some embodiments of the compounds of formula IA-3, each R² is independently H, or —NR^{2b}R^{2c}, wherein each R^{2b} and each R^{2c} is independently H, substituted or unsubstituted —C₁-C₆alkyl, or —C₁-C₆alk-O—C₁-C₆alkyl.

In some embodiments, each R² is independently H, —NH₂, —NH—C₂-C₆alkOH, or —NH—C₂-C₆alk-OC₁-C₆alkyl.

In some aspects, the compounds of formula (IA) are compounds of formula (IA-4):

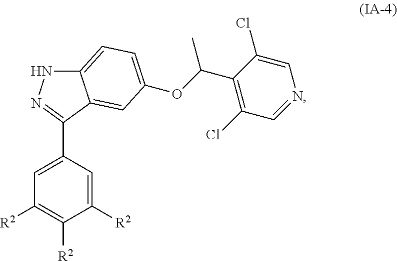

wherein each R² is independently as described above for formula (I).

In some embodiments, the compounds of formula (IA-4) are compounds of formula (IA-4-1):

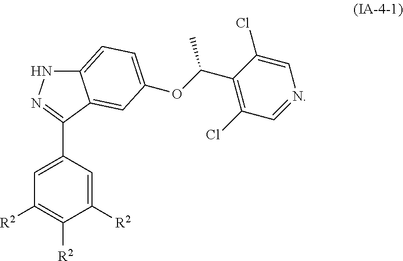

In other embodiments, the compounds of formula (IA-4) are compounds of formula (IA-4-2):

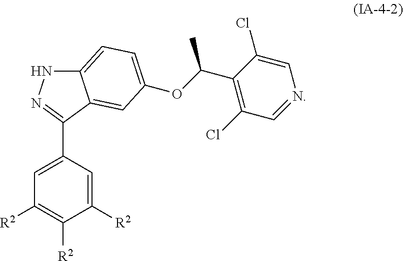

In some embodiments of the compounds of formula IA-4, each R² is independently H, —OR^{2a}; or —NR^{2b}R^{2c}, wherein each R^{2a} is independently-C₁-C₆alkyl; and each R^{2b} and each R^{2c} is independently H, —C(O)-(5- to 10-membered heteroaryl), or substituted or unsubstituted —C₁-C₆alkyl.

In some embodiments of the compounds of formula IA-4, each R² is independently H, —NH₂, —OCH₃; —NC(O)-pyridinyl, —NHCH₂-pyrindinyl,

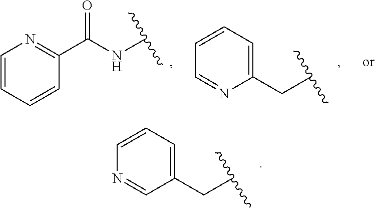 [SMILES: *NC(=O)c1ccccn1 ; *Cc1ccccn1 ; *Cc1cccnc1]

In some aspects, the compounds of formula (IA) are compounds of formula (IA-5):

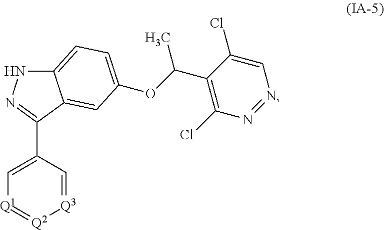

wherein Q¹, Q², and Q³ are as described above with respect to formula (I).

In some embodiments, the compounds of formula (LA-5) are compounds of formula (LA-5-1):

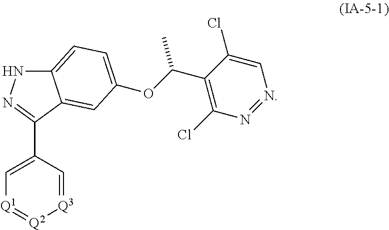

In other embodiments, the compounds of formula (LA-5) are compounds of formula (LA-5-2):

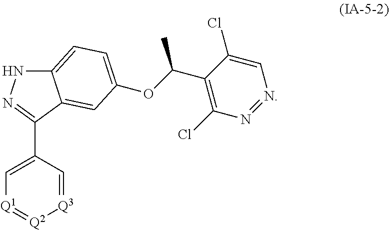

In some aspects, the compounds of formula (LA) are compounds of formula (LA-6):

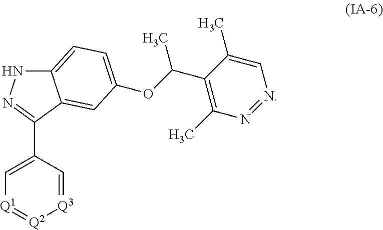

In some embodiments, the compound of formula (IA-6) are compounds of formula (IA-6-1):

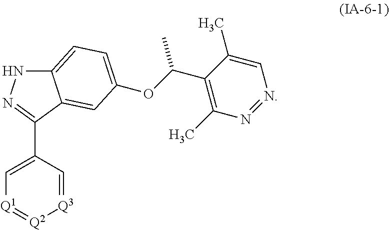

In other embodiments, the compounds of formula (LA-6) are compounds of formula (IA-6-2):

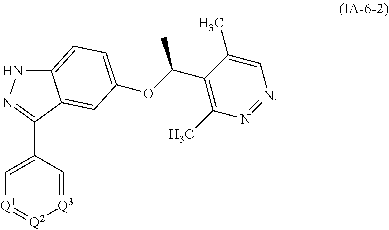

In some aspects, the compounds of formula (IA) are compounds of formula (IA-7):

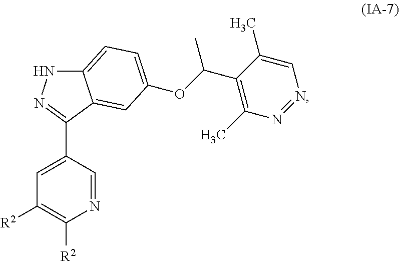

wherein each R² is independently as described above with respect to formula I.

In some embodiments, the compounds of formula (IA-7) are compounds of formula (IA-7-1):

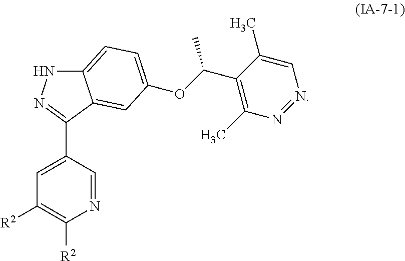

In some embodiments, the compounds of formula (LA-7) are compounds of formula (IA-7-2):

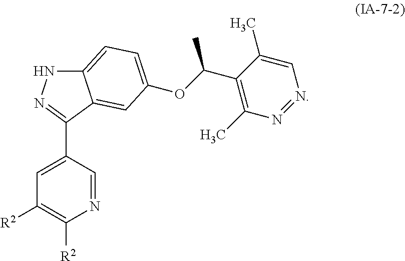

In some aspects, the compounds of formula (LA) are compounds of formula (LA-8):

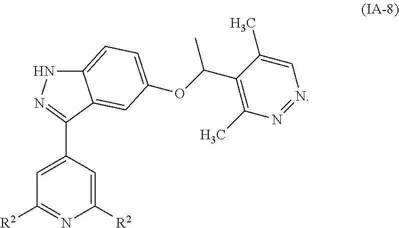

In some aspects, the compounds of formula (LA-8) are compounds of formula (IA-8-1):

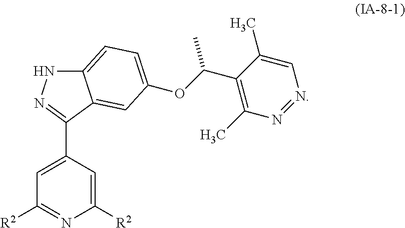

In some aspects, the compounds of formula (IA-8) are compounds of formula (IA-8-2):

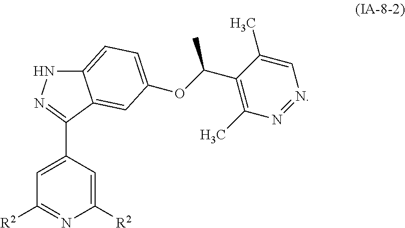

In some embodiments of the compounds of formula IA-8, each R² is independently-C₁-C₆alkyl, —CH₃, or —CN.

In some aspects, the compounds of formula (IA) are compounds of formula (IA-9):

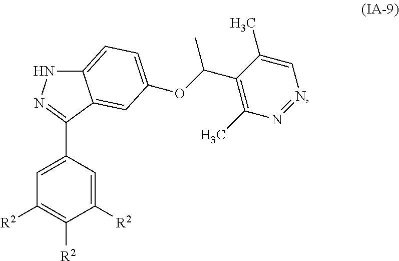

wherein each R² is independently as described above with respect to formula I.

In some embodiments, the compounds of formula (LA-9) are compounds of formula (IA-9-1):

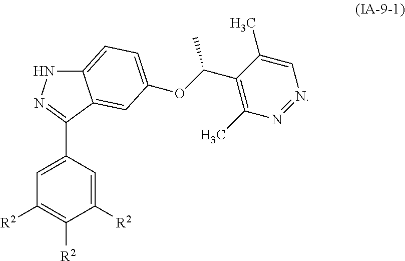

In other embodiments, the compounds of formula (IA-9) are compounds of formula (IA-9-2):

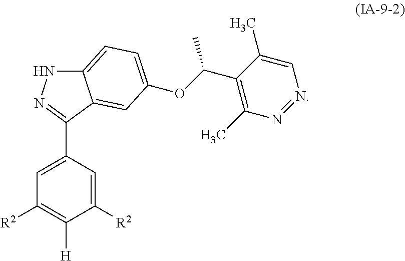

In other embodiments, the compounds of formula (IA-9) are compounds of formula (IA-9-3):

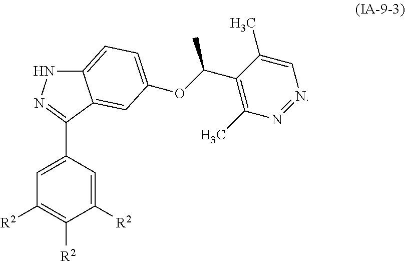

In some embodiments of the compounds of formula (IA-9), each R² is independently H, halogen, substituted or unsubstituted —C₁-C₆alkyl, —OR^{2a}; —CN, substituted or unsubstituted —C₃-C₆cycloalkyl, substituted or unsubstituted 3-6-membered heterocycloalkyl, or substituted or unsubstituted spiroheterocycloalkyl; and wherein each R^{2a} is independently substituted or unsubstituted —C₁-C₆alkyl, —C₁-C₆haloalkyl, substituted or unsubstituted —C₃-C₆cycloalkyl, substituted or unsubstituted —C₂-C₅heterocycloalkyl, substituted or unsubstituted —CH₂—C₃-C₆cycloalkyl, or substituted or unsubstituted —CH₂CH₂O—C₁-C₆alkyl.

In some embodiments of the compounds of formula (IA-9), each R² is independently H, F, —CH₃, —CN, —OCH₃, —OCH₂CH₃, —OCH(CH₃)₂, —OCF₃, —OCH₂CH₂OH, —OCH₂CH₂OCH₃, —OCH₂CH₂OCH₂CH₃,

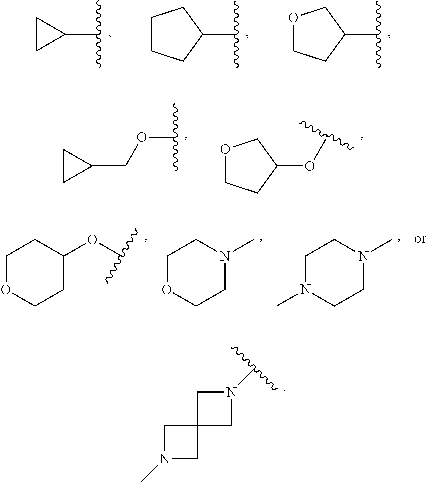

In some aspects, the compounds of formula (IA) are compounds of formula (IA-9A):

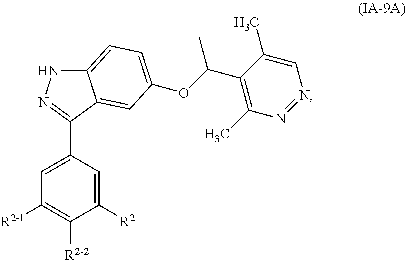

wherein R² is as described above for formula (I), and R²⁻¹ and R²⁻², together with the atoms to which they are attached form a substituted or unsubstituted 5-7 membered heterocycloalkyl ring.

In some embodiments, the compounds of formula IA-9A are compounds of formula (IA-9A-1):

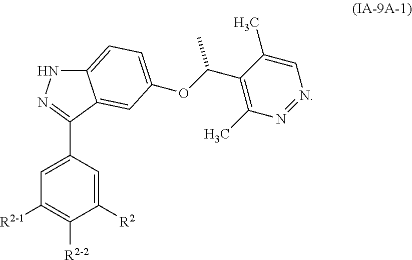

In other embodiments, the compounds of formula IA-9A are compounds of formula (IA-9A-2):

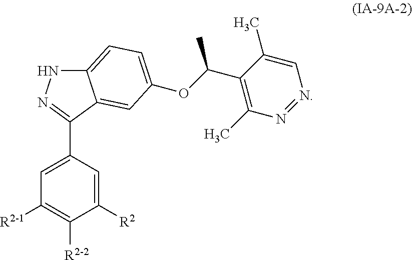

In some embodiments of the compounds of formula (IA-9A), R²⁻¹ and R²⁻², together with the atoms to which they are attached, form a substituted or unsubstituted 5-7 membered cycloalkyl ring or a substituted or unsubstituted 5-7 membered heterocycloalkyl ring.

In some embodiments, R²⁻¹ and R²⁻², together with the atoms to which they are attached, form substituted or unsubstituted 1,3-dioxanyl or tetrahydropyranyl group.

In some embodiments of the compounds of formula (IA-9A), R²⁻¹ and R²⁻², together with the atoms to which they are attached, form a substituted 5-7 membered heterocycloalkyl ring that is substituted with a substituted or unsubstituted 5-7 membered spiroheterocycloalkyl ring.

In some embodiments of the compounds of formula (IA-9A) in which R²⁻¹ and R²⁻², together with the atoms to which they are attached, form a substituted 5-7 membered heterocycloalkyl ring that is substituted with a substituted or unsubstituted 5-7 membered spiroheterocycloalkyl ring, the 5-7 membered spiroheterocycloalkyl ring is a 5-membered spiroheterocycloalkyl ring (e.g., a spiropyrrolidinyl ring) or a 6-membered spiroheterocycloalkyl ring (e.g., a spiropiperidinyl ring).

In some embodiments of the compounds of formula (IA-9A) in which R²⁻¹ and R²⁻², together with the atoms to which they are attached, form a substituted 5-7 membered heterocycloalkyl ring that is substituted with a substituted or unsubstituted 6-membered spiroheterocycloalkyl ring, the 6-membered spiroheterocycloalkyl ring is

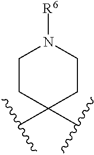

wherein R⁶ is C₁-C₆alkyl, —C₃-Ccycloalkyl, 3-7-membered heterocycloalkyl, —C(O)C₁-C₆alkyl, —C(O)C₃-C₆cycloalkyl, —C(O)N(C₁-C₆alkyl)₂, —C(O)NH(C₁-C₆alkyl), —C(O)OC₁-C₆alkyl, or —C(O)OC₃-C₆cycloalkyl.

In some embodiments of the compounds of formula (IA-9A), R²⁻¹ and R²⁻², together with the atoms to which they are attached, is

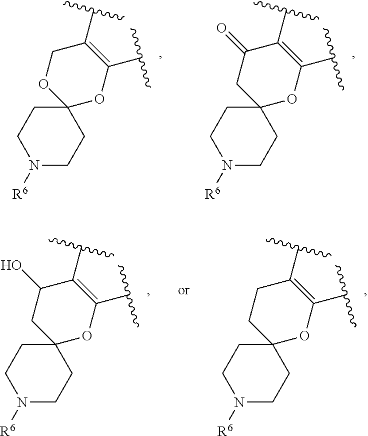

wherein the R⁶ is H, C₁-C₆alkyl, C₁-C₆haloalkyl, —C₃-C₆cycloalkyl, 3-7-membered heterocycloalkyl, —C(O)C₁-C₆alkyl, —C(O)C₃-C₆cycloalkyl, —C(O)N(C₁-C₆alkyl)₂, —C(O)NH(C₁-C₆alkyl), —C(O)OC₁-C₆alkyl, —C(O)OC₃-C₆cycloalkyl, or —SO₂C₁-C₆alkyl.

In some embodiments of the compounds of formula (IA-9A), R²⁻¹ and R²⁻², together with the atoms to which they are attached, is

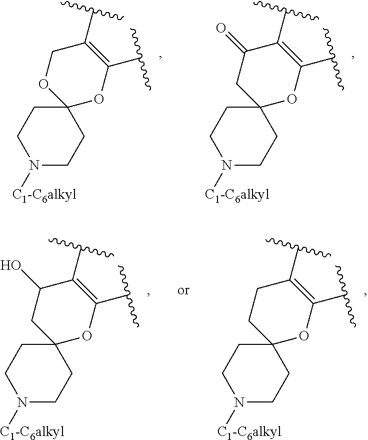

wherein the C₁-C₆alkyl is, for example, C₆alkyl, C₅alkyl, C₄alkyl, C₃alkyl, C₂alkyl, C₁alkyl, methyl, ethyl, or isopropyl.

In some embodiments of the compounds of formula (IA-9A), R²⁻¹ and R²⁻², together with the atoms to which they are attached, is

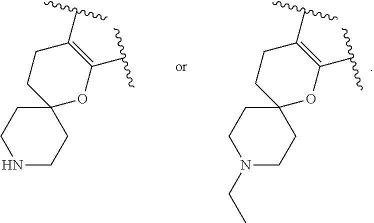 [SMILES: *C1=C(*)OC2(CCNCC2)CC1 ; *C1=C(*)OC2(CC1)CCN(CC)CC2]

In some embodiments of the compounds of formula (IA-9A), R²⁻¹ and R²⁻², together with the atoms to which they are attached, is

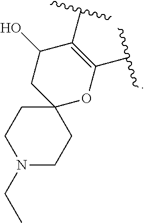 [SMILES: *C1=C(*)C(O)CC2(CCN(CC)CC2)O1]

In some embodiments of the compounds of formula (IA-9A), R²⁻¹ and R²⁻², together with the atoms to which they are attached, is

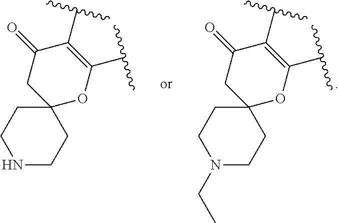 [SMILES: *C1=C(*)C(=O)CC2(CCNCC2)O1 ; *C1=C(*)C(=O)CC2(CCN(CC)CC2)O1]

In some embodiments of the compounds of formula (IA-9A), R²⁻¹ and R²⁻², together with the atoms to which they are attached, is

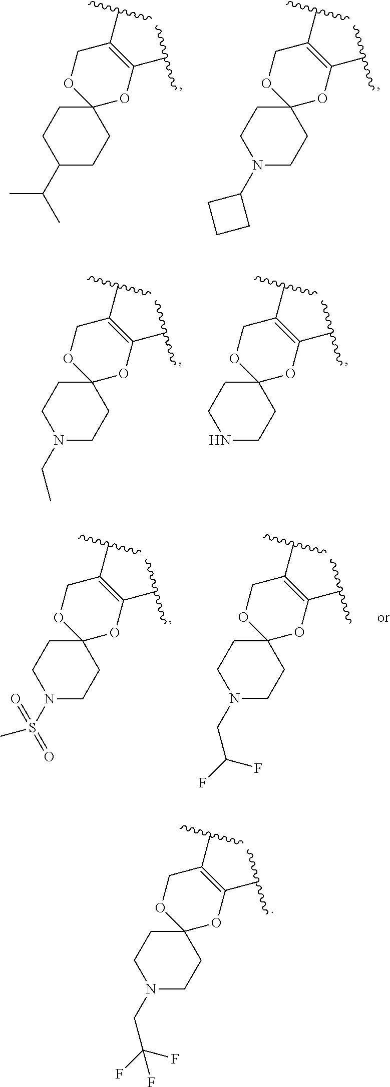

In some aspects, the compounds of formula (I) are compounds of formula (IB):

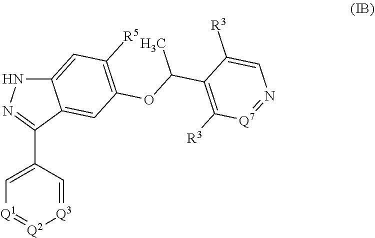

wherein Q¹, Q², and Q³ are as described above for formula (I), Q⁷ is N or C—R³; and each R³ is independently H, halogen, or C₁-C₃alkyl, R⁵ is halogen, —C₁-C₆alkyl, or —C₁-C₆ alkoxyl.

In some embodiments of the compounds of formula (IB), Q⁷ is N.

In other embodiments of the compounds of formula (IB), Q⁷ is C—R³.

In some embodiments of the compounds of formula (IB), R³ is H.

In some embodiments of the compounds of formula (IB), R³ is halogen, such as, for example, —F, —Cl, —Br, or —I. In some embodiments, R³ is —Cl. In some embodiments, R³ is —F.

In some embodiments of the compounds of formula (IB), R³ is C₁-C₃alkyl.

In some embodiments of the compounds of formula (IB), R³ is CH₃.

In some embodiments of the compounds of formula (IB), R⁵ is halogen, such as, for example, —F, —Cl, —Br, or —I. In some embodiments, R⁵ is —Cl. In other embodiments, R⁵ is —F.

In some embodiments of the compounds of formula (IB), R⁵ is —C₁-C₆alkyl.

In some embodiments, R⁵ is CH₃.

In some embodiments of the compounds of formula (IB), R⁵ is —C₁-C₆ alkoxyl. In some embodiments, R⁵ is OCH₃.

In some embodiments of the compound of formula (IB), Q⁷ is C—H and each R³ is —C₁, and the compounds of formula (IB) have the formula (IB-1):

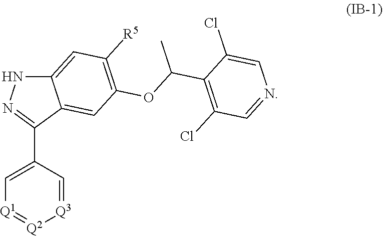

In some embodiments, the compounds of formula (IB-1) are compound of formula (IB-1-1):

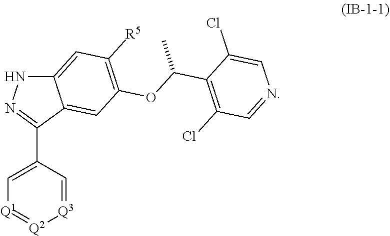

In some embodiments, the compounds of formula (IB-1) are compound of formula (IB-1-2):

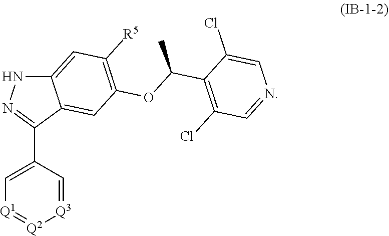

In some embodiments of the compounds of formula (IB) is a compound of formula (IB-2):

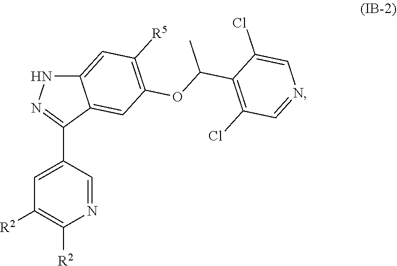

wherein each R² is independently as described above for formula (I).

In some embodiments, the compounds of formula (IB-2) are compounds of formula (IB-2-1):

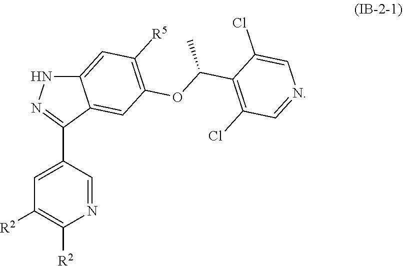

In other embodiments, the compounds of formula (IB-2) are compounds of formula (IB-2-2):

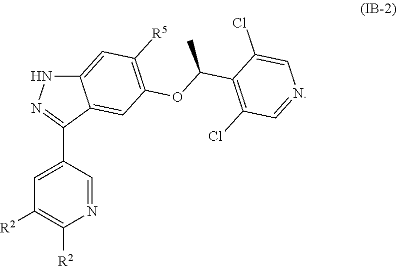

In some embodiments of the compounds of formula (IB-2), each R² is independently H, halogen, substituted or unsubstituted —C₁-C₆alkyl, —OR^{2a}, —NR^{2b}R^{2c}, —C(O)NR^{2d}R^{2e}, —SO₂C₁-C₆alkyl, or —CN:
wherein each R^{2a} is independently-C₁-C₆alkyl, —C₁-C₆haloalkyl, —C₃-C₆cycloalkyl, —C₂-C₅heterocycloalkyl, —CH₂—C₃-C₆cycloalkyl, —CH₂CH₂O—C₁-C₆alkyl;
each R^{2b} is independently H, —C(O)-(substituted or unsubstituted 5- to 10-membered heteroaryl), —C(O)-(substituted or unsubstituted 5- to 6-membered heterocycloalkyl), substituted or unsubstituted —C₁-C₆alkyl, —C₁-C₆alk-O—C₁-C₆alkyl, —C(O)—(C₃-C₆cycloalkyl), —C(O)N(C₁-C₆alkyl)₂, —C(O)C₁-C₃alkN(C₁-C₆alkyl)₂, —C(O)—OC₁-C₆alkyl, —C₃-C₆cycloalkyl, —C(O)CH═CH₂; and
each R^{2c} is independently H, —C(O)-(substituted or unsubstituted 5- to 10-membered heteroaryl), —C(O)-(substituted or unsubstituted 5- to 6-membered heterocycloalkyl), substituted or unsubstituted —C₁-C₆alkyl, —C₁-C₆alk-O—C₁-C₆alkyl, —C(O)—(C₃-C₆cycloalkyl), —C(O)N(C₁-C₆alkyl)₂, —C(O)C₁-C₃alkN(C₁-C₆alkyl)₂, —C(O)—OC₁-C₆alkyl, —C₃-C₆cycloalkyl, —C(O)CH═CH₂.

In some embodiments, each R² is independently H, —CH₂CH₂N(CH₃)₂, —NH₂, —NHCH₂ (substituted or unsubstituted heteroaryl), —OCH₃, —OCH₂CH₃, —OCH₂CF₃, —OCF₃, —F, substituted or unsubstituted-NH—C(O)-(5- to 10-membered heteroaryl), substituted or unsubstituted-NH—C(O)-(5- to 6-membered heterocycloalkyl), —NHC(O)N(CH₃)₂, —NHC(O)CH₂N(CH₃)₂, —NHC(O)CH₂CH₂N(CH₃)₂, —C(O)NH—C₁-C₆alkyl, —NHC(O)—OC₁-C₆alkyl, —C(O)NH₂, —C(O)—(C₃-C₆cycloalkyl), —SO₂CH₃, or —CN.

In some embodiments, each R² is independently H, —CH₂CH₂N(CH₃)₂, —NH₂, —OCH₃, —OCH₂CH₃, —OCH₂CF₃, —OCF₃, —F, —NHC(O)N(CH₃)₂, —NHC(O)CH₂N(CH₃)₂, —NHC(O)CH₂CH₂N(CH₃)₂, —C(O)NHCH₃, —C(O)NH₂, —SO₂CH₃, —CN,

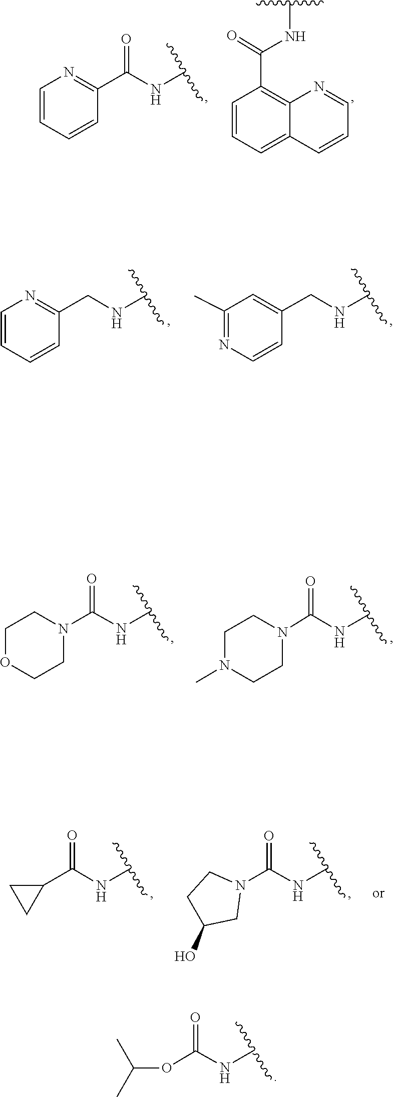

In some embodiments of the compounds of formula IB-2, the two R² groups together with the atoms to which they are attached, form a substituted or unsubstituted 5-7 membered cycloalkyl ring or a substituted or unsubstituted 5-7 membered heterocycloalkyl ring.

In some embodiments, the two R² groups together with the atoms to which they are attached, form a substituted or unsubstituted morpholino ring.

In some embodiments, the compounds of formula IB-2 are compounds of formula (IB-2-3):

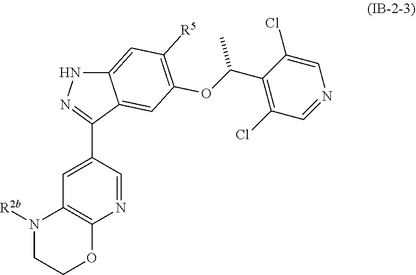

wherein R^{2b} is H, —C(O)-(substituted or unsubstituted 5- to 10-membered heteroaryl), —C(O)-(5- to 6-membered heterocycloalkyl), substituted or unsubstituted —C₁-C₆alkyl, —CH₂CH₂O—C₁-C₆alkyl, —C(O)—(C₃-C₆cycloalkyl), —C(O)N(C₁-C₆alkyl)₂, —C(O)C₁-C₃alkN(C₁-C₆alkyl)₂, substituted or unsubstituted —C(O)—OC₁-C₆alkyl, —C₃-C₆cycloalkyl, —SO₂(C₁-C₆alkyl), —CH₂CHF₂, —CH₂CF₃, or —C(O)CH═CH₂.

In some embodiments of the compounds of formula IB-2, R^{2b} is H,

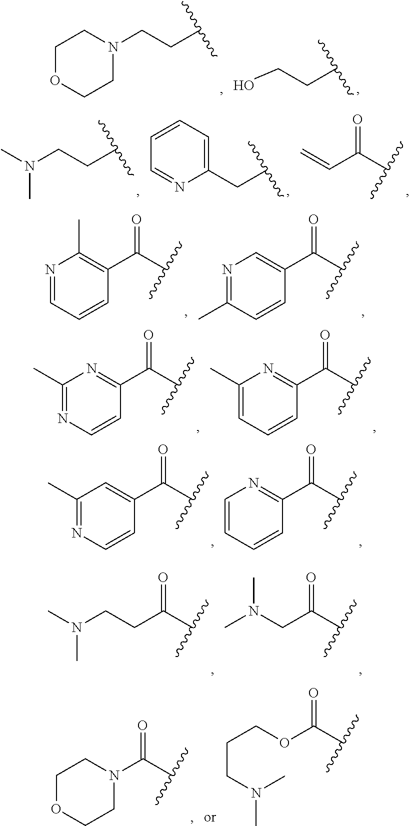

In some embodiments of the compounds of formula IB-2, R²⁰ is H, —CH(CH₃)₂, —SO₂(C₁-C₆alkyl), —SO₂(CH₃), —SO₂(CH₂CH₃), —CH₂CHF₂, or —CHCF₃.

In some embodiments, the compounds of formula IB-2 are compounds of formula (IB-2-4):

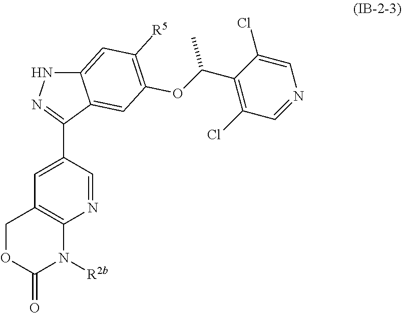

wherein R^{2b} is H, —C(O)-(substituted or unsubstituted 5- to 10-membered heteroaryl), —C(O)-(5- to 6-membered heterocycloalkyl), substituted or unsubstituted —C₁-C₆alkyl, —C₁-C₆alk-O—C₁-C₆alkyl, —C(O)—(C₃-C₆cycloalkyl), —C(O)N(C₁-C₆alkyl)₂, —C(O)C₁-C₃alkN(C₁-C₆alkyl)₂, substituted or unsubstituted —C(O)—OC₁-C₆alkyl, —C₃-C₆cycloalkyl, or —C(O)CH═CH₂.

In some embodiments of the compounds of formula IB-2-4, R^{2b} is H.

In some embodiments, the compounds of formula (IB) are compounds of formula (IB-3):

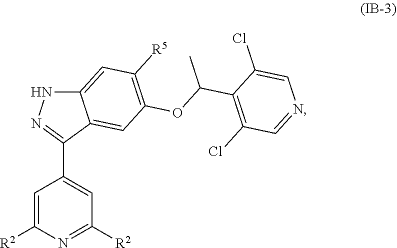

wherein each R² is independently as described above for formula (I).

In some embodiments, the compounds of formula (IB-3) are compounds of formula (IB-3-1):

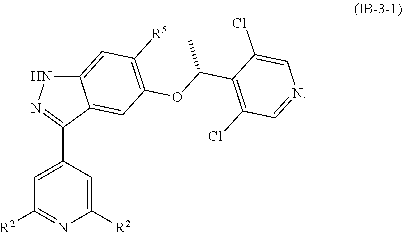

In other embodiments, the compounds of formula (IB-3) are compounds of formula (IB-3-2):

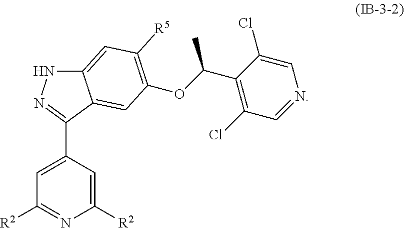

In some embodiments of the compounds of formula IB-3, each R² is independently H, or —NR^{2b}R^{2c}, wherein each R^{2b} and each R^{2c} is independently H, substituted or unsubstituted —C₁-C₆alkyl, or —C₁-C₆alk-O—C₁-C₆alkyl.

In some embodiments, each R² is independently H, —NH₂, —NH—C₂-C₆alkOH, or —NH—C₂-C₆alk-OC₁-C₆alkyl.

In some aspects, the compounds of formula (IB) are compounds of formula (IB-4):

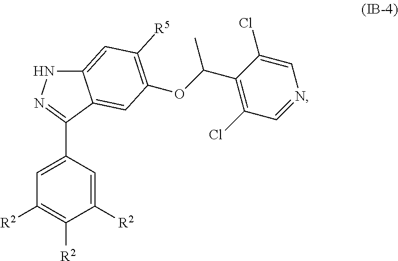

wherein each R² is independently as described above for formula (I).

In some embodiments, the compounds of formula (IB-4) are compounds of formula (IB-4-1):

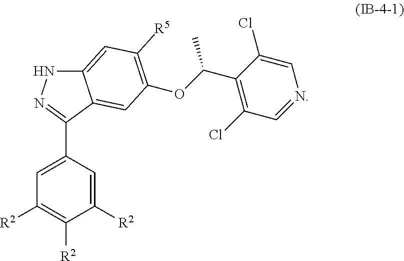

In other embodiments, the compounds of formula (IB-4) are compounds of formula (IB-4-2):

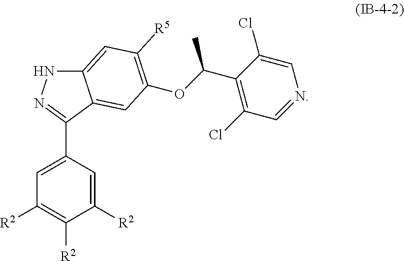

In some embodiments of the compounds of formula IB-4, each R² is independently H, —CN, —OR^{2a}; or —NR^{2b}R^{2c}, wherein each R^{2a} is independently-C₁-C₆alkyl, and each R^{2b} and each R^{2c} is independently H, —C(O)-(5- to 10-membered heteroaryl), or substituted or unsubstituted —C₁-C₆alkyl.

In some embodiments of the compounds of formula IB-4, each R² is independently H, —NH₂, —OCH₃; —NC(O)-pyridinyl, —NHCH₂-pyrindinyl,

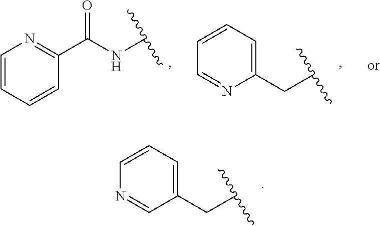

In some aspects, the compounds of formula (IB) are compounds of formula

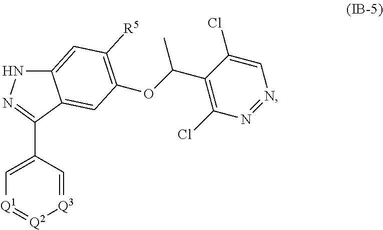

wherein Q¹, Q², and Q³ are as described above with respect to formula (I).

In some embodiments, the compounds of formula (IB-5) are compounds of formula (IB-5-1):

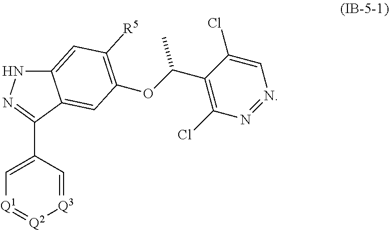

In other embodiments, the compounds of formula (IB-5) are compounds of formula (IB-5-2):

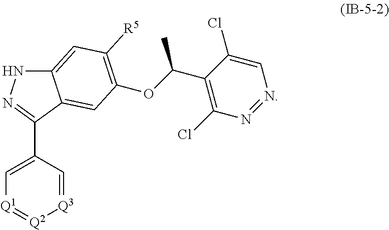

In some aspects, the compounds of formula (IB) are compounds of formula (IB-6):

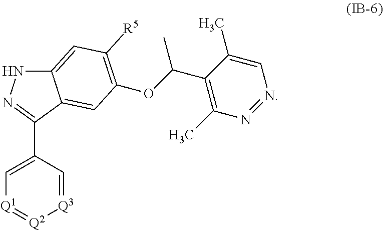

In some embodiments, the compound of formula (IB-6) are compounds of formula (IB-6-1):

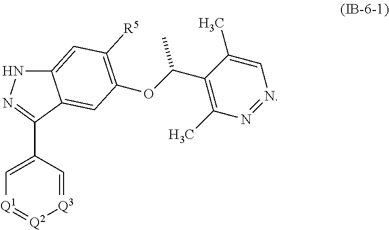

In other embodiments, the compounds of formula (IB-6) are compounds of formula (IB-6-2):

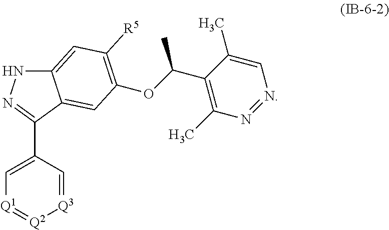

In some aspects, the compounds of formula (IB) are compounds of formula (IB-7):

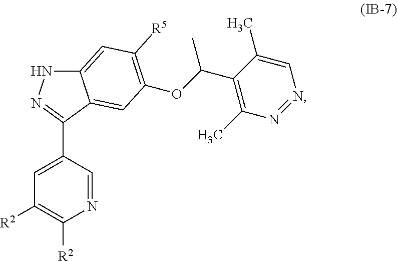

wherein each R² is independently as described above with respect to formula I.

In some embodiments, the compounds of formula (IB-7) are compounds of formula (IB-7-1):

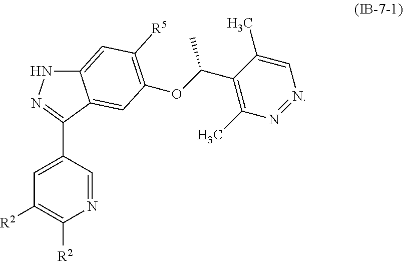

In some embodiments, the compounds of formula (IB-7) are compounds of formula (IB-7-2):

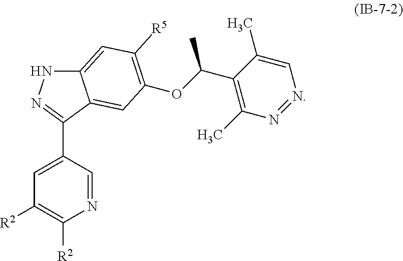

In some embodiments of the compounds of formula (IB-7), each R² is independently H, —C₁-C₆alkyl, —N(C₁-C₆alkyl)₂, —CH₃, —CN,

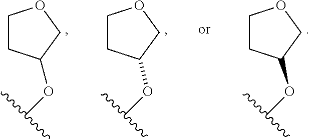

In some aspects, the compounds of formula (IB) are compounds of formula (IB-8):

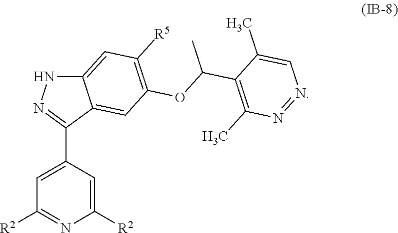

In some aspects, the compounds of formula (IB-8) are compounds of formula (IB-8-1):

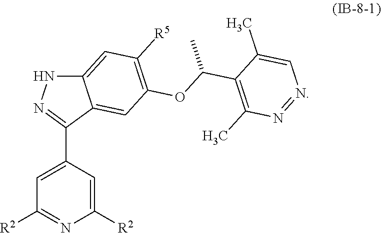

In some aspects, the compounds of formula (IB-8) are compounds of formula (IB-8-2):

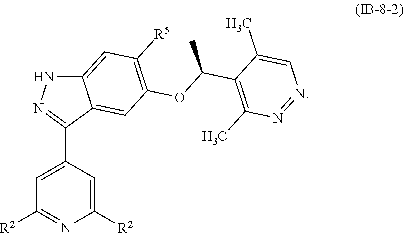

In some embodiments of the compounds of formula IB-8, each R² is independently-C₁-C₆alkyl, —CH₃, —OCH₃, or —CN.

In some aspects, the compounds of formula (IB) are compounds of formula (IB-9):

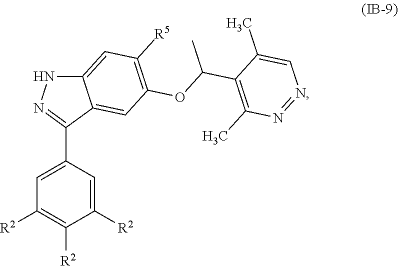

wherein each R² is independently as described above with respect to formula I.

In some embodiments, the compounds of formula (IB-9) are compounds of formula (IB-9-1):

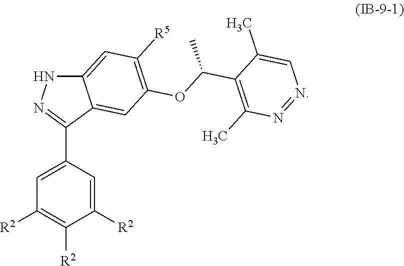

In other embodiments, the compounds of formula (IB-9) are compounds of formula (IB-9-2):

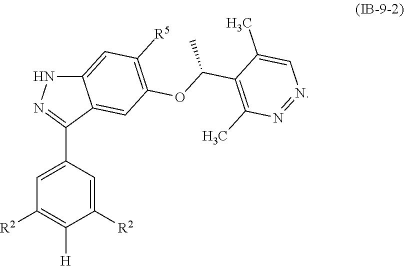

In other embodiments, the compounds of formula (IB-9) are compounds of formula (IB-9-3):

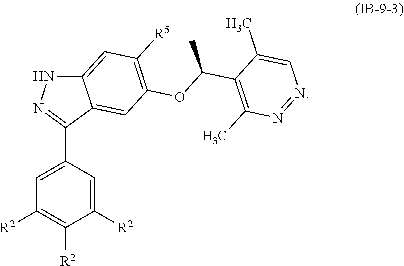

In some embodiments of the compounds of formula (IB-9), each R² is independently H, halogen, substituted or unsubstituted —C₁-C₆alkyl, —OR^{2a}; —CN, substituted or unsubstituted —C₃-C₆cycloalkyl, substituted or unsubstituted 3-6-membered heterocycloalkyl, or substituted or unsubstituted spiroheterocycloalkyl; and wherein each R^{2a} is independently substituted or unsubstituted —C₁-C₆alkyl, —C₁-C₆haloalkyl, substituted or unsubstituted —C₃-C₆cycloalkyl, substituted or unsubstituted —C₂-C₅heterocycloalkyl, substituted or unsubstituted —CH₂—C₃-C₆cycloalkyl, or substituted or unsubstituted —CH₂CH₂O—C₁-C₆alkyl.

In some embodiments of the compounds of formula (IB-9), each R² is independently H, —C₁-C₆alkyl, —N(C₁-C₆alkyl)₂, cyclopropyl, —CN, or —OCH₃.

In some embodiments of the compounds of formula (IB-9), each R² is independently H, F, —CH₃, —CN, —OCH₃, —OCH₂CH₃, —OCH(CH₃)₂, —OCF₃, —OCH₂CH₂OH, —OCH₂CH₂OCH₃, —OCH₂CH₂OCH₂CH₃,

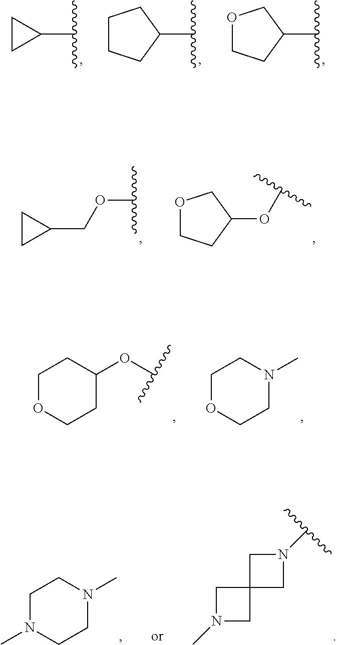

In some aspects, the compounds of formula (IB) are compounds of formula (IB-9A):

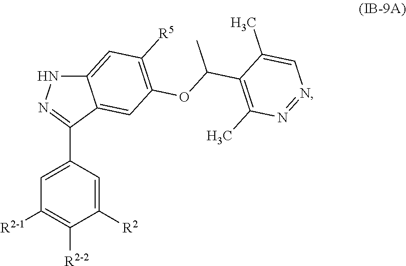

wherein R² is as described above for formula (I), and R²⁻¹ and R²⁻², together with the atoms to which they are attached form a substituted or unsubstituted 5-7 membered heterocycloalkyl ring.

In some embodiments, the compounds of formula IB-9A are compounds of formula (IB-9A-1):

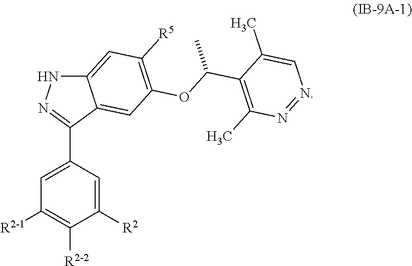

In other embodiments, the compounds of formula IB-9A are compounds of formula (IB-9A-2):

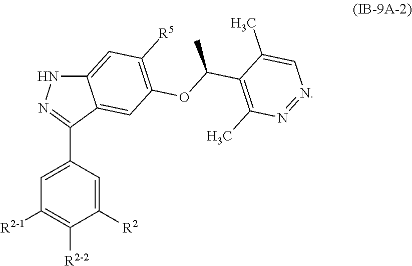

In some embodiments of the compounds of formula (IB-9A), R²⁻¹ and R²⁻², together with the atoms to which they are attached, form a substituted or unsubstituted 5-7 membered cycloalkyl ring or a substituted or unsubstituted 5-7 membered heterocycloalkyl ring.

In some embodiments, R²⁻¹ and R²⁻², together with the atoms to which they are attached, form substituted or unsubstituted 1,3-dioxanyl or tetrahydropyranyl group.

In some embodiments of the compounds of formula (IB-9A), R²⁻¹ and R²⁻², together with the atoms to which they are attached, form a substituted 5-7 membered heterocycloalkyl ring that is substituted with a substituted or unsubstituted 5-7 membered spiroheterocycloalkyl ring.

In some embodiments of the compounds of formula (IB-9A) in which R²⁻¹ and R²⁻², together with the atoms to which they are attached, form a substituted 5-7 membered heterocycloalkyl ring that is substituted with a substituted or unsubstituted 5-7 membered spiroheterocycloalkyl ring, the 5-7 membered spiroheterocycloalkyl ring is a 5-membered spiroheterocycloalkyl ring (e.g., a spiropyrrolidinyl ring) or a 6-membered spiroheterocycloalkyl ring (e.g., a spiropiperidinyl ring).

In some embodiments of the compounds of formula (IB-9A) in which R²⁻¹ and R²⁻², together with the atoms to which they are attached, form a substituted 5-7 membered heterocycloalkyl ring that is substituted with a substituted or unsubstituted 6-membered spiroheterocycloalkyl ring, the 6-membered spiroheterocycloalkyl ring is

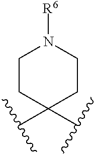

wherein R⁶ is C₁-C₆alkyl, —C₃-Ccycloalkyl, 3-7-membered heterocycloalkyl, —C(O)C₁-C₆alkyl, —C(O)C₃-C₆cycloalkyl, —C(O)N(C₁-C₆alkyl)₂, —C(O)NH(C₁-C₆alkyl), —C(O)OC₁-C₆alkyl, or —C(O)OC₃-C₆cycloalkyl.

In some embodiments of the compounds of formula (IB-9A), R²⁻¹ and R²⁻² together with the atoms to which they are attached, is

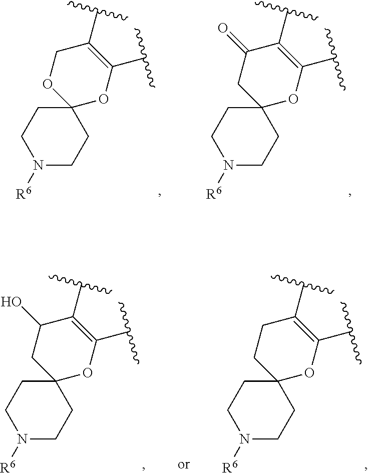

wherein the R⁶ is H, C₁-C₆alkyl, C₁-C₆haloalkyl, —C₃-C₆cycloalkyl, 3-7-membered heterocycloalkyl, —C(O)C₁-C₆alkyl, —C(O)C₃-C₆cycloalkyl, —C(O)N(C₁-C₆alkyl)₂, —C(O)NH(C₁-C₆alkyl), —C(O)OC₁-C₆alkyl, —C(O)OC₃-C₆cycloalkyl, or —SO₂C₁-C₆alkyl.

In some embodiments of the compounds of formula (IB-9A), R²⁻¹ and R²⁻², together with the atoms to which they are attached, is

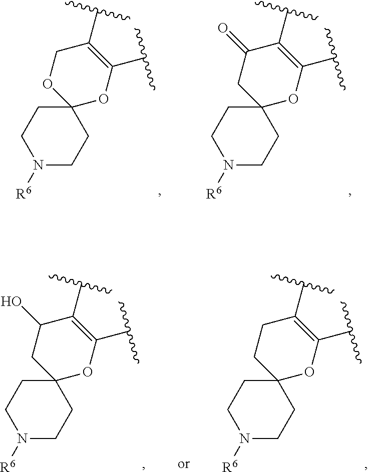

wherein the R⁶ is C₁-C₆alkyl, —C₃-C₆cycloalkyl, 3-7-membered heterocycloalkyl, —C(O)C₁-C₆alkyl, —C(O)C₃-C₆cycloalkyl, —C(O)N(C₁-C₆alkyl)₂, —C(O)NH(C₁-C₆alkyl), —C(O)OC₁-C₆alkyl, —C(O)OC₃-C₆cycloalkyl, or —SO₂C₁-C₆alkyl.

In some embodiments of the compounds of formula (IB-9A), R²⁻¹ and R²⁻², together with the atoms to which they are attached, is

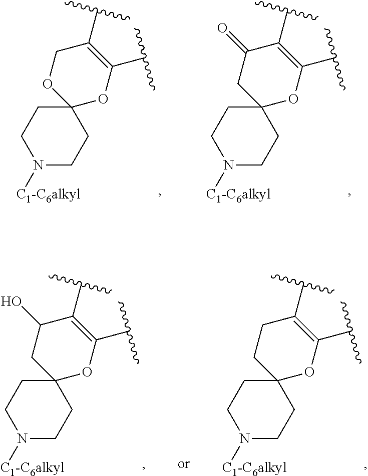

wherein the C₁-C₆alkyl is, for example, C₆alkyl, C₅alkyl, C₄alkyl, C₃alkyl, C₂alkyl, C₁alkyl, methyl, ethyl, or isopropyl.

In some embodiments of the compounds of formula (IB-9A), when Q¹ and Q² are both C—R², the R² groups together with the atoms to which they are attached is

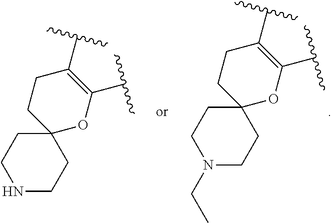 [SMILES: *C1=C(*)OC2(CCNCC2)CC1 ; *C1=C(*)OC2(CC1)CCN(CC)CC2]

In some embodiments of the compounds of formula (IB-9A), R²⁻¹ and R²⁻², together with the atoms to which they are attached, is

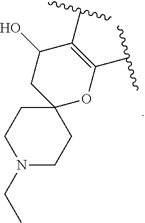 [SMILES: *C1=C(*)C(O)CC2(CCN(CC)CC2)O1]

In some embodiments of the compounds of formula (IB-9A), R²⁻¹ and R²⁻², together with the atoms to which they are attached, is

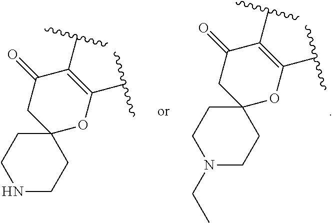 [SMILES: *C1=C(*)C(=O)CC2(CCNCC2)O1 ; *C1=C(*)C(=O)CC2(CCN(CC)CC2)O1]

In some embodiments of the compounds of formula (IB-9A), R²⁻¹ and R²⁻², together with the atoms to which they are attached, is

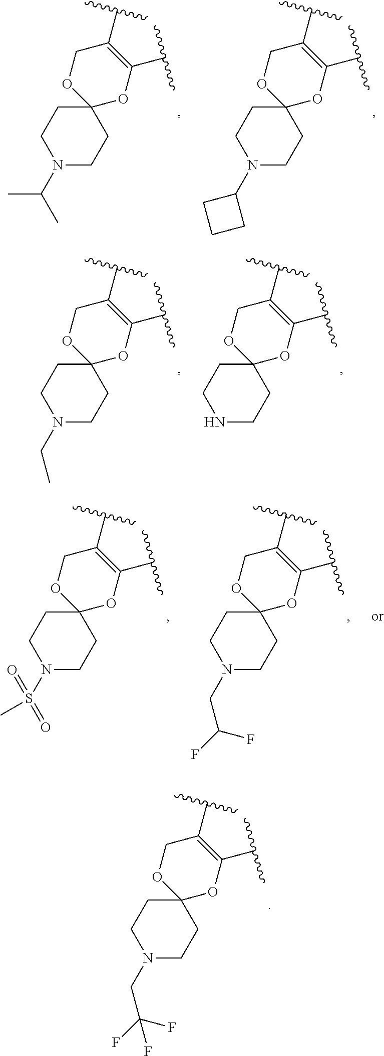

In some aspects, the genus of formula I and subgenera thereof exclude the following compounds or salts thereof:

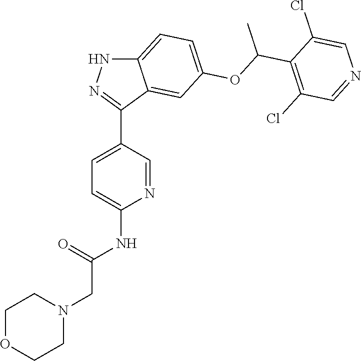 [SMILES: CC(Oc1ccc2[nH]nc(-c3ccc(NC(=O)CN4CCOCC4)nc3)c2c1)c1c(Cl)cncc1Cl]

N-(5-(5-(1-(3,5-
dichloropyridin-4-
yl)ethoxy)-1H-indazol-
3-yl)pyridin-2-yl)-2-
morpholinoacetamide

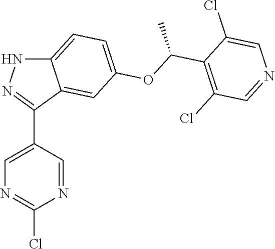 [SMILES: C[C@@H](Oc1ccc2[nH]nc(-c3cnc(Cl)nc3)c2c1)c1c(Cl)cncc1Cl]

(R)-3-(2-
chloropyrimidin-5-
yl)-5-(1-(3,5-
dichloropyridin-4-
yl)ethoxy)-1H-
indazole

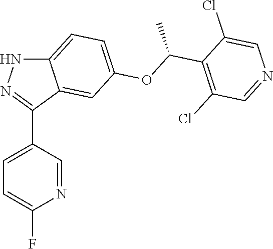 [SMILES: C[C@@H](Oc1ccc2[nH]nc(-c3ccc(F)nc3)c2c1)c1c(Cl)cncc1Cl]

(R)-5-(1-(3,5-
dichloropyridin-4-
yl)ethoxy)-3-(6-
fluoropyridin-3-yl)-
1H-indazole

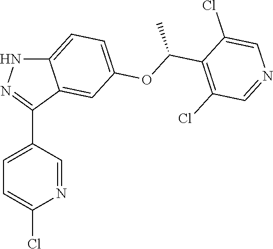 [SMILES: C[C@@H](Oc1ccc2[nH]nc(-c3ccc(Cl)nc3)c2c1)c1c(Cl)cncc1Cl]

(R)-3-(6-
chloropyridin-3-yl)-5-
(1-(3,5-
dichloropyridin-4-
yl)ethoxy)-1H-
indazole

In some aspects, the disclosure is directed to the following compounds, or pharmaceutically acceptable salts thereof:

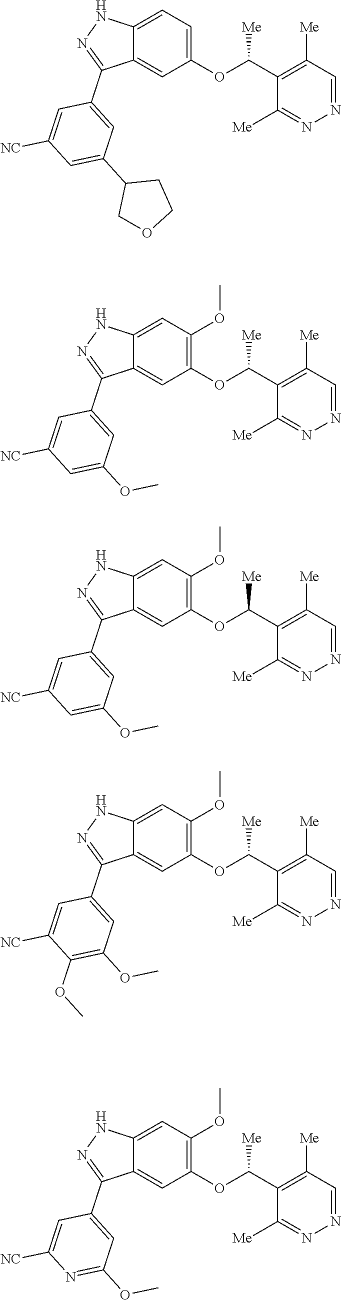 [SMILES: Cc1cnnc(C)c1[C@@H](C)Oc1ccc2[nH]nc(-c3cc(C#N)cc(C4CCOC4)c3)c2c1 ; COc1cc(C#N)cc(-c2n[nH]c3cc(OC)c(O[C@H](C)c4c(C)cnnc4C)cc23)c1 ; COc1cc(C#N)cc(-c2n[nH]c3cc(OC)c(O[C@@H](C)c4c(C)cnnc4C)cc23)c1 ; COc1cc2[nH]nc(-c3cc(C#N)c(OC)c(OC)c3)c2cc1O[C@H](C)c1c(C)cnnc1C ; COc1cc(-c2n[nH]c3cc(OC)c(O[C@H](C)c4c(C)cnnc4C)cc23)cc(C#N)n1]

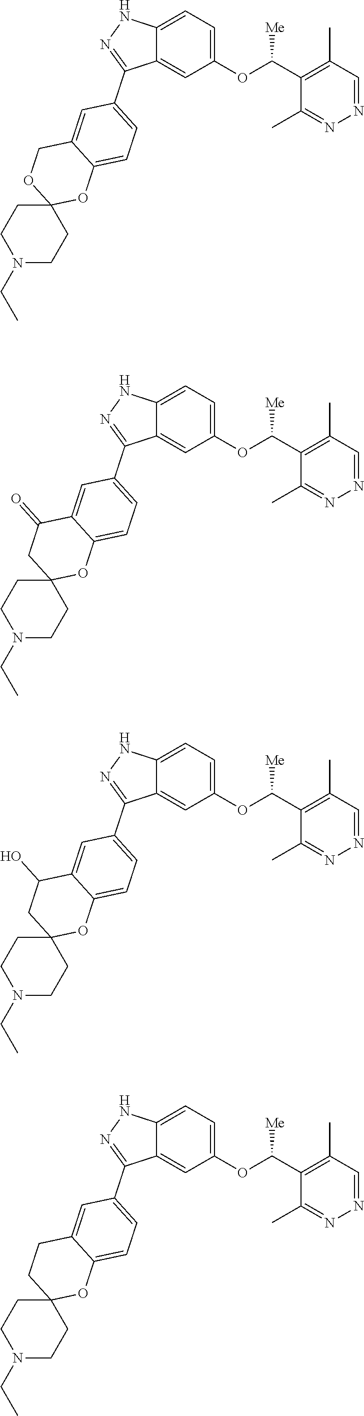 [SMILES: CCN1CCC2(CC1)OCc1cc(-c3n[nH]c4ccc(O[C@H](C)c5c(C)cnnc5C)cc34)ccc1O2 ; CCN1CCC2(CC1)CC(=O)c1cc(-c3n[nH]c4ccc(O[C@H](C)c5c(C)cnnc5C)cc34)ccc1O2 ; CCN1CCC2(CC1)CC(O)c1cc(-c3n[nH]c4ccc(O[C@H](C)c5c(C)cnnc5C)cc34)ccc1O2 ; CCN1CCC2(CCc3cc(-c4n[nH]c5ccc(O[C@H](C)c6c(C)cnnc6C)cc45)ccc3O2)CC1]

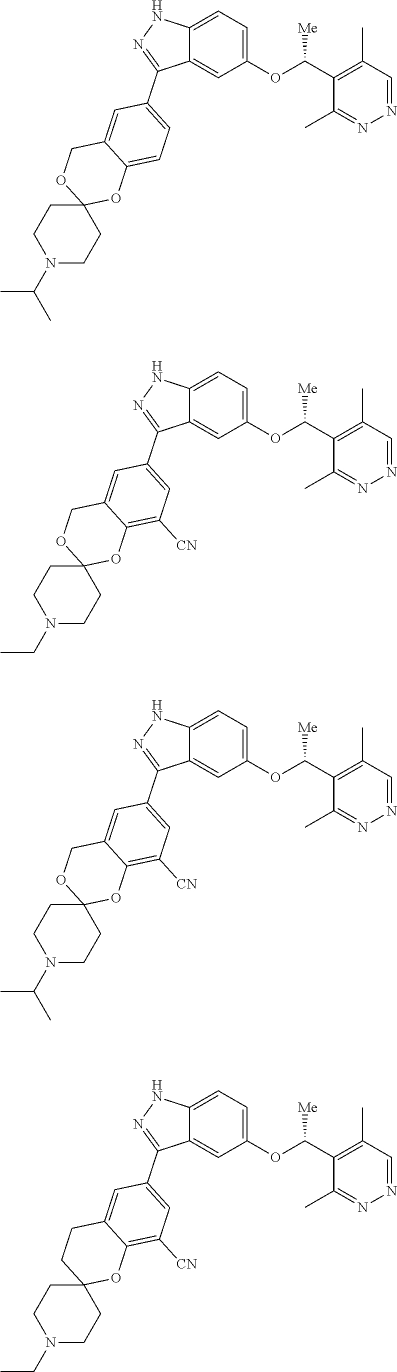 [SMILES: Cc1cnnc(C)c1[C@@H](C)Oc1ccc2[nH]nc(-c3ccc4c(c3)COC3(CCN(C(C)C)CC3)O4)c2c1 ; CCN1CCC2(CC1)OCc1cc(-c3n[nH]c4ccc(O[C@H](C)c5c(C)cnnc5C)cc34)cc(C#N)c1O2 ; Cc1cnnc(C)c1[C@@H](C)Oc1ccc2[nH]nc(-c3cc(C#N)c4c(c3)COC3(CCN(C(C)C)CC3)O4)c2c1 ; CCN1CCC2(CCc3cc(-c4n[nH]c5ccc(O[C@H](C)c6c(C)cnnc6C)cc45)cc(C#N)c3O2)CC1]

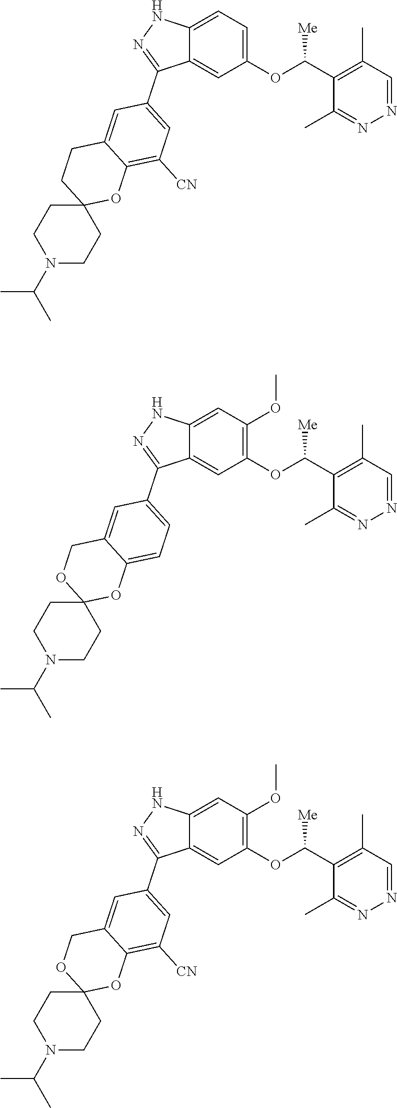 [SMILES: Cc1cnnc(C)c1[C@@H](C)Oc1ccc2[nH]nc(-c3cc(C#N)c4c(c3)CCC3(CCN(C(C)C)CC3)O4)c2c1 ; COc1cc2[nH]nc(-c3ccc4c(c3)COC3(CCN(C(C)C)CC3)O4)c2cc1O[C@H](C)c1c(C)cnnc1C ; COc1cc2[nH]nc(-c3cc(C#N)c4c(c3)COC3(CCN(C(C)C)CC3)O4)c2cc1O[C@H](C)c1c(C)cnnc1C]

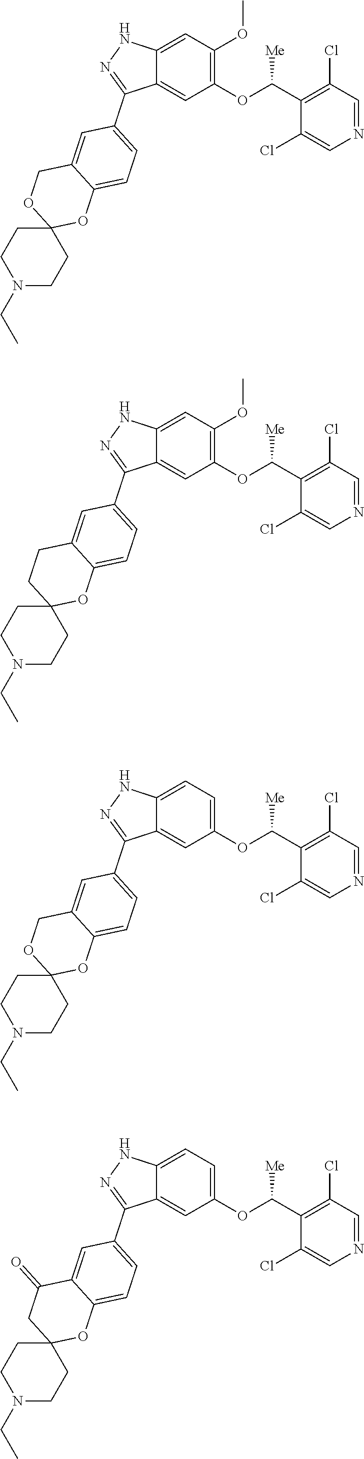 [SMILES: CCN1CCC2(CC1)OCc1cc(-c3n[nH]c4cc(OC)c(O[C@H](C)c5c(Cl)cncc5Cl)cc34)ccc1O2 ; CCN1CCC2(CCc3cc(-c4n[nH]c5cc(OC)c(O[C@H](C)c6c(Cl)cncc6Cl)cc45)ccc3O2)CC1 ; CCN1CCC2(CC1)OCc1cc(-c3n[nH]c4ccc(O[C@H](C)c5c(Cl)cncc5Cl)cc34)ccc1O2 ; CCN1CCC2(CC1)CC(=O)c1cc(-c3n[nH]c4ccc(O[C@H](C)c5c(Cl)cncc5Cl)cc34)ccc1O2]

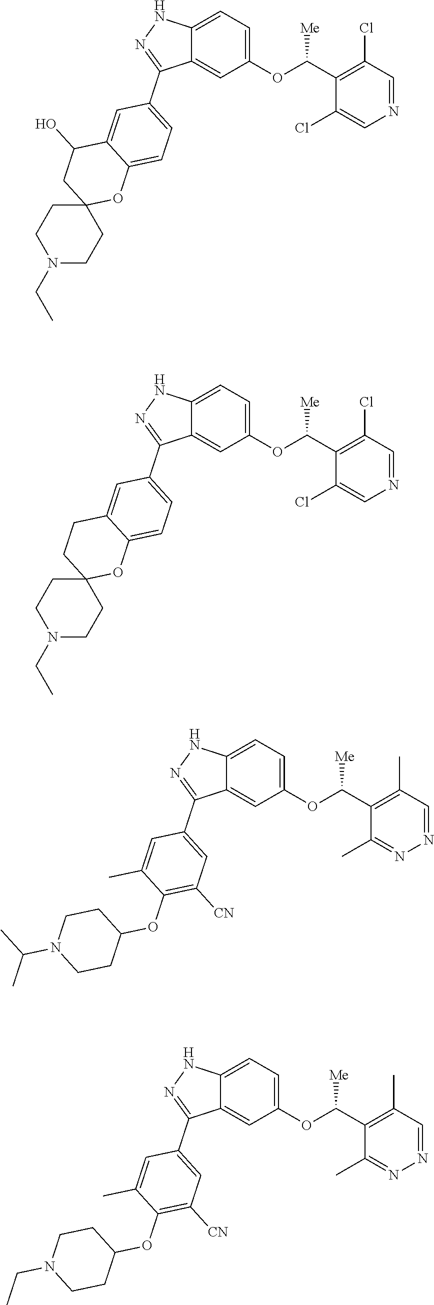 [SMILES: CCN1CCC2(CC1)CC(O)c1cc(-c3n[nH]c4ccc(O[C@H](C)c5c(Cl)cncc5Cl)cc34)ccc1O2 ; CCN1CCC2(CCc3cc(-c4n[nH]c5ccc(O[C@H](C)c6c(Cl)cncc6Cl)cc45)ccc3O2)CC1 ; Cc1cc(-c2n[nH]c3ccc(O[C@H](C)c4c(C)cnnc4C)cc23)cc(C#N)c1OC1CCN(C(C)C)CC1 ; CCN1CCC(Oc2c(C)cc(-c3n[nH]c4ccc(O[C@H](C)c5c(C)cnnc5C)cc34)cc2C#N)CC1]

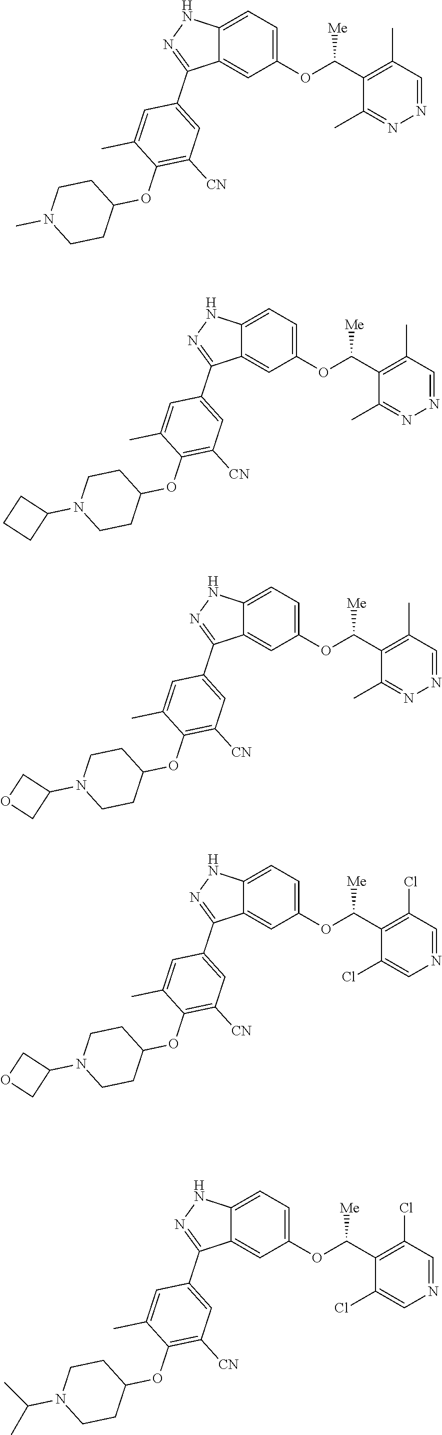 [SMILES: Cc1cc(-c2n[nH]c3ccc(O[C@H](C)c4c(C)cnnc4C)cc23)cc(C#N)c1OC1CCN(C)CC1 ; Cc1cc(-c2n[nH]c3ccc(O[C@H](C)c4c(C)cnnc4C)cc23)cc(C#N)c1OC1CCN(C2CCC2)CC1 ; Cc1cc(-c2n[nH]c3ccc(O[C@H](C)c4c(C)cnnc4C)cc23)cc(C#N)c1OC1CCN(C2COC2)CC1 ; Cc1cc(-c2n[nH]c3ccc(O[C@H](C)c4c(Cl)cncc4Cl)cc23)cc(C#N)c1OC1CCN(C2COC2)CC1 ; Cc1cc(-c2n[nH]c3ccc(O[C@H](C)c4c(Cl)cncc4Cl)cc23)cc(C#N)c1OC1CCN(C(C)C)CC1]

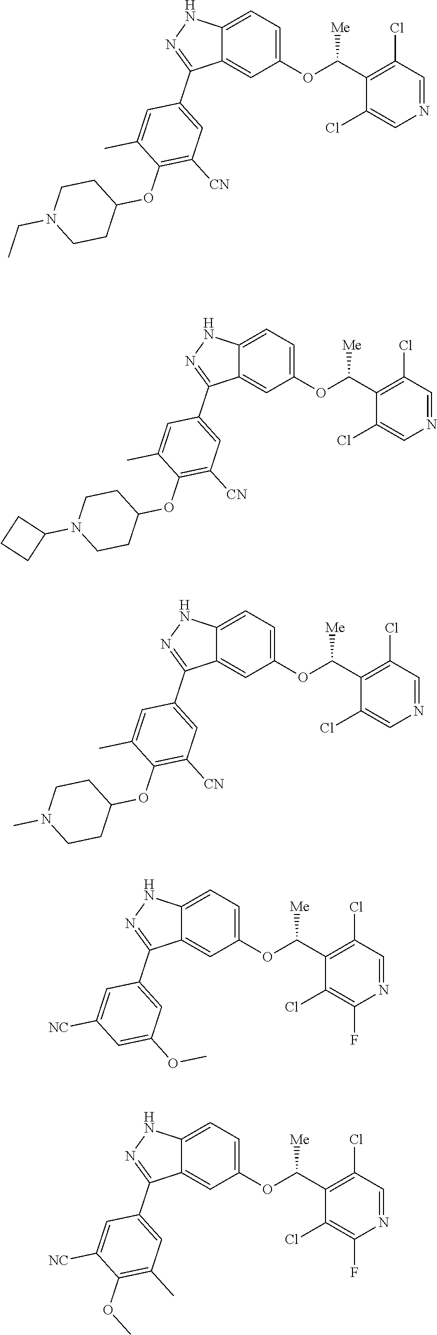 [SMILES: CCN1CCC(Oc2c(C)cc(-c3n[nH]c4ccc(O[C@H](C)c5c(Cl)cncc5Cl)cc34)cc2C#N)CC1 ; Cc1cc(-c2n[nH]c3ccc(O[C@H](C)c4c(Cl)cncc4Cl)cc23)cc(C#N)c1OC1CCN(C2CCC2)CC1 ; Cc1cc(-c2n[nH]c3ccc(O[C@H](C)c4c(Cl)cncc4Cl)cc23)cc(C#N)c1OC1CCN(C)CC1 ; COc1cc(C#N)cc(-c2n[nH]c3ccc(O[C@H](C)c4c(Cl)cnc(F)c4Cl)cc23)c1 ; COc1c(C)cc(-c2n[nH]c3ccc(O[C@H](C)c4c(Cl)cnc(F)c4Cl)cc23)cc1C#N]

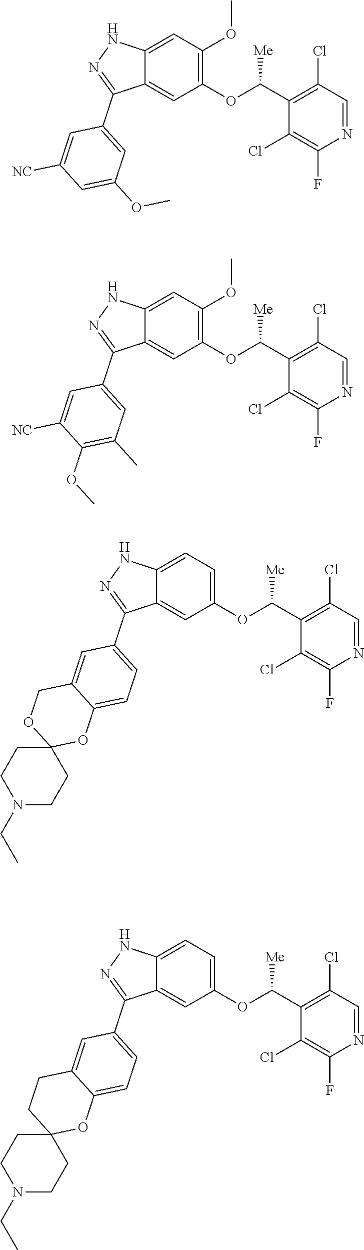 [SMILES: COc1cc(C#N)cc(-c2n[nH]c3cc(OC)c(O[C@H](C)c4c(Cl)cnc(F)c4Cl)cc23)c1 ; COc1cc2[nH]nc(-c3cc(C)c(OC)c(C#N)c3)c2cc1O[C@H](C)c1c(Cl)cnc(F)c1Cl ; CCN1CCC2(CC1)OCc1cc(-c3n[nH]c4ccc(O[C@H](C)c5c(Cl)cnc(F)c5Cl)cc34)ccc1O2 ; CCN1CCC2(CCc3cc(-c4n[nH]c5ccc(O[C@H](C)c6c(Cl)cnc(F)c6Cl)cc45)ccc3O2)CC1]

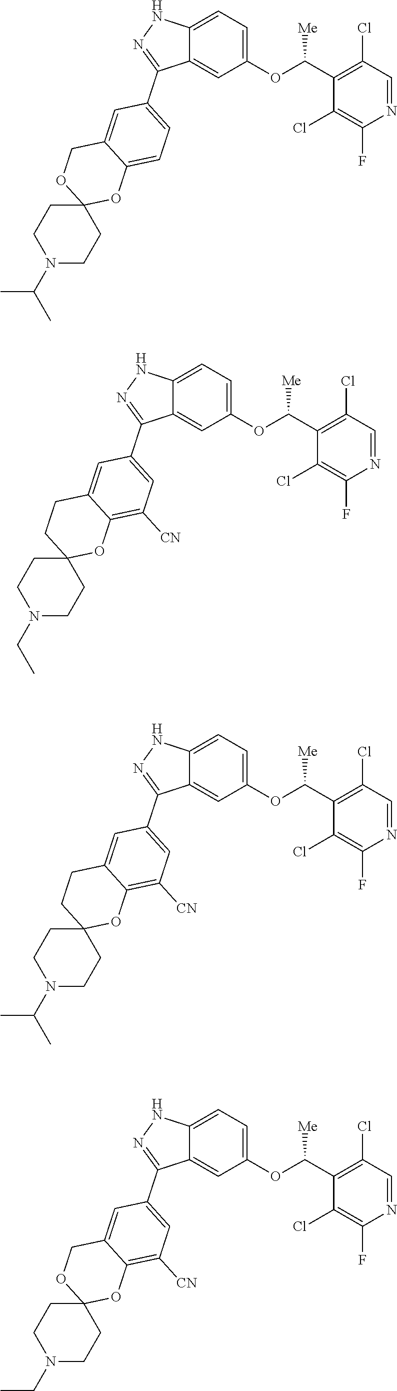 [SMILES: CC(C)N1CCC2(CC1)OCc1cc(-c3n[nH]c4ccc(O[C@H](C)c5c(Cl)cnc(F)c5Cl)cc34)ccc1O2 ; CCN1CCC2(CCc3cc(-c4n[nH]c5ccc(O[C@H](C)c6c(Cl)cnc(F)c6Cl)cc45)cc(C#N)c3O2)CC1 ; CC(C)N1CCC2(CCc3cc(-c4n[nH]c5ccc(O[C@H](C)c6c(Cl)cnc(F)c6Cl)cc45)cc(C#N)c3O2)CC1 ; CCN1CCC2(CC1)OCc1cc(-c3n[nH]c4ccc(O[C@H](C)c5c(Cl)cnc(F)c5Cl)cc34)cc(C#N)c1O2]

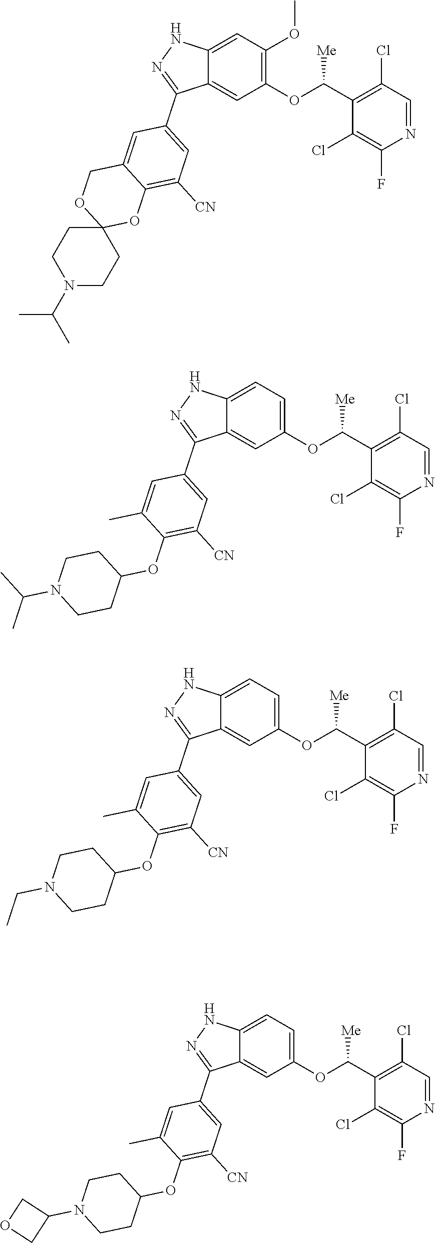 [SMILES: COc1cc2[nH]nc(-c3cc(C#N)c4c(c3)COC3(CCN(C(C)C)CC3)O4)c2cc1O[C@H](C)c1c(Cl)cnc(F)c1Cl ; Cc1cc(-c2n[nH]c3ccc(O[C@H](C)c4c(Cl)cnc(F)c4Cl)cc23)cc(C#N)c1OC1CCN(C(C)C)CC1 ; CCN1CCC(Oc2c(C)cc(-c3n[nH]c4ccc(O[C@H](C)c5c(Cl)cnc(F)c5Cl)cc34)cc2C#N)CC1 ; Cc1cc(-c2n[nH]c3ccc(O[C@H](C)c4c(Cl)cnc(F)c4Cl)cc23)cc(C#N)c1OC1CCN(C2COC2)CC1]

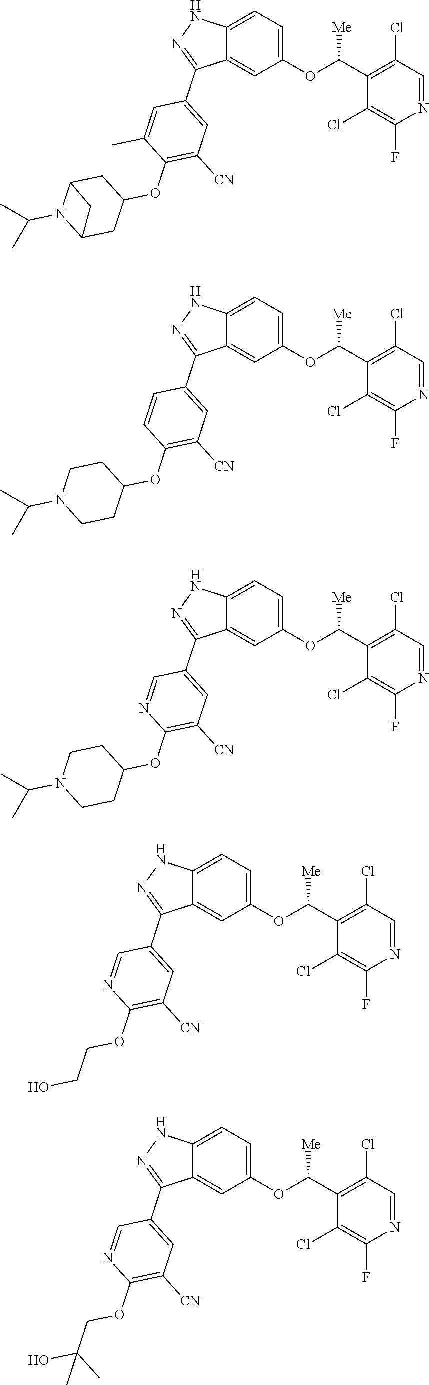 [SMILES: Cc1cc(-c2n[nH]c3ccc(O[C@H](C)c4c(Cl)cnc(F)c4Cl)cc23)cc(C#N)c1OC1CC2CC(C1)N2C(C)C ; CC(C)N1CCC(Oc2ccc(-c3n[nH]c4ccc(O[C@H](C)c5c(Cl)cnc(F)c5Cl)cc34)cc2C#N)CC1 ; CC(C)N1CCC(Oc2ncc(-c3n[nH]c4ccc(O[C@H](C)c5c(Cl)cnc(F)c5Cl)cc34)cc2C#N)CC1 ; C[C@@H](Oc1ccc2[nH]nc(-c3cnc(OCCO)c(C#N)c3)c2c1)c1c(Cl)cnc(F)c1Cl ; C[C@@H](Oc1ccc2[nH]nc(-c3cnc(OCC(C)(C)O)c(C#N)c3)c2c1)c1c(Cl)cnc(F)c1Cl]

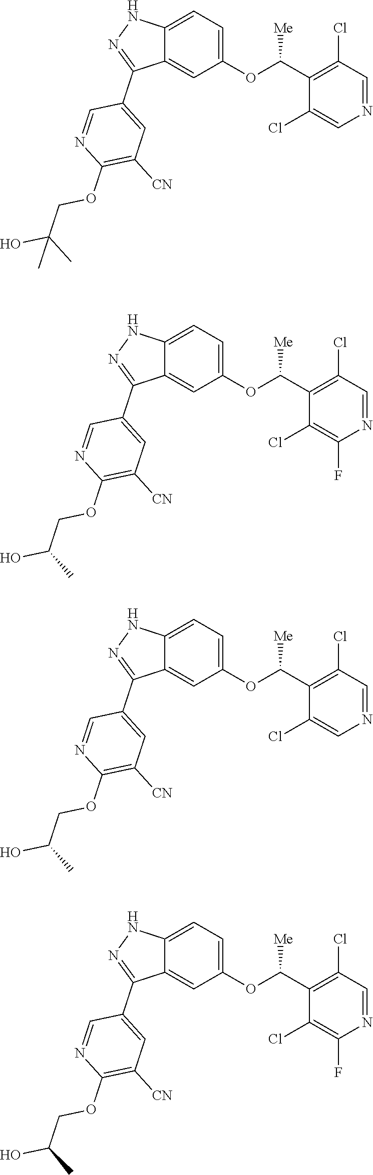 [SMILES: C[C@@H](Oc1ccc2[nH]nc(-c3cnc(OCC(C)(C)O)c(C#N)c3)c2c1)c1c(Cl)cncc1Cl ; C[C@H](O)COc1ncc(-c2n[nH]c3ccc(O[C@H](C)c4c(Cl)cnc(F)c4Cl)cc23)cc1C#N ; C[C@H](O)COc1ncc(-c2n[nH]c3ccc(O[C@H](C)c4c(Cl)cncc4Cl)cc23)cc1C#N ; C[C@@H](O)COc1ncc(-c2n[nH]c3ccc(O[C@H](C)c4c(Cl)cnc(F)c4Cl)cc23)cc1C#N]

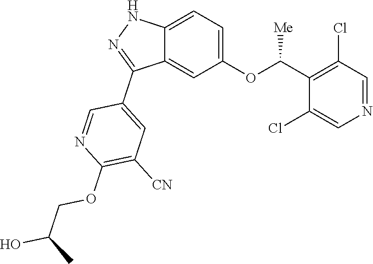 [SMILES: C[C@@H](O)COc1ncc(-c2n[nH]c3ccc(O[C@H](C)c4c(Cl)cncc4Cl)cc23)cc1C#N]

In some aspects, the disclosure is directed to the compounds shown in the Examples below, or pharmaceutically acceptable salts thereof.

References herein to formula (I) or subgenera thereof are meant to encompass the identified formula and any subgenera of those formula disclosed herein.

Stereoisomers of compounds of formula (I) are also contemplated by the present disclosure. Thus, the disclosure encompasses all stereoisomers and constitutional isomers of any compound disclosed or claimed herein, including all enantiomers and diastereomers, or mixtures thereof.

Pharmaceutically acceptable salts and solvates of the compounds of formula (I) are also within the scope of the disclosure.

Isotopic variants of the compounds of formula (I) are also contemplated by the present disclosure.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. While an embodiment may be described as part of a series of steps or part of a more general structure, each said step may also be considered an independent embodiment in itself, combinable with others

### Pharmaceutical Compositions and Methods of Administration

In some aspects, the disclosure is directed to pharmaceutical compositions comprising a compound of the disclosure, or a pharmaceutically acceptable salt thereof, and a pharmaceutically acceptable excipient.

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. In some embodiments, the pharmaceutical compositions contain a compound of the present disclosure or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25%18%, 17.75%, 17.50%, 17.25%17%, 16.75%, 16.50%, 16.25%, 16%, 15.75%, 15.50%, 15.25%15%, 14.75%, 14.50%, 14.25%14%, 13.75%, 13.50%, 13.25%, 13%, 12.75%, 12.50%, 12.25%, 12%, 11.75%, 11.50%, 11.25% 11%, 10.75%, 10.50%, 10.25%10%, 9.75%, 9.50%, 9.25%, 9%, 8.75%, 8.50%, 8.25%8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

Unless otherwise noted, the amounts of the compounds described herein are set forth on a free base basis. That is, the amounts indicate that amount of the compound administered, exclusive of, for example, solvent (such as in solvates) or counterions (such as in pharmaceutically acceptable salts).

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, -caprolactone and isomers thereof, 8-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%0, 50%), 100%, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%>to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical compositions for topical (e.g. transdermal) delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art.

See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intraarterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. poly hydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The FGFR receptors (FGFR1, FGFR2, FGFR3, and FGFR4) share several structural features in common, including three extracellular immunoglobulin-like (Ig) domains, a hydrophobic transmembrane domain, and an intracellular tyrosine kinase domain split by a kinase insert domain, followed by a cytoplasmic c-terminal tail (Johnson et al., Adv. Cancer Res. 60:1-40, 1993; and Wilkie et al., Curr. Biol. 5:500-507, 1995). In FGFR1, the kinase insert domain spans positions 582 to 595 of the alpha A1 isoform of FGFR1. In FGFR2, the kinase insert domain spans positions 585 to 598 of the FGFR2 Ille isoform. In FGFR3, the kinase insert domain spans positions 576 to 589 of the FGFR3 Ille isoform. In FGFR4, the kinase insert domain spans positions 571 to 584 of FGFR4 isoform 1. The c-terminal tail of FGFRs begins following the end of the tyrosine kinase domain and extends to the c-terminus of the protein. Several isoforms of each FGFR have been identified and are the result of alternative splicing of their mRNAs (Johnson et al., Mol. Cell. Biol. 11:4627-4634, 1995; and Chellaiah et al., J. Biol. Chem. 269:11620-11627, 1994).

Compounds of the disclosure have been found to inhibit FGFR1, FGFR2, FGFR3 and/or FGFR4. Preferred compounds of the disclosure will inhibit FGFR4. In other preferred embodiments, the compounds of the disclosure will be selective for FGFR4. For example, compounds of the disclosure can be useful in treating FGFR-associated diseases and disorders, e.g., proliferative disorders such as cancers, including hematological cancers and solid tumor, and angiogenesis-related disorders.

Compounds of the disclosure inhibit wild-type FGFR1, FGFR2, FGFR3, and/or FGFR4. In other aspects, compounds of the disclosure inhibit a mutated FGFR1, FGFR2, FGFR3, and/or FGFR4. In other aspects, compounds of the disclosure inhibit FGFR1, FGFR2, FGFR3, and/or FGFR4 that includes an FGFR kinase inhibitor mutation.

In some embodiments of any of the methods or uses described herein, the cancer (e.g., FGFR-associated cancer) is a hematological cancer. In some embodiments of any of the methods or uses described herein, the cancer (e.g., FGFR-associated cancer) is a solid tumor.

In some embodiments of any of the methods or uses described herein, the cancer (e.g., FGFR-associated cancer) is a lung cancer (e.g., small cell lung carcinoma, non-small cell lung carcinoma, squamous cell carcinoma, lung adenocarcinoma, large cell carcinoma, mesothelioma, lung neuroendocrine carcinoma, smoking-associated lung cancer), prostate cancer, colorectal cancer (e.g., rectal adenocarcinoma), endometrial cancer (e.g., endometrioid endometrial cancer, endometrial adenocarcinoma), breast cancer (e.g., hormone-receptor-positive breast cancer, triple-negative breast cancer, neuroendodrine carcinoma of the breast), skin cancer (e.g., melanoma, cutaneous squamous cell carcinoma, basal cell carcinoma, large squamous cell carcinoma), gallbladder cancer, liposarcoma (e.g., dedifferentiated liposarcoma, myxoid liposarcoma), pheochromocytoma, myoepithelial carcinoma, urothelial carcinoma, spermatocytic seminoma, stomach cancer, head and neck cancer (e.g., head and neck (squamous) carcinoma, head and neck adenoid cystic adenocarcinoma), brain cancer (e.g., glialneural tumors, glioma, neuroblastoma, glioblastoma, pilocytic astrocytoma, Rosette forming glioneural tumor, dysembryoplastic neuroepithelial tumor, anaplastic astrocytoma, medulloblastoma, ganglioglioma, oligodendroglioma), malignant peripheral nerve sheath tumor, sarcoma (e.g., soft tissue sarcoma (e.g., leiomyosarcoma), osteosarcoma), esophageal cancer (e.g., esophageal adenocarcinoma), lymphoma, bladder cancer (e.g., bladder urothelial (transition cell) carcinoma), cervical cancer (e.g., cervical squamous cell carcinoma, cervical adenocarcinoma), fallopian tube cancer (e.g., fallopian tube carcinoma), ovarian cancer (e.g., ovarian serous cancer, ovarian mucinous carcinoma), cholangiocarcinoma, adenoid cystic carcinoma, pancreatic cancer (e.g., pancreatic exocrine carcinoma, pancreatic ductal adenocarcinoma, pancreatic cancer intraepithelial neoplasia), salivary gland cancer (e.g., pleomorphic salivary gland adenocarcinoma, salivary adenoid cystic cancer), oral cancer (e.g., oral squamous cell carcinoma), uterine cancer, gastric or stomach cancer (e.g., gastric adenocarcinoma), gastrointestinal stromal tumors, myeloma (e.g., multiple myeloma), lymphoepithelioma, anal cancer (e.g., anal squamous cell carcinoma), prostate cancer (e.g., prostate adenocarcinoma), renal cell carcinoma, thymic cancer, gastroesophogeal junction adenocarcinoma, testicular cancer, rhabdomyosarcoma (e.g., alveolar rhabdomyosarcoma, embryonic rhabomyosarcoma), renal papillary carcinoma, liver cancer (e.g., hepatocellular carcinoma, intrahepatic cholangiocarcinoma), carcinoid, myeloid proliferative disorders (also called myeloid proliferative neoplasms (MPN); e.g., 8pll myeloproliferative syndrome (EMS, also called stem cell leukemia/lymphoma), acute myeloid leukemia (AML), chronic myeloid leukemia (CML)), lymphoma (e.g., T-cell lymphoma, T-lymphoblastic lymphoma, acute lymphoblastic leukemia (ALL), B-cell lymphoma), myeloid and lymphoid neoplasms, chronic neutrophilic leukemia, phosphaturic mesenchymal tumor, thyroid cancer (e.g. anaplastic thyroid carcinoma), or biliary duct cancer.

In some embodiments of any of the methods or uses described herein, the cancer (e.g., FGFR-associated cancer) is selected from the group of: acute lymphoblastic leukemia (ALL), acute myeloid leukemia (AML), cancer in adolescents, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, neoplasms by site, neoplasms, colon cancer, colorectal cancer, craniopharyngioma, cutaneous T-cell lymphoma, cutaneous angiosarcoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, thoracic neoplasms, head and neck neoplasms, CNS tumor, primary CNS tumor, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, neoplasms by site, neoplasms, myelogenous leukemia, myeloid leukemia, multiple myeloma, myeloproliferative neoplasms, nasal cavity and para nasal sinus cancer, nasopharyngeal cancer, neuroblastoma, non-Hodgkin's lymphoma, non-small cell lung cancer, lung neoplasm, pulmonary cancer, pulmonary neoplasms, respiratory tract neoplasms, bronchogenic carcinoma, bronchial neoplasms, oral cancer, oral cavity cancer, lip cancer, oropharyngeal cancer, osteosarcoma, ovarian cancer, pancreatic cancer, papillomatosis, paraganglioma, para nasal sinus and nasal cavity cancer, parathyroid cancer, penile cancer, pharyngeal cancer, pheochromosytoma, pituitary cancer, plasma cell neoplasm, pleuropulmonary blastoma, pregnancy-associated breast cancer, primary central nervous system lymphoma, primary peritoneal cancer, prostate cancer, rectal cancer, colon cancer, colonic neoplasms, renal cell cancer, retinoblastoma, rhabdomyosarcoma, salivary gland cancer, sarcoma, Sezary syndrome, skin cancer, Spitz tumors, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, and Wilms' tumor.

In some embodiments, a hematological cancer (e.g., hematological cancers that are FGFR associated cancers) is selected from the group consisting of leukemias, lymphomas (non-Hodgkin's lymphoma), Hodgkin's disease (also called Hodgkin's lymphoma), and myeloma, for instance, acute lymphocytic leukemia (ALL), acute myeloid leukemia (AML), acute promyelocytic leukemia (APL), chronic lymphocytic leukemia (CLL), chronic myeloid leukemia (CML), chronic myelomonocytic leukemia (CMML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult Tcell ALL, AML with trilineage myelodysplasia (AML/TMDS), mixed lineage leukemia (MLL), myelodysplastic syndromes (MDSs), myeloproliferative disorders (MPD), and multiple myeloma (MM).

Additional examples of hematological cancers include myeloproliferative disorders (MPD) such as polycythemia vera (PV), essential thrombocytopenia (ET) and idiopathic primary myelofibrosis (IMF/IPF/PMF). In some embodiments, the hematological cancer (e.g., the hematological cancer that is a FGFR-associated cancer) is AML or CMML.

In some embodiments, the cancer (e.g., the FGFR-associated cancer) is a solid tumor. Examples of solid tumors (e.g., solid tumors that are FGFR-associated cancers) include, for example, lung cancer (e.g., lung adenocarcinoma, non-small-cell lung carcinoma, squamous cell lung cancer), bladder cancer, colorectal cancer, brain cancer, testicular cancer, bile duct cancer cervical cancer, prostate cancer, and sparmatocytic seminomas. See, for example, Turner and Grose, Nat. Rev. Cancer. 10(2): 116-129, 2010.

In some aspects, the compounds of the disclosure are useful in treating cancers associated with amplification of FGFR4, for example, Rhabdomyosarcoma, prostate cancer or carcinoma, breast cancer, urothelial cancer, carcinoid, carcinoma of unknown primary, esophageal adenocarcinoma, head and neck carcinoma, hepatocellular carcinoma, non-small cell lung carcinoma, ovarian cancer, fallopian tube carcinoma, peritoneal carcinoma, renal cell carcinoma.

In some aspects, the compounds of the disclosure are useful in treating cancers associated with upregulation of activity of FGFR4, for example, Colorectal cancer, hepatocellular carcinoma, adrenal carcinoma, breast cancer.

In some aspects, the compounds of the disclosure are useful in treating cancers associated with overexpression of activity of FGFR4, for example, Pancreatic intraepithelial neoplasia, and pancreatic ductal adenocarcinoma.

In some aspects, the compounds of the disclosure are more selective for one FGFR than for another. As used herein, the “selectivity” of a compound for a first target over a second target means that the compound has more potent activity at the first target than the second target. A fold selectivity can be calculated by any method known in the art. For example, a fold selectivity can be calculated by dividing the IC50 value (or Kd value) of a compound for the second target (e.g., FGFR1) by the IC50 value of the same compound for the first target (e.g., FGFR2, FGFR3 or FGFR4). An IC50 value can be determined by any method known in the art. In some embodiments, a compound is first determined to have an activity of less than 500 nM for the first target. In some embodiments, a compound is first determined to have an activity of less than 500 nM for the second target.

For example, in some aspects, the compounds of the disclosure are more selective for FGFR4 than for FGFR1. In some aspects, the compounds are at least 3-fold more selective for FGFR4 than for FGFR1. In some aspects, the compounds are 4, 5, 6, 7, 8, 9, 10, 15, 20, 30, 40, 50, 75, 100, 200, 500, or 1000 fold more selective for FGFR3 than for FGFR1.

In some aspects, the compounds of the disclosure are more selective for an FGFR kinase over another kinase that is not an FGFR kinase. For example, the compounds of the disclosure are at least 3-fold more selective for an FGFR kinase over another kinase that is not an FGFR kinase. In some aspects, the compounds of the disclosure are at least 10, 20, 30, 40, 50, 60, 70, 80, 90, 200, 300, 400, 500, 600, 700, 800, 900, or at least 1000 fold more selective for an FGFR kinase over another kinase that is not an FGFR kinase. Kinases that are not FGFR kinases include, for example, KDR kinase and Aurora B kinase.

In some embodiments, the compounds of the disclosure exhibit brain and/or central nervous system (CNS) penetrance. Such compounds are capable of crossing the blood brain barrier and inhibiting a FGFR kinase in the brain and/or other CNS structures. In some embodiments, the compounds provided herein are capable of crossing the blood brain barrier in a therapeutically effective amount. For example, treatment of a subject with cancer (e.g., a FGFR-associated cancer such as a FGFR-associated brain or CNS cancer) can include administration (e.g., oral administration) of the compound to the subject. In some such embodiments, the compounds provided herein are useful for treating a primary brain tumor or metastatic brain tumor. For example, a FGFR-associated primary brain tumor or metastatic brain tumor.

In some embodiments, the compounds of the disclosure, exhibit one or more of high GI absorption, low clearance, and low potential for drug-drug interactions.

In some aspects, compounds of the disclosure can be used for treating a subject diagnosed with (or identified as having) a FGFR-associated disease or disorder (e.g., a FGFR-associated cancer) that include administering to the subject a therapeutically effective amount of a compound of the disclosure. Also provided herein are methods for treating a subject identified or diagnosed as having a FGFR-associated disease or disorder (e.g., a FGFR-associated cancer) that include administering to the subject a therapeutically effective amount of a compound of the disclosure. In some embodiments, the subject that has been identified or diagnosed as having a FGFR-associated disease or disorder (e.g., a FGFR-associated cancer) through the use of a regulatory agency-approved, e.g., FDA-approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit. In some embodiments, the FGFR-associated disease or disorder is a FGFR-associated cancer. For example, the FGFR-associated cancer can be a cancer that includes one or more FGFR inhibitor resistance mutations.

Also provided are methods for treating a disease or disorder in a subject in need thereof, the method comprising: (a) detecting a FGFR-associated disease or disorder in the subject; and (b) administering to the subject a therapeutically effective amount of a compound of the disclosure. Some embodiments of these methods further include administering to the subject an additional therapy or therapeutic agent (e.g., a second FGFR inhibitor, a second compound of the disclosure, or an immunotherapy. In some embodiments, the subject was previously treated with a first FGFR inhibitor or previously treated with another treatment. In some embodiments, the subject is determined to have a FGFR-associated disease or disorder through the use of a regulatory agency-approved, e.g., FDA approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a FGFR-associated cancer in the subject; and (b) administering to the subject a therapeutically effective amount of a compound of the disclosure. Some embodiments of these methods further include administering to the subject an additional therapy or therapeutic agent (e.g., a second FGFR inhibitor, a second compound of the disclosure, or an immunotherapy). In some embodiments, the subject was previously treated with a first FGFR inhibitor or previously treated with another anticancer treatment, e.g., at least partial resection of the tumor or radiation therapy. In some embodiments, the subject is determined to have a FGFR-associated cancer through the use of a regulatory agency-approved, e.g., FDA-approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is a FGFR associated cancer. For example, the FGFR-associated cancer can be a cancer that includes one or more FGFR inhibitor resistance mutations. In some embodiments, the cancer is a FGFR associated cancer. For example, the FGFR-associated cancer can be a cancer that includes one or more FGFR activating mutations.

Also provided are methods of treating a subject that include performing an assay on a sample obtained from the subject to determine whether the subject has a dysregulation of a FGFR gene, a FGFR kinase, or expression or activity or level of any of the same, and administering (e.g., specifically or selectively administering) a therapeutically effective amount of a compound of the disclosure or pharmaceutically acceptable salt or solvate thereof to the subject determined to have a dysregulation of a FGFR gene, a FGFR kinase, or expression or activity or level of any of the same. Some embodiments of these methods further include administering to the subject an additional therapy or therapeutic agent (e.g., a second FGFR inhibitor, a second compound of the disclosure, or immunotherapy). In some embodiments of these methods, the subject was previously treated with a first FGFR inhibitor or previously treated with another anticancer treatment, e.g., at least partial resection of a tumor or radiation therapy. In some embodiments, the subject is a subject suspected of having a FGFR-associated disease or disorder (e.g., a FGFR-associated cancer), a subject presenting with one or more symptoms of a FGFR-associated disease or disorder (e.g., a FGFR-associated cancer), or a subject having an elevated risk of developing a FGFR-associated disease or disorder (e.g., a FGFR-associated cancer). In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art. In some embodiments, the dysregulation of a FGFR gene, a FGFR kinase, or expression or activity or level of any of the same includes one or more FGFR inhibitor resistance mutations.

Also provided herein are methods of selecting a treatment for a subject, wherein the methods include a step of performing an assay on a sample obtained from the subject to determine whether the subject has a dysregulation of a FGFR gene, a FGFR kinase, or expression or activity or level of any of the same (e.g., one or more FGFR inhibitor resistance mutations), and identifying or diagnosing a subject determined to have a dysregulation of a FGFR gene, a FGFR kinase, or expression or activity or level of any of the same, as having a FGFR-associated cancer. Some embodiments further include administering the selected treatment to the subject identified or diagnosed as having a FGFR-associated cancer. For example, in some embodiments, the selected treatment can include administration of a therapeutically effective amount of a compound of the disclosure to the subject identified or diagnosed as having a FGFR-associated cancer. In some embodiments, the assay is an in vitro assay. For example, an assay that utilizes the next generation sequencing, immunohistochemistry, or break apart FISH analysis. In some embodiments, the assay is a regulatory agency-approved, e.g., FDA-approved, kit. In some embodiments, the assay is a liquid biopsy.

Also provided herein are methods of treating a FGFR-associated cancer in a subject that include (a) administering one or more (e.g., two or more, three or more, four or more, five or more, or ten or more) doses of a first FGFR kinase inhibitor to a subject identified or diagnosed as having a FGFR associated cancer (e.g., any of the types of FGFR-associated cancers described herein) (e.g., identified or diagnosed as having a FGFR-associated cancer using any of the exemplary methods described herein or known in the art); (b) after step (a), determining a level of circulating tumor DNA in a biological sample (e.g., a biological sample comprising blood, serum, or plasma) obtained from the subject; (c) administering a therapeutically effective amount of a second FGFR inhibitor or a compound of the disclosure as a monotherapy or in conjunction with an additional therapy or therapeutic agent to a subject identified as having about the same or an elevated level of circulating tumor DNA as compared to a reference level of circulating tumor DNA (e.g., any of the reference levels of circulating tumor DNA described herein). In some examples of these methods, the reference level of circulating tumor DNA is a level of circulating tumor DNA in a biological sample obtained from the subject prior to step (a). Some embodiments of these methods further include determining the level of circulating tumor DNA in the biological sample obtained from the subject prior to step (a). In some examples of these methods, the reference level of circulating tumor DNA is a threshold level of circulating tumor DNA (e.g., an average level of circulating tumor DNA in a population of subjects having a similar FGFR-associated cancer and having a similar stage of the FGFR-associated cancer, but receiving a non-effective treatment or a placebo, or not yet receiving therapeutic treatment, or a level of circulating tumor DNA in a subject having a similar FGFR-associated cancer and having a similar stage of the FGFR-associated cancer, but receiving a non-effective treatment or a placebo, or not yet receiving therapeutic treatment). In some examples of these methods, the first FGFR inhibitor is: ARQ-087, ASP5878, AZD4547, B-701, BAY1179470, BAY1187982, BGJ398, brivanib, Debio 1347, dovitinib, E7090, erdafitinib, FPA144, HMPL-453, INCB054828, lenvatinib, lucitanib, LY3076226, MAX-40279, nintedanib, orantinib, pemigatinib, ponatinib, PRN1371, rogaratinib, sulfatinib, futibatinib or RLY-4008.

Compounds of the disclosure can also be administered with additional therapy or therapeutic agents. In some aspects, the additional therapy or therapeutic agent includes one or more of radiation therapy, a chemotherapeutic agent (e.g., any of the exemplary chemotherapeutic agents described herein or known in the art), a checkpoint inhibitor (e.g., any of the exemplary checkpoint inhibitors described herein or known in the art), surgery (e.g., at least partial resection of the tumor), and one or more other kinase inhibitors (e.g., any of the kinase inhibitors described herein or known in the art).

Compounds of the disclosure may also be useful as adjuvants to cancer treatment, that is, they can be used in combination with one or more additional therapies or therapeutic agents, for example a chemotherapeutic agent that works by the same or by a different mechanism of action. In some embodiments, a compound of the disclosure can be used prior to administration of an additional therapeutic agent or additional therapy. For example, a subject in need thereof can be administered one or more doses of a compound of the disclosure for a period of time and then under go at least partial resection of the tumor. In some embodiments, the treatment with one or more doses of a compound of the disclosure reduces the size of the tumor (e.g., the tumor burden) prior to the at least partial resection of the tumor. In some embodiments, a subject has a cancer (e.g., a locally advanced or metastatic tumor) that is refractory or intolerant to standard therapy (e.g., administration of a chemotherapeutic agent, such as a first FGFR inhibitor or a multikinase inhibitor, immunotherapy, radiation, or a platinum-based agent (e.g., cisplatin)). In some embodiments, a subject has a cancer (e.g., a locally advanced or metastatic tumor) that is refractory or intolerant to prior therapy (e.g., administration of a chemotherapeutic agent, such as a first FGFR inhibitor or a multikinase inhibitor, immunotherapy, radiation, or a platinum-based agent (e.g., cisplatin)).

In some embodiments of any the methods described herein, the compound of the disclosure is administered in combination with a therapeutically effective amount of at least one additional therapeutic agent selected from one or more additional therapies or therapeutic (e.g., chemotherapeutic) agents. Non-limiting examples of additional therapeutic agents include: other FGFR-targeted therapeutic agents (i.e. a first or second FGFR kinase inhibitor), other kinase inhibitors (e.g., receptor tyrosine kinase targeted therapeutic agents (e.g., Trk inhibitors or EGFR inhibitors)), signal transduction pathway inhibitors, checkpoint inhibitors, modulators of the apoptosis pathway (e.g. obataclax); cytotoxic chemotherapeutics, angiogenesis-targeted therapies, immune-targeted agents, including immunotherapy, and radiotherapy.

Also provided herein are methods of treating a disease or disorder, comprising administering to a subject in need thereof a pharmaceutical combination for treating the disease or disorder which comprises (a) a compound of the disclosure, (b) an additional therapeutic agent, and (c) optionally at least one pharmaceutically acceptable carrier for simultaneous, separate or sequential use for the treatment of the disease or disorder, wherein the amounts of the compound of the disclosure and the additional therapeutic agent are together effective in treating the disease or disorder. In some embodiments, the compound of the disclosure, and the additional therapeutic agent are administered simultaneously as separate dosages. In some embodiments, the compound of the disclosure, and the additional therapeutic agent are administered as separate dosages sequentially in any order, in jointly therapeutically effective amounts, e.g. in daily or intermittently dosages. In some embodiments, the compound of the disclosure, and the additional therapeutic agent are administered simultaneously as a combined dosage. In some embodiments, the disease or disorder is a FGFR-associated disease or disorder. In some embodiments, the subject has been administered one or more doses of a compound of of the disclosure, prior to administration of the pharmaceutical composition.

In some embodiments, the treatment period is at least 7 days (e.g., at least or about 8 days, at least or about 9 days, at least or about 10 days, at least or about 11 days, at least or about 12 days, at least or about 13 days, at least or about 14 days, at least or about 15 days, at least or about 16 days, at least or about 17 days, at least or about 18 days, at least or about 19 days, at least or about 20 days, at least or about 21 days, at least or about 22 days, at least or about 23 days, at least or about 24 days, at least or about 25 days, at least or about 26 days, at least or about 27 days, at least or about 28 days, at least or about 29 days, or at least or about 30 days).

In some embodiments, the treatment period is at least 21 days (e.g., at least or about 22 days, at least or about 23 days, at least or about 24 days, at least or about 25 days, at least or about 26 days, at least or about 27 days, at least or about 28 days, at least or about 29 days, at least or about 30 days, at least or about 31 days, at least or about 32 days, at least or about 33 days, at least or about 34 days, at least or about 35 days, at least or about 36 days, at least or about 37 days, at least or about 38 days, at least or about 39 days, or at least or about 40) days).

Also provided herein are pharmaceutical compositions that contain, as the active ingredient, a compound of the disclosure, in combination with one or more pharmaceutically acceptable carriers (excipients). In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is formulated as a tablet or capsule.

The compositions comprising a compound of the disclosure can be formulated in a unit dosage form, each dosage containing from about 5 to about 1,000 mg (1 g), more usually about 100 mg to about 500 mg, of the active ingredient. The term “unit dosage form” refers to physically discrete units for human subjects and other subjects, each unit containing a predetermined quantity of active material (i.e., a compound of the disclosure) to produce the desired therapeutic effect, with a suitable pharmaceutical excipient.

In some embodiments, the compositions provided herein contain from about 5 mg to about 50 mg of the active ingredient, i.e., the compound of the disclosure. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient. In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850) mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80) mg/kg, about 85 mg/kg, about 90 mg/kg, about 95 mg/kg, or about 100 mg/kg. In some embodiments, such administration can be once-daily or twice-daily (BID) administration.

### EXAMPLES

The examples and preparations provided below further illustrate and exemplify the compounds of the present invention and methods of preparing such compounds. It is to be understood that the scope of the present invention is not limited in any way by the scope of the following examples and preparations.

In several embodiments, where single enantiomers are provided, the enantiomers may be separated by conventional means (chiral chromatography, preparing diastereomeric salts, chiral derivatization, crystallization, enzymatic reactions, etc.). In several embodiments, a chiral intermediate compound is purified to prepare an enantiomerically pure (or substantially enantiomerically pure, enantiomerically enriched, etc.) intermediate.

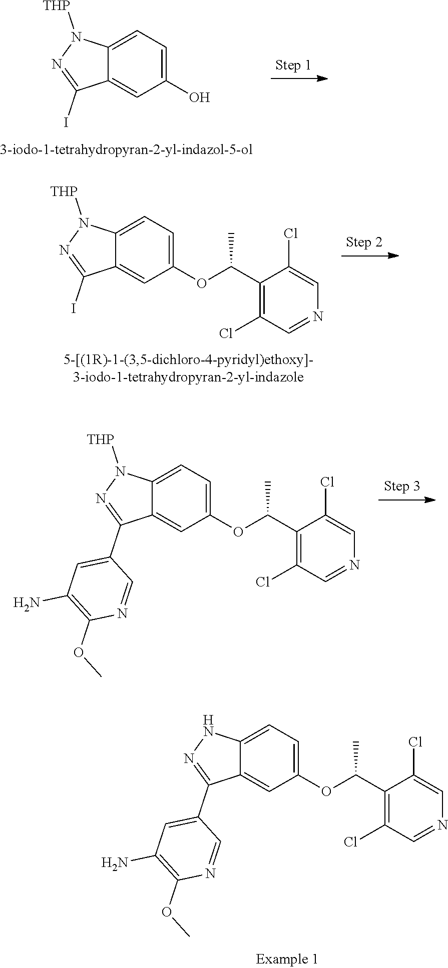

### Example 1. 5-[5-[(1R)-1-(3,5-dichloro-4-pyridyl)ethoxy]-1H-indazol-3-yl]-2-methoxy-pyridin-3-amine

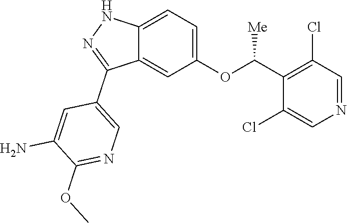 [SMILES: COc1ncc(-c2n[nH]c3ccc(O[C@H](C)c4c(Cl)cncc4Cl)cc23)cc1N]

Step 1. 5-((R)-1-(3,5-Dichloropyridin-4-yl)ethoxy)-3-iodo-1-(tetrahydro-2H-pyran-2-yl)-1H-indazole. A mixture of 3-iodo-1-tetrahydropyran-2-yl-indazol-5-ol (1.0 g, 2.90 mmol, 1.0 equiv), [(1S)-1-(3,5-dichloro-4-pyridyl)ethyl]methanesulfonate (780 mg, 2.90 mmol, 1.0 equiv) and cesium carbonate (1.41 g, 14.45 mmol, 1.5 equiv) in N,N-dimethylformamide (20 mL) was heated at 130° C. for 16 h. The volatiles were removed under reduced pressure and the residue was suspended in saturated ammonium chloride (50 mL). The solution was extracted with ethyl acetate (3×50 mL). The combined organic layers were dried over sodium sulfate, filtered and concentrated under reduced pressure. The crude product was purified on a Büchi automated chromatography system (Sorbtech 40 g silica gel column), eluting with a gradient of 0 to 30% ethyl acetate in heptanes to give a white solid (1.01 g, 88% yield). Analysis: LCMS m/z=517.2 (M+H).

Step 2. 5-(5-((R)-1-(3,5-Dichloropyridin-4-yl)ethoxy)-1-(tetrahydro-2H-pyran-2-yl)-1H-indazol-3-yl)-2-methoxypyridin-3-amine. A solution of 5-[(1R)-1-(3,5-dichloro-4-pyridyl)ethoxy]-3-iodo-1-tetrahydropyran-2-yl-indazole (1.0 g, 1.93 mmol, 1 equiv), 2-methoxy-5-(4,4,5,5-tetramethyl-1,3,2-dioxaborolan-2-yl)pyridin-3-amine (580 mg, 2.32 mmol, 1.2 equiv), [1,1′-bis(diphenylphosphino)ferrocene]dichloropalladium(II) (140 mg, 0.20 mmol, 0.1 equiv) and potassium carbonate (530 mg, 3.86 mmol, 2 equiv) in 1,4-dioxane (20 mL) and water (2 mL) was sparged with nitrogen for 15 minutes and then heated at 100° C. for 16 hours. After cooling to room temperature, the reaction mixture was filtered through Celite and the filtrate was concentrated under reduced pressure. The residue was purified on a Büchi automated chromatography system (Sorbtech 40 g silica gel column), eluting with a gradient of 0 to 70% ethyl acetate in heptanes to give a brown oil (900 mg, 91% yield). Analysis: LCMS m/z=514.1 (M+H).

Step 3. (R)-5-(5-(1-(3,5-Dichloropyridin-4-yl)ethoxy)-1H-indazol-3-yl)-2-methoxypyridin-3-amine. Product step 2 (137 mg, 0.27 mmol, 1 equiv) was treated with a 1 to 1 mixture of dichloromethane and trifluoracetic acid (4 mL) at room temperature for 2 hours. The volatiles were removed under reduced pressure. The crude was diluted with saturated sodium bicarbonate (10 mL) and extracted with ethyl acetate (3×20 mL). The combined organic layers were dried over sodium sulfate, filtered and concentrated under reduced pressure. The residue was purified on a Büchi automated chromatography system (RediSep Rf Gold HP C18, 50 g column), eluting with a gradient of 0 to 100% acetonitrile in water. The clean product fractions were lyophilized to give a white solid (50 mg, 64%). Analysis: LCMS m/z=430.1 (M+H); ¹H NMR (400 MHZ, CD3OD) δ=8.47 (s, 2H), 7.79 (d, J=2.1 Hz, 1H), 7.46-7.40 (m, 1H), 7.35 (d, J=2.1 Hz, 1H), 7.19-7.12 (m, 2H), 6.14 (q, J=6.7 Hz, 1H), 4.05 (s, 3H), 1.82 (d, J=6.7 Hz, 3H).

### Example 2. N-(5-(5-((R)-1-(3,5-Dichloropyridin-4-yl)ethoxy)-1-(tetrahydro-2H-pyran-2-yl)-1H-indazol-3-yl)-2-methoxypyridin-3-yl)picolinamide

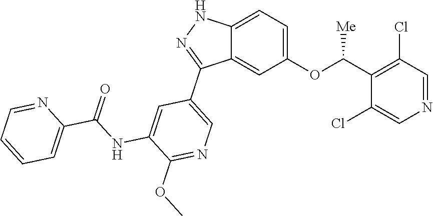 [SMILES: COc1ncc(-c2n[nH]c3ccc(O[C@H](C)c4c(Cl)cncc4Cl)cc23)cc1NC(=O)c1ccccn1]

Step 1. A solution of example 1 step 2 (86 mg, 0.70 mmol, 2.4 equiv) in N,N-dimethylformamide (3 mL) was treated with 1-[bis(dimethylamino)-methylene]-1H-1,2,3-triazolo[4,5-b]pyridinium 3-oxid hexafluorophosphate (HATU) (266 mg, 0.70 mmol, 2.4 equiv) and N,N diisopropylethylamine (0.3 mL, 1.34 mmol, 6.0 equiv) at room temperature for 20 minutes, followed by the addition of pyridine-2-carbonyl chloride (150 mg, 0.29 mmol, 1 equiv). After stirring at room temperature for 16 hours, water (5 mL) was added and the resulting solids were stirred for 30 minutes at room temperature. The solids were filtered to give an off-white solid, which was used directly in the next step (140 mg, 72%). Analysis: LCMS m/z=619.2 (M+H).

Step 2. (R)—N-(5-(5-(1-(3,5-Dichloropyridin-4-yl)ethoxy)-1H-indazol-3-yl)-2-methoxypyridin-3-yl)picolinamide. Product step 1 (140 mg, 0.22 mmol, 1.0 equiv) was treated with a 1 to 1 mixture of dichloromethane-trifluoracetic acid (2.8 mL) at room temperature for 2 hours. The volatiles were removed under reduced pressure. The residue was diluted with saturated sodium bicarbonate (5 mL) and extracted with dichloromethane (2×10 mL). The combined organic layers were dried over sodium sulfate, filtered and concentrated under reduced pressure. The residue was purified on a Büchi automated chromatography system (RediSep Rf Gold HP C18, 50 g column), eluting with a gradient of 0 to 100% acetonitrile in water. Product containing fractions were lyophilized to give a yellow solid (54.0 mg, 44%). Analysis: LCMS m/z=535.1 (M+H); ¹H NMR (400 MHZ, DMSO-d6) δ=13.22 (br s, 1H), 10.43 (s, 1H), 9.18 (d, J=2.1 Hz, 1H), 8.81 (ddd, J=1.0, 1.6, 4.8 Hz, 1H), 8.55 (s, 2H), 8.35 (d, J=2.2 Hz, 1H), 8.25 (td, J=1.1, 7.8 Hz, 1H), 8.15 (dt, J=1.6, 7.7 Hz, 1H), 7.75 (ddd, J=1.2, 4.8, 7.6 Hz, 1H), 7.52 (d, J=9.0 Hz, 1H), 7.30 (d, J=2.1 Hz, 1H), 7.13 (dd, J=2.3, 9.0 Hz, 1H), 6.13 (q, J=6.7 Hz, 1H), 4.12 (s, 3H), 1.76 (d, J=6.6 Hz, 3H).

### Example 3. (R)—N-(5-(5-(1-(3,5-Dichloropyridin-4-yl)ethoxy)-1H-indazol-3-yl)-2-methoxypyridin-3-yl)quinoline-8-carboxamide

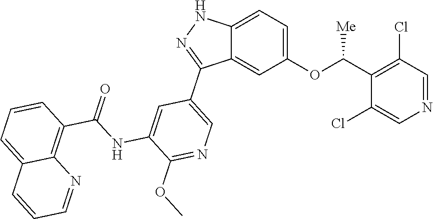 [SMILES: COc1ncc(-c2n[nH]c3ccc(O[C@H](C)c4c(Cl)cncc4Cl)cc23)cc1NC(=O)c1cccc2cccnc12]

Step 1. N-(5-(5-((R)-1-(3,5-Dichloropyridin-4-yl)ethoxy)-1-(tetrahydro-2H-pyran-2-yl)-1H-indazol-3-yl)-2-methoxypyridin-3-yl)quinoline-8-carboxamide. A solution of example 1 step 2 (154 mg, 0.29 mmol, 1 equiv) in pyridine (2 mL) was treated with quinoline-8-carbonyl chloride (97 mg, 0.43 mmol, 1.5 equiv) at room temperature for 16 hours. The volatiles were removed under reduced pressure. The residue was diluted with saturated brine (10 mL) and extracted with ethyl acetate (2×15 mL). The combined organic layers were dried over sodium sulfate, filtered and concentrated under reduced pressure to give a pale-yellow oil, which was used directly in the next step (130 mg). Analysis: LCMS m/z=535.1 (M+H).

Step 2. (R)—N-(5-(5-(1-(3,5-Dichloropyridin-4-yl)ethoxy)-1H-indazol-3-yl)-2-methoxypyridin-3-yl)quinoline-8-carboxamide. Product step 1 (130 mg, 0.19 mmol, 1 equiv) was treated with a 1 to 1 mixture of dichloromethane and trifluoracetic acid (2.6 mL) at room temperature for 2 hours. The volatiles were removed under reduced pressure. The residue was diluted with saturated sodium bicarbonate (5 mL) and extracted with dichloromethane (2×10 mL). The combined organic layers were dried over sodium sulfate, filtered and concentrated under reduced pressure. The residue was purified on a Büchi automated chromatography system (RediSep Rf Gold HP C18, 50 g column), eluting with a gradient of 0 to 100% acetonitrile in water. Product containing fractions were lyophilized to give a yellow solid (71.0 mg, 62.0%). Analysis: LCMS m/z=585.2 (M+H); ¹H NMR (400 MHz, DMSO-d6) δ=14.23 (s, 1H), 13.19 (s, 1H), 9.37 (d, J=2.2 Hz, 1H), 9.23 (dd, J=1.7, 4.3 Hz, 1H), 8.82 (dd, J=1.6, 7.3 Hz, 1H), 8.68 (dd, J=1.8, 8.4 Hz, 1H), 8.54 (s, 2H), 8.33 (dd, J=1.5, 8.2 Hz, 1H), 8.31 (d, J=2.2 Hz, 1H), 7.88 (t, J=7.8 Hz, 1H), 7.80 (dd, J=4.2, 8.4 Hz, 1H), 7.52 (d, J=8.9 Hz, 1H), 7.32 (d, J=1.8 Hz, 1H), 7.13 (dd, J=2.3, 9.0 Hz, 1H), 6.14 (q, J=6.6 Hz, 1H), 4.22 (s, 3H), 1.77 (d, J=6.6 Hz, 3H).

### Example 4. (R)-5-(5-(1-(3,5-Dichloropyridin-4-yl)ethoxy)-1H-indazol-3-yl)-2-methoxy-N-(pyridin-2-ylmethyl)pyridin-3-amine

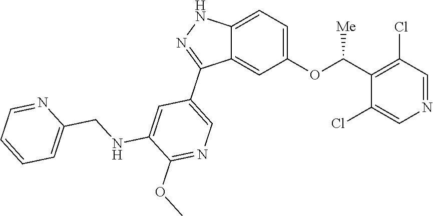 [SMILES: COc1ncc(-c2n[nH]c3ccc(O[C@H](C)c4c(Cl)cncc4Cl)cc23)cc1NCc1ccccn1]

Step 1. 5-(5-((R)-1-(3,5-Dichloropyridin-4-yl)ethoxy)-1-(tetrahydro-2H-pyran-2-yl)-1H-indazol-3-yl)-2-methoxy-N-(pyridin-2-ylmethyl)pyridin-3-amine. A mixture of product example 1 step 2 (0.15 g, 0.29 mmol, 1 equiv), pyridine-2-carboxaldehyde (0.105 g, 0.98 mmol, 3.3 equiv) and sodium triacetoxy borohydride (0.36 g, 1.74 mmol, 6 equiv) in dichloromethane (4 mL) was stirred at room temperature for 2.5 days. After completion, the reaction mixture was partitioned between saturated sodium carbonate (15 mL) and dichloromethane (20 mL). The aqueous layer was extracted with dichloromethane (2×15 mL). The combined organic layers were washed with saturated brine (20 mL), dried over sodium sulfate, filtered and concentrated under reduced pressure. The crude product was purified on a Büchi automated chromatography system (Biotage Sfär 25 g, 25 μm silica gel column), eluting with a gradient of 0 to 100% ethyl acetate in heptanes to give a light-yellow oil (97 mg, 55%). Analysis: LCMS m/z=605 (M+H); ¹H NMR (400 MHZ, CDCl3) δ=8.64-8.60 (m, 1H), 8.40 (d, J=0.9 Hz, 2H), 7.93 (d, J=1.7 Hz, 1H), 7.65 (dt, J=1.8, 7.7 Hz, 1H), 7.46 (dd, J=1.5, 9.0 Hz, 1H), 7.35 (d, J=7.8 Hz, 1H), 7.29-7.26 (m, 1H), 7.22 (d, J=1.5 Hz, 1H), 7.19 (ddd, J=1.0, 5.0, 7.5 Hz, 1H), 7.09 (ddd, J=0.9, 2.3, 9.0 Hz, 1H), 6.05 (q, J=6.7 Hz, 1H), 5.65 (td, J=2.8, 9.4 Hz, 1H), 5.29 (s, 1H), 4.55 (s, 2H), 4.10 (s, 3H), 4.06-3.97 (m, 1H), 3.75-3.66 (m, 1H), 2.62-2.48 (m, 1H), 2.18-2.09 (m, 1H), 2.04 (s, 3H), 1.80 (d, J=6.7 Hz, 3H), 1.77-1.69 (m, 2H), 1.66-1.59 (m, 1H).

Step 2. (R)-5-(5-(1-(3,5-Dichloropyridin-4-yl)ethoxy)-1H-indazol-3-yl)-2-methoxy-N-(pyridin-2-ylmethyl)pyridin-3-amine trifluoroacetic acid salt. A solution of product step 1 (95 mg, 0.16 mmol) in dichloromethane (1.5 mL) was treated with trifluoroacetic acid (1.5 mL, 19.73 mmol, 126 equiv) for 2 hours. The reaction mixture was azeotroped with toluene (2×15 mL) under reduced pressure and purified on a Gilson automated chromatography system (Waters Atlantis T3 Prep OBD column, 5 μm, 19×250 mm), eluting with a gradient of 0 to 100% acetonitrile in water with 0.1% formic acid. Product containing fractions were lyophilized to give an off-white solid as a TFA salt (40 mg, 40%). Analysis: LCMS m/z=521 (M+H); ¹H NMR (400 MHZ, CDCl3) δ=12.99 (s, 1H), 8.61-8.50 (m, 3H), 7.79-7.73 (m, 2H), 7.44 (d, J=8.9 Hz, 1H), 7.39 (d, J=7.8 Hz, 1H), 7.26 (ddd, J=1.0, 4.9, 7.4 Hz, 1H), 7.21 (d, J=1.8 Hz, 1H), 7.10-7.05 (m, 2H), 6.17 (t, J=5.9 Hz, 1H), 6.11 (q, J=6.6 Hz, 1H), 4.49 (d, J=5.9 Hz, 2H), 4.02 (s, 3H), 1.76 (d, J=6.7 Hz, 3H); 19F NMR (376 MHz, DMSO-d6) δ=−73.40 (s, 3F).

### Example 5. (R)—N-(5-(5-(1-(3,5-Dichloropyridin-4-yl)ethoxy)-1H-indazol-3-yl)-2-methoxypyridin-3-yl)morpholine-4-carboxamide

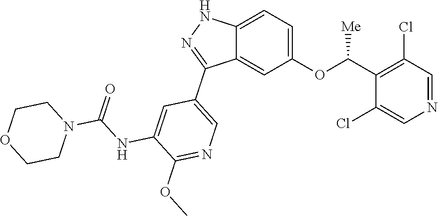 [SMILES: COc1ncc(-c2n[nH]c3ccc(O[C@H](C)c4c(Cl)cncc4Cl)cc23)cc1NC(=O)N1CCOCC1]

Step 1. N-(5-(5-((R)-1-(3,5-Dichloropyridin-4-yl)ethoxy)-1-(tetrahydro-2H-pyran-2-yl)-1H-indazol-3-yl)-2-methoxypyridin-3-yl)morpholine-4-carboxamide. Triethylamine (61 uL, 0.44 mmol, 1.5 equiv) and morpholine-4-carbonyl chloride (66 mg, 0.44 mmol, 1.5 equiv) were sequentially added to a solution of example 1 step 2 (150 mg, 0.292 mmol, 1 equiv) in anhydrous THF at room temperature. After stirring for 2 hours, additional morpholine-4-carbonyl chloride (218 mg, 1.46 mmol, 5 equiv) and triethylamine (201 uL, 1.46 mmol, 5 equiv) were added, and the reaction mixture was heated at 50° C. for 20 hours. Additional morpholine-4-carbonyl chloride (2×436 mg, 2.92 mmol, 10 equiv) and triethylamine (2×402 uL, 2.92 mmol, 10 equiv) were added in 24 hours intervals, and the reaction mixture was heated at 50° C. for 48 hours and then at reflux for 48 hours. After cooling to room temperature, the reaction was diluted with saturated brine (20 mL) and ethyl acetate (20 mL). The layers were separated. The organic layer was dried over sodium sulfate, filtered and concentrated under reduced pressure to give a yellow solid which was used directly in the next step (180 mg). Analysis: LCMS m/z=627 (M+H).

Step 2. (R)—N-(5-(5-(1-(3,5-Dichloropyridin-4-yl)ethoxy)-1H-indazol-3-yl)-2-methoxypyridin-3-yl)morpholine-4-carboxamide. Trifluoroacetic acid (1 mL, 13 mmol, 44.5 equiv) was added to a solution of product step 1 (180 mg, 0.292 mmol, 1 equiv) in anhydrous dichloromethane (5 mL). After stirring at room temperature for 16 hours, the reaction mixture was concentrated under reduced pressure. The residue was dissolved in methanol (10 mL), treated with MP-carbonate resin (3.2 mmol/g, 3.0 g), stirred for 30 minutes, filtered and concentrated under reduced pressure. The residue was absorbed onto Celite (1 g) and purified on an Interchim automated chromatography system (RediSep Rf Gold HP C18, 15.5 g cartridge), eluting with a gradient of 0 to 100% acetonitrile in water. The fractions containing pure product were collected and lyophilized to give a white solid (33 mg, 21%). Analysis: LCMS m/z=543.2 (M+H); ¹H NMR (400 MHz, DMSO-d6) δ=13.14 (s, 1H), 8.55 (s, 2H), 8.42 (d, J=2.2 Hz, 1H), 8.25 (d, J=2.2 Hz, 1H), 7.95 (s, 1H), 7.49 (d, J=8.9 Hz, 1H), 7.22 (d, J=2.0 Hz, 1H), 7.11 (dd, J=2.3, 9.0 Hz, 1H), 6.11 (q, J=6.6 Hz, 1H), 3.99 (s, 3H), 3.69-3.62 (m, 4H), 3.51-3.44 (m, 4H), 1.76 (d, J=6.7 Hz, 3H).

### Example 6. (R)-5-(5-(1-(3,5-Dichloropyridin-4-yl)ethoxy)-1H-indazol-3-yl)-2-methoxy-N-((2-methylpyridin-4-yl)methyl)pyridin-3-amine

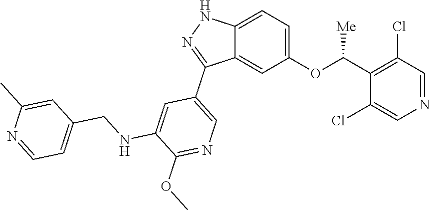 [SMILES: COc1ncc(-c2n[nH]c3ccc(O[C@H](C)c4c(Cl)cncc4Cl)cc23)cc1NCc1ccnc(C)c1]

Step 1. 5-(5-((R)-1-(3,5-Dichloropyridin-4-yl)ethoxy)-1-(tetrahydro-2H-pyran-2-yl)-1H-indazol-3-yl)-2-methoxy-N-((2-methylpyridin-4-yl)methyl)pyridin-3-amine. A mixture of example 1 step 2 (0.15 g, 0.29 mmol, 1 equiv), 2-methylpyridine-4-carbaldehyde (0.159 g, 1.31 mmol, 4.5 equiv), sodium triacetoxy borohydride (0.623 g, 2.93 mmol, 10 equiv) and acetic acid (0.12 g) in dichloromethane (3 mL) was stirred at room temperature for 30 hours. After completion, the reaction mixture was partitioned between saturated sodium carbonate (15 mL) and dichloromethane (20 mL). The aqueous layer was extracted with dichloromethane (2×10 mL). The combined organic layers were washed with saturated brine (10 mL), dried over sodium sulfate, filtered and concentrated under reduced pressure. The crude product was purified on a Büchi automated chromatography system (Biotage Sfär 25 g, 25 μm silica gel column), eluting with a gradient of 0 to 2% methanol in ethyl acetate to give a light-yellow oil (123 mg, 68%). Analysis: LCMS m/z=619 (M+H); ¹H NMR (400 MHZ, CDCl3) δ=8.45 (d, J=5.0 Hz, 1H), 8.39 (d, J=0.7 Hz, 2H), 7.93 (d, J=1.8 Hz, 1H), 7.46 (dd, J=1.6, 9.0 Hz, 1H), 7.25 (d, J=13.0 Hz, 1H), 7.17 (d, J=14.1 Hz, 2H), 7.13-7.05 (m, 2H), 6.03 (q, J=6.6 Hz, 1H), 5.63 (td, J=2.8, 9.3 Hz, 1H), 4.71 (br t, J=5.3 Hz, 1H), 4.40 (br d, J=5.4 Hz, 2H), 4.09 (s, 3H), 4.04-3.95 (m, 1H), 3.75-3.64 (m, 1H), 2.59-2.45 (m, 4H), 2.17-2.07 (m, 1H), 2.06-1.97 (m, 2H), 1.80 (d, J=6.7 Hz, 3H), 1.72 (br s, 3H).

Step 2. (R)-5-(5-(1-(3,5-Dichloropyridin-4-yl)ethoxy)-1H-indazol-3-yl)-2-methoxy-N-((2-methylpyridin-4-yl)methyl)pyridin-3-amine. A solution of product step 1 (120 mg, 0.194 mmol) in dichloromethane (1.5 mL) was treated with trifluoroacetic acid (1.5 mL, 19.73 mmol, 100 equiv) for 2 hours. The reaction mixture was azeotroped with toluene (2×15 mL) under reduced pressure and purified on a Gilson automated chromatography system (Waters Atlantis T3 Prep OBD column, 5 μm, 19×250 mm), eluting with a gradient of 0 to 100% acetonitrile in water with 0.1% formic acid. Product fractions were lyophilized to give an off-white solid as the TFA salt (53 mg, 42%). Analysis: LCMS m/z=535 (M+H); ¹H NMR (400 MHZ, CDCl3) δ=12.97 (s, 1H), 8.56 (s, 2H), 8.35 (d, J=5.1 Hz, 1H), 7.74 (d, J=2.0 Hz, 1H), 7.42 (d, J=8.9 Hz, 1H), 7.20 (s, 1H), 7.15-7.11 (m, 2H), 7.05 (dd, J=2.2, 9.0 Hz, 1H), 6.92 (d, J=1.8 Hz, 1H), 6.19 (t, J=6.3 Hz, 1H), 6.07 (q, J=6.6 Hz, 1H), 4.45-4.35 (m, 2H), 4.02 (s, 3H), 2.41 (s, 3H), 1.75 (d, J=6.6 Hz, 3H).

### Example 7. (R)—N-(5-(5-(1-(3,5-Dichloropyridin-4-yl)ethoxy)-1H-indazol-3-yl)-2-methoxypyridin-3-yl)-4-methylpiperazine-1-carboxamide

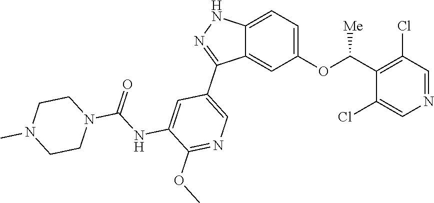 [SMILES: COc1ncc(-c2n[nH]c3ccc(O[C@H](C)c4c(Cl)cncc4Cl)cc23)cc1NC(=O)N1CCN(C)CC1]

Step 1. N-(5-(5-((R)-1-(3,5-Dichloropyridin-4-yl)ethoxy)-1-(tetrahydro-2H-pyran-2-yl)-1H-indazol-3-yl)-2-methoxypyridin-3-yl)-4-methylpiperazine-1-carboxamide. A solution of example 1 step 2 (150 mg, 0.29 mmol, 1 equiv), 4-methylpiperazine-1-carbonyl chloride HCl salt (115 mg, 0.58 mmol, 2 equiv) and potassium carbonate (161 mg, 1.17 mmol, 4 equiv) in THF (10 mL) was heated at 60° C. for 16 hours. Additional-methylpiperazine-1-carbonyl chloride HCl salt (460 mg, 2.32 mmol, 8 equiv) and potassium carbonate (644 mg, 4.68 mmol, 16 equiv) were added. The mixture was heated at 86° C. for 40 hours. After cooling to room temperature, the reaction mixture was filtered and washed with THF (100 mL). The filtrate was concentrated under reduced pressure. The residue was dissolved in DMSO (8 mL) and purified on an InterChim automated chromatography system (RediSep Rf GOLD 100 g HP C18 column), eluting with a gradient of 0 to 100% acetonitrile in water to give a yellow oil (60 mg, 32%). Analysis: LCMS m/z=640.2 (M+H).

Step 2. (R)—N-(5-(5-(1-(3,5-Dichloropyridin-4-yl)ethoxy)-1H-indazol-3-yl)-2-methoxypyridin-3-yl)-4-methylpiperazine-1-carboxamide. Trifluoroacetic acid (1 mL) was added to a solution of product step 2 (60 mg, 0.094 mmol, 1 equiv) in dichloromethane (1 mL). After stirring at room temperature for 2 hours, the volatiles were removed under reduced pressure. The residue was diluted with ethyl acetate (15 mL), washed with saturated sodium carbonate (3 mL), dried over sodium sulfate, filtered and concentrated under reduced pressure. The residue was redissolved in DMSO (8 mL) and purified on an InterChim automated chromatography system (RediSep Rf GOLD 100 g HP C18 column), eluting with a gradient of 0 to 100% acetonitrile in water to give an off-white solid (17 mg, 33%). Analysis: LCMS m/z=556.2 (M+H); ¹H NMR (400 MHZ, DMSO-d6) δ=13.12 (br s, 1H), 8.55 (s, 2H), 8.40 (d, J=2.2 Hz, 1H), 8.24 (d, J=2.1 Hz, 1H), 7.89 (br s, 1H), 7.50 (d, J=9.0 Hz, 1H), 7.20 (d, J=2.1 Hz, 1H), 7.10 (dd, J=2.3, 9.0 Hz, 1H), 6.11 (q, J=6.7 Hz, 1H), 3.99 (s, 3H), 3.51-3.46 (m, 4H), 2.39-2.33 (m, 4H), 2.22 (s, 3H), 1.76 (d, J=6.6 Hz, 3H).

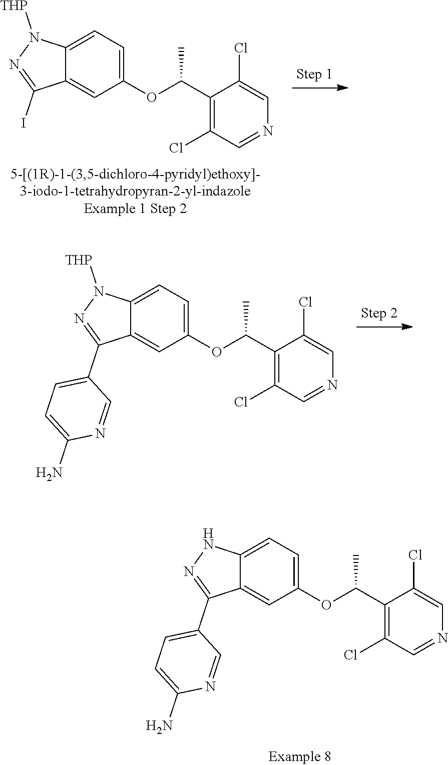

### Example 8. (R)-5-(5-(1-(3,5-Dichloropyridin-4-yl)ethoxy)-1H-indazol-3-yl)pyridin-2-amine

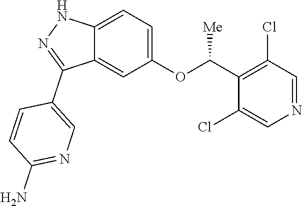 [SMILES: C[C@@H](Oc1ccc2[nH]nc(-c3ccc(N)nc3)c2c1)c1c(Cl)cncc1Cl]

Step 1. 5-(5-((R)-1-(3,5-Dichloropyridin-4-yl)ethoxy)-1-(tetrahydro-2H-pyran-2-yl)-1H-indazol-3-yl)pyridin-2-amine. A mixture of example 1 step 2 (1 g, 1.92 mmol, 1 equiv), 5-(4,4,5,5-tetramethyl-1,3,2-dioxaborolan-2-yl)pyridin-2-amine (552 mmg, 2.51 mmol, 1.3 equiv), [1,1′-bis(diphenylphosphino)ferrocene]dichloropalladium(II) (140 mg, 0.192 mmol, 0.1 equiv), potassium carbonate (530 mg, 3.84 mmol, 2 equiv) and water (2 mL) in 1,4-dioxane (20 mL) was sparged with nitrogen gas for 15 minutes. The dark red suspension was rigorously stirred and heated at 90° C. overnight. The black reaction mixture was cooled to room temperature and was diluted with water (10 mL) and ethyl acetate (10 mL). The organic layer was separated and the aqueous layer was extracted with ethyl acetate (3×10 mL). The combined organic layers were dried over sodium sulfate and concentrated under reduced pressure. The crude oil was absorbed onto Celite (10 g) and purified on an Interchim automated chromatography system (Sorbtech, 80 g silica gel cartridge), eluting with a gradient of 0 to 10% methanol in dichloromethane to give a white solid (654 mg, 70%). Analysis: LCMS 484.1 m/z (M+H).

Step 2. (R)-5-(5-(1-(3,5-Dichloropyridin-4-yl)ethoxy)-1H-indazol-3-yl)pyridin-2-amine. Trifluoroacetic acid (2 mL) was slowly added to a solution of product step 2 (100 mg, 0.207 mmol, 1 equiv) in dichloromethane (2 mL) and the red solution stirred at room temperature. After 3 hours, the reaction mixture was concentrated under reduced pressure. The residue was diluted with saturated sodium bicarbonate solution (5 mL) and ethyl acetate (5 mL). The organic layer was separated and the aqueous layer was extracted with ethyl acetate (3×5 mL). The combined organic layers were dried over sodium sulfate and concentrated under reduced pressure. The red crude residue was absorbed onto Celite (1 g) and purified on an Interchim automated chromatography system (RediSep Rf Gold HP C18, 15.5 g cartridge), eluting with a gradient of 0 to 100% acetonitrile in water. The fractions containing pure product were collected and lyophilized to give a white solid (53 mg, 64%). Analysis: LCMS 400.1 m/z (M+H); ¹H NMR (400 MHZ, DMSO-d6) δ=12.91 (s, 1H), 8.58 (s, 2H), 8.33 (d, J=2.1 Hz, 1H), 7.75 (dd, J=2.3, 8.6 Hz, 1H), 7.58-7.47 (m, 1H), 7.44 (d, J=8.9 Hz, 1H), 7.14-7.05 (m, 2H), 6.57 (d, J=8.7 Hz, 1H), 6.18-6.05 (m, 3H), 1.75 (d, J=6.7 Hz, 3H).

### Example 9. (R)-5-(5-(1-(3,5-Dichloropyridin-4-yl)ethoxy)-1H-indazol-3-yl)-N-methylpicolinamide

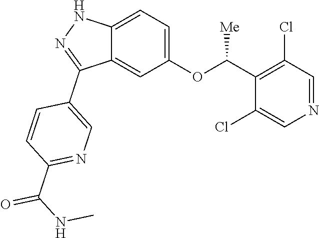 [SMILES: CNC(=O)c1ccc(-c2n[nH]c3ccc(O[C@H](C)c4c(Cl)cncc4Cl)cc23)cn1]

Step 1. 5-(5-((R)-1-(3,5-Dichloropyridin-4-yl)ethoxy)-1-(tetrahydro-2H-pyran-2-yl)-1H-indazol-3-yl)-N-methylpicolinamide. N-methyl-5-(4,4,5,5-tetramethyl-1,3,2-dioxaborolan-2-yl)pyridine-2-carboxamide (68 mg, 0.26 mmol, 1.3 equiv), [1,1′-bis(diphenylphosphino)-ferrocene]dichloropalladium(II) (15 mg, 0.02 mmol, 0.1 equiv) and potassium carbonate (55 mg, 0.4 mmol, 2 equiv) were added sequentially to a solution of example 1 step 2 product (104 mg, 0.2 mmol, 1 equiv) in a 10 to 1 mixture of 1,4-dioxane and water (16 mL) in a sealed tube. After sparging with nitrogen for 10 minutes, the reaction was heated at 95° C. for 16 hours. After cooling to room temperature, the reaction mixture was filtered and washed with ethyl acetate (100 mL). The filtrate was concentrated under reduced pressure. The residue was purified on an InterChim automated chromatography system (Sorbtech, 25 g silica gel column), eluting with a gradient of 0 to 100% ethyl acetate in heptanes to give a colorless oil (104 mg, 98%). Analysis: LCMS m/z=526.1 (M+H).

Step 2. (R)-5-(5-(1-(3,5-Dichloropyridin-4-yl)ethoxy)-1H-indazol-3-yl)-N-methylpicolinamide. Trifluoroacetic acid (1 mL) was added to a solution of product step 1 (104 mg, 0.2 mmol, 1 equiv) in dichloromethane (1 mL). After stirring at room temperature for 3 hours, the volatiles were removed under reduced pressure. The residue was diluted with ethyl acetate (15 mL), washed with saturated sodium carbonate (3 mL), dried over sodium sulfate, filtered and concentrated under reduced pressure. The residue was redissolved in DMSO (8 mL) and purified on an InterChim automated chromatography system (RediSep Rf GOLD HP C18, 100 g column), eluting with a gradient of 0 to 100% acetonitrile in water to give an off-white solid (28 mg, 32%). Analysis: LCMS m/z=442.1 (M+H); ¹H NMR (400 MHz, DMSO-d6) δ=13.48 (br s, 1H), 9.05 (dd, J=0.8, 2.1 Hz, 1H), 8.79 (q, J=4.5 Hz, 1H), 8.61 (s, 2H), 8.32 (dd, J=2.2, 8.2 Hz, 1H), 8.17 (dd, J=0.7, 8.1 Hz, 1H), 7.56 (d, J=9.2 Hz, 1H), 7.27 (d, J=2.2 Hz, 1H), 7.15 (dd, J=2.3, 9.0 Hz, 1H), 6.15 (q, J=6.5 Hz, 1H), 2.87 (d, J=4.9 Hz, 3H), 1.78 (d, J=6.6 Hz, 3H).

### Example 10. (R)-5-(1-(3,5-Dichloropyridin-4-yl)ethoxy)-3-(6-(methylsulfonyl)pyridin-3-yl)-1H-indazole

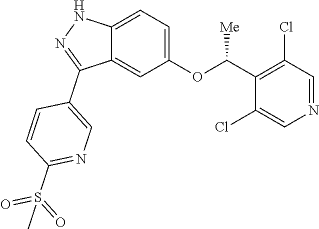 [SMILES: C[C@@H](Oc1ccc2[nH]nc(-c3ccc(S(C)(=O)=O)nc3)c2c1)c1c(Cl)cncc1Cl]

Step 1. 5-((R)-1-(3,5-Dichloropyridin-4-yl)ethoxy)-3-(6-(methylthio)pyridin-3-yl)-1-(tetrahydro-2H-pyran-2-yl)-1H-indazole. 2-Methylsulfanyl-5-(4,4,5,5-tetramethyl-1,3,2-dioxaborolan-2-yl)pyridine (373.5 mg, 1.49 mmol, 1.1 equiv), tetrakis(triphenylphosphine)palladium(0) (156.2 mg, 0.14 mmol, 0.1 equiv) and 1M sodium carbonate solution (2.7 mL, 2.7 mmol, 2 equiv) were sequentially added to a solution of product example 1 step 2 (700 mg, 1.35 mmol, 1 equiv) in 1,4-dioxane (13.5 mL) at room temperature. The resulting mixture was heated at 100° C. overnight. After cooling to room temperature, the solvent was removed under reduced pressure. The residue was dissolved in dichloromethane (50 mL), washed with water (2×20 mL), dried over sodium sulfate and filtered. The filtrate was concentrated under reduced pressure onto silica gel and purified on a Biotage automated chromatography system (Biotage® Sfär Silica HC, 25 g silica gel column), eluting with a gradient of 0 to 50% ethyl acetate in heptanes to give a dark yellow solid (650 mg, 93%). Analysis: LCMS m/z=515.1 (M+H).

Step 2. 5-((R)-1-(3,5-Dichloropyridin-4-yl)ethoxy)-3-(6-(methylsulfonyl)pyridin-3-yl)-1-(tetrahydro-2H-pyran-2-yl)-1H-indazole. Potassium carbonate (460.5 mg, 3.33 mmol, 5.2 equiv) and m-chloroperoxy benzoic acid (287.3 mg, 1.66 mmol, 2.0 equiv) were sequentially added to a solution of the product from step 1 (330 mg, 0.64 mmol, 1 equiv) in a mixture of ethanol (6.4 mL) and water (3.2 mL). The resulting mixture was warmed to room temperature and stirred overnight. The reaction mixture was concentrated under reduced pressure onto Celite and purified on Biotage automated chromatography (RediSep Rf Gold® C18, 50 g column), eluting with a gradient of 0 to 100% acetonitrile in water to give a white solid (270 mg, 77%). Analysis: LCMS m/z=547.1 (M+H).

Step 3. (R)-5-(1-(3,5-Dichloropyridin-4-yl)ethoxy)-3-(6-(methylsulfonyl)pyridin-3-yl)-1H-indazole. Product step 2 (270.0 mg, 0.49 mmol, 1.0 equiv) in dichloromethane (2.7 mL) was treated with trifluoroacetic acid (2.7 mL, 35.3 mmol, 10 v) at 0° C. The resulting mixture was stirred at room temperature overnight. Solvent was removed under reduced pressure. The residue was dissolved in a mixture of dichloromethane (4.0 mL) and methanol (1.0 mL), treated with MP-Carbonate® (0.15 g) at room temperature for 2 hours. The mixture was filtered and the filtrate was concentrated was concentrated under reduced pressure onto Celite (1.0 g). The material was purified on a Biotage automated chromatography (RediSep Rf Gold R) Reversed-phase C18, 50 g), eluting a gradient of 0 to 100% acetonitrile in water to give a white solid (110 mg, 66%). Analysis: LCMS m/z=463.0 (M+H); ¹H NMR (400 MHz, CDCl₃) δ=10.40 (br s, 1H), 9.25 (dd, J=0.8, 2.1 Hz, 1H), 8.44 (s, 2H), 8.36 (dd, J=2.1, 8.2 Hz, 1H), 8.19 (dd, J=0.8, 8.1 Hz, 1H), 7.46 (d, J=9.7 Hz, 1H), 7.23-7.19 (m, 2H), 6.08 (q, J=6.7 Hz, 1H), 3.31 (s, 3H), 1.84 (d, J=6.7 Hz, 3H).

### Example 11. (R)—N-(5-(5-(1-(3,5-Dichloropyridin-4-yl)ethoxy)-1H-indazol-3-yl)pyridin-2-yl)cyclopropanecarboxamide

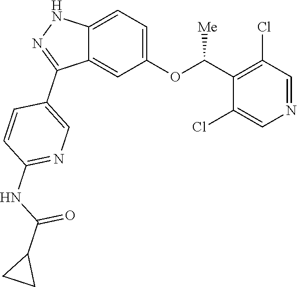 [SMILES: C[C@@H](Oc1ccc2[nH]nc(-c3ccc(NC(=O)C4CC4)nc3)c2c1)c1c(Cl)cncc1Cl]

Step 1. N-(5-(5-((R)-1-(3,5-Dichloropyridin-4-yl)ethoxy)-1-(tetrahydro-2H-pyran-2-yl)-1H-indazol-3-yl)pyridin-2-yl)cyclopropanecarboxamide. A mixture of cyclopropane carbonyl chloride (150 mg, 0.3 mmol, 1 equiv), example 8 step 1 (62 mg, 0.6 mmol, 2 equiv), and 4-dimethylaminopyridine (38 mg, 0.3 mmol, 1 equiv) in pyridine (3.75 mL) was stirred at room temperature for 18 hours. The volatiles were removed under reduced pressure. The residue was dissolved in dichloromethane (5 mL), washed with water (3×7 mL), dried over sodium sulfate, filtered and concentrated under reduced pressure. The residue (140 mg) was purified on an InterChim automated chromatography system (Sfär Biotage HC 5 g silica-gel column, previously flushed with 30 mL of 1% triethylamine in heptanes), eluting with a gradient of 0 to 100% ethyl acetate in heptanes to give an off-white foam (130 mg, 94%). Analysis: LCMS m/z 552.2 m/z (M+H).

Step 2. (R)—N-(5-(5-(1-(3,5-Dichloropyridin-4-yl)ethoxy)-1H-indazol-3-yl)pyridin-2-yl)cyclopropanecarboxamide. Trifluoroacetic acid (1.3 mL, 17 mmol, 68 equiv) was added to a solution of product step 1 (130 mg, 0.25 mmol, 1 equiv) in dichloromethane (1.3 mL). After stirring at room temperature for 2 hours, the volatiles were removed under reduced pressure. The residue was diluted with dichloromethane (5 mL), washed with saturated sodium carbonate (5 mL), dried over sodium sulfate, filtered and concentrated under reduced pressure. The residue was purified on an InterChim automated chromatography system (RediSep Rf GOLD HP C18, 4 g cartridge), eluting with a gradient of 0 to 100% acetonitrile in water to give a tan solid (50 mg, 45%). Analysis: LCMS m/z 468.1 m/z (M+H); ¹H NMR (400 MHZ, CDCl3) δ=8.93-8.54 (m, 2H), 8.44 (s, 2H), 8.36 (d, J=8.6 Hz, 1H), 8.18-8.13 (m, 1H), 7.41 (d, J=9.7 Hz, 1H), 7.17 (qd, J=2.4, 4.8 Hz, 2H), 6.07 (q, J=6.7 Hz, 1H), 1.83 (d, J=6.6 Hz, 3H), 1.67-1.61 (m, 1H), 1.18-1.14 (m, 2H), 0.97-0.92 (m, 2H).

Examples 12-16 were synthesized using procedures previously described.

| | | |
|---|---|---|
| Example 12 5-[5-[(1R)-1-(3,5-dichloro- 4-pyridyl)ethoxy]-1H- indazol-3-yl]pyridine-2- carboxamide | 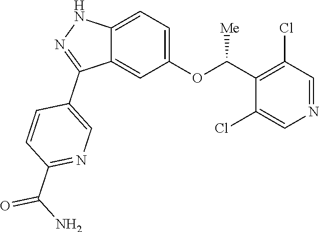 [SMILES: C[C@@H](Oc1ccc2[nH]nc(-c3ccc(C(N)=O)nc3)c2c1)c1c(Cl)cncc1Cl] | LCMS m/z = 428 (M + H); 1H NMR (400 MHz, DMSO-d6) δ = 13.48 (br s, 1H), 9.06 (d, J = 1.5 Hz, 1H), 8.61 (s, 2H), 8.32 (dd, J = 2.2, 8.2 Hz, 1H), 8.21-8.12 (m, 2H), 7.69 (br s, 1H), 7.56 (d, J = 8.9 Hz, 1H), 7.28 (d, J = 2.1 Hz, 1H), 7.15 (dd, J = 2.3, 9.0 Hz, 1H), 6.15 (q, J = 6.6 Hz, 1H), 1.78 (d, J = 6.6 Hz, 3H) |
| Example 13 N-[5-[5-[(1R)-1-(3,5- dichloro-4-pyridyl)ethoxy]- 1H-indazol-3-yl]-2- pyridyl]-2- (dimethylamino)- acetamide | 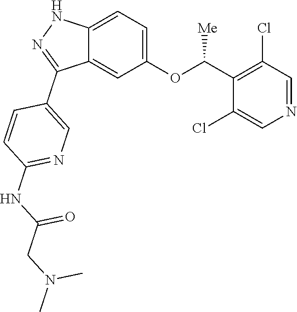 [SMILES: C[C@@H](Oc1ccc2[nH]nc(-c3ccc(NC(=O)CN(C)C)nc3)c2c1)c1c(Cl)cncc1Cl] | LCMS m/z = 485 (M + H); 1H NMR (400 MHz, CDCl3) δ = 8.93-8.54 (m, 2H), 8.44 (s, 2H), 8.36 (d, J = 8.6 Hz, 1H), 8.18-8.13 (m, 1H), 7.41 (d, J = 9.7 Hz, 1H), 7.17 (qd, J = 2.4, 4.8 Hz, 2H), 6.07 (q, J = 6.7 Hz, 1H), 1.83 (d, J = 6.6 Hz, 3H), 1.67-1.61 (m, 1H), 1.18-1.14 (m, 2H), 0.97-0.92 (m, 2H) |
| Example 14 3-[5-[5-[(1R)-1-(3,5- dichloro-4-pyridyl)ethoxy]- 1H-indazol-3-yl]-2- pyridyl]-1,1-dimethyl-urea | 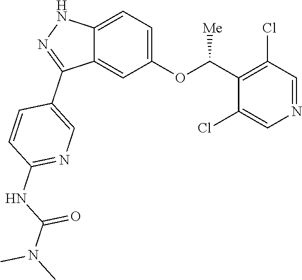 [SMILES: C[C@@H](Oc1ccc2[nH]nc(-c3ccc(NC(=O)N(C)C)nc3)c2c1)c1c(Cl)cncc1Cl] | LCMS m/z = 471 (M + H); 1H NMR (400 MHz, DMSO-d6) δ = 13.19 (br s, 1H), 9.01 (s, 1H), 8.66 (d, J = 1.7 Hz, 1H), 8.58 (s, 2H), 8.06 (dd, J = 2.2, 8.7 Hz, 1H), 7.95 (d, J = 8.7 Hz, 1H), 7.50 (d, J = 8.9 Hz, 1H), 7.21 (d, J = 1.5 Hz, 1H), 7.12 (dd, J = 2.1, 9.0 Hz, 1H), 6.14 (q, J = 6.6 Hz, 1H), 2.98 (s, 6H), 1.76 (d, J = 6.6 Hz, 3H) |
| Example 15 N-[5-[5-[(1R)-1-(3,5- dichloro-4-pyridyl)ethoxy]- 1H-indazol-3-yl]-2- methoxy-3-pyridyl]-2- (dimethylamino)acetamide | 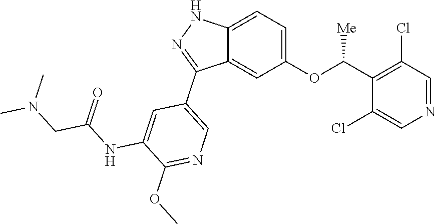 [SMILES: COc1ncc(-c2n[nH]c3ccc(O[C@H](C)c4c(Cl)cncc4Cl)cc23)cc1NC(=O)CN(C)C] | LCMS m/z = 515 (M + H); 1H NMR (400 MHz, DMSO-d6) δ = 13.19 (br s, 1H), 9.46 (s, 1H), 8.98 (d, J = 2.1 Hz, 1H), 8.55 (s, 2H), 8.28 (d, J = 2.2 Hz, 1H), 7.50 (d, J = 8.9 Hz, 1H), 7.25 (d, J = 2.0 Hz, 1H), 7.11 (dd, J = 2.2, 9.0 Hz, 1H), 6.11 (q, J = 6.7 Hz, 1H), 4.05 (s, 3H), 3.16 (d, J = 1.8 Hz, 2H), 2.35 (s, 6H), 1.75 (d, J = 6.6 Hz, 3H) |
| Example 16 N-[5-[5-[(1R)-1-(3,5- dichloro-4-pyridyl)ethoxy]- 1H-indazol-3-yl]-2- pyridyl]morpholine-4- carboxamide | 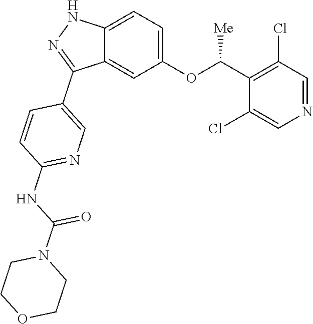 [SMILES: C[C@@H](Oc1ccc2[nH]nc(-c3ccc(NC(=O)N4CCOCC4)nc3)c2c1)c1c(Cl)cncc1Cl] | LCMS m/z 513 m/z (M + H); 1H NMR (400 MHz, DMSO-d6) δ = 13.18 (br s, 1H), 9.38 (s, 1H), 8.67 (dd, J = 0.7, 2.3 Hz, 1H), 8.58 (s, 2H), 8.08 (dd, J = 2.4, 8.7 Hz, 1H), 7.94 (dd, J = 0.7, 8.7 Hz, 1H), 7.50 (d, J = 9.2 Hz, 1H), 7.21 (d, J = 2.1 Hz, 1H), 7.12 (dd, J = 2.3, 9.0 Hz, 1H), 6.15 (q, J = 6.6 Hz, 1H), 3.64-3.60 (m, 4H), 3.52-3.47 (m, 4H), 2.07 (s, 1H), 1.76 (d, J = 6.7 Hz, 3H) |

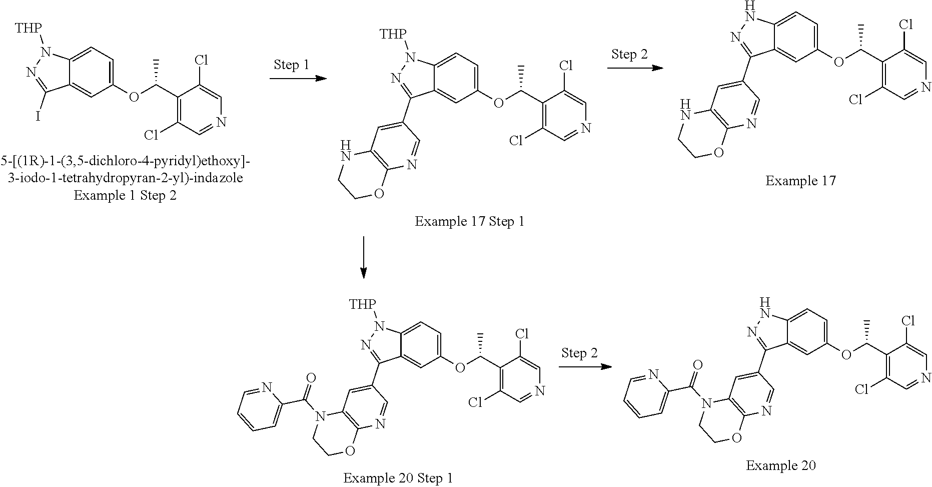

### Example 17. 7-[5-[(1R)-1-(3,5-dichloro-4-pyridyl)ethoxy]-1H-indazol-3-yl]-2,3-dihydro-1H-pyrido[2,3-b][1,4]oxazine

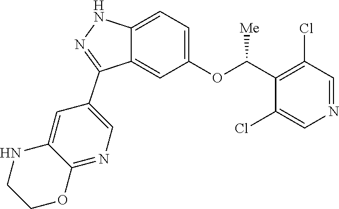 [SMILES: C[C@@H](Oc1ccc2[nH]nc(-c3cnc4c(c3)NCCO4)c2c1)c1c(Cl)cncc1Cl]

Step 1. 7-(5-((R)-1-(3,5-Dichloropyridin-4-yl)ethoxy)-1-(tetrahydro-2H-pyran-2-yl)-1H-indazol-3-yl)-2,3-dihydro-1H-pyrido[2,3-b][1,4]oxazine. A mixture of product step 2 example 1 (1.0 g, 1.93 mmol, 1 equiv), 7-(4,4,5,5-tetramethyl-1,3,2-dioxaborolan-2-yl)-2,3-dihydro-1H-pyrido[2,3-b][1,4]oxazine (0.66 g, 2.52 mmol, 1.3 equiv), potassium carbonate (0.55 g, 3.98 mmol, 2 equiv), (1,1′-bis(diphenylphosphino)ferrocene palladium(II) dichloride (0.15 g, 0.20 mmol, 0.10 equiv) and water (2 mL) in 1,4-dioxane (20 mL) was sparged with nitrogen for 15 minutes. After heating overnight at 90° C. under a nitrogen atmosphere, the mixture was cooled to room temperature, diluted with water (30 mL) and extracted with dichloromethane (2×30 mL). The combined organic layers were washed with saturated brine (30 mL) and concentrated onto silica gel (12 g). The crude product was purified on a Büchi automated chromatography system (Biotage Sfär 50 g, 25 μm silica gel column), eluting with a gradient of 20 to 100% ethyl acetate in heptanes then with 0 to 10% methanol in ethyl acetate to give a tan solid (0.77, 76%). Analysis: LCMS m/z=526 (M+H); ¹H NMR (400 MHZ, DMSO-d6) δ=8.57 (s, 2H), 7.75 (dd, J=2.1, 3.7 Hz, 1H), 7.67 (dd, J=2.1, 9.0 Hz, 1H), 7.35 (dd, J=0.9, 2.1 Hz, 1H), 7.20 (d, J=2.0 Hz, 1H), 7.15 (td, J=2.0, 9.0 Hz, 1H), 6.21 (s, 1H), 6.13 (dd, J=1.3, 6.7 Hz, 1H), 5.81-5.76 (m, 1H), 4.33 (t, J=4.3 Hz, 2H), 3.91-3.79 (m, 1H), 3.79-3.60 (m, 1H), 3.37-3.32 (m, 2H), 2.45-2.29 (m, 1H), 2.10-2.00 (m, 1H), 1.98-1.90 (m, 1H), 1.76 (d, J=6.6 Hz, 3H), 1.72 (br d, J=12.5 Hz, 1H), 1.62-1.52 (m, 2H).

Step 2. (R)-(7-(5-(1-(3,5-Dichloropyridin-4-yl)ethoxy)-1H-indazol-3-yl)-2,3-dihydro-1H-pyrido[2,3-b][1,4]oxazin-1-yl)(pyridin-2-yl)methanone. A solution of product step 1 (0.13 mg, 0.21 mmol) in dichloromethane (1 mL) was treated with trifluoroacetic acid (1 mL, 13.1 mmol, 62 equiv) for 3 hours. The mixture was concentrated under reduced pressure. The residue was dissolved in saturated sodium bicarbonate (5 mL) and extracted with dichloromethane (2×5 mL). The organic extract was concentrated onto silica gel (6 g). The crude product was purified on a Büchi automated chromatography system (Biotage Sfär 25 g, 20 μm silica gel column), eluting with a gradient of 0 to 10% methanol in ethyl acetate to give a tan solid (86 mg, 76%). Analysis: LCMS m/z=547 (M+H); ¹H NMR (400 MHZ, DMSO-d6) δ=13.18 (s, 1H), 9.15-8.11 (m, 5H), 8.03 (dt, J=1.7, 7.7 Hz, 1H), 7.83 (td, J=1.0, 7.8 Hz, 1H), 7.56 (dd, J=5.3, 7.0 Hz, 1H), 7.49 (d, J=9.0 Hz, 1H), 7.15 (br s, 1H), 7.10 (dd, J=2.2, 9.0 Hz, 1H), 6.09 (q, J=6.6 Hz, 1H), 4.47 (br t, J=4.3 Hz, 2H), 4.00 (br s, 2H), 1.75 (d, J=6.6 Hz, 3H).

### Example 20. [7-[5-[(1R)-1-(3,5-dichloro-4-pyridyl)ethoxy]-1H-indazol-3-yl]-2,3-dihydropyrido[2,3-b][1,4]oxazin-1-yl]-(2-pyridyl)methanone

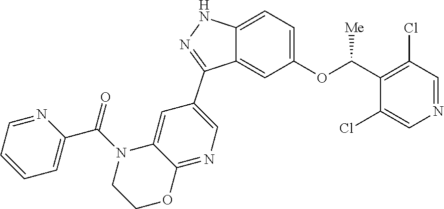 [SMILES: C[C@@H](Oc1ccc2[nH]nc(-c3cnc4c(c3)N(C(=O)c3ccccn3)CCO4)c2c1)c1c(Cl)cncc1Cl]

Step 1. (7-(5-((R)-1-(3,5-Dichloropyridin-4-yl)ethoxy)-1-(tetrahydro-2H-pyran-2-yl)-1H-indazol-3-yl)-2,3-dihydro-1H-pyrido[2,3-b][1,4]oxazin-1-yl)(pyridin-2-yl)methanone. A solution of example 17 step 1 (0.13 g, 0.25 mmol), 2-picolinic acid (99 mg, 0.80 mmol, 3 equiv), and N,N-diisopropylethylamine (0.75 mL, 4.3 mmol, 17 equiv) in THF (6 mL) was treated with 1-propanephosphonic anhydride in ethyl acetate (1.1 mL, 1.8 mmol, 7 equiv; 50% w/w solution) and heated at 50° C. overnight. The mixture was cooled to room temperature, diluted with water (10 mL) and extracted with ethyl acetate (2×10 mL). The combined organic layers were concentrated onto silica gel (5 g). The crude product was purified on a Büchi automated chromatography system (Biotage Sfär 25 g, 25 μm silica gel column), eluting with a gradient of 0 to 10% methanol in ethyl acetate to give a tan solid (0.13 g, 83%). Analysis: LCMS m/z=631 (M+H).

Step 2. (R)-(7-(5-(1-(3,5-Dichloropyridin-4-yl)ethoxy)-1H-indazol-3-yl)-2,3-dihydro-1H-pyrido[2,3-b][1,4]oxazin-1-yl)(pyridin-2-yl)methanone. A solution of product step 1 (0.13 mg, 0.21 mmol) in dichloromethane (1 mL) was treated with trifluoroacetic acid (1 mL, 13.1 mmol, 62 equiv) for 3 hours. The mixture was concentrated under reduced pressure. The residue was dissolved in saturated sodium bicarbonate (5 mL) and extracted with dichloromethane (2×5 mL). The organic extract was concentrated onto silica gel (6 g). The crude product was purified on a Büchi automated chromatography system (Biotage Sfär 25 g, 20 μm silica gel column), eluting with a gradient of 0 to 10% methanol in ethyl acetate to give a tan solid (86 mg, 76%). Analysis: LCMS m/z=547 (M+H); ¹H NMR (400 MHZ, DMSO-d6) δ=13.18 (s, 1H), 9.15-8.11 (m, 5H), 8.03 (dt, J=1.7, 7.7 Hz, 1H), 7.83 (td, J=1.0, 7.8 Hz, 1H), 7.56 (dd, J=5.3, 7.0 Hz, 1H), 7.49 (d, J=9.0 Hz, 1H), 7.15 (br s, 1H), 7.10 (dd, J=2.2, 9.0 Hz, 1H), 6.09 (q, J=6.6 Hz, 1H), 4.47 (br t, J=4.3 Hz, 2H), 4.00 (br s, 2H), 1.75 (d, J=6.6 Hz, 3H).

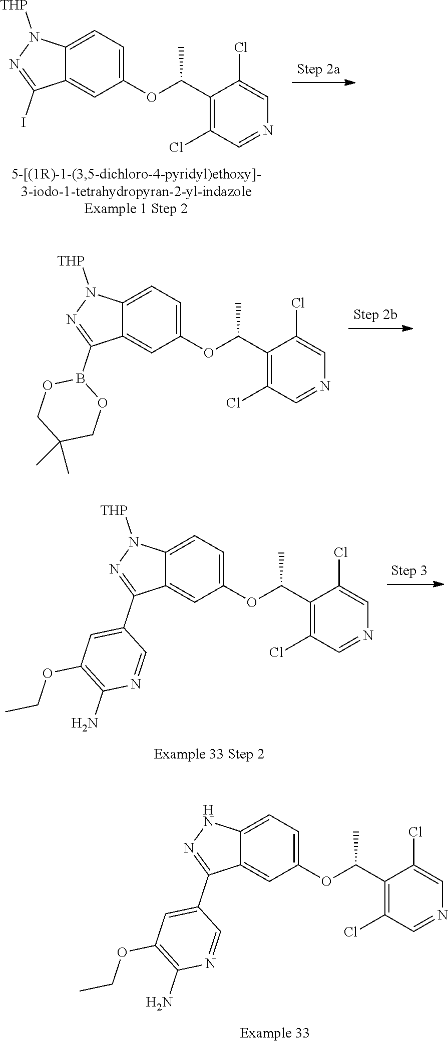

### Example 33. 5-[5-[(1R)-1-(3,5-Dichloro-4-pyridyl)ethoxy]-1H-indazol-3-yl]-3-ethoxy-pyridin-2-amine

Step 1. Bromo-3-ethoxypyridin-2-amine. 2-Amino-5-bromo-pyridin-3-ol (1.25 g, 6.6 mmol, 1 equiv) was treated with iodoethane (1 mL, 12.4 mmol, 1.9 equiv), methyltrioctylammonium chloride (0.5 mL, 1.1 mmol, 0.16 equiv) and 6M aqueous sodium hydroxide (8 mL, 48 mmol, 7.3 equiv) in dichloromethane (8 mL) at room temperature overnight. The reaction was poured into water (100 mL) and extracted with dichloromethane (3×100 mL). The combined organic extracts were concentrated under reduced pressure onto silica gel (12 g). The residue was purified on a Büchi automated chromatography system (Biotage Sfär 50 g, 60 μm column), eluting with ethyl acetate to give an off-white solid (0.95 g, 67%). Analysis: LCMS m/z=217 (M+H); ¹H NMR (400 MHZ, DMSO-d6) δ=7.56 (d, J=2.0 Hz, 1H), 7.15 (d, J=2.0 Hz, 1H), 5.86 (s, 2H), 4.04 (q, J=7.0 Hz, 2H), 1.34 (t, J=7.0 Hz, 3H).

Step 2. 5-(5-((R)-1-(3,5-Dichloropyridin-4-yl)ethoxy)-1-(tetrahydro-2H-pyran-2-yl)-1H-indazol-3-yl)-3-ethoxypyridin-2-amine. Product step 2 example 1 (0.25 g, 0.48 mmol, 1 equiv) was combined with bis(neopentylglycolato)diboron (0.13 g, 0.57 mmol, 1.2 equiv), [1,1′-bis(diphenylphosphino)ferrocene]palladium(II) dichloride (39 mg, 0.05 mmol, 0.1 equiv) and potassium acetate (0.15 g, 1.5 mmol, 3 equiv) in 1,4-dioxane (10 mL). The mixture was sparged with nitrogen for 5 minutes then heated at 90° C. for 16 hours to give a mixture of the boronic acid and boronic ester 292-2. Bromo-3-ethoxypyridin-2-amine (217 mg, 1 mmol, 2 equiv), potassium carbonate (0.23 g, 1.65 mmol, 3 equiv), [1,1′-bis(diphenylphosphino)-ferrocene]palladium(II) dichloride (39 mg, 0.05 mmol, 0.1 equiv) and water (1.5 mL) were added and the mixture was sparged with nitrogen for an additional 5 minutes, then heated at 90° C. for 4 hours. The mixture was cooled to room temperature, diluted with water (20 mL) and extracted with dichloromethane (3×20 mL). The combined organic layers were concentrated under reduced pressure onto silica gel (6 g). The crude product was purified on a Büchi automated chromatography system (Biotage Sfär 50 g, 60 μm column), eluting with a gradient of 0 to 100% ethyl acetate in heptanes to give a brown solid (75 mg, 29%). Analysis: LCMS m/z=528 (M+H); ¹H NMR (400 MHZ, DMSO-d6) δ=8.56 (s, 2H), 7.91 (t, J=1.5 Hz, 1H), 7.71-7.61 (m, 1H), 7.32 (d, J=1.5 Hz, 1H), 7.19-7.14 (m, 2H), 6.18-6.11 (m, 1H), 5.94 (s, 2H), 5.79-5.73 (m, 1H), 4.30 (t, J=5.4 Hz, 3H), 4.15-3.99 (m, 2H), 3.88 (br s, 1H), 3.76-3.66 (m, 1H), 3.14 (d, J=5.4 Hz, 6H), 2.47-2.29 (m, 1H), 2.09-1.89 (m, 2H), 1.79-1.67 (m, 4H), 1.57 (br s, 2H), 1.40 (t, J=6.9 Hz, 3H), 0.75 (s, 9H).

Step 3. (R)-5-(5-(1-(3,5-Dichloropyridin-4-yl)ethoxy)-1H-indazol-3-yl)-3-ethoxypyridin-2-amine formic acid salt. Product step 2 (72 mg, 0.14 mmol) and trifluoroacetic acid (0.6 mL, 7.8 mmol, 58 equiv) in dichloromethane (0.6 mL) was stirred at room temperature for 2 hours. The mixture was concentrated under reduced pressure. The residue was purified on a Gilson automated chromatography system (Waters Atlantis T3 Prep OBD column, 5 μm, 19×250 mm), eluting with a gradient of 0 to 100% acetonitrile in water with 0.1% formic acid. Product containing fractions were lyophilized to give an off-white solid (35 mg, 52%). Analysis: LCMS m/z=444 (M+H); ¹H NMR (400 MHZ, DMSO-d6) δ=12.96 (br s, 1H), 8.56 (s, 2H), 8.14 (s, 1H), 7.92 (d, J=1.7 Hz, 1H), 7.45 (d, J=9.0 Hz, 1H), 7.37 (d, J=1.7 Hz, 1H), 7.15 (d, J=2.1 Hz, 1H), 7.10 (dd, J=2.3, 9.0 Hz, 1H), 6.11 (q, J=6.6 Hz, 1H), 5.88 (s, 2H), 4.10 (q, J=7.0 Hz, 2H), 1.75 (d, J=6.7 Hz, 3H), 1.40 (t, J=7.0 Hz, 3H).

Examples 18-34 were synthesized using procedures described previously.

| | | |
|---|---|---|
| Example 18 [7-[5-[(1R)-1-(3,5- dichloro-4- pyridyl)ethoxy]-1H- indazol-3-yl]-2,3- dihydropyrido[2,3- b][1,4]oxazin-1-yl]- morpholino-methanone | 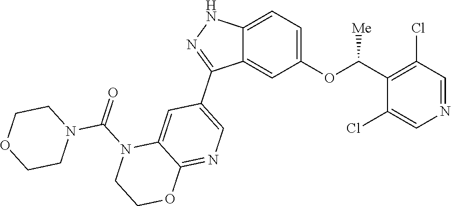 [SMILES: C[C@@H](Oc1ccc2[nH]nc(-c3cnc4c(c3)N(C(=O)N3CCOCC3)CCO4)c2c1)c1c(Cl)cncc1Cl] | LCMS m/z = 555 (M + H); 1H NMR (400 MHz, DMSO-d6) δ = 13.19 (br s, 1H), 8.57 (s, 2H), 8.18 (d, J = 2.1 Hz, 1H), 8.00 (d, J = 2.1 Hz, 1H), 7.49 (d, J = 9.0 Hz, 1H), 7.22 (d, J = 2.1 Hz, 1H), 7.11 (dd, J = 2.3, 9.0 Hz, 1H), 6.12 (q, J = 6.7 Hz, 1H), 4.41 (t, J = 4.4 Hz, 2H), 3.71 (br dd, J = 2.7, 4.9 Hz, 2H), 3.67-3.59 (m, 4H), 3.43-3.34 (m, 4H), 1.76 (d, J = 6.6 Hz, 3H) |
| Example 19 N-[5-[5-[(1R)-1-(3,5- dichloro-4-pyridyl) ethoxy]-1H-indazol-3- yl]-2-pyridyl]-4- methyl-piperazine-1- carboxamide | 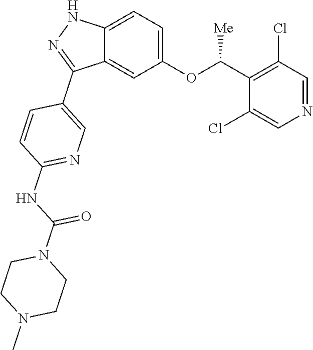 [SMILES: C[C@@H](Oc1ccc2[nH]nc(-c3ccc(NC(=O)N4CCN(C)CC4)nc3)c2c1)c1c(Cl)cncc1Cl] | LCMS m/z 526 (M + H); 1H NMR (400 MHz, DMSO-d6) δ = 13.17 (s, 1H), 9.39 (br s, 1H), 8.66 (dd, J = 0.6, 2.3 Hz, 1H), 8.58 (s, 2H), 8.07 (dd, J = 2.3, 8.7 Hz, 1H), 7.93 (dd, J = 0.7, 8.7 Hz, 1H), 7.50 (d, J = 9.0 Hz, 1H), 7.21 (d, J = 2.0 Hz, 1H), 7.12 (dd, J = 2.2, 9.0 Hz, 1H), 6.15 (q, J = 6.7 Hz, 1H), 3.53 (br s, 3H), 3.46-3.33 (m, 2H), 2.45- 2.21 (m, 6H), 1.76 (d, J = 6.6 Hz, 3H) |
| Example 20 [7-[5-[(1R)-1-(3,5- dichloro-4-pyridyl) ethoxy]-1H-indazol- 3-yl]-2,3- dihydropyrido[2,3- b][1,4]oxazin-1-yl]-(2- pyridyl)methanone | 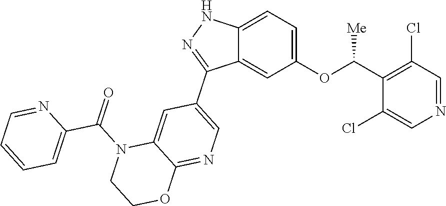 [SMILES: C[C@@H](Oc1ccc2[nH]nc(-c3cnc4c(c3)N(C(=O)c3ccccn3)CCO4)c2c1)c1c(Cl)cncc1Cl] | LCMS m/z = 547 (M + H); 1H NMR (400 MHz, DMSO-d6) δ = 13.18 (s, 1H), 9.15-8.11 (m, 5H), 8.03 (dt, J = 1.7, 7.7 Hz, 1H), 7.83 (td, J = 1.0, 7.8 Hz, 1H), 7.56 (dd, J = 5.3, 7.0 Hz, 1H), 7.49 (d, J = 9.0 Hz, 1H), 7.15 (br s, 1H), 7.10 (dd, J = 2.2, 9.0 Hz, 1H), 6.09 (q, J = 6.6 Hz, 1H), 4.47 (br t, J = 4.3 Hz, 2H), 4.00 (br s, 2H), 1.75 (d, J = 6.6 Hz, 3H) |
| Example 21 1-[7-[5-[(1R)-1-(3,5- dichloro-4-pyridyl) ethoxy]-1H-indazol-3- yl]-2,3-dihydropyrido [2,3-b][1,4]oxazin-1- yl]-2-(dimethylamino) ethanone | 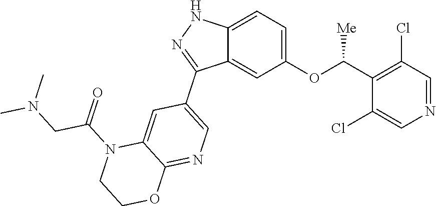 [SMILES: C[C@@H](Oc1ccc2[nH]nc(-c3cnc4c(c3)N(C(=O)CN(C)C)CCO4)c2c1)c1c(Cl)cncc1Cl] | LCMS m/z = 527 (M + H); 1H NMR (400 MHz, CDCl3) δ = 10.25 (br s, 1H), 8.95 (br s, 1H), 8.47 (d, J = 1.3 Hz, 1H), 8.41 (s, 2H), 7.38 (d, J = 8.9 Hz, 1H), 7.27-7.26 (m, 1H), 7.14 (dd, J = 2.2, 9.0 Hz, 1H), 6.08 (q, J = 6.6 Hz, 1H), 4.52 (t, J = 4.6 Hz, 2H), 4.21- 4.05 (m, 2H), 3.32 (s, 2H), 2.34 (s, 6H), 1.81 (d, J = 6.6 Hz, 3H) |
| Example 22 N-[5-[5-[(1R)-1-(3,5- dichloro-4-pyridyl) ethoxy]-1H-indazol-3- yl]-3-fluoro-2- pyridyl]-2- (dimethylamino) acetamide | 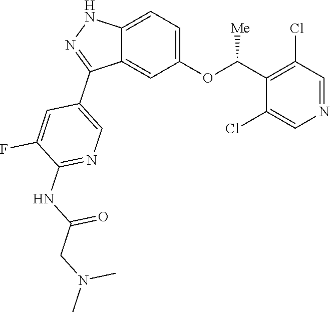 [SMILES: C[C@@H](Oc1ccc2[nH]nc(-c3cnc(NC(=O)CN(C)C)c(F)c3)c2c1)c1c(Cl)cncc1Cl] | LCMS m/z = 503 (M + H); 1H NMR (400 MHz, DMSO-d6) δ = 13.41 (br s, 1H), 10.11 (br s, 1H), 8.72-8.67 (m, 1H), 8.57 (s, 2H), 8.05 (dd, J = 1.8, 10.9 Hz 1H), 7.54 (d, J = 9.0 Hz, 1H), 7.28 (d, J = 2.0 Hz, 1H), 7.14 (dd, J = 2.2, 9.0 Hz, 1H), 6.18 (q, J = 6.7 Hz, 1H), 3.19-3.16 (m, 2H), 2.33 (s, 6H), 1.77 (d, J = 6.6 Hz, 3H) |
| Example 23 [7-[5-[(1R)-1-(3,5- dichloro-4-pyridyl) ethoxy]-1H-indazol-3- yl]-2,3-dihydro- pyrido[2,3-b][1,4] oxazin-1-yl]-(2- methyl-4-pyridyl) methanone | 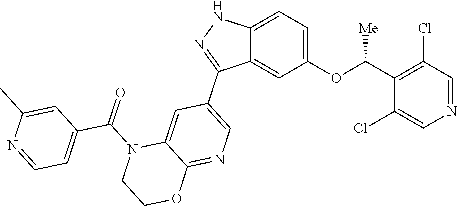 [SMILES: Cc1cc(C(=O)N2CCOc3ncc(-c4n[nH]c5ccc(O[C@H](C)c6c(Cl)cncc6Cl)cc45)cc32)ccn1] | LCMS m/z = 561 (M + H); 1H NMR (400 MHz, DMSO-d6) δ = 13.20 (s, 1H), 9.05-8.20 (m, 5H), 7.53-7.46 (m, 2H), 7.42 (d, J = 4.9 Hz, 1H), 7.18 (br s, 1H), 7.11 (dd, J = 2.2, 9.0 Hz, 1H), 6.10 (q, J = 6.6 Hz, 1H), 4.46 (br s, 2H), 3.88 (br s, 2H), 2.54 (s, 3H), 1.75 (d, J = 6.7 Hz, 3H) |
| Example 24 N-[5-[5-[(1R)-1-(3,5- dichloro-4-pyridyl) ethoxy]-1H-indazol- 3-yl]-2-pyridyl]- 3-hydroxy- pyrrolidine-1- carboxamide | 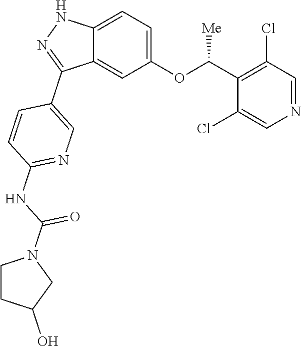 [SMILES: C[C@@H](Oc1ccc2[nH]nc(-c3ccc(NC(=O)N4CCC(O)C4)nc3)c2c1)c1c(Cl)cncc1Cl] | LCMS m/z = 490 (M + H); 1H NMR (400 MHz, DMSO-d6) δ = 13.05 (s, 1H), 8.43 (s, 1H), 8.42 (d, J = 2.1 Hz, 1H), 7.88 (dd, J = 2.4, 8.6 Hz, 1H), 7.47 (d, J = 9.0 Hz, 1H), 7.09 (dd, J = 2.3, 8.9 Hz, 1H), 7.09 (d, J = 1.7 Hz, 1H), 6.77 (dd, J = 0.7, 8.5 Hz, 1H), 6.09 (q, J = 6.7 Hz, 1H), 3.45 (s, 3H), 3.44 (s, 3H), 2.57 (s, 3H), 1.75 (d, J = 6.6 Hz, 3H) |
| Example 25 1-[7-[5-[(1R)-1-(3,5- dichloro-4-pyridyl) ethoxy]-1H-indazol- 3-yl]-2,3-dihydro- pyrido[2,3-b][1,4] oxazin-1-yl]prop-2- en-1-one | 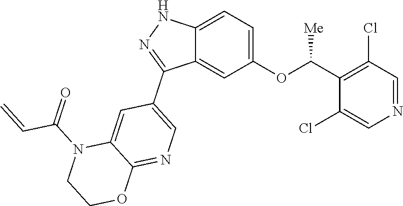 [SMILES: C=CC(=O)N1CCOc2ncc(-c3n[nH]c4ccc(O[C@H](C)c5c(Cl)cncc5Cl)cc34)cc21] | LCMS m/z = 496 (M + H); 1H NMR (400 MHz, CDCl3) δ = 10.89-9.49 (m, 1H), 8.50 (d, J = 2.1 Hz, 1H), 8.42 (s, 2H), 8.13 (br s, 1H), 7.40 (dd, J = 0.7, 8.9 Hz, 1H), 7.20-7.15 (m, 2H), 6.77-6.68 (m, 1H), 6.55 (dd, J = 1.7, 16.9 Hz, 1H), 6.06 (q, J = 6.6 Hz, 1H), 5.89-5.84 (m, 1H), 4.56 (dt, J = 0.9, 4.8 Hz, 2H), 4.18-4.05 (m, 2H), 1.82 (d, J = 6.6 Hz, 3H) |
| Example 26 1-[7-[5-[(1R)-1-(3,5- dichloro-4-pyridyl) ethoxy]-1H-indazol-3- yl]-2,3-dihydropyrido [2,3-b][1,4]oxazin-1- yl]-3-(dimethyl- amino)propan-1-one | 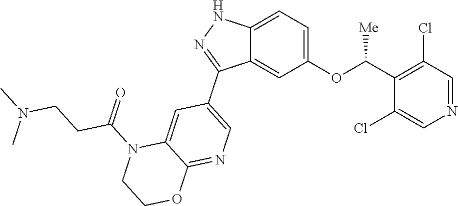 [SMILES: C[C@@H](Oc1ccc2[nH]nc(-c3cnc4c(c3)N(C(=O)CCN(C)C)CCO4)c2c1)c1c(Cl)cncc1Cl] | LCMS m/z = 541 (M + H); 1H NMR (400 MHz, CDCl3) δ = 10.80 (br s, 1H), 8.46 (d, J = 1.5 Hz, 1H), 8.41 (s, 2H), 7.39 (d, J = 9.0 Hz, 1H), 7.20 (s, 1H), 7.14 (dd, J = 2.3, 9.0 Hz, 1H), 6.07 (q, J = 6.7 Hz, 1H), 4.50 (t, J = 4.8 Hz, 2H), 4.06-3.96 (m, 2H), 2.82- 2.70 (m, 4H), 2.25 (br s, 6H), 1.81 (d, J = 6.7 Hz, 3H) |
| Example 27 isopropyl N-[5-[5- [(1R)-1-(3,5-dichloro- 4-pyridyl)ethoxy]-1H- indazol-3-yl]-2- pyridyl]carbamate | 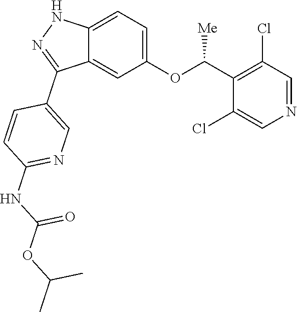 [SMILES: CC(C)OC(=O)Nc1ccc(-c2n[nH]c3ccc(O[C@H](C)c4c(Cl)cncc4Cl)cc23)cn1] | LCMS m/z = 486 (M + H); 1H NMR (400 MHz, CDCl3) δ = 10.93-9.93 (m, 1H), 8.81 (t, J = 1.5 Hz, 1H), 8.43 (s, 2H), 8.34 (s, 1H), 8.14 (d, J = 1.2 Hz, 2H), 7.39 (dd, J = 0.5, 8.9 Hz, 1H), 7.20 (d, J = 2.1 Hz, 1H), 7.16 (dd, J = 2.3, 9.0 Hz, 1H), 6.06 (q, J = 6.6 Hz, 1H), 5.09 (spt, J = 6.3 Hz, 1H), 1.82 (d, J = 6.7 Hz, 3H), 1.34 (dd, J = 2.3, 6.2 Hz, 6H) |
| Example 28 5-[5-[(1R)-1-(3,5- dichloro-4-pyridyl) ethoxy]-1H-indazol- 3-yl]-3- (difluoromethoxy) pyridin-2-amine | 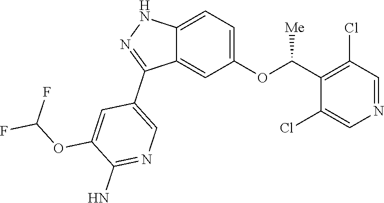 [SMILES: C[C@@H](Oc1ccc2[nH]nc(-c3cnc(N)c(OC(F)F)c3)c2c1)c1c(Cl)cncc1Cl] | LCMS m/z = 466 (M + H); 1H NMR (400 MHz, DMSO-d6) δ = 13.05 (s, 1H), 8.56 (s, 2H), 8.24 (d, J = 2.0 Hz, 1H), 7.70 (s, 1H), 7.47 (d, J = 9.0 Hz, 1H), 7.41-7.03 (m, 3H), 6.32 (s, 2H), 6.13 (q, J = 6.6 Hz, 1H), 1.76 (d, J = 6.6 Hz, 3H) |
| Example 29 4-[5-[(1R)-1-(3,5- dichloro-4-pyridyl) ethoxy]-1H-indazol- 3-yl]pyridin-2-amine | 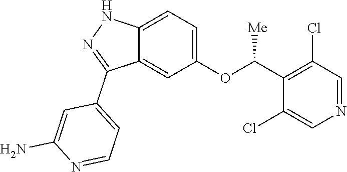 [SMILES: C[C@@H](Oc1ccc2[nH]nc(-c3ccnc(N)c3)c2c1)c1c(Cl)cncc1Cl] | LCMS m/z = 400 (M + H); 1H NMR (400 MHz, DMSO-d6) δ = 13.32 (br s, 1H), 8.60 (s, 2H), 8.25 (s, 1H), 8.01 (dd, J = 0.4, 5.3 Hz, 1H), 7.50 (d, J = 9.0 Hz, 1H), 7.25 (d, J = 2.1 Hz, 1H), 7.09 (dd, J = 2.3, 9.0 Hz, 1H), 6.95 (s, 1H), 6.84 (dd, J = 1.5, 5.4 Hz, 1H), 6.11 (q, J = 6.6 Hz, 1H), 5.96 (s, 2H), 1.77 (d, J = 6.6 Hz, 3H). |
| Example 30 2-amino-5-[5-[(1R)-1- (3,5-dichloro-4- pyridyl)ethoxy]- 1H-indazol-3-yl] pyridine-3-carbonitrile | 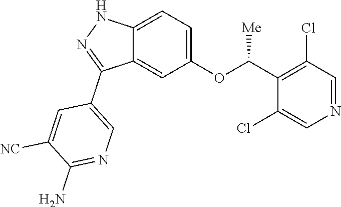 [SMILES: C[C@@H](Oc1ccc2[nH]nc(-c3cnc(N)c(C#N)c3)c2c1)c1c(Cl)cncc1Cl] | LCMS m/z = 425 (M + H)+, 1H NMR (400 MHz, DMSO-d6) δ = 13.15 (s, 1H), 8.65 (d, J = 2.4 Hz, 1H), 8.57 (s, 2H), 8.09 (d, J = 2.4 Hz, 1H), 7.48 (d, J = 9.0 Hz, 1H), 7.16-7.07 (m, 4H), 6.14 (q, J = 6.6 Hz, 1H), 1.76 (d, J = 6.7 Hz, 3H) |
| Example 31 N-[5-[5-[(1R)-1-(3,5- dichloro-4-pyridyl) ethoxy]-1H-indazol-3- yl]-2-methoxy-3- pyridyl]-3- (dimethylamino) propanamido | 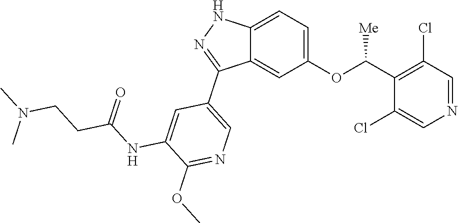 [SMILES: COc1ncc(-c2n[nH]c3ccc(O[C@H](C)c4c(Cl)cncc4Cl)cc23)cc1NC(=O)CCN(C)C] | LCMS m/z = 529 (M + H); 1H NMR (400 MHz, CD3OD) δ = 8.90 (d, J = 2.2 Hz, 1H), 8.53 (s, 1H), 8.45 (s, 2H), 8.21 (d, J = 2.1 Hz, 1H), 7.48-7.43 (m, 1H), 7.19-7.15 (m, 2H), 6.14 (q, J = 6.6 Hz, 1H), 4.10 (s, 3H), 3.04 (br t, J = 6.1 Hz, 2H), 2.81 (br t, J = 6.4 Hz, 2H), 2.60 (s, 6H), 1.82 (d, J = 6.7 Hz, 3H) |
| Example 32 5-[5-[(1R)-1-(3,5- dichloro-4-pyridyl) ethoxy]-1H-indazol-3- yl]-3-(trifluoro- methoxy)pyridin-2- amine | 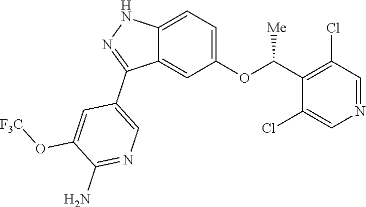 [SMILES: C[C@@H](Oc1ccc2[nH]nc(-c3cnc(N)c(OC(F)(F)F)c3)c2c1)c1c(Cl)cncc1Cl] | LCMS m/z = 484 (M + H). 1H NMR (400 MHz, DMSO-d6) δ = 13.10 (br s, 1H), 8.55 (s, 2H), 8.38 (d, J = 2.0 Hz, 1H), 7.82-7.78 (m, 1H), 7.48 (d, J = 8.9 Hz, 1H), 7.14 (d, J = 1.7 Hz, 1H), 7.11 (dd, J = 2.2, 8.9 Hz, 1H), 6.66 (s, 2H), 6.12 (q, J = 6.6 Hz, 1H), 1.74 (d, J = 6.6 Hz, 3H). |
| Example 33 5-[5-[(1R)-1-(3,5- dichloro-4-pyridyl) ethoxy]-1H-indazol- 3-yl]-3-ethoxy- pyridin-2-amine | 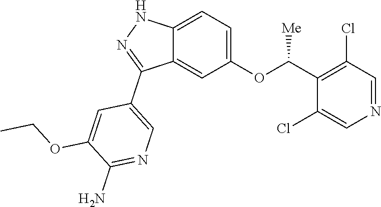 [SMILES: CCOc1cc(-c2n[nH]c3ccc(O[C@H](C)c4c(Cl)cncc4Cl)cc23)cnc1N] | LCMS m/z = 444 (M + H); 1H NMR (400 MHz, DMSO-d6) δ = 12.96 (br s, 1H), 8.56 (s, 2H), 8.14 (s, 1H), 7.92 (d, J = 1.7 Hz, 1H), 7.45 (d, J = 9.0 Hz, 1H), 7.37 (d, J = 1.7 Hz, 1H), 7.15 (d, J = 2.1 Hz, 1H), 7.10 (dd, J = 2.3, 9.0 Hz, 1H), 6.11 (q, J = 6.6 Hz, 1H), 5.88 (s, 2H), 4.10 (q, J = 7.0 Hz, 2H), 1.75 (d, J = 6.7 Hz, 3H), 1.40 (t, J = 7.0 Hz, 3H) |
| Example 34 5-[5-[(1R)-1-(3,5- dichloro-4-pyridyl) ethoxy]-1H-indazol- 3-yl]-3-methoxy- pyridin-2-amine | 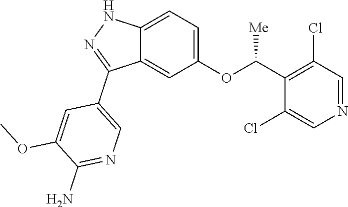 [SMILES: COc1cc(-c2n[nH]c3ccc(O[C@H](C)c4c(Cl)cncc4Cl)cc23)cnc1N] | LCMS m/z = 430 (M + H); 1H NMR (400 MHz, CDCl3) δ = 10.06 (br s, 1H), 8.42 (s, 2H), 8.12 (d, J = 1.7 Hz, 1H), 7.47 (d, J = 1.7 Hz, 1H), 7.36 (d, J = 9.0 Hz, 1H), 7.19-7.10 (m, 1H), 6.08 (q, J = 6.7 Hz, 1H), 4.98 (br s, 2H), 3.93 (s, 3H), 1.82 (d, J = 6.6 Hz, 3H) |

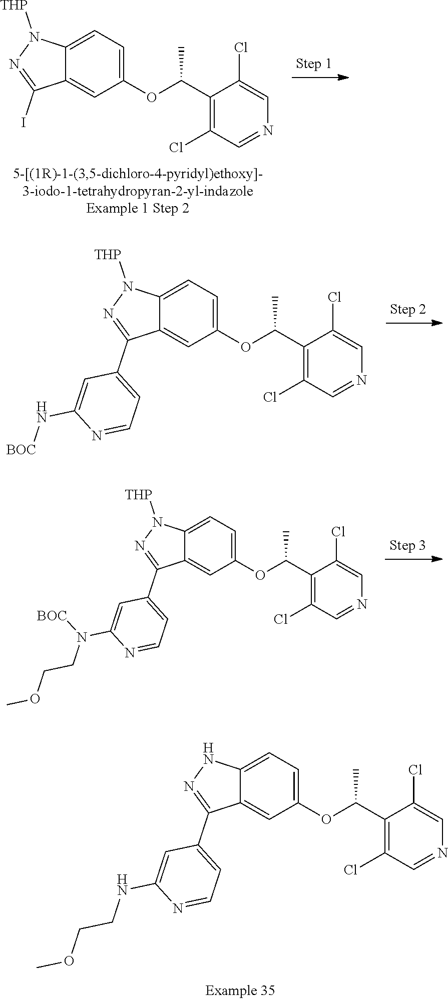

### Example 35. (R)-4-(5-(1-(3,5-Dichloropyridin-4-yl)ethoxy)-1H-indazol-3-yl)-N-(2-methoxyethyl)pyridin-2-amine

Step 1. tert-Butyl (4-(5-((R)-1-(3,5-dichloropyridin-4-yl)ethoxy)-1-(tetrahydro-2H-pyran-2-yl)-1H-indazol-3-yl)pyridin-2-yl)carbamate. Example 1 step 2 product (565.2 mg, 0.98 mmol, 1.0 equiv), tert-butyl N-[4-(4,4,5,5-tetramethyl-1,3,2-dioxaborolan-2-yl)-2-pyridyl]carbamate (384.2 mg, 1.08 mmol, 1.1 equiv), [1,1′-bis(diphenylphosphino)-ferrocene]dichloropalladium(II) (40.5 mg, 0.1 mmol, 0.1 equiv) and potassium carbonate (301.5 mg, 1.96 mmol, 2.0 equiv) in a mixture of 10 to 1 mixture of 1,4-dioxane and water was heated at 100° C. under a nitrogen atmosphere for 3 hours. The mixture was cooled to room temperature and the solvent was removed under reduced pressure. The residue was diluted with water (10 mL), extracted with ethyl acetate (2×20 mL). The combined organic layers were concentrated under reduced pressure onto silica gel (6 g). The crude product was purified on a Biotage automated chromatography system (Biotage Sfär 25 g, 20 μm column), eluting with a gradient of 0 to 50% ethyl acetate in heptanes to give a white solid (404 mg, 64%). Analysis: LCMS m/z=584 (M+H).

Step 2. tert-Butyl (4-(5-((R)-1-(3,5-dichloropyridin-4-yl)ethoxy)-1-(tetrahydro-2H-pyran-2-yl)-1H-indazol-3-yl)pyridin-2-yl)(2-methoxyethyl)carbamate. A mixture of product step 1 (210 mg, 0.36 mmol, 1.0 equiv), a 60% dispersion of sodium hydride in mineral oil (67.3 mg, 1.5 mmol, 4.5 equiv) and 1-bromo-2-methoxyethane (101.2 μL, 1.08 mol, 3.0 equiv) in N-methyl-2-pyrrolidone (3.6 mL) was heated at 75° C. for 4 hours. The reaction was cooled to room temperature and the volatiles were removed under reduced pressure. The residue was diluted with ethyl acetate (15 mL), washed with water (2×10 mL), dried over sodium sulfate and concentrated onto silica gel (2 g). The crude product was purified on a Biotage automated chromatography system (Sorbtech, 12 g silica gel column), eluting with a gradient of 0 to 60% ethyl acetate in heptanes to give a light-yellow oil (0.24 g, 100%). Analysis: LCMS m/z=642 (M+H).

Step 3. (R)-4-(5-(1-(3,5-Dichloropyridin-4-yl)ethoxy)-1H-indazol-3-yl)-N-(2-methoxyethyl)pyridin-2-amine. The product from step 2 (0.24 g, 0.37 mmol, 1.0 equiv) was treated with a 1 to 1 mixture of dichloromethane and trifluoroacetic acid (4.8 mL) at room temperature for 3 hours. The volatiles were removed under reduced pressure. The residue was dissolved in a 1 to 1 mixture of dichloromethane and methanol (24 mL) and treated with MP-Carbonate® resin (9.7 g) at room temperature for 2 hours. The resin was filtered and the filtrate was concentrated onto Celite® (2 g) under reduced pressure. The product was purified on a Biotage automated chromatography system (RediSep Rf Gold R) Reversed-phase C18, 50 g), eluting with a gradient of 0 to 70% acetonitrile in water. The product was further purified on a Biotage automated chromatography system (RediSep Rf Gold® Reversed-phase C18, 50 g), eluting with a gradient of 0 to 70% acetonitrile in water to give an off-white solid (14.6 mg, 9%). Analysis: LCMS m/z=458 (M+H). ¹H NMR (400 MHz, DMSO-d6) δ=13.28 (br s, 1H), 8.59 (s, 2H), 8.06 (d, J=5.3 Hz, 1H), 7.50 (d, J=8.9 Hz, 1H), 7.29 (d, J=2.2 Hz, 1H), 7.09 (dd, J=2.3, 9.0 Hz, 1H), 7.01 (d, J=0.4 Hz, 1H), 6.84 (dd, J=1.4, 5.3 Hz, 1H), 6.63-6.54 (m, 1H), 6.15-6.09 (m, 1H), 3.52-3.47 (m, 4H), 3.08 (s, 1H), 1.77 (d, J=6.6 Hz, 3H).

### Example 36. 2-[[4-[5-[(1R)-1-(3,5-Dichloro-4-pyridyl)ethoxy]-1H-indazol-3-yl]-2-pyridyl]amino]ethanol

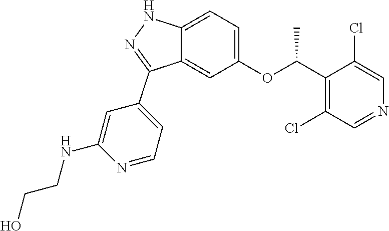 [SMILES: C[C@@H](Oc1ccc2[nH]nc(-c3ccnc(NCCO)c3)c2c1)c1c(Cl)cncc1Cl]

Step 1. tert-Butyl (4-(5-((R)-1-(3,5-dichloropyridin-4-yl)ethoxy)-1-(tetrahydro-2H-pyran-2-yl)-1H-indazol-3-yl)pyridin-2-yl)(2-((tetrahydro-2H-pyran-2-yl)oxy)ethyl)carbamate. Example 35 step 1 product (194.3 mg, 0.33 mmol, 1.0 equiv), a 60% dispersion of sodium hydride in mineral oil (60.0 mg, 1.5 mmol, 4.5 equiv) and 2-(2-bromoethoxy) tetrahydro-2H-pyran (150.7 μL, 1.0 mol, 3.0 equiv) in N-methyl-2-pyrrolidone (3.3 mL) was heated at 75° C. for 3.5 hours. The reaction was cooled to room temperature and the volatiles were removed under reduced pressure. The residue was diluted with ethyl acetate (30 mL), washed with water (30 mL), dried over sodium sulfate and concentrated onto silica gel (2 g). The crude product was purified on a Biotage automated chromatography system (Sorbtech, 12 g silica gel column), eluting with a gradient of 0 to 60% ethyl acetate in heptanes to give a light yellow oil (185 mg, 78%). Analysis: LCMS m/z=712.2 (M+H).

Step 2. (R)-2-((4-(5-(1-(3,5-Dichloropyridin-4-yl)ethoxy)-1H-indazol-3-yl)pyridin-2-yl)amino)ethan-1-ol. Product from step 1 (0.24 g, 0.34 mmol, 1.0 equiv) was treated with a 1 to 1 mixture of dichloromethane and trifluoroacetic acid (4.8 mL) at room temperature overnight. The volatiles were removed under reduced pressure and the residue was dissolved in a 1 to 1 mixture of dichloromethane and methanol (24 mL) and treated with MP-Carbonate® resin (9.0 g) at room temperature for 2 hours. The resin was filtered and the filtrate was concentrated onto Celite (2 g) under reduced pressure. The product was purified on a Biotage automated chromatography system (RediSep Rf Gold® Reversed-phase C18, 50 g), eluting with a gradient of 0 to 70% acetonitrile in water. This material was further purified on a Biotage automated chromatography system (RediSep Rf Gold® Reversed-phase C18, 50 g), eluting with a gradient of 0 to 70% acetonitrile in water to give an off-white solid (14.8 mg, 10%). Analysis: LCMS m/z=444 (M+H); ¹H NMR (400 MHZ, DMSO-d6) δ=13.30 (br s, 1H), 8.60 (s, 2H), 8.05 (d, J=5.3 Hz, 1H), 7.50 (d, J=9.0 Hz, 1H), 7.28 (d, J=2.2 Hz, 1H), 7.09 (dd, J=2.3, 9.0 Hz, 1H), 6.99 (s, 1H), 6.84 (dd, J=1.4, 5.3 Hz, 1H), 6.55 (t, J=5.6 Hz, 1H), 6.15-6.08 (m, 1H), 4.77 (br s, 1H), 3.61-3.54 (m, 2H), 3.42-3.35 (m, 2H), 3.23-3.12 (m, 1H), 1.77 (d, J=6.6 Hz, 3H).

### Example 37. 5-[5-[(1R)-1-(3,5-Dichloro-4-pyridyl)ethoxy]-1H-indazol-3-yl]-3-(2,2,2-trifluoroethoxy)pyridin-2-amine

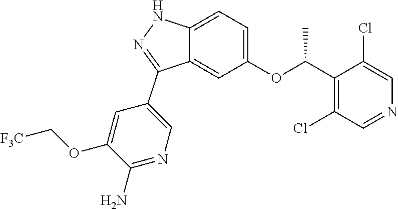 [SMILES: C[C@@H](Oc1ccc2[nH]nc(-c3cnc(N)c(OCC(F)(F)F)c3)c2c1)c1c(Cl)cncc1Cl]

Step 1. 5-Bromo-3-(2,2,2-trifluoroethoxy)pyridin-2-amine. A mixture of 2-amino-5-bromo-pyridin-3-ol (1.0 g, 5.3 mmol, 1.1 equiv) and cesium carbonate (1.72 g, 5.29 mmol, 1.1 equiv) in N,N-dimethylformamide (22 mL) was stirred for 1 hour. 1,1,1-Trifluoro-2-iodoethane (1.0 g, 4.76 mmol) in N,N-dimethylformamide (1 mL) was added at room temperature. The mixture was heated at 90° C. overnight then cooled to room temperature. The mixture was diluted with ethyl acetate (100 mL), washed with water (50 mL) and half-saturated sodium bicarbonate (100 mL). The organic layer was dried over sodium sulfate, filtered and concentrated under reduced pressure. The residue was purified on a Büchi automated chromatography system (Biotage Sfär 50 g, 60 μm column), eluting with a gradient of 0 to 30% ethyl acetate in heptanes to give a white solid (0.72 g, 56%). Analysis: LCMS m/z=271 (M+H); ¹H NMR (400 MHZ, DMSO-d6) δ=7.68 (d, J=2.0 Hz, 1H), 7.42 (d, J=2.0 Hz, 1H), 5.97 (s, 2H), 4.80 (q, J=8.9 Hz, 2H).

Step 2. 5-(5-((R)-1-(3,5-Dichloropyridin-4-yl)ethoxy)-1-(tetrahydro-2H-pyran-2-yl)-1H-indazol-3-yl)-3-(2,2,2-trifluoroethoxy)pyridin-2-amine. Compound from example 1 step 2 (0.25 g, 0.48 mmol, 1 equiv) was combined with bis(neopentylglycolato)diboron (0.13 g, 0.57 mmol, 1.2 equiv), [1,1′-bis(diphenylphosphino)ferrocene]palladium(II) dichloride (39 mg, 0.05 mmol, 0.1 equiv) and potassium acetate (0.15 g, 1.5 mmol, 3 equiv) in 1,4-dioxane (10 mL) and sparged with nitrogen for 5 minutes. The reaction was heated at 90° C. for 16 hours. Additional bis(neopentylglycolato)diboron (0.098 g, 0.43 mmol, 0.9 equiv) and [1,1′-bis(diphenylphosphino)ferrocene]palladium(II) dichloride (30 mg, 0.04 mmol, 0.08 equiv) were added and the mixture was sparged with nitrogen for an additional 5 minutes, then heated for an additional 6 hours. The mixture was cooled to room temperature and 5-Bromo-3-(2,2,2-trifluoroethoxy)pyridin-2-amine (0.31 g, 1.1 mmol, 2.3 equiv), potassium carbonate (0.32 g, 2.3 mmol, 4.8 equiv), [1,1′-bis(diphenylphosphino) ferrocene]palladium(II) dichloride (39 mg, 0.05 mmol, 0.1 equiv) and water (1.5 mL) were added. After sparging with nitrogen for an additional 5 minutes, the reaction was heated at 90° C. for 24 hours. The mixture was cooled to room temperature, diluted with water (20 mL) and extracted with dichloromethane (3×20 mL). The combined organic layers were concentrated onto silica gel (6 g). The crude product was purified on a Büchi automated chromatography system (Biotage Sfär 50 g, 60 μm column), eluting with a gradient of 0 to 100% ethyl acetate in heptanes to give a beige solid (127 mg, 45%). Analysis: LCMS m/z=582 (M+H); ¹H NMR (400 MHZ, DMSO-d6) δ=8.55 (s, 2H), 8.02 (dd, J=1.7, 3.5 Hz, 1H), 7.67 (dd, J=2.6, 9.0 Hz, 1H), 7.55 (d, J=1.7 Hz, 1H), 7.21 (s, 1H), 7.16 (d, J=9.2 Hz, 1H), 6.17 (dd, J=1.7, 6.7 Hz, 1H), 6.04 (s, 2H), 5.77 (td, J=2.9, 10.3 Hz, 1H), 4.87 (q, J=9.0 Hz, 2H), 4.30 (t, J=5.4 Hz, 1H), 3.88 (br s, 1H), 3.73 (br d, J=6.5 Hz, 1H), 3.14 (d, J=5.4 Hz, 2H), 2.47-2.36 (m, 1H), 2.05-1.88 (m, 2H), 1.79-1.70 (m, 4H), 1.57 (br s, 2H), 0.75 (s, 3H).

Step 3. (R)-5-(5-(1-(3,5-Dichloropyridin-4-yl)ethoxy)-1H-indazol-3-yl)-3-(2,2,2-trifluoroethoxy)pyridin-2-amine. The product from step 2 (125 mg, 0.21 mmol) and trifluoroacetic acid (1 mL, 12.5 mmol, 60 equiv) in dichloromethane (1 mL) was stirred at room temperature for 2 hours. The mixture was concentrated under reduced pressure. The residue was purified on a Gilson automated chromatography system (Waters Atlantis T3 Prep OBD column, 5 μm, 19×250 mm), eluting with a gradient of 0 to 100% acetonitrile in water with 0.1% formic acid. Product fractions were lyophilized to give a tan solid as the TFA salt (77 mg, 60%). Analysis: LCMS m/z=498 (M+H); ¹H NMR (400 MHZ, DMSO-d6) δ=13.27 (br s, 1H), 8.57 (s, 2H), 8.02 (d, J=1.7 Hz, 1H), 7.84 (br s, 1H), 7.50 (d, J=9.0 Hz, 1H), 7.27-7.24 (m, 1H), 7.11 (dd, J=2.3, 9.0 Hz, 1H), 6.15 (q, J=6.6 Hz, 1H), 5.00 (q, J=8.6 Hz, 2H), 1.76 (d, J=6.7 Hz, 3H).

### Example 38. 7-[5-[(1R)-1-(3,5-dichloro-4-pyridyl)ethoxy]-1H-indazol-3-yl]-4H-pyrido[3,2-b][1,4]oxazin-3-one

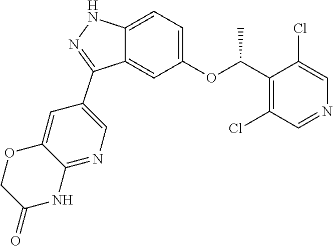 [SMILES: C[C@@H](Oc1ccc2[nH]nc(-c3cnc4c(c3)OCC(=O)N4)c2c1)c1c(Cl)cncc1Cl]

Step 1. (2-Amino-5-(5-((R)-1-(3,5-dichloropyridin-4-yl)ethoxy)-1-(tetrahydro-2H-pyran-2-yl)-1H-indazol-3-yl)pyridin-3-yl)methanol. Product example 1 step 2 (0.25 g, 0.48 mmol) was combined with bis(neopentylglycolato)diboron (0.13 g, 0.57 mmol, 1.2 equiv), [1,1′-bis(diphenylphosphino)ferrocene]palladium(II) dichloride (39 mg, 0.05 mmol, 0.1 equiv) and potassium acetate (0.15 g, 1.5 mmol, 3 equiv) in 1,4-dioxane (10 mL). After sparging with nitrogen for 5 minutes, the reaction was heated at 90° C. for 16 hours and cooled to rt. (2-Amino-5-bromo-3-pyridyl)methanol (0.22 g, 1.2 mmol, 2.4 equiv), potassium carbonate (0.16 g, 1.2 mmol, 2.4 equiv), [1,1′-bis(diphenyl phosphino)ferrocene]palladium(II) dichloride (50 mg, 0.07 mmol, 0.14 equiv) and water (5 mL) were added and the mixture was sparged with nitrogen for an additional 5 minutes. After heating at 90° C. for 2 hours, the mixture was cooled to room temperature, diluted with water (20 mL) and extracted with dichloromethane (3×20 mL). The combined organic layers were concentrated under reduced pressure onto silica gel (6 g). The crude product was purified on a Büchi automated chromatography system (Biotage Sfär 50 g, 60 μm column), eluting with a gradient of 0 to 100% ethyl acetate in heptanes to give a brown oil (113 mg, 46%). Analysis: LCMS m/z=514 (M+H).

Step 2. 6-(5-((R)-1-(3,5-Dichloropyridin-4-yl)ethoxy)-1-(tetrahydro-2H-pyran-2-yl)-1H-indazol-3-yl)-1,4-dihydro-2H-pyrido[2,3-d][1,3]oxazin-2-one. The product from step 1 (113 mg, 0.22 mmol, 1 equiv) in THF (1 mL) was treated with di-tert-butyldicarbonate (72 mg, 0.32 mmol, 1.5 equiv) at room temperature for 2 days then at 50° C. for 16 hours. The reaction mixture was evaporated to dryness. The residue was diluted with THF (3 mL) and ethyl acetate (10 mL), then washed with water (2 mL). The organic extract was concentrated under reduced pressure onto silica gel (6 g). The crude product was purified on a Büchi automated chromatography system (Biotage Sfär 25 g, 60 μm column), eluting with a gradient of 0 to 100% ethyl acetate in heptanes to give a beige solid (71 mg, 60%). Analysis: LCMS m/z=540 (M+H).

Step 3. (R)-6-(5-(1-(3,5-Dichloropyridin-4-yl)ethoxy)-1H-indazol-3-yl)-1,4-dihydro-2H-pyrido[2,3-d][1,3]oxazin-2-one. A mixture of the product from step 2 (70 mg, 0.12 mmol, 1 equiv) and trifluoroacetic acid (0.5 mL, 6.5 mmol, 52 equiv) in dichloromethane (0.5 mL) was stirred at room temperature for 3 hours. The mixture was concentrated under reduced pressure. The residue was purified on a Gilson automated chromatography system (Waters Atlantis T3 Prep OBD column, 5 μm, 19×250 mm), eluting with a gradient of 0 to 100% acetonitrile in water with 0.1% formic acid. Product containing fractions were diluted with saturated aqueous sodium bicarbonate (100 mL) and extracted with dichloromethane (3×20 mL). The organic extract was dried over sodium sulfate, filtered and concentrated under reduced pressure to give an off-white solid (21 mg, 38%). Analysis: LCMS m/z=456 (M+H); ¹H NMR (400 MHZ, DMSO-d6) δ=13.24 (s, 1H), 10.86 (s, 1H), 8.66-8.53 (m, 3H), 8.03 (s, 1H), 7.50 (d, J=9.0 Hz, 1H), 7.22 (d, J=1.6 Hz, 1H), 7.11 (dd, J=2.1, 9.0 Hz, 1H), 6.13 (q, J=6.5 Hz, 1H), 5.45 (s, 2H), 1.76 (d, J=6.6 Hz, 3H).

Examples 39-51 were synthesized using procedures described in the previous examples.

| | | |
|---|---|---|
| Example 39 [7-[5-[(1R)-1- (3,5-dichloro- 4-pyridyl) ethoxy]-1H- indazol-3-yl]- 2,3-dihydro- pyrido[2,3-b] [1,4]oxazin- 1-yl]-(6- methyl-2- pyridyl) methanone | 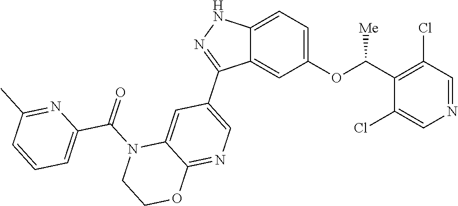 [SMILES: Cc1cccc(C(=O)N2CCOc3ncc(-c4n[nH]c5ccc(O[C@H](C)c6c(Cl)cncc6Cl)cc45)cc32)n1] | LCMS m/z = 561 (M + H); 1H NMR (400 MHz, CDCl3) δ = 10.22 (br s, 1H), 8.71 (br s, 1H), 8.48 (d, J = 2.1 Hz, 1H), 8.39 (s, 2H), 7.74 (t, J = 7.8 Hz, 1H), 7.62 (d, J = 7.6 Hz, 1H), 7.36 (d, J = 9.0 Hz, 1H), 7.26-7.16 (m, 2H), 7.12 (dd, J = 2.3, 9.0 Hz, 1H), 6.07 (q, J = 6.7 Hz, 1H), 4.54 (t, J = 4.5 Hz, 2H), 4.20-4.10 (m, 2H), 2.56 (s, 3H), 1.80 (d, J = 6.6 Hz, 3H) |
| Example 40 [7-[5-[(1R)-1- (3,5-dichloro- 4-pyridyl) ethoxy]-1H- indazol-3-yl]- 2,3-dihydro- pyrido[2,3-b] [1,4]oxazin-1- yl]-(2- methyl- pyrimidin-4- yl)methanone | 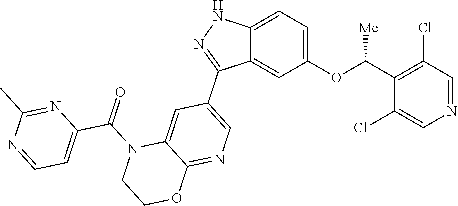 [SMILES: Cc1nccc(C(=O)N2CCOc3ncc(-c4n[nH]c5ccc(O[C@H](C)c6c(Cl)cncc6Cl)cc45)cc32)n1] | LCMS m/z = 562 (M + H); 1H NMR (400 MHz, CDCl3) δ = 10.29 (br s, 1H), 9.08 (br s, 1H), 8.86 (br d, J = 4.6 Hz, 1H), 8.51 (s, 1H), 8.40 (s, 2H), 7.55 (br d, J = 2.8 Hz, 1H), 7.40-7.34 (m, 1H), 7.21 (br s, 1H), 7.14 (dd, J = 2.1, 9.0 Hz, 1H), 6.07 (q, J = 6.6 Hz, 1H), 4.57 (br s, 2H), 4.13 (br s, 2H), 2.75 (br s, 3H), 1.81 (d, J = 6.7 Hz, 3H) |
| Example 41 [7-[5-[(1R)-1- (3,5-dichloro- 4-pyridyl) ethoxy]-1H- indazol-3-yl]- 2,3-dihydro- pyrido[2,3-b] [1,4]oxazin-1- yl]-6-methyl- 3-pyridyl) methanone | 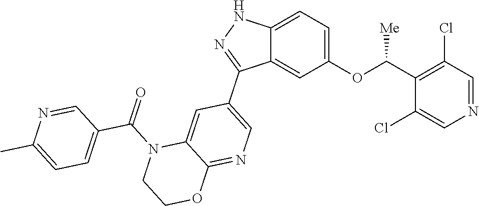 [SMILES: Cc1ccc(C(=O)N2CCOc3ncc(-c4n[nH]c5ccc(O[C@H](C)c6c(Cl)cncc6Cl)cc45)cc32)cn1] | LCMS m/z = 561 (M + H); 1H NMR (400 MHz, CD3OD) δ = 8.71 (d, J = 2.1 Hz, 1H), 8.57-8.41 (m, 1H), 8.39 (s, 2H), 8.32 (s, 1H), 8.04-7.93 (m, 1H), 7.41 (t, J = 7.8 Hz, 2H), 7.15-7.11 (m, 1H), 7.06 (s, 1H), 6.09 (q, J = 6.6 Hz, 1H), 4.53 (br t, J = 4.3 Hz, 2H), 4.16-3.99 (m, 2H), 2.58 (s, 3H), 1.78 (d, J = 6.6 Hz, 3H), 1.39- 1.18 (m, 2H) |
| Example 42 [7-[5-[(1R)-1- (3,5-dichloro- 4-pyridyl) ethoxy]-1H- indazol-3-yl]- 2,3-dihydro- pyrido[2,3-b] [1,4]oxazin-1- yl]-(2-methyl- 3-pyridyl) methanone | 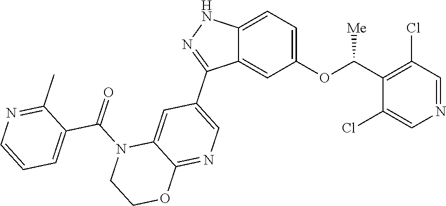 [SMILES: Cc1ncccc1C(=O)N1CCOc2ncc(-c3n[nH]c4ccc(O[C@H](C)c5c(Cl)cncc5Cl)cc34)cc21] | LCMS m/z = 561 (M + H); 1H NMR (400 MHz, DMSO-d6) δ = 13.21 (br s, 1H), 8.58-8.49 (m, 3H), 8.38 (d, J = 1.3 Hz, 1H), 7.89 (dd, J = 1.5, 7.6 Hz, 1H), 7.50 (d, J = 8.9 Hz, 1H), 7.35 (br s, 1H), 7.23 (br s, 1H), 7.11 (dd, J = 1.3, 9.0 Hz, 1H), 6.13 (q, J = 6.6 Hz, 1H), 4.47 (br s, 2H), 3.79 (br s, 2H), 1.76 (d, J = 6.7 Hz, 3H) |
| Example 43 3-(dimethyl- amino)propyl 7-[5-[(1R)-1- (3,5-dichloro- 4-pyridyl) ethoxy]-1H- indazol-3-yl]- 2,3-dihydro- pyrido[2,3-b] [1,4]oxazine- 1-carboxylate | 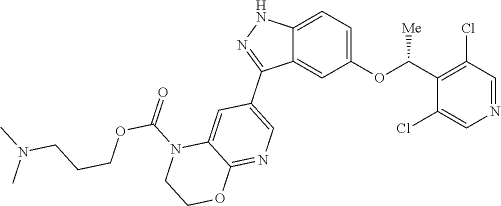 [SMILES: C[C@@H](Oc1ccc2[nH]nc(-c3cnc4c(c3)N(C(=O)OCCCN(C)C)CCO4)c2c1)c1c(Cl)cncc1Cl] | LCMS m/z = 571 (M + H); 1H NMR (400 MHz, DMSO-d6) δ = 13.24 (br s, 1H), 8.69 (br s, 1H), 8.56 (s, 2H), 8.29-8.22 (m, 2H), 7.51 (d, J = 9.0 Hz, 1H), 7.18 (d, J = 2.1 Hz, 1H), 7.12 (dd, J = 2.3, 9.0 Hz, 1H), 6.09 (q, J = 6.6 Hz, 1H), 4.44 (br t, J = 4.2 Hz, 2H), 4.26-4.19 (m, 2H), 4.02- 3.89 (m, 3H), 2.44-2.33 (m, 2H), 2.13 (br s, 6H), 1.81 (quin, J = 6.8 Hz, 2H), 1.75 (d, J = 6.6 Hz, 3H) |
| Example 44 3-[5-[(1R)-1- (3,5-dichloro- 4-pyridyl) ethoxy]-1H- indazol-3-yl]- 5-methoxy- aniline | 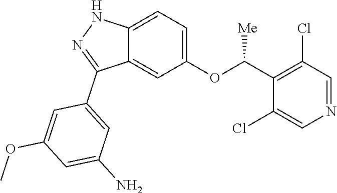 [SMILES: COc1cc(N)cc(-c2n[nH]c3ccc(O[C@H](C)c4c(Cl)cncc4Cl)cc23)c1] | LCMS m/z = 429 (M + H); 1H NMR (400 MHz, DMSO-d6) δ = 12.97 (s, 1H), 8.57 (s, 2H), 7.45 (d, J = 8.9 Hz, 1H), 7.20 (s, 1H), 7.07 (dd, J = 2.0, 8.9 Hz, 1H), 6.66 (s, 1H), 6.45 (s, 1H), 6.19 (s, 1H), 6.08 (q, J = 6.7 Hz, 1H), 5.16 (s, 2H), 3.75 (s, 3H), 1.76 (d, J = 6.7 Hz, 3H) |
| Example 45 N-[3-[5- [(1R)-1-(3,5- dichloro-4- pyridyl) ethoxy]-1H- indazol-3-yl]- 5-methoxy- phenyl] pyridine-2- carboxamide | 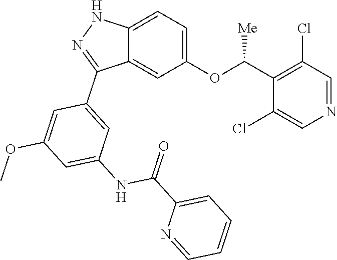 [SMILES: COc1cc(NC(=O)c2ccccn2)cc(-c2n[nH]c3ccc(O[C@H](C)c4c(Cl)cncc4Cl)cc23)c1] | LCMS m/z = 534 (M + H); 1H NMR (400 MHz, DMSO-d6) δ = 13.15 (s, 1H), 10.72 (s, 1H), 8.82-8.78 (m, 1H), 8.54 (s, 2H), 8.22 (td, J = 1.1, 7.8 Hz, 1H), 8.12 (dt, J = 1.7, 7.7 Hz, 1H), 7.99 (t, J = 1.5 Hz, 1H), 7.72 (ddd, J = 1.3, 4.8, 7.5 Hz, 1H), 7.65 (t, J = 2.1 Hz, 1H), 7.50 (d, J = 8.9 Hz, 1H), 7.33 (d, J = 2.1 Hz, 1H), 7.11 (dd, J = 2.2, 9.0 Hz, 1H), 7.08 (dd, J = 1.3, 2.3 Hz, 1H), 6.13 (q, J = 6.6 Hz, 1H), 3.31 (s, 3H), 1.76 (d, J = 6.7 Hz, 3H) |
| Example 46 3-[5-[(1R)-1- (3,5-dichloro- 4-pyridyl) ethoxy]-1H- indazol-3-yl]- 5-methoxy-N- (2-pyridyl- methyl) aniline | 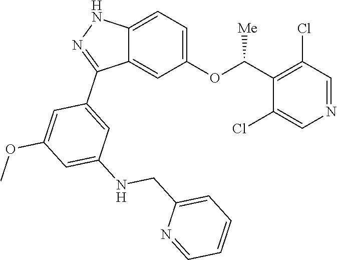 [SMILES: COc1cc(NCc2ccccn2)cc(-c2n[nH]c3ccc(O[C@H](C)c4c(Cl)cncc4Cl)cc23)c1] | LCMS m/z = 520 (M + H); 1H NMR (400 MHz, DMSO-d6) δ = 12.99 (s, 1H), 8.60- 8.51 (m, 3H), 7.77 (dt, J = 1.7, 7.7 Hz, 1H), 7.43 (dd, J = 8.9, 10.2 Hz, 2H), 7.26 (dd, J = 5.3, 6.7 Hz, 1H), 7.20 (s, 1H), 7.07 (dd, J = 2.2, 9.0 Hz, 1H), 6.70 (s, 1H), 6.51-6.43 (m, 2H), 6.18 (t, J = 1.9 Hz, 1H), 6.09 (q, J = 6.7 Hz, 1H), 4.43 (d, J = 6.0 Hz, 2H), 3.73 (s, 3H), 1.76 (d, J = 6.6 Hz, 3H) |
| Example 47 3-[5-[(1R)-1- (3,5-dichloro- 4-pyridyl) ethoxy]-1H- indazol-3-yl]- 5-methoxy-N- (3-pyridyl- methyl) aniline | 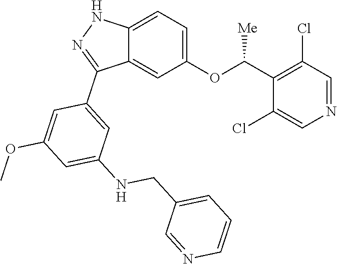 [SMILES: COc1cc(NCc2cccnc2)cc(-c2n[nH]c3ccc(O[C@H](C)c4c(Cl)cncc4Cl)cc23)c1] | LCMS m/z = 520.1 (M + H); 1H NMR (400 MHz, DMSO-d6) δ = 13.00 (s, 1H), 8.62 (d, J = 1.6 Hz, 1H), 8.55 (s, 2H), 8.45 (dd, J = 1.6, 4.8 Hz, 1H), 7.79 (td, J = 1.9, 7.9 Hz, 1H), 7.45 (d, J = 9.0 Hz, 1H), 7.40- 7.34 (m, 1H), 7.18 (d, J = 1.6 Hz, 1H), 7.07 (dd, J = 2.2, 9.0 Hz, 1H), 6.71 (s, 1H), 6.47 (s, 1H), 6.40 (t, J = 6.1 Hz, 1H), 6.18 (t, J = 2.1 Hz, 1H), 6.07 (q, J = 6.5 Hz, 1H), 4.37 (d, J = 6.1 Hz, 2H), 3.74 (s, 3H), 1.75 (d, J = 6.7 Hz, 3H) |
| Example 48 7-[5-[(1R)-1- (3,5-dichloro- 4-pyridyl) ethoxy]-1H- indazol-3-yl]- 1-(2-pyridyl- methyl)-2,3- dihydro- pyrido[2,3- b][1,4] oxazine | 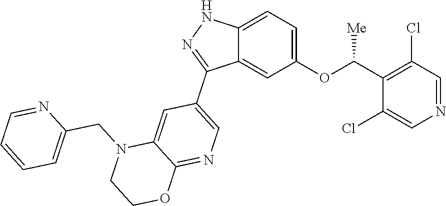 [SMILES: C[C@@H](Oc1ccc2[nH]nc(-c3cnc4c(c3)N(Cc3ccccn3)CCO4)c2c1)c1c(Cl)cncc1Cl] | LCMS m/z = 533 (M + H); 1H NMR (400 MHz, DMSO-d6) δ = 13.06 (br s, 1H), 8.64 (d, J = 4.3 Hz, 1H), 8.56 (s, 2H), 7.96 (t, J = 7.2 Hz, 1H), 7.79 (d, J = 2.0 Hz, 1H), 7.56 (d, J = 7.8 Hz, 1H), 7.47-7.42 (m, 2H), 7.32 (d, J = 2.0 Hz, 1H), 7.13 (d, J = 2.1 Hz, 1H), 7.06 (dd, J = 2.3, 9.0 Hz, 1H), 6.08 (q, J = 6.5 Hz, 1H), 4.77 (s, 2H), 4.53-4.46 (m, 2H), 3.65-3.58 (m, 2H), 1.75 (d, J = 6.7 Hz, 3H) |
| Example 49 2-[7-[5-[(1R)- 1-(3,5- dichloro-4- pyridyl) ethoxy]-1H- indazol-3-yl]- 2,3-dihydro- pyrido[2,3-b] [1,4]oxazin- 1-yl]ethanol | 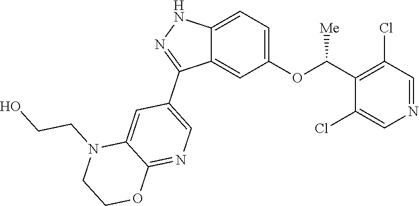 [SMILES: C[C@@H](Oc1ccc2[nH]nc(-c3cnc4c(c3)N(CCO)CCO4)c2c1)c1c(Cl)cncc1Cl] | LCMS m/z = 486 (M + H); 1H NMR (400 MHz, DMSO-d6) δ = 13.05 (s, 1H), 8.57 (s, 2H), 7.71 (d, J = 2.0 Hz, 1H), 7.48 (d, J = 8.9 Hz, 1H), 7.38 (d, J = 2.0 Hz, 1H), 7.18 (d, J = 2.0 Hz, 1H), 7.11 (dd, J = 2.3, 9.0 Hz, 1H) 6.11 (q, J = 6.6 Hz, 1H), 4.78 (t, J = 5.4 Hz, 1H), 4.37-4.30 (m, 2H), 3.64 (q, J = 5.7 Hz, 2H), 3.48-3.45 (m, 2H), 3.45-3.38 (m, 2H), 1.75 (d, J = 6.7 Hz, 3H) |
| Example 50 7-[5-[(1R)-1- (3,5-dichloro- 4-pyridyl) ethoxy]-1H- indazol-3-yl]- 1-(2- morpholino- ethyl)-2,3- dihydropyrido [2,3-b][1,4] oxazine | 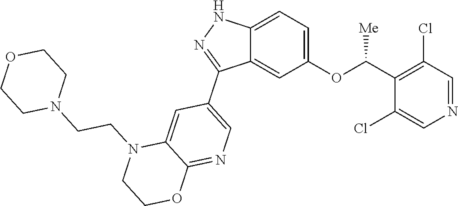 [SMILES: C[C@@H](Oc1ccc2[nH]nc(-c3cnc4c(c3)N(CCN3CCOCC3)CCO4)c2c1)c1c(Cl)cncc1Cl] | LCMS m/z = 555 (M + H); 1H NMR (400 MHz, DMSO-d6) δ = 13.07 (s, 1H), 8.58 (s, 2H), 7.74 (d, J = 1.8 Hz, 1H), 7.48 (d, J = 9.0 Hz, 1H), 7.38 (d, J = 1.8 Hz, 1H), 7.19 (d, J = 2.1 Hz, 1H), 7.10 (dd, J = 2.2, 9.0 Hz, 1H), 6.11 (q, J = 6.6 Hz, 1H), 4.37- 4.31 (m, 2H), 3.58-3.53 (m, 4H), 3.51- 3.43 (m, 4H), 2.56-2.52 (m, 2H), 2.47- 2.38 (m, 4H), 1.75 (d, J = 6.6 Hz, 3H) |
| Example 51 2-[7-[5-[(1R)- 1-(3,5- dichloro-4- pyridyl) ethoxy]-1H- indazol-3-yl]- 2,3-dihydro- pyrido[2,3-b] [1,4]oxazin-1- yl]-N,N- dimethyl- ethanamine | 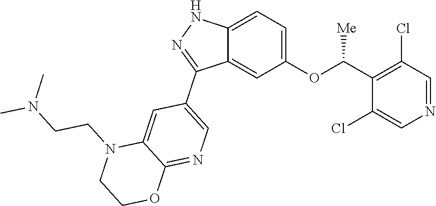 [SMILES: C[C@@H](Oc1ccc2[nH]nc(-c3cnc4c(c3)N(CCN(C)C)CCO4)c2c1)c1c(Cl)cncc1Cl] | LCMS m/z = 513 (M + H); 1H NMR (400 MHz, DMSO-d6) δ = 13.08 (br s, 1H), 8.57 (s, 2H), 7.74 (d, J = 1.8 Hz, 1H), 7.48 (d, J = 9.0 Hz, 1H), 7.35 (d, J = 2.0 Hz, 1H), 7.20 (d, J = 2.1 Hz, 1H), 7.10 (dd, J = 2.2, 9.0 Hz, 1H), 6.11 (q, J = 6.6 Hz, 1H), 4.37-4.31 (m, 2H), 3.45-3.42 (m, 4H), 2.49-2.42 (m, 3H), 2.19 (s, 6H), 1.75 (d, J = 6.6 Hz, 3H) |

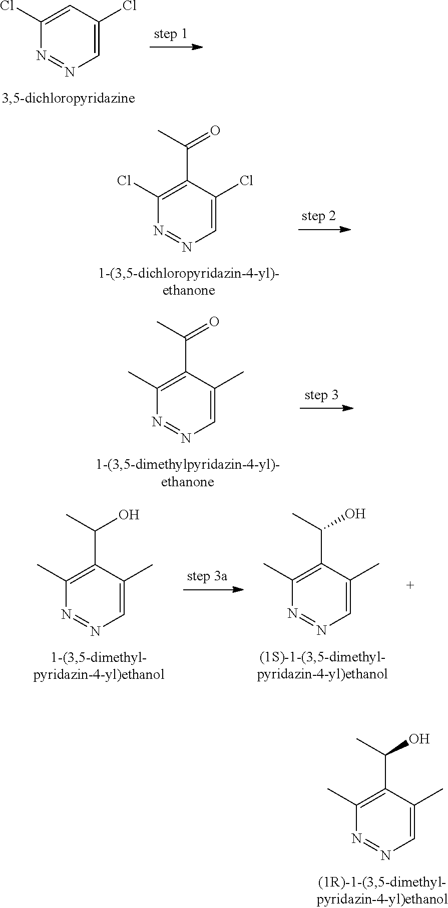

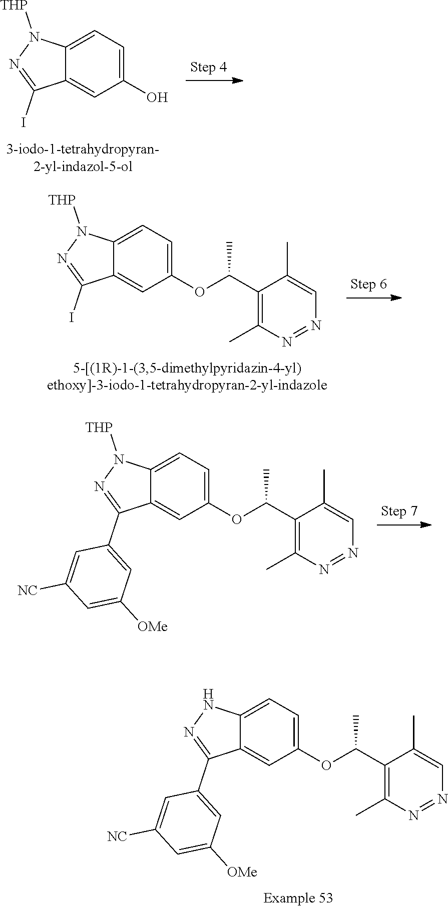

### Example 53. 3-[5-[(1R)-1-(3,5-dimethylpyridazin-4-yl)ethoxy]-1H-indazol-3-yl]-5-methoxy-benzonitrile

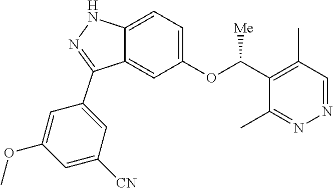 [SMILES: COc1cc(C#N)cc(-c2n[nH]c3ccc(O[C@H](C)c4c(C)cnnc4C)cc23)c1]

Step 1. 1-(3,5-dichloropyridazin-4-yl)ethanone. To a solution of 3,5-dichloropyridazine (50.0 g, 0.336 mol) in CH₃CN (500 mL) and water (500 mL) was added 2-oxopropanoic acid (37.0 g, 0.403 mol), K₂S₂O₈ (136.0 g, 0.504 mol) and AgNO₃ (11.4 g, 0.067 mol). The reaction mixture was stirred at 70° C. for 6 h. After completion, the reaction was cooled to room temperature, and the ACN was removed. The aqueous solution was extracted with EtOAc (3×500 mL). The combined organic layers were washed with brine (2×300 mL), dried over Na₂SO₄ and concentrated. The residue was purified by silica gel flash column chromatography (petroleum Ether/EtOAc=4/1) to give 1-(3,5-dichloropyridazin-4-yl)ethanone as a light yellow solid (35.0 g, 56%). LCMS m/z=191 (M+1).

Step 2. 1-(3,5-Dimethylpyridazin-4-yl)ethanone. To a solution of 1-(3,5-dichloropyridazin-4-yl)ethanone (35.0 g, 0.184 mol) in dioxane (700 mL) and water (35 mL) was added 2,4,6-trimethyl-1,3,5,2,4,6-trioxatriborinane (157 mL, 3.5 mol/L in THF, 0.552 mol), K₂CO₃ (63.5 g, 0.460 mol) and Pd(dppf)Cl₂ (5.3 g, 0.0074 mol). The mixture was stirred for 36 h at 110° C. under a N₂ atmosphere. After completion, the reaction was cooled to room temperature and concentrated. The crude product was purified by silica gel flash column chromatography (DCM/MeOH=60/1) to give a brown oil (17.5 g, 63.2%). LCMS m/z—151 (M+1).

Step 3. 1-(3,5-Dimethylpyridazin-4-yl)ethanol. To a solution of 1-(3,5-dimethylpyridazin-4-yl)ethanone (17.5 g, 0.12 mol) in THF (180 mL) was added NaBH₄ (5.06 g, 0.132 mol) at 0° C., then MeOH (18 mL) was added dropwise at the same temperature. The reaction mixture was stirred for 2 h at 0° C. When the reaction was completed, silica (80 g) was added to the mixture at 0° C. and concentrated in vacuum. The crude product was purified by silica gel flash column chromatography (DCM/MeOH=40/1) to give a light brown solid afford the racemic product 1-6 (9.5 g, 52.0% yield). The racemate was separated by SFC to afford Peak 1 (4.0 g, 23% yield, S-enantiomer) as a white solid LCMS m/z=153[M+1]; ¹H NMR (400 MHZ, CDCl₃) δ 8.65 (s, 1H), 5.31-5.26 (m, 1H), 3.37 (s, 1H), 2.67 (s, 3H), 2.43 (s, 3H), 1.49 (d, J=6.8 Hz, 3H), [α]D=47.092. Peak 2 (4.3 g, 24% yield, R-enantiomer) as a white solid. LCMS m/z=153 (M+1); ¹H NMR (400 MHZ, CDCl₃) δ 8.61 (s, 1H), 5.30-5.25 (m, 1H), 3.71 (s, 1H), 2.68 (s, 3H), 2.41 (s, 3H), 1.48 (d, J=6.8 Hz, 3H), [α]D=+44.534.

Step 4. 5-[(1R)-1-(3,5-Dimethylpyridazin-4-yl)ethoxy]-3-iodo-1-tetrahydropyran-2-yl-indazole. To a solution of 3-iodo-1-tetrahydropyran-2-yl-indazol-5-ol (678 mg, 1.97 mmol) and (S)-1-(3,5-dimethylpyridazin-4-yl)ethanol (200 mg, 1.31 mmol), in dry THF (5 mL) was added PPh₃ (517 mg, 2.62 mmol) at 0° C. under N₂. Then, a solution of DIAD (398 mg, 1.70 mmol, 1.3 eq) in THF (2.5 mL) was added dropwise and the resulting mixture was stirred at rt for 16 h. After completion, the reaction solvent was removed. The residue was purified by silica gel flash column chromatography (Petroleum Ether/THF=3/1) to give a white solid (53% yield). LCMS m/z=479 (M+1); ¹H NMR (400 MHZ, DMSO-d6) δ 8.85 (s, 1H), 7.67 (d, J=8.8 Hz, 1H), 7.19 (d, J=9.2 Hz, 1H), 6.46 (s, 1H), 5.86 (q, J=4.0 Hz, 1H), 5.76 (t, J=7.2 Hz, 1H), 3.83-3.81 (m, 1H), 3.70-3.65 (m, 1H), 2.79 (s, 3H), 2.44 (s, 3H), 2.40-2.21 (m, 1H), 1.99-1.88 (m, 2H), 1.68-1.66 (m, 4H), 1.65-1.54 (m, 2H).

Step 5. 3-Methoxy-5-(4,4,5,5-tetramethyl-1,3,2-dioxaborolan-2-yl)benzonitrile. To a solution of 3-bromo-5-methoxy-benzonitrile (1.0 g, 4.7 mmol, 1.0 eq), BPD (1.56 g, 6.1 mmol, 1.5 eq) in dioxane (15 mL) were added KOAc (1.39 g, 14.1 mmol, 3.0 eq), Pd(dppf)Cl₂ (380 mg, 0.47 mmol, 0.1 eq). The reaction mixture was stirred for 4 h at 110° C. under N₂ protection. After cooling to room temperature, the mixture was concentrated in vacuum. The crude product was purified by silica gel flash column chromatography (Petroleum Ether/EtOAc=10/1) to give a white solid (770 mg, 57%). LCMS m/z=206 (M+1). ¹H NMR (400 MHZ, DMSO-d6) δ 7.57 (dd, J=2.4 Hz, 1.2 Hz, 1H), 7.53 (d, J=1.6 Hz, 1H), 7.42-7.41 (m, 1H), 3.83 (s, 3H), 1.30 (s, 12H).

Step 6. 3-[5-[(1R)-1-(3,5-Dimethylpyridazin-4-yl)ethoxy]-1-tetrahydropyran-2-yl-indazol-3-yl]-5-methoxy-benzonitrile. To a solution of 5-[(1R)-1-(3,5-dimethylpyridazin-4-yl)ethoxy]-3-iodo-1-tetrahydropyran-2-yl-indazole (100 mg, 0.21 mmol, 1.0 eq), 3-methoxy-5-(4,4,5,5-tetramethyl-1,3,2-dioxaborolan-2-yl)benzonitrile (81 mg, 0.31 mmol, 1.5 eq) in dioxane (3 mL) and water (0.3 mL) were added K₂CO₃ (86 mg, 0.63 mmol, 3.0 eq) and Pd(dppf)Cl₂ (17 mg, 0.021 mmol, 0.1 eq). The reaction mixture was stirred for 3 h at 100° C. under N₂ protection. After cooled to room temperature, the solvent was removed. The crude product was purified by prep-TLC (DCM/MeOH=30/1) to give a white solid (72 mg, 71%). LCMS m/z=484 (M+1).

Step 7. 3-[5-[(1R)-1-(3,5-dimethylpyridazin-4-yl)ethoxy]-1H-indazol-3-yl]-5-methoxy-benzonitrile. To a solution of 3-[5-[(1R)-1-(3,5-dimethylpyridazin-4-yl)ethoxy]-1-tetrahydropyran-2-yl-indazol-3-yl]-5-methoxy-benzonitrile (72 mg, 0.128 mmol, 1.0 eq) in DCM (4 mL) was added TFA (1 mL). The reaction mixture was stirred at rt for 3 h. After completion, the reaction mixture was concentrated. The crude product was treated with MeOH (3 mL), and NaHCO₃ (excess) was added to the solution, which was stirred for 20 min at rt, and then DCM (20 mL) was added. The solid was filtered out and the filtrate was concentrated. The residue was purified by Prep-TLC (DCM/MeOH=20/1) to give a white solid (28 mg, 58%). LCMS m/z=400 (M+1); ¹H NMR (400 MHZ, DMSO-d6) δ 13.35 (s, 1H), 8.82 (s, 1H), 7.69 (d, J=0.8 Hz, 1H), 7.58-7.40 (m, 1H), 7.53 (d, J=8.8 Hz, 1H), 7.49-7.45 (m, 1H), 7.15-7.09 (m, 1H), 7.03-7.00 (m, 1H), 5.93 (q, J=6.0 Hz, 1H), 3.90 (s, 3H), 2.79 (s, 3H), 2.47 (s, 3H), 1.68 (d, J=6.4 Hz, 3H).

### Example 55. 3-[5-[(1S)-1-(3,5-dimethylpyridazin-4-yl)ethoxy]-1H-indazol-3-yl]-5-methoxy-benzonitrile

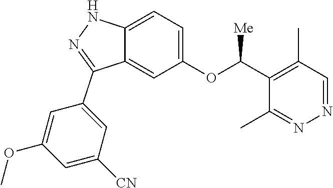 [SMILES: COc1cc(C#N)cc(-c2n[nH]c3ccc(O[C@@H](C)c4c(C)cnnc4C)cc23)c1]

Step 1. 5-[(1S)-1-(3,5-Dimethylpyridazin-4-yl)ethoxy]-3-iodo-1-tetrahydropyran-2-yl-indazole. To a solution of (R)-1-(3,5-dimethylpyridazin-4-yl)ethanol (200 mg, 1.31 mmol, 1.0 eq) and 3-iodo-1-tetrahydropyran-2-yl-indazol-5-ol (678 mg, 1.97 mmol, 1.5 eq) in DCM (7.5 mL) was added PPhs (862 mg, 3.28 mmol, 2.5 eq) at 0° C. under N2. Then, a solution of DIAD (398 mg, 1.97 mmol, 1.5 eq) in DCM (2.5 mL) was added dropwise and the resulting mixture was stirred at rt for 3 h. After completion, the reaction mixture was concentrated. The residue was purified by silica gel flash column chromatography (petroleum Ether/THF=3/1) to give a white solid (56% yield). LCMS m/z=479 (M+1); 1H NMR (400 MHZ, DMSO-d6) δ 8.85 (s, 1H), 7.66 (d, J=8.8 Hz, 1H), 7.19 (dd, J=9.2 Hz, 2.4 Hz, 1H), 6.47 (s, 1H), 5.86 (q, J=4.0 Hz, 1H), 5.76 (t, J=7.2 Hz, 1H), 3.83-3.81 (m, 1H), 3.70-3.65 (m, 1H), 2.79 (s, 3H), 2.44 (s, 3H), 2.32-2.25 (m, 1H), 1.99-1.87 (m, 2H), 1.68-1.66 (m, 4H), 1.65-1.54 (m, 2H).

Step 2. 3-[5-[(1S)-1-(3,5-Dimethylpyridazin-4-yl)ethoxy]-1-tetrahydropyran-2-yl-indazol-3-yl]-5-methoxy-benzonitrile. To a solution of 5-[(1S)-1-(3,5-dimethylpyridazin-4-yl)ethoxy]-3-iodo-1-tetrahydropyran-2-yl-indazole (130 mg, 0.27 mmol, 1.0 eq), 3-methoxy-5-(4,4,5,5-tetramethyl-1,3,2-dioxaborolan-2-yl)benzonitrile (106 mg, 0.41 mmol, 1.5 eq) in dioxane (3 mL) and water (0.3 mL) were added K₂CO₃ (112 mg, 0.82 mmol, 3.0 eq) and Pd(dppf)Cl₂ (22 mg, 0.027 mmol, 0.1 eq). The reaction mixture was stirred for 2 h at 100° C. under N₂ protection. After cooling to room temperature, the reaction mixture was concentrated in vacuum. The crude product was purified by prep-TLC (DCM/MeOH=30/1) to give a brown oil (110 mg, 84.1% yield). LCMS m/z=484 (M+1).

Step 3. 3-[5-[(1S)-1-(3,5-dimethylpyridazin-4-yl)ethoxy]-1H-indazol-3-yl]-5-methoxy-benzonitrile. To a solution of 3-[5-[(1S)-1-(3,5-dimethylpyridazin-4-yl)ethoxy]-1-tetrahydropyran-2-yl-indazol-3-yl]-5-methoxy-benzonitrile (110 mg, 0.23 mmol, 1.0 eq) in DCM (4 mL) was added TFA (1 mL). The reaction mixture was stirred at rt for 3 h. After completion, the reaction mixture was concentrated in vacuum. The crude product was treated with MeOH (3 mL), NaHCO₃ (excess) was added to the solution, which was stirred for 20 min at rt, then DCM (30 mL) was added. The solid was filtered out and the filtrate was concentrated. The residue was purified by Prep-TLC (DCM/MeOH=20/1) to give a white solid (51 mg, 56%). LCMS m/z=400 (M+1); ¹H NMR (400 MHZ, DMSO-d6) δ 13.36 (s, 1H), 8.84 (s, 1H), 7.70-7.68 (m, 1H), 7.59-7.55 (m, 1H), 7.53 (d, J=9.6 Hz, 1H), 7.49-7.45 (m, 1H), 7.13 (dd, J=9.4 Hz, 2.0 Hz, 1H), 7.03 (d, J=1.6 Hz, 1H), 5.93 (q, J=6.8 Hz, 1H), 3.90 (s, 3H), 2.79 (s, 3H), 2.47 (s, 3H), 1.68 (d, J=6.8 Hz, 3H).

### Example 64. 3-[5-[(1R)-1-(3,5-Dimethylpyridazin-4-yl)ethoxy]-1H-indazol-3-yl]-5-morpholino-benzonitrile

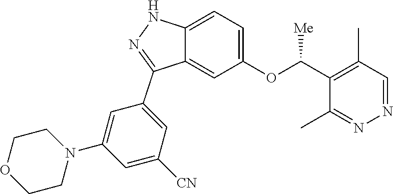 [SMILES: Cc1cnnc(C)c1[C@@H](C)Oc1ccc2[nH]nc(-c3cc(C#N)cc(N4CCOCC4)c3)c2c1]

Step 1. 3-Bromo-5-morpholino-benzonitrile. A mixture of 3-bromo-5-fluoro-benzonitrile (2.0 g, 10 mmol, 1.0 eq) and morpholine (8.7 g, 100 mmol, 10.0 eq) was stirred for 5 h at 120° C. Water (100 mL) was added to the mixture, and the solid was collected. The filter cake was washed with water (30 mL), and then dissolved in EtOAc (100 mL), dried over Na₂SO₄ and concentrated in vacuum to give an off-white solid (1.92 g, 72%) LCMS m/z=267 (M+1).

Step 2. 3-Morpholino-5-(4,4,5,5-tetramethyl-1,3,2-dioxaborolan-2-yl)benzonitrile. To a solution of 3-Bromo-5-morpholino-benzonitrile (920 mg, 3.5 mmol, 1.0 eq), BPD (CAS: 73183-34-3, 1.06 g, 4.2 mmol, 1.2 eq) in dioxane (20 mL) were added KOAc (1.02 g, 10.4 mmol, 3.0 eq) and Pd(dppf)Cl₂ (227 mg, 0.3 mmol, 0.08 eq). The reaction mixture was stirred for 4 h at 100° C. under N₂ protection. After cooled to room temperature, the solvent was removed, and the crude product was purified by silica gel flash column chromatography (Petroleum Ether/EtOAc=2/1) to give a yellow solid (810 mg, 74.0%). LCMS m/z=315 (M+1); ¹H NMR (400 MHZ, DMSO-d6) δ 7.49-7.48 (m, 1H), 7.41 (d, J=2.4 Hz, 1H), 7.34 (s, 1H), 3.73 (t, J=4.4 Hz, 4H), 3.18 (t, J=4.8 Hz, 4H), 1.30 (s, 12H).

Step 3. 3-[5-[(1R)-1-(3,5-dimethylpyridazin-4-yl)ethoxy]-1-tetrahydropyran-2-yl-indazol-3-yl]-5-morpholino-benzonitrile. To a solution of 5-[(1R)-1-(3,5-dimethylpyridazin-4-yl)ethoxy]-3-iodo-1-tetrahydropyran-2-yl-indazole (120 mg, 0.25 mmol, 1.0 eq) and 3-morpholino-5-(4,4,5,5-tetramethyl-1,3,2-dioxaborolan-2-yl)benzonitrile (119 mg, 0.38 mmol, 1.5 eq) in dioxane (2 mL) and water (0.2 mL) were added K₂CO₃ (104 mg, 0.75 mmol, 3.0 eq) and Pd(dppf)Cl₂ (20 mg, 0.025 mmol, 0.1 eq). The reaction mixture was heated to 90° C. and stirred for 4 h under N₂ protection. After completion, the mixture was cooled to rt and concentrated in vacuum. The crude product was purified by silica gel flash column chromatography (EtOAc) to give a red solid (160 mg, 80%). LCMS m/z=539 (M+1).

Step 4. 3-[5-[(1R)-1-(3,5-dimethylpyridazin-4-yl)ethoxy]-1H-indazol-3-yl]-5-morpholino-benzonitrile. To a solution of 3-[5-[(1R)-1-(3,5-dimethylpyridazin-4-yl)ethoxy]-1-tetrahydropyran-2-yl-indazol-3-yl]-5-morpholino-benzonitrile (100 mg, 0.19 mmol, 1.0 eq) in DCM (2.0 mL) was added TFA (0.5 mL), which was stirred at rt for 5 h. After completion, the reaction mixture was concentrated in vacuum. The crude product was treated with DCM/MeOH (15 mL, V/V=5/1). NaHCO₃ (excess) was added to the solution, which was stirred at rt for 20 min, then, DCM (20 mL) was added. The solid was filtered out and the filtrate was concentrated. The residue was purified by Prep-HPLC (Prep-C18, 5 μM Triart column, 20×150 mm, YMC-Actus; gradient elution of 50% MeCN in water to 60% MeCN in water over a 10 min period, where both solvents contain 0.05% NH₃·H₂O) to give an off-white solid (40 mg, 46.0%). LCMS m/z=455 (M+1); ¹H NMR (400 MHZ, DMSO-d6) δ 13.25 (s, 1H), 8.78 (s, 1H), 7.53-7.52 (m, 1H), 7.49 (d, J=8.8 Hz, 1H), 7.40 (s, 1H), 7.38-7.37 (m, 1H), 7.09 (dd, J=8.8 Hz, 2.0 Hz, 1H), 6.95 (d, J=2.0 Hz, 1H), 5.89 (q, J=6.8 Hz, 1H), 3.74 (t, J=4.8 Hz, 4H), 3.21 (t, J=4.8 Hz, 4H), 2.75 (s, 3H), 2.42 (s, 3H), 1.64 (d, J=6.8 Hz, 3H).

### Example 65. 3-[5-[(1S)-1-(3,5-Dimethylpyridazin-4-yl)ethoxy]-1H-indazol-3-yl]-5-morpholino-benzonitrile

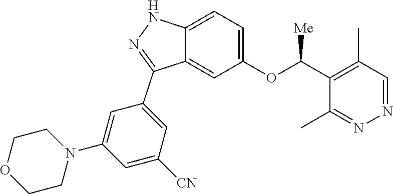 [SMILES: Cc1cnnc(C)c1[C@H](C)Oc1ccc2[nH]nc(-c3cc(C#N)cc(N4CCOCC4)c3)c2c1]

Step 1. 3-[5-[(1S)-1-(3,5-Dimethylpyridazin-4-yl)ethoxy]-1-tetrahydropyran-2-yl-indazol-3-yl]-5-morpholino-benzonitrile. To a solution of 5-[(1S)-1-(3,5-dimethylpyridazin-4-yl)ethoxy]-3-iodo-1-tetrahydropyran-2-yl-indazole (180 mg, 0.38 mmol, 1.0 eq), 3-morpholino-5-(4,4,5,5-tetramethyl-1,3,2-dioxaborolan-2-yl)benzonitrile (177 mg, 0.56 mmol, 1.5 eq) in dioxane (10 mL) and water (1 mL) were added K₂CO₃ (156 mg, 1.13 mmol, 3.0 eq) and Pd(dppf)Cl₂ (25 mg, 0.03 mmol, 0.08 eq). The reaction mixture was stirred for 4 h at 100° C. under N₂ protection. After cooled to room temperature, the reaction mixture was concentrated in vacuum. The crude product was purified by prep-TLC (EtOAc) to give a yellow solid (135 mg, 67%). LCMS m/z=539 (M+1).

Step 2. 3-[5-[(1S)-1-(3,5-Dimethylpyridazin-4-yl)ethoxy]-1H-indazol-3-yl]-5-morpholino-benzonitrile. To a solution of 3-[5-[(1S)-1-(3,5-dimethylpyridazin-4-yl)ethoxy]-1-tetrahydropyran-2-yl-indazol-3-yl]-5-morpholino-benzonitrile (135 mg, 0.25 mmol, 1.0 eq) in DCM (10 mL) was added TFA (2 mL). The reaction mixture was stirred at rt for 6 h. After completion, the reaction mixture was concentrated. The crude product was treated with MeOH (3 mL), and NaHCO₃ (excess) was added to the solution, which was stirred at rt for 20 min. Then, DCM (20 mL) was added. The solid was filtered out and filtrate was concentrated. The residue was purified by Prep-TLC (DCM/MeOH=20/1) to give an off-white solid (61 mg, 53%). LCMS m/z=455 (M+1); ¹H NMR (400 MHZ, DMSO-d6) δ 13.28 (s, 1H), 8.82 (s, 1H), 7.57 (s, 1H), 7.52 (d, J=8.8 Hz, 1H), 7.43 (s, 1H), 7.41-7.40 (m, 1H), 7.12 (dd, J=8.8 Hz, 2.0 Hz, 1H), 6.99 (d, J=2.4 Hz, 1H), 5.92 (q, J=6.4 Hz, 1H), 3.77 (t, J=4.8 Hz, 4H), 3.24 (t, J=4.8 Hz, 4H), 2.79 (s, 3H), 2.46 (s, 3H), 1.67 (d, J=6.4 Hz, 3H).

### Example 69. 4-[5-[(1R)-1-(3,5-Dimethylpyridazin-4-yl)ethoxy]-1H-indazol-3-yl]-6-methyl-pyridine-2-carbonitrile

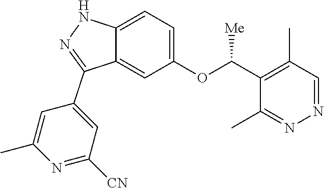 [SMILES: Cc1cc(-c2n[nH]c3ccc(O[C@H](C)c4c(C)cnnc4C)cc23)cc(C#N)n1]

Step 1. 4-[5-[(1R)-1-(3,5-Dimethylpyridazin-4-yl)ethoxy]-1-tetrahydropyran-2-yl-indazol-3-yl]-6-methyl-pyridine-2-carbonitrile. To a solution of 5-[(1R)-1-(3,5-dimethylpyridazin-4-yl)ethoxy]-3-iodo-1-tetrahydropyran-2-yl-indazole (170 mg, 0.36 mmol, 1.0 eq) and 6-methyl-4-(4,4,5,5-tetramethyl-1,3,2-dioxaborolan-2-yl)pyridine-2-carbonitrile (104 mg, 0.64 mmol, 1.8 eq) in dioxane (5.0 mL) and water (0.5 mL) were added K₂CO₃ (149 mg, 1.08 mmol, 3.0 eq) and Pd(dppf)Cl₂ (29 mg, 0.036 mmol, 0.1 eq). The reaction mixture was stirred for 4 h at 90° C. under N₂ protection. After the reaction was completed, the solid was filtered out and the filtrate was concentrated in vacuum. The crude product was purified by silica gel flash column chromatography (Petroleum Ether/EtOAc=1/1) to give a yellow solid (140 mg, 83%). LCMS m/z=469 (M+1).

Step 2. 4-[5-[(1R)-1-(3,5-Dimethylpyridazin-4-yl)ethoxy]-1H-indazol-3-yl]-6-methyl-pyridine-2-carbonitrile. To a solution of 4-[5-[(1R)-1-(3,5-dimethylpyridazin-4-yl)ethoxy]-1-tetrahydropyran-2-yl-indazol-3-yl]-6-methyl-pyridine-2-carbonitrile (130 mg, 0.28 mmol, 1.0 eq) in DCM (3 mL) was added TFA (1.0 mL). The reaction mixture was stirred at rt for 16 h. After completion, the reaction mixture was concentrated in vacuum. The crude product was treated with DCM/MeOH (15 mL, V/V=5/1). NaHCO₃ (excess) was added to the solution, which was stirred for 20 min at rt, then, DCM (20 mL) was added. The solid was filtered out and the filtrate was concentrated. The crude product was purified by Prep-TLC (DCM/MeOH=15/1) to give an off-white solid (42 mg, 39%). LCMS m/z=385 (M+1); ¹H NMR (400 MHz, DMSO-d6) δ 13.69 (s, 1H), 8.82 (s, 1H), 8.15 (s, 1H), 7.90 (s, 1H), 7.58 (d, J=8.8 Hz, 1H), 7.17-7.13 (m, 2H), 5.95 (q, J=6.8 Hz, 1H), 2.82 (s, 3H), 2.63 (s, 3H), 2.47 (s, 3H), 1.70 (d, J=6.8 Hz, 3H).

### Example 70. 4-[5-[(1S)-1-(3,5-Dimethylpyridazin-4-yl)ethoxy]-1H-indazol-3-yl]-6-methyl-pyridine-2-carbonitrile

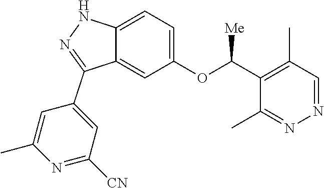 [SMILES: Cc1cc(-c2n[nH]c3ccc(O[C@@H](C)c4c(C)cnnc4C)cc23)cc(C#N)n1]

Step 1. 4-[5-[(1S)-1-(3,5-Dimethylpyridazin-4-yl)ethoxy]-1-tetrahydropyran-2-yl-indazol-3-yl]-6-methyl-pyridine-2-carbonitrile. To a solution of 5-[(1S)-1-(3,5-dimethylpyridazin-4-yl)ethoxy]-3-iodo-1-tetrahydropyran-2-yl-indazole (180 mg, 0.38 mmol, 1.0 eq) and 6-methyl-4-(4,4,5,5-tetramethyl-1,3,2-dioxaborolan-2-yl)pyridine-2-carbonitrile (119 mg, 0.49 mmol, 1.3 eq) in dioxane (5.0 mL) and water (0.5 mL) were added K₂CO₃ (130 mg, 0.95 mmol, 2.5 eq) and Pd(dppf)Cl₂ (31.2 mg, 0.038 mmol, 0.1 eq). The reaction mixture was stirred for 3 h at 90° C. under N₂ protection. After the reaction was completed, the solid was filtered out and the filtrate was concentrated in vacuum. The crude product was purified by silica gel flash column chromatography (Petroleum Ether/EtOAc=1/1) to give a yellow solid (130 mg, 74%). LCMS m/z=469 (M+1).

Step 2. 4-[5-[(1S)-1-(3,5-Dimethylpyridazin-4-yl)ethoxy]-1H-indazol-3-yl]-6-methyl-pyridine-2-carbonitrile. To a solution of 4-[5-[(1S)-1-(3,5-dimethylpyridazin-4-yl)ethoxy]-1-tetrahydropyran-2-yl-indazol-3-yl]-6-methyl-pyridine-2-carbonitrile (120 mg, 0.26 mmol, 1.0 eq) in DCM (5 mL) was added TFA (1.0 mL). The reaction mixture was stirred at rt for 16 h. After completion, the reaction mixture was concentrated in vacuum. The crude product was treated with DCM/MeOH (15 mL, V/V=5/1). NaHCO₃ (excess) was added to the solution, which was stirred at rt for 20 min. DCM (20 mL) was added, the solid was filtered out and the filtrate was concentrated. The crude product was purified by Prep-HPLC (Prep-C18, 5 μM Triart column, 20×150 mm, YMC-Actus; gradient elution of 50% MeCN in water to 60% MeCN in water over an 8 min period, where both solvents contain 0.05% NH₃H₂O) to give a white solid (49 mg, 49%). LCMS m/z=385 (M+1); ¹H NMR (400 MHZ, DMSO-d6) δ 13.72 (s, 1H), 8.83 (s, 1H), 8.15 (d, J=1.2 Hz, 1H), 7.90 (s, 1H), 7.58 (d, J=9.2 Hz, 1H), 7.18 (d, J=1.2 Hz, 1H), 7.15-7.12 (m, 1H), 5.95 (q, J=7.2 Hz, 1H), 2.82 (s, 3H), 2.63 (s, 3H), 2.47 (s, 3H), 1.70 (d, J=6.4 Hz, 3H).

Examples 52-87 were synthesized using procedures described in the previous examples.

| | | |
|---|---|---|
| Example 52 5-[(1R)-1-(3,5- dimethylpyridazin-4- yl)ethoxy]-3-(3-ethoxy-5- methoxy-phenyl)-1H- indazole | 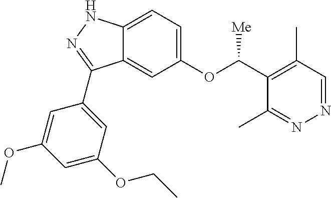 [SMILES: CCOc1cc(OC)cc(-c2n[nH]c3ccc(O[C@H](C)c4c(C)cnnc4C)cc23)c1] | LCMS m/z = 419 (M + H); 1H NMR (400 MHz, DMSO-d6) δ: 13.12 (s, 1H), 8.83 (s, 1H), 7.51 (d, J = 9.2 Hz, 1H), 7.11 (dd, J = 8.8, 2.4 Hz, 1H), 6.95 (d, J = 2.0 Hz, 1H), 6.82-6.78 (m, 1H), 6.52 (t, J = 2.4 Hz, 1H), 5.87 (q, J = 6.8 Hz, 1H), 4.08 (q, J = 6.8 Hz, 2H), 3.81 (s, 3H), 2.76 (s, 3H), 2.45 (s, 3H), 1.67 (d, J = 6.0 Hz, 3H), 1.40 (t, J = 6.8 Hz, 3H) |
| Example 53 3-[5-[(1R)-1-(3,5- dimethylpyridazin-4- yl)ethoxy]-1H-indazol-3- yl]-5-methoxy- benzonitrile | 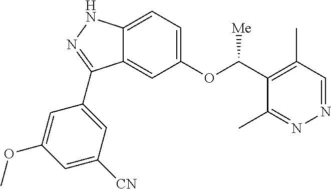 [SMILES: COc1cc(C#N)cc(-c2n[nH]c3ccc(O[C@H](C)c4c(C)cnnc4C)cc23)c1] | LCMS m/z = 400 (M + H); 1H NMR (400 MHz, DMSO-d6) δ 13.35 (s, 1H), 8.82 (s, 1H), 7.68 (s, 1H), 7.57 (t, J = 1.6 Hz, 1H), 7.54 (d, J = 9.2 Hz, 1H), 7.47 (t, J = 1.6 Hz, 1H), 7.13 (d, J = 9.2 Hz, 1H), 7.02 (d, J = 2.4 Hz, 1H), 5.94 (q, J = 6.8 Hz, 1H), 3.90 (s, 3H), 2.78 (s, 3H), 2.49 (s, 3H), 1.69 (d, J = 6.8 Hz, 3H) |
| Example 54 3-(3,5-dimethoxyphenyl)- 5-[(1R)-1-(3,5- dimethylpyridazin-4- yl)ethoxy]-1H-indazole | 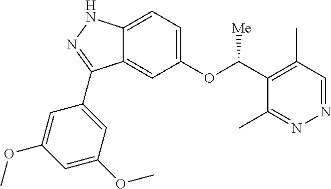 [SMILES: COc1cc(OC)cc(-c2n[nH]c3ccc(O[C@H](C)c4c(C)cnnc4C)cc23)c1] | LCMS m/z = 405 (M + H); 1H NMR (400 MHz, DMSO-d6) δ 13.12 (s, 1H), 8.84 (s, 1H), 7.50 (d, J = 9.2 Hz, 1H), 7.10 (dd, J = 9.6 Hz, J = 2.8 Hz, 1H), 6.97 (d, J = 2.0 Hz, 1H), 6.82 (d, J = 2.0 Hz, 2H), 6.54 (t, J = 2.0 Hz, 1H), 5.86 (q, J = 6.0 Hz, 1H), 3.82 (s, 6H), 2.76 (s, 3H), 2.44 (s, 3H), 1.66 (d, J = 6.8 Hz, 3H) |
| Example 55 3-[5-[(1S)-1-(3,5- dimethylpyridazin-4- yl)ethoxy]-1H-indazol-3- yl]-5-methoxy- benzonitrile | 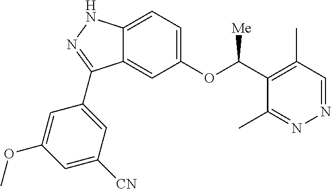 [SMILES: COc1cc(C#N)cc(-c2n[nH]c3ccc(O[C@@H](C)c4c(C)cnnc4C)cc23)c1] | LCMS m/z = 400 (M + H); 1H NMR (400 MHz, DMSO-d6) δ 13.36 (s, 1H), 8.84 (s, 1H), 7.70-7.68 (m, 1H), 7.59-7.55 (m, 1H), 7.53 (d, J = 9.6 Hz, 1H), 7.49-7.45 (m, 1H), 7.13 (dd, J = 9.4 Hz, 2.0 Hz, 1H), 7.03 (d, J = 1.6 Hz, 1H), 5.93 (q, J = 6.8 Hz, 1H), 3.90 (s, 3H), 2.79 (s, 3H), 2.47 (s, 3H), 1.68 (d, J = 6.8 Hz, 3H) |
| Example 56 3-[5-[(1R)-1-(3,5- dimethylpyridazin-4- yl)ethoxy]-1H-indazol-3- yl]-5-ethoxy-benzonitrile | 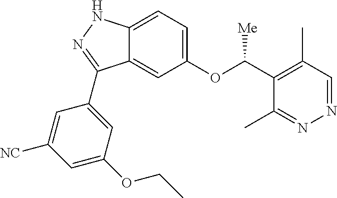 [SMILES: CCOc1cc(C#N)cc(-c2n[nH]c3ccc(O[C@H](C)c4c(C)cnnc4C)cc23)c1] | LCMS m/z = 414 (M + H); 1H NMR (400 MHz, DMSO-d6) δ 13.32 (s, 1H), 8.79 (s, 1H), 7.64 (s, 1H), 7.51-7.49 (m, 2H), 7.41-7.40 (m, 1H), 7.09 (dd, J = 8.4 Hz, 1.6 Hz, 1H), 6.96 (d, J = 2.0 Hz, 1H), 5.89 (q, J = 6.8 Hz, 1H), 4.14 (q, J = 6.8 Hz, 2H), 2.76 (s, 3H), 2.44 (s, 3H), 1.65 (d, J = 6.8 Hz, 3H), 1.37 (t, J = 6.8 Hz, 3H) |
| Example 57 3-[5-[(1R)-1-(3,5- dimethylpyridazin-4- yl)ethoxy]-1H-indazol-3- yl]-5-isopropoxy- benzonitrile | 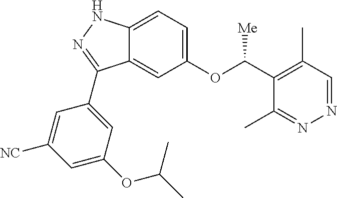 [SMILES: Cc1cnnc(C)c1[C@@H](C)Oc1ccc2[nH]nc(-c3cc(C#N)cc(OC(C)C)c3)c2c1] | LCMS m/z = 428 (M + H); 1H NMR (400 MHz, DMSO-d6) δ 13.35 (s, 1H), 8.82 (s, 1H), 7.65 (s, 1H), 7.53 (d, J = 8.8 Hz, 1H), 7.51-7.50 (m, 1H), 7.45-7.44 (m, 1H), 7.12 (dd, J = 9.2, 2.4 Hz, 1H), 6.98 (d, J = 2.0 Hz, 1H), 5.92 (q, J = 6.0 Hz, 1H), 4.81- 4.78 (m, 1H), 2.79 (s, 3H), 2.47 (s, 3H), 1.68 (d, J = 6.8 Hz, 3H), 1.34 (d, J = 6.0 Hz, 6H) |
| Example 58 3-(3,5-dimethoxyphenyl)- 5-[(1S)-1-(3,5- dimethylpyridazin-4- yl)ethoxy]-1H-indazole | 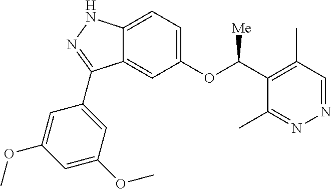 [SMILES: COc1cc(OC)cc(-c2n[nH]c3ccc(O[C@@H](C)c4c(C)cnnc4C)cc23)c1] | LCMS m/z = 405 (M + H); 1H NMR (400 MHz, DMSO-d6) δ 13.12 (s, 1H), 8.83 (s, 1H), 7.50 (d, J = 9.2 Hz, 1H), 7.10 (dd, J = 9.6 Hz, 2.8 Hz, 1H), 6.97 (d, J = 2.0 Hz, 1H), 6.82 (d, J = 2.4 Hz, 2H), 6.54 (t, J = 2.0 Hz, 1H), 5.86 (q, J = 6.8 Hz, 1H), 3.82 (s, 6H), 2.76 (s, 3H), 2.44 (s, 3H), 1.66 (d, J = 6.8 Hz, 3H) |
| Example 59 5-[(1S)-1-(3,5- dimethylpyridazin-4- yl)ethoxy]-3-(3-ethoxy- 5-methoxy-phenyl)-1H- indazole | 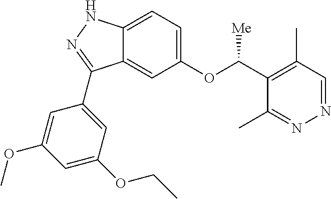 [SMILES: CCOc1cc(OC)cc(-c2n[nH]c3ccc(O[C@H](C)c4c(C)cnnc4C)cc23)c1] | LCMS m/z = 419 (M + H); 1H NMR (400 MHz, DMSO-d6) δ 13.12 (s, 1H), 8.83 (s, 1H), 7.50 (d, J = 8.8 Hz, 1H), 7.10 (dd, J = 8.8 Hz, 2.0 Hz, 1H), 6.95 (d, J = 2.0 Hz, 1H), 6.83-6.82 (m, 1H), 6.79-6.78 (m, 1H), 6.52 (t, J = 2.4 Hz, 1H), 5.87 (q, J = 6.8 Hz, 1H), 4.07 (q, J = 7.2 Hz, 2H), 3.81 (s, 3H), 2.76 (s, 3H), 2.45 (s, 3H), 1.66 (d, J = 6.0 Hz, 3H), 1.40 (t, J = 6.8 Hz, 3H) |
| Example 60 5-[(1R)-1-(3,5- dimethylpyridazin-4- yl)ethoxy]-3-(3- isopropoxy-5-methoxy- phenyl)-1H-indazole | 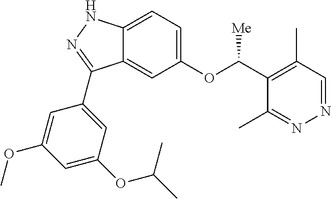 [SMILES: COc1cc(OC(C)C)cc(-c2n[nH]c3ccc(O[C@H](C)c4c(C)cnnc4C)cc23)c1] | LCMS m/z = 433 (M + H); 1H NMR (400 MHz, DMSO-d6) δ 13.11 (s, 1H), 8.83 (s, 1H), 7.51 (d, J = 8.8 Hz, 1H), 7.11 (d, J = 2.8 Hz, 1H), 6.94 (s, 1H), 6.80-6.77 (m, 2H), 6.51 (s, 1H), 5.89 (q, J = 6.8 Hz, 1H), 4.69-4.65 (m, 1H), 3.81 (s, 3H), 2.77 (s, 3H), 2.45 (s, 3H), 1.66 (d, J = 6.8 Hz, 3H), 1.34-1.31 (m, 6H) |
| Example 61 3-[5-[(1R)-1-(3,5- dimethylpyridazin-4- yl)ethoxy]-1H-indazol-3- yl]-5-(trifluoromethoxy)- benzonitrile | 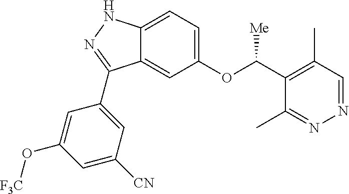 [SMILES: Cc1cnnc(C)c1[C@@H](C)Oc1ccc2[nH]nc(-c3cc(C#N)cc(OC(F)(F)F)c3)c2c1] | LCMS m/z = 454 (M + H); 1H NMR (400 MHz, DMSO-d6) δ 13.53 (s, 1H), 8.81 (s, 1H), 8.21 (s, 1H), 8.03 (d, J = 4.0 Hz, 2H), 7.56 (d, J = 9.2 Hz, 1H), 7.15-7.13 (m, 2H), 5.95 (q, J = 6.4 Hz, 1H), 2.80 (s, 3H), 2.47 (s, 3H), 1.69 (d, J = 6.8 Hz, 3H) |
| Example 62 3-[5-[(1R)-1-(3,5- dimethylpyridazin-4- yl)ethoxy]-1H-indazol-3- yl]-5-(4-methylpiperazin- 1-yl)benzonitrile | 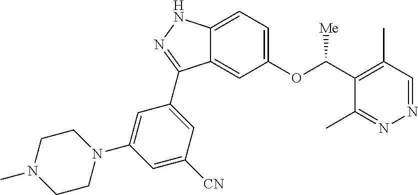 [SMILES: Cc1cnnc(C)c1[C@@H](C)Oc1ccc2[nH]nc(-c3cc(C#N)cc(N4CCN(C)CC4)c3)c2c1] | LCMS m/z = 468 (M + H); 1H NMR (400 MHz, DMSO-d6) δ 13.27 (s, 1H), 8.82 (s, 1H), 7.56-7.51 (m, 2H), 7.39 (s, 2H), 7.12 (dd, J = 8.8 Hz, 2.0 Hz, 1H), 6.98 (s, 1H), 5.93 (q, J = 6.8 Hz, 1H), 3.27 (t, J = 4.4 Hz, 4H), 2.78 (s, 3H), 2.47-2.46 (m, 7H), 2.24 (s, 3H), 1.67 (d, J = 6.8 Hz, 3H) |
| Example 63 5-[(1R)-1-(3,5- dimethylpyridazin-4- yl)ethoxy]-3-[3- methoxy-5- (trifluoromethoxy) phenyl]-1H-indazole | 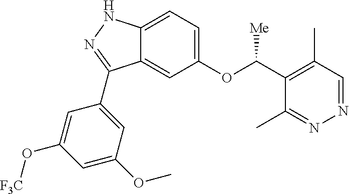 [SMILES: COc1cc(OC(F)(F)F)cc(-c2n[nH]c3ccc(O[C@H](C)c4c(C)cnnc4C)cc23)c1] | LCMS m/z = 459 (M + H); 1H NMR (400 MHz, DMSO-d6) δ 13.30 (s, 1H), 8.82 (s, 1H), 7.54 (d, J = 8.8 Hz, 1H), 7.27 (q, J = 1.6 Hz, 1H), 7.23 (s, 1H), 7.13 (dd, J = 8.8 Hz, 1.6 Hz, 1H), 7.00 (d, J = 2.4 Hz, 1H), 6.96-6.95 (m, 1H), 5.90 (q, J = 6.8 Hz, 1H), 3.89 (s, 3H), 2.77 (s, 3H), 2.44 (s, 3H), 1.67 (d, J = 6.8 Hz, 3H) |
| Example 64 3-[5-[(1R)-1-(3,5- dimethylpyridazin-4- yl)ethoxy]-1H-indazol-3- yl]-5-morpholino- benzonitrile | 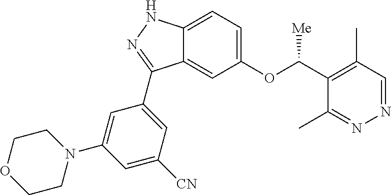 [SMILES: Cc1cnnc(C)c1[C@@H](C)Oc1ccc2[nH]nc(-c3cc(C#N)cc(N4CCOCC4)c3)c2c1] | LCMS m/z = 455 (M + H); 1H NMR (400 MHz, DMSO-d6) δ 13.25 (s, 1H), 8.78 (s, 1H), 7.53-7.52 (m, 1H), 7.49 (d, J = 8.8 Hz, 1H), 7.40 (s, 1H), 7.38-7.37 (m, 1H), 7.09 (dd, J = 8.8 Hz, 2.0 Hz, 1H), 6.95 (d, J = 2.0 Hz, 1H), 5.89 (q, J = 6.8 Hz, 1H), 3.74 (t, J = 4.8 Hz, 4H), 3.21 (t, J = 4.8 Hz, 4H), 2.75 (s, 3H), 2.42 (s, 3H), 1.64 (d, J = 6.8 Hz, 3H) |
| Example 65 3-[5-[(1S)-1-(3,5- dimethylpyridazin-4- yl)ethoxy]-1H-indazol-3- yl]-5-morpholino- benzonitrile | 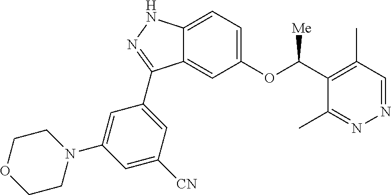 [SMILES: Cc1cnnc(C)c1[C@H](C)Oc1ccc2[nH]nc(-c3cc(C#N)cc(N4CCOCC4)c3)c2c1] | LCMS m/z = 455 (M + H); 1H NMR (400 MHz, DMSO-d6) δ 13.28 (s, 1H), 8.82 (s, 1H), 7.57 (s, 1H), 7.52 (d, J = 8.8 Hz, 1H), 7.43 (s, 1H), 7.41-7.40 (m, 1H), 7.12 (dd, J = 8.8 Hz, 2.0 Hz, 1H), 6.99 (d, J = 2.4 Hz, 1H), 5.92 (q, J = 6.4 Hz, 1H), 3.77 (t, J = 4.8 Hz, 4H), 3.24 (t, J = 4.8 Hz, 4H), 2.79 (s, 3H), 2.46 (s, 3H), 1.67 (d, J = 6.4 Hz, 3H) |
| Example 66 3-[5-[(1S)-1-(3,5- dimethylpyridazin-4- yl)ethoxy]-1H-indazol-3- yl]-5-(4-methylpiperazin- 1-yl)benzonitrile | 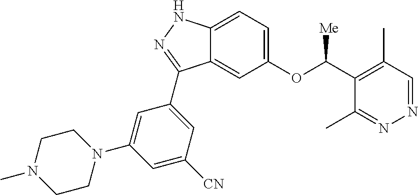 [SMILES: Cc1cnnc(C)c1[C@H](C)Oc1ccc2[nH]nc(-c3cc(C#N)cc(N4CCN(C)CC4)c3)c2c1] | LCMS m/z = 468 (M + H); 1H NMR (400 MHz, DMSO-d6) δ 13.27 (s, 1H), 8.82 (s, 1H), 7.56 (s, 1H), 7.52 (d, J = 9.2 Hz, 1H), 7.39 (s, 2H), 7.12 (dd, J = 8.8 Hz, 0.2 Hz, 1H), 6.98 (d, J = 1.2 Hz, 1H), 5.93 (q, J = 6.8 Hz, 1H), 3.27 (t, J = 4.4 Hz, 4H), 2.79 (s, 3H), 2.47-2.46 (m, 7H), 2.24 (s, 3H), 1.66 (d, J = 6.4 Hz, 3H) |
| Example 67 3-[5-[(1R)-1-(3,5- dimethylpyridazin-4- yl)ethoxy]-1H-indazol-3- yl]-5-tetrahydrofuran-3- yloxy-benzonitrile | 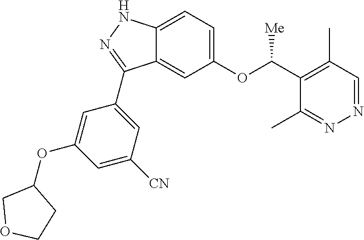 [SMILES: Cc1cnnc(C)c1[C@@H](C)Oc1ccc2[nH]nc(-c3cc(C#N)cc(OC4CCOC4)c3)c2c1] | LCMS m/z = 456 (M + H); 1H NMR (400 MHz, DMSO-d6) δ 13.38 (s, 1H), 8.82 (s, 1H), 7.71 (s, 1H), 7.55-7.47 (m, 3H), 7.12 (dd, J = 9.2 Hz, 2.4 Hz, 1H), 6.98 (s, 1H), 5.93 (q, J = 6.8 Hz, 1H), 5.22-5.21 (m, 1H), 3.94-3.90 (m, 3H), 3.81-3.80 (m, 1H), 2.79 (s, 3H), 2.47 (s, 3H), 2.32-2.28 (m, 1H), 2.07-2.01 (m, 1H), 1.68 (d, J = 7.2 Hz, 3H) |
| Example 68 3-[3-(cyclopropyl- methoxy)-5-methoxy- phenyl]-5-[(1R)-1-(3,5- dimethylpyridazin-4- yl)ethoxy]-1H-indazole | 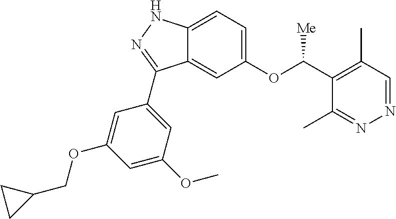 [SMILES: COc1cc(OCC2CC2)cc(-c2n[nH]c3ccc(O[C@H](C)c4c(C)cnnc4C)cc23)c1] | LCMS m/z = 445 (M + H); 1H NMR (400 MHz, DMSO-d6) δ 13.09 (brs, 1H), 8.84 (s, 1H), 7.50 (d, J = 9.2 Hz, 1H), 7.10 (dd, J = 9.2 Hz, 2.4 Hz, 1H), 6.94 (d, J = 2.0 Hz, 1H), 6.81-6.78 (m, 2H), 6.52 (t, J = 2.0 Hz, 1H), 5.87 (q, J = 6.8 Hz, 1H), 3.87 (d, J = 7.6 Hz, 2H), 3.81 (s, 3H), 2.76 (s, 3H), 2.46 (s, 3H), 1.67 (d, J = 6.4 Hz, 3H), 1.29-1.33 (m, 1H), 0.64-0.61 (m, 2H), 0.41-0.39 (m, 2H) |
| Example 69 4-[5-[(1R)-1-(3,5- dimethylpyridazin-4- yl)ethoxy]-1H-indazol-3- yl]-6-methyl-pyridine-2- carbonitrile | 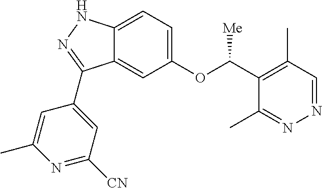 [SMILES: Cc1cc(-c2n[nH]c3ccc(O[C@H](C)c4c(C)cnnc4C)cc23)cc(C#N)n1] | LCMS m/z = 385 (M + H); 1H NMR (400 MHz, DMSO-d6) δ 13.69 (s, 1H), 8.82 (s, 1H), 8.15 (s, 1H), 7.90 (s, 1H), 7.58 (d, J = 8.8 Hz, 1H), 7.17-7.13 (m, 2H), 5.95 (q, J = 6.8 Hz, 1H), 2.82 (s, 3H), 2.63 (s, 3H), 2.47 (s, 3H), 1.70 (d, J = 6.8 Hz, 3H) |
| Example 70 4-[5-[(1S)-1-(3,5- dimethylpyridazin-4- yl)ethoxy]-1H-indazol- 3-yl]-6-methyl-pyridine- 2-carbonitrile | 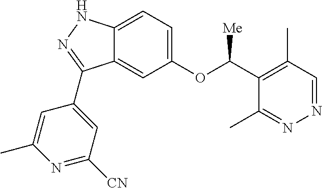 [SMILES: Cc1cc(-c2n[nH]c3ccc(O[C@@H](C)c4c(C)cnnc4C)cc23)cc(C#N)n1] | LCMS m/z = 385 (M + H); 1H NMR (400 MHz, DMSO-d6) δ 13.72 (s, 1H), 8.83 (s, 1H), 8.15 (d, J = 1.2 Hz, 1H), 7.90 (s, 1H), 7.58 (d, J = 9.2 Hz, 1H), 7.18 (d, J = 1.2 Hz, 1H), 7.15-7.12 (m, 1H), 5.95 (q, J = 7.2 Hz, 1H), 2.82 (s, 3H), 2.63 (s, 3H), 2.47 (s, 3H), 1.70 (d, J = 6.4 Hz, 3H) |
| Example 71 3-[5-[(1R)-1-(3,5- dimethylpyridazin-4- yl)ethoxy]-1H-indazol- 3-yl]-5-tetrahydropyran- 4-yloxy-benzonitrile | 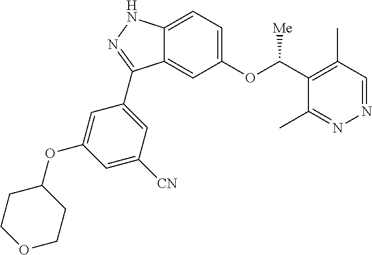 [SMILES: Cc1cnnc(C)c1[C@@H](C)Oc1ccc2[nH]nc(-c3cc(C#N)cc(OC4CCOCC4)c3)c2c1] | LCMS m/z = 470 (M + H); 1H NMR (400 MHz, DMSO-d6) δ 13.37 (s, 1H), 8.83 (s, 1H), 7.69 (s, 1H), 7.57-7.53 (m, 3H), 7.13 (dd, J = 8.8 Hz, 2.0 Hz, 1H), 6.98 (d, J = 2.0 Hz, 1H), 5.94 (q, J = 6.8 Hz, 1H), 4.80-4.76 (m, 1H), 3.92-3.89 (m, 2H), 3.56-3.53 (m, 2H), 2.80 (s, 3H), 2.48 (s, 3H), 2.05-2.03 (m, 2H), 1.69-1.64 (m, 5H) |
| Example 72 3-[5-[(1R)-1-(3,5- dimethylpyridazin-4- yl)ethoxy]-1H-indazol- 3-yl]-5-(2- hydroxyethoxy) benzonitrile | 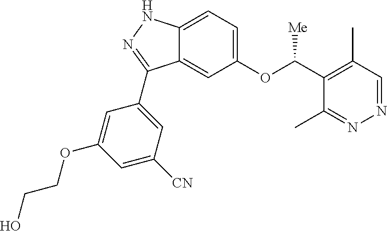 [SMILES: Cc1cnnc(C)c1[C@@H](C)Oc1ccc2[nH]nc(-c3cc(C#N)cc(OCCO)c3)c2c1] | LCMS m/z = 430 (M + H); 1H NMR (400 MHz, DMSO-d6) δ 13.37 (s, 1H), 8.86 (s, 1H), 7.69 (s, 1H), 7.52 (d, J = 8.8 Hz, 1H), 7.49-7.45 (m, 2H), 7.14-7.11 (m, 1H), 6.97 (d, J = 1.2 Hz, 1H), 5.93 (q, J = 6.0 Hz, 1H), 5.07 (t, J = 5.6 Hz, 1H), 4.14 (t, J = 4.4 Hz, 2H), 3.80 (q, J = 5.2 Hz, 2H), 2.78 (s, 3H), 2.47 (s, 3H), 1.69 (d, J = 6.4 Hz, 3H) |
| Example 73 3-[5-[(1R)-1-(3,5- dimethylpyridazin-4- yl)ethoxy]-1H-indazol-3- yl]-5-(2-ethoxyethoxy)- benzonitrile | 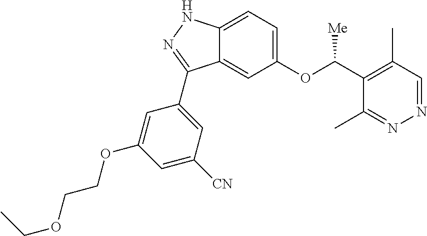 [SMILES: CCOCCOc1cc(C#N)cc(-c2n[nH]c3ccc(O[C@H](C)c4c(C)cnnc4C)cc23)c1] | LCMS m/z = 458 (M + H); 1H NMR (400 MHz, DMSO-d6) δ 13.41 (s, 1H), 8.85 (s, 1H), 7.70 (s, 1H), 7.55-7.51 (m, 2H), 7.48- 7.47 (m, 1H), 7.13 (dd, J = 9.2 Hz, 2.4 Hz, 1H), 6.98 (d, J = 2.0 Hz, 1H), 5.93 (q, J = 6.8 Hz, 1H), 4.25 (t, J = 8.4 Hz, 2H), 3.80- 3.77 (m, 2H), 3.55 (q, J = 6.8 Hz, 2H), 2.78 (s, 3H), 2.49 (s, 3H), 1.68 (d, J = 10.4 Hz, 3H), 1.16 (t, J = 7.2 Hz, 3H) |
| Example 74 5-[5-[(1R)-1-(3,5- dichloro-4-pyridyl) ethoxy]-1H-indazol-3- yl]-2-methoxy-pyridine- 3-carbonitrile | 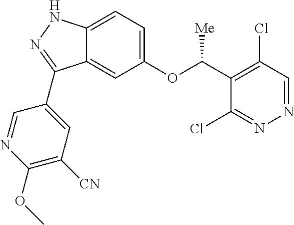 [SMILES: COc1ncc(-c2n[nH]c3ccc(O[C@H](C)c4c(Cl)cnnc4Cl)cc23)cc1C#N] | LCMS m/z = 440 (M + H); 1H NMR (400 MHz, DMSO-d6) δ = 13.34 (s, 1H), 8.89 (d, J = 2.4 Hz, 1H), 8.57 (s, 2H), 8.52 (d, J = 2.4 Hz, 1H), 7.52 (d, J = 9.0 Hz, 1H), 7.25 (d, J = 2.2 Hz, 1H), 7.12 (dd, J = 2.3, 9.0 Hz, 1H), 6.16 (q, J = 6.6 Hz, 1H), 4.09 (s, 3H), 1.77 (d, J = 6.6 Hz, 3H) |
| Example 75 3-[5-[(1R)-1-(3,5- dimethylpyridazin-4- yl)ethoxy]-1H-indazol-3- yl]-5-(2-methoxyethoxy)- benzonitrile | 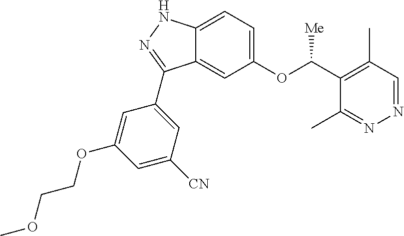 [SMILES: COCCOc1cc(C#N)cc(-c2n[nH]c3ccc(O[C@H](C)c4c(C)cnnc4C)cc23)c1] | LCMS m/z = 444 (M + H); 1H NMR (400 MHz, DMSO-d6) δ 13.37 (s, 1H), 8.84 (s, 1H), 7.70 (s, 1H), 7.54-7.52 (m, 2H), 7.48 (s, 1H), 7.12 (dd, J = 9.2 Hz, 2.0 Hz, 1H), 6.99 (d, J = 2.0 Hz, 1H), 5.92 (q, J = 6.8 Hz, 1H), 4.25 (t, J = 4.4 Hz, 2H), 3.74 (t, J = 5.2 Hz, 2H), 3.36 (s, 3H), 2.78 (s, 3H), 2.48 (s, 3H), 1.68 (d, J = 7.2 Hz, 3H) |
| Example 76 5-[5-[(1R)-1-(3,5- dimethylpyridazin-4- yl)ethoxy]-1H-indazol-3- yl]-2,3-dimethoxy- benzonitrile | 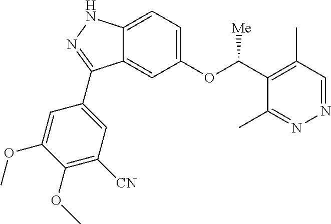 [SMILES: COc1cc(-c2n[nH]c3ccc(O[C@H](C)c4c(C)cnnc4C)cc23)cc(C#N)c1OC] | LCMS m/z = 430.5 (M + H); 1H NMR (400 MHz, DMSO-d6) δ 13.34 (s, 1H), 8.85 (s, 1H), 7.52 (d, J = 9.6 Hz, 1H), 7.48 (d, J = 8.8 Hz, 1H), 7.26 (d, J = 8.8 Hz, 1H), 7.11 (dd, J = 8.8 Hz, 2.0 Hz, 1H), 6.82 (d, J = 2.4 Hz, 1H), 5.78 (q, J = 6.8 Hz, 1H), 3.97 (s, 3H), 3.96 (s, 3H), 2.75 (s, 3H), 2.42 (s, 3H), 1.66 (d, J = 6.8 Hz, 3H) |
| Example 77 5-[5-[(1R)-1-(3,5- dimethylpyridazin-4- yl)ethoxy]-1H-indazol-3- yl]-2-fluoro-3-methyl- benzonitrile | 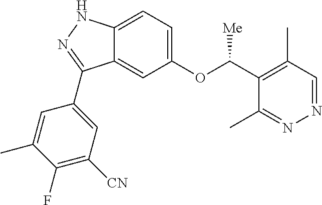 [SMILES: Cc1cc(-c2n[nH]c3ccc(O[C@H](C)c4c(C)cnnc4C)cc23)cc(C#N)c1F] | LCMS m/z = 402 (M + H); 1H NMR (400 MHz, DMSO-d6) δ 13.33 (s, 1H), 8.82 (s, 1H), 8.02-7.99 (m, 2H), 7.52 (d, J = 8.8 Hz, 1H), 7.11 (dd, J = 9.2 Hz, 2.4 Hz, 1H), 7.03 (d, J = 2.0 Hz, 1H), 5.91 (q, J = 6.8 Hz, 1H), 2.80 (s, 3H), 2.45 (s, 3H), 2.40 (s, 3H), 1.69 (d, J = 6.4 Hz, 3H) |
| Example 78 3-(cyclopropylmethoxy)- 5-[5-[(1R)-1-(3,5- dimethylpyridazin-4- yl)ethoxy]-1H-indazol-3- yl]benzonitrile | 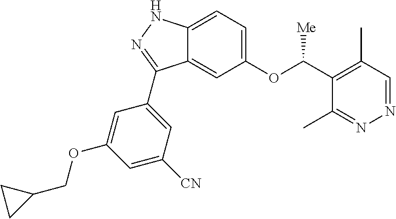 [SMILES: Cc1cnnc(C)c1[C@@H](C)Oc1ccc2[nH]nc(-c3cc(C#N)cc(OCC4CC4)c3)c2c1] | LCMS m/z = 440 (M + H); 1H NMR (400 MHz, DMSO-d6) δ 13.35 (s, 1H), 8.84 (s, 1H), 7.67 (s, 1H), 7.54-7.52 (m, 2H), 7.43-7.42 (m, 1H), 7.12 (dd, J = 8.8 Hz, 2.0 Hz, 1H), 7.00 (s, 1H), 5.93 (q, J = 6.8 Hz, 1H), 3.97 (d, J = 7.2 Hz, 2H), 2.79 (s, 3H), 2.48 (s, 3H), 1.68 (d, J = 7.2 Hz, 3H), 1.33-1.27 (m, 1H), 0.65-0.61 (m, 2H), 0.41-0.38 (m, 2H) |
| Example 79 3-[5-[(1R)-1-(3,5- dimethylpyridazin-4- yl)ethoxy]-1H-indazol-3- yl]-5-methyl-benzonitrile | 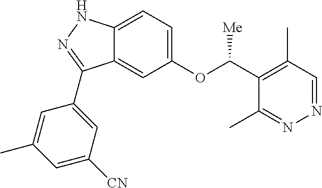 [SMILES: Cc1cc(C#N)cc(-c2n[nH]c3ccc(O[C@H](C)c4c(C)cnnc4C)cc23)c1] | LCMS m/z = 384 (M + H); 1H NMR (400 MHz, DMSO-d6) δ 13.34 (s, 1H), 8.82 (s, 1H), 7.92 (s, 1H), 7.85 (s, 1H), 7.67 (s, 1H), 7.53 (d, J = 9.6 Hz, 1H), 7.12 (dd, J = 8.8 Hz, 2.0 Hz, 1H), 7.01-7.00 (m, 1H), 5.91 (q, J = 6.4 Hz, 1H), 2.80 (s, 3H), 2.46 (s, 6H), 1.69 (d, J = 6.8 Hz, 3H) |
| Example 80 5-[5-[(1R)-1-(3,5- dimethylpyridazin-4- yl)ethoxy]-1H-indazol-3- yl]-2-methoxy-3-methyl- benzonitrile | 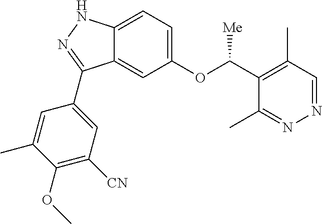 [SMILES: COc1c(C)cc(-c2n[nH]c3ccc(O[C@H](C)c4c(C)cnnc4C)cc23)cc1C#N] | LCMS m/z = 414.5 (M + H); 1H NMR (400 MHz, DMSO-d6) δ 13.27 (s, 1H), 8.83 (s, 1H), 7.89 (s, 2H), 7.51 (d, J = 9.6 Hz, 1H), 7.11 (dd, J = 8.8 Hz, 2.0 Hz, 1H), 6.99 (d, J = 2.0 Hz, 1H), 5.91 (q, J = 6.8 Hz, 1H), 3.97 (s, 3H), 2.80 (s, 3H), 2.46 (s, 3H), 2.37 (s, 3H), 1.68 (d, J = 6.8 Hz, 3H) |
| Example 81 5-[5-[(1R)-1-(3,5- dichloro-4-pyridyl) ethoxy]-1H-indazol-3- yl]-2,3-dimethoxy- benzonitrile | 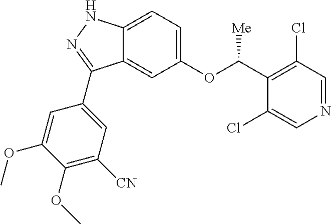 [SMILES: COc1cc(-c2n[nH]c3ccc(O[C@H](C)c4c(Cl)cncc4Cl)cc23)cc(C#N)c1OC] | LCMS m/z = 469 (M + H); 1H NMR (400 MHz, DMSO-d6) δ = 13.28 (s, 1H), 8.57 (s, 2H), 7.76 (d, J = 1.8 Hz, 1H), 7.52 (d, J = 9.0 Hz, 1H), 7.47 (d, J = 2.0 Hz, 1H), 7.17-7.10 (m, 2H), 6.13 (q, J = 6.6 Hz, 1H), 3.99 (s, 3H), 3.96 (s, 3H), 1.76 (d, J = 6.7 Hz, 3H) |
| Example 82 5-[5-[(1R)-1-(3,5- dimethylpyridazin-4- yl)ethoxy]-1H-indazol-3- yl]-3-fluoro-2-methoxy- benzonitrile | 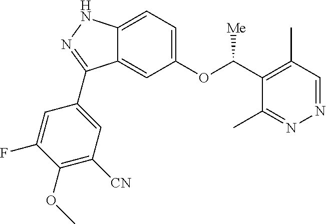 [SMILES: COc1c(F)cc(-c2n[nH]c3ccc(O[C@H](C)c4c(C)cnnc4C)cc23)cc1C#N] | LCMS m/z = 418 (M + H); 1H NMR (400 MHz, DMSO-d6) δ 13.37 (s, 1H), 8.82 (s, 1H), 7.96 (d, J = 12.8 Hz, 1H), 7.90 (s, 1H), 7.52 (d, J = 8.8 Hz, 1H), 7.11 (d, J = 9.2 Hz, 1H), 7.08 (s, 1H), 5.94 (q, J = 6.8 Hz, 1H), 4.14 (s, 3H), 2.80 (s, 3H), 2.47 (s, 3H), 1.68 (d, J = 6.4 Hz, 3H) |
| Example 83 5-[5-[(1R)-1-(3,5- dimethylpyridazin-4- yl)ethoxy]-1H-indazol-3- yl]benzene-1,3- dicarbonitrile | 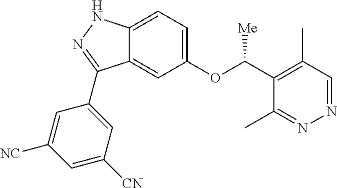 [SMILES: Cc1cnnc(C)c1[C@@H](C)Oc1ccc2[nH]nc(-c3cc(C#N)cc(C#N)c3)c2c1] | LCMS m/z = 395 (M + H); 1H NMR (400 MHz, DMSO-d6) δ 13.57 (s, 1H), 8.81 (s, 1H), 8.50-8.49 (m, 2H), 8.46-8.45 (m, 1H), 7.55 (d, J = 9.2 Hz, 1H), 7.18 (d, J = 2.0 Hz, 1H), 7.13 (dd, J = 9.2 Hz, 2.4 Hz, 1H), 5.96 (q, J = 6.8 Hz, 1H), 2.81 (s, 3H), 2.48 (s, 3H), 1.69 (d, J = 6.4 Hz, 3H) |
| Example 84 3-cyclopropyl-5-[5-[(1R)- 1-(3,5-dimethyl- pyridazin-4-yl)ethoxy]- 1H-indazol-3-yl] benzonitrile | 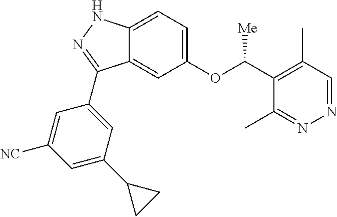 [SMILES: Cc1cnnc(C)c1[C@@H](C)Oc1ccc2[nH]nc(-c3cc(C#N)cc(C4CC4)c3)c2c1] | LCMS m/z = 410 (M + H); 1H NMR (400 MHz, DMSO-d6) δ 13.32 (brs, 1H), 8.82 (s, 1H), 7.85 (s, 1H), 7.82 (s, 1H), 7.53 (d, J = 9.2 Hz, 1H), 7.49 (s, 1H), 7.12 (dd, J = 8.8 Hz, 2.0 Hz, 1H), 7.01 (s, 1H), 5.93 (q, J = 6.8 Hz, 1H), 2.80 (s, 3H), 2.46 (s, 3H), 2.15-2.08 (m, 1H), 1.68 (d, J = 6.4 Hz, 3H), 1.10-1.05 (m, 2H), 0.88-0.81 (m, 2H) |
| Example 85 5-[5-[(1R)-1-(3,5- dimethylpyridazin-4- yl)ethoxy]-1H-indazol- 3-yl]-2-methoxy- benzene-1,3- dicarbonitrile | 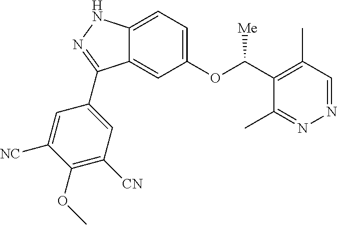 [SMILES: COc1c(C#N)cc(-c2n[nH]c3ccc(O[C@H](C)c4c(C)cnnc4C)cc23)cc1C#N] | LCMS m/z = 425 (M + H); 1H NMR (400 MHz, DMSO-d6) δ 13.45 (s, 1H), 8.81 (s, 1H), 8.41 (s, 2H), 7.53 (d, J = 9.2 Hz, 1H), 7.13-7.10 (m, 2H), 5.95 (q, J = 6.8 Hz, 1H), 4.29 (s, 3H), 2.81 (s, 3H), 2.48 (s, 3H), 1.69 (d, J = 6.8 Hz, 3H) |
| Example 86 3-cyclopentyl-5-[5-[(1R)- 1-(3,5-dimethylpyridazin- 4-yl)ethoxy]-1H-indazol- 3-yl]benzonitrile | 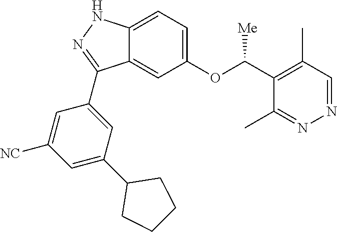 [SMILES: Cc1cnnc(C)c1[C@@H](C)Oc1ccc2[nH]nc(-c3cc(C#N)cc(C4CCCC4)c3)c2c1] | LCMS m/z = 438 (M + H); 1H NMR (400 MHz, DMSO-d6) δ 13.33 (s, 1H), 8.82 (s, 1H), 7.93-7.90 (m, 2H), 7.74 (s, 1H), 7.54 (d, J = 9.2 Hz, 1H), 7.13 (dd, J = 8.8 Hz, 1.6 Hz, 1H), 7.01 (d, J = 2.0 Hz, 1H), 5.92 (q, J = 6.8 Hz, 1H), 3.16-3.07 (m, 1H), 2.80 (s, 3H), 2.46 (s, 3H), 2.13-2.04 (m, 2H), 1.83-1.80 (m, 2H), 1.72-1.54 (m, 7H) |
| Example 87 3-[5-[(1R)-1-(3,5- dimethylpyridazin-4- yl)ethoxy]-1H-indazol- 3-yl]-5-(6-methyl-2,6- diazaspiro[3.3]heptan-2- yl)benzonitrile | 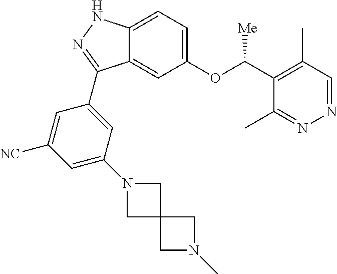 [SMILES: Cc1cnnc(C)c1[C@@H](C)Oc1ccc2[nH]nc(-c3cc(C#N)cc(N4CC5(CN(C)C5)C4)c3)c2c1] | LCMS m/z = 480 (M + H); 1H NMR (400 MHz, DMSO-d6) δ 13.26 (s, 1H), 8.82 (s, 1H), 7.52 (d, J = 8.8 Hz, 1H), 7.30 (s, 1H), 7.11 (d, J = 8.8 Hz, 1H), 7.03 (s, 1H), 6.97 (s, 1H), 6.85 (s, 1H), 5.91 (q, J = 6.4 Hz, 1H), 3.98 (s, 4H), 3.33 (s, 4H), 2.79 (s, 3H), 2.46 (s, 3H), 2.22 (s, 3H), 1.67 (d, J = 6.0 Hz, 3H) |

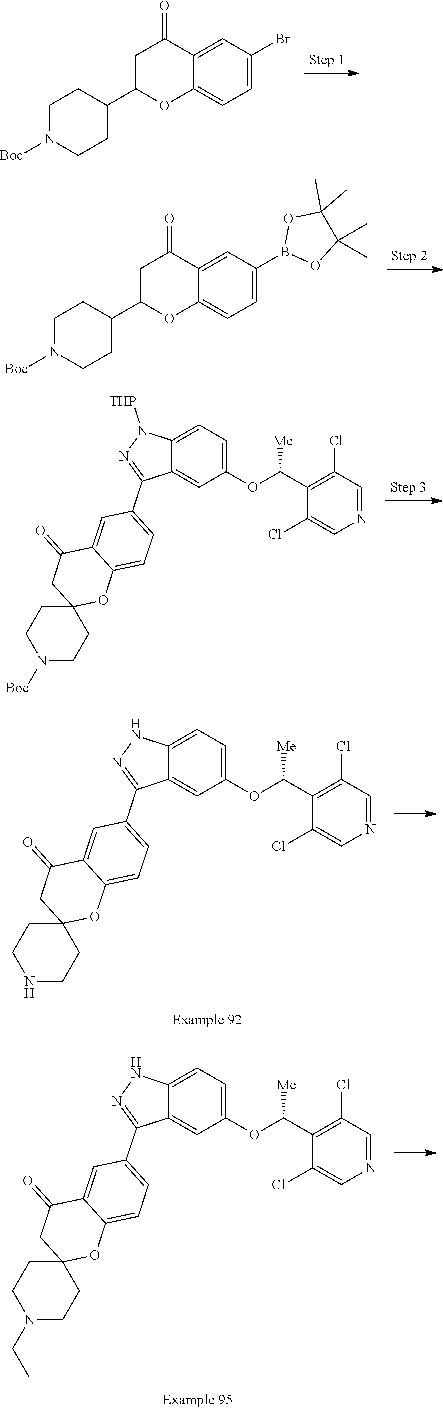

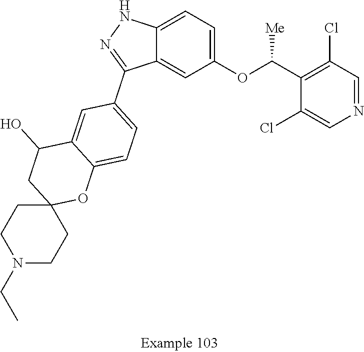 [SMILES: CCN1CCC2(CC1)CC(O)c1cc(-c3n[nH]c4ccc(O[C@H](C)c5c(Cl)cncc5Cl)cc34)ccc1O2]

### Example 92. (R)-6-(5-(1-(3,5-Dichloropyridin-4-yl)ethoxy)-1H-indazol-3-yl)spiro[chromane-2,4′-piperidin]-4-one

Step 1. tert-Butyl 4-oxo-6-(4,4,5,5-tetramethyl-1,3,2-dioxaborolan-2-yl)spiro[chromane-2,4′-piperidine]-1′-carboxylate. A mixture of tert-butyl 6-bromo-4-oxospiro[chromane-2,4′-piperidine]-1′-carboxylate (500 mg, 1.26 mmol, 1 equiv), potassium acetate (148 mg, 1.51 mmol, 1.2 equiv), [1,1′-bis(diphenylphosphino)ferrocene]dichloropalladium(II) (65 mg, 0.088 mmol, 0.07 equiv) and bis(pinacolato)diboron (352 mg, 1.39 mmol, 1. 1 equiv) in dimethylsulfoxide (5 mL) was sparged with nitrogen for 10 minutes then heated at 100° C. for 20 h. The reaction was cooled to room temperature and diluted with methanol (5 mL). The resulting solid was filtered and washed with methanol (5 mL) to give an off-white solid (220 mg, 36%). LCMS: m/z=909.5 (2M+Na).

Step 2. tert-Butyl 6-(5-((R)-1-(3,5-dichloropyridin-4-yl)ethoxy)-1-(tetrahydro-2H-pyran-2-yl)-1H-indazol-3-yl)-4-oxospiro[chromane-2,4′-piperidine]-1′-carboxylate. 5-[(1R)-1-(3,5-Dichloro-4-pyridyl)ethoxy]-3-iodo-1-tetrahydropyran-2-yl-indazole (214 mg, 0.42 mmol, 1 equiv), [1,1′-bis(diphenylphosphino)ferrocene]dichloropalladium(II) (31 mg, 0.042 mmol, 0.1 equiv) and potassium carbonate (115 mg, 0.83 mmol, 2.0 equiv) were added sequentially to a solution of tert-Butyl 4-oxo-6-(4,4,5,5-tetramethyl-1,3,2-dioxaborolan-2-yl)spiro[chromane-2,4′-piperidine]-1′-carboxylate (220 mg, 0.50 mmol, 1.2 equiv) in a 10 to 1 mixture of 1,4-dioxane and water (11 mL) in a sealed tube. After sparging with nitrogen for 10 minutes, the reaction was heated at 95° C. for 16 hours. After cooling to room temperature, the reaction mixture was filtered through Celite (5 g), which was washed with tetrahydrofuran (30 mL). The filtrate was concentrated under reduced pressure. The residue was purified on an InterChim automated chromatography system (Sorbtech, 25 g silica gel column), eluting with a gradient of 0 to 100% ethyl acetate in heptanes to give an off-white solid (292 mg, 99% yield). LCMS: m/z=707.2 (M+H).

Step 3. (R)-6-(5-(1-(3,5-Dichloropyridin-4-yl)ethoxy)-1H-indazol-3-yl)spiro[chromane-2,4′-piperidin]-4-one. Trifluoroacetic acid (1 mL) was added to a solution of tert-butyl 6-(5-((R)-1-(3,5-dichloropyridin-4-yl)ethoxy)-1-(tetrahydro-2H-pyran-2-yl)-1H-indazol-3-yl)-4-oxospiro[chromane-2,4′-piperidine]-1′-carboxy late (290 mg, 0.55 mmol, 1.0 equiv) in dichloromethane (1 mL) at room temperature. After stirring at room temperature for 2 hours, the volatiles were removed under reduced pressure. The residue was diluted with ethyl acetate (15 mL), washed with saturated sodium carbonate (10 mL), dried over sodium sulfate, filtered, and concentrated under reduced pressure. The residue was redissolved in dimethyl sulfoxide (10 mL) and purified on an InterChim automated chromatography system (RediSep Rf GOLD 100 g HP C18 column), eluting with a gradient of 0 to 100% acetonitrile in water to give an off-white solid (120 mg, 56% yield, 99.2% purity) after lyophilization. LCMS: m/z=523.1 (M+H); 1H NMR (400 MHZ, DMSO-d6) δ=13.13 (s, 1H), 8.61 (s, 2H), 8.08 (d, J=2.2 Hz, 1H), 7.94 (dd, J=2.3, 8.6 Hz, 1H), 7.49 (d, J=9.4 Hz, 1H), 7.21 (d, J=8.6 Hz, 1H), 7.13 (s, 1H), 7.13-7.09 (m, 1H), 6.08 (q, J=6.6 Hz, 1H), 2.91-2.81 (m, 4H), 2.76-2.69 (m, 2H), 1.86 (br d, J=15.7 Hz, 2H), 1.76 (d, J=6.6 Hz, 3H), 1.71-1.58 (m, 2H).

Example 95. (R)-6-(5-(1-(3,5-Dichloropyridin-4-yl)ethoxy)-1H-indazol-3-yl)-1′-ethylspiro[chromane-2,4′-piperidin]-4-one. Acetaldehyde (11 mg, 0.25 mmol, 1.2 equiv) was added to a solution of (R)-6-(5-(1-(3,5-dichloropyridin-4-yl)ethoxy)-1H-indazol-3-yl)spiro[chromane-2,4′-piperidin]-4-one (110 mg, 0.21 mmol, 1 equiv) in 1,2-dichloroethane (10 mL) at room temperature. Sodium triacetoxy borohydride (66 mg, 0.32 mmol, 1.5 equiv) was added and the mixture was stirred for 16 hours. The two batches were combined and the solvent was removed under reduced pressure. The residue was redissolved in dimethyl sulfoxide (10 mL) and purified on an InterChim automated chromatography system (RediSep Rf GOLD 100 g HP C18 column), eluting with a gradient of 0 to 100% acetonitrile in water to give an off-white solid (67 mg, 58%) after lyophilization. LCMS: m/z=551.2 (M+H); 1H NMR (400 MHZ, DMSO-d6) δ=13.13 (br s, 1H), 8.61 (s, 2H), 8.08 (d, J=2.3 Hz, 1H), 7.94 (dd, J=2.3, 8.6 Hz, 1H), 7.49 (d, J=9.3 Hz, 1H), 7.20 (d, J=8.6 Hz, 1H), 7.13 (s, 1H), 7.11 (d, J=9.1 Hz, 1H), 6.08 (q, J=6.6 Hz, 1H), 2.89 (s, 2H), 2.61 (br d, J=11.7 Hz, 2H), 2.42-2.30 (m, 4H), 1.96 (br d, J=13.7 Hz, 2H), 1.76 (d, J=6.7 Hz, 5H), 1.02 (t, J=7.2 Hz, 3H).

Example 103. 6-[5-[(1R)-1-(3,5-Dichloro-4-pyridyl)ethoxy]-1H-indazol-3-yl]-1′-ethyl-spiro[chromane-2,4′-piperidine]-4-ol. Sodium borohydride (3 mg, 0.071 mmol, 1 equiv) was added to a solution of (R)-6-(5-(1-(3,5-dichloropyridin-4-yl)ethoxy)-1H-indazol-3-yl)-1′-ethylspiro[chromane-2,4′-piperidin]-4-one (39 mg, 0.071 mmol, 1 equiv) in ethanol (10 mL) at 0° C. After 30 minutes additional sodium borohydride (26 mg, 0.64 mmol) was added and the mixture was stirred at room temperature for 18 hours. Water (1 mL) and saturated ammonium chloride solution (2 mL) were added. The solvent was removed under reduced pressure. The residue was redissolved in dimethyl sulfoxide (10 mL) and purified on an InterChim automated chromatography system (RediSep Rf GOLD 100 g HP C18 column), eluting with a gradient of 0 to 100% acetonitrile in water to give an off-white solid (2 mg, 5%) after lyophilization. LCMS: m/z=553.2 (M+H); 1H NMR (400 MHZ, DMSO-d6) δ=13.00 (br s, 1H), 9.56 (br s, 1H), 8.69-8.53 (m, 2H), 8.00-7.83 (m, 1H), 7.57-7.45 (m, 2H), 7.20-7.07 (m, 2H), 6.97 (br d, J=6.7 Hz, 1H), 6.14-6.03 (m, 1H), 5.53 (br d, J=15.8 Hz, 1H), 4.89-4.79 (m, 1H), 3.46-3.35 (m, 1H), 3.17 (br s, 5H), 2.30-2.04 (m, 3H), 1.91 (br d, J=5.1 Hz, 4H), 1.77 (dd, J=2.0, 6.7 Hz, 3H), 1.24 (br s, 3H).

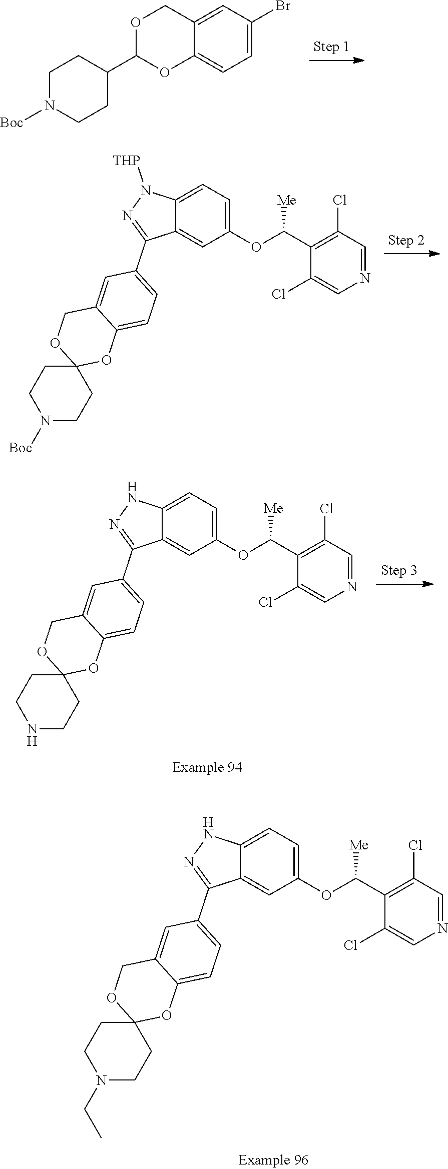

### Example 94. (R)-6-(5-(1-(3,5-Dichloropyridin-4-yl)ethoxy)-1H-indazol-3-yl)-4H-spiro[benzo[d][1,3]dioxine-2,4′-piperidine] TFA salt

Step 1. tert-Butyl 6-(5-((R)-1-(3,5-dichloropyridin-4-yl)ethoxy)-1-(tetrahydro-2H-pyran-2-yl)-1H-indazol-3-yl)-4H-spiro[benzo[d][1,3]dioxine-2,4′-piperidine]-1′-carboxylate. [1,1′-Bis(diphenylphosphino)ferrocene]palladium(II) dichloride (35.2 mg, 0.05 mmol, 0.07 equiv), bis(pinacolato)diboron (191.9 mg, 0.8 mmol, 1.1 equiv) and potassium acetate (80.9 mg, 0.82 mmol, 1.2 equiv) were added to a solution of compound tert-butyl 6-bromospiro[4H-1,3-benzodioxine-2,4′-piperidine]-1′-carboxylate (264 mg, 0.69 mmol, 1.0 equiv) in 1,4-dioxane (6.9 mL) at room temperature. The resulting mixture was sparged with nitrogen for 10 minutes then heated at 100° C. overnight. The mixture was cooled to room temperature, [1,1′-bis(diphenylphosphino)ferrocene]palladium(II) dichloride (65.4 mg, 0.07 mmol, 0.1 equiv), potassium carbonate (0.19 g, 1.37 mmol, 2.0 equiv), water (1.2 mL, 4.0 v) and 5-[(1R)-1-(3,5-dichloro-4-pyridyl)ethoxy]-3-iodo-1-tetrahydropyran-2-yl-indazole (356.1 mg, 0.69 mmol, 1.0 equiv) were added. The resulting mixture was sparged with nitrogen for 10 minutes then heated at 100° C. overnight. The mixture was cooled to room temperature, diluted with ethyl acetate (20 mL), washed with water (2×10 mL). The organic layer was dried over sodium sulfate and concentrated onto silica gel (4 g) directly. The crude material was purified on a Biotage automated chromatography system (Sorbtech, 25 g, silica gel column), eluting with a gradient of 0% to 100% ethyl acetate in heptane to give a as yellow solid (0.36 g, 76%2 steps). LCMS: m/z=695.2 (M+H).

Step 2. (R)-6-(5-(1-(3,5-Dichloropyridin-4-yl)ethoxy)-1H-indazol-3-yl)-4H-spiro[benzo[d][1,3]dioxine-2,4′-piperidine]. Trifluoroacetic acid (3.6 mL) was added to a solution of product step 1 (0.36 g, 0.52 mmol, 1.0 equiv) in dichloromethane (3.6 mL) at room temperature. The resulting mixture was stirred at room temperature overnight. The volatiles were removed under reduced pressure and the residue was diluted with dichloromethane (15 mL) and saturated sodium bicarbonate (5 mL). The mixture was stirred at room temperature for 1 hour, the organic layer was separated, dried over sodium sulfate, filtered and concentrated under reduced pressure to give a red oil (0.28 g, 90%). A 25 mg portion was concentrated onto Celite (3 g) and purified on a Biotage automated chromatography system (RediSep Rf Gold 50 g HP C18 column), eluting with a gradient of 0% to 100% acetonitrile in water to give a white solid (13.6 mg, 97%). LCMS: m/z=511.1 (M+H); 1H NMR (400 MHZ, CDCl3) δ=13.05 (s, 1H), 8.80-8.22 (m, 4H), 7.59 (dd, J=2.0, 8.4 Hz, 1H), 7.47 (dd, J=4.0, 5.8 Hz, 2H), 7.12-7.06 (m, 2H), 7.03 (d, J=8.4 Hz, 1H), 6.08 (q, J=6.6 Hz, 1H), 5.10-4.96 (m, 2H), 3.26-3.17 (m, 4H), 2.17-2.05 (m, 4H), 1.76 (d, J=6.6 Hz, 3H).

### Example 96. (R)-6-(5-(1-(3,5-Dichloropyridin-4-yl)ethoxy)-1H-indazol-3-yl)-1′-ethyl-4H-spiro[benzo[d][1,3]dioxine-2,4′-piperidine]

A solution of (R)-6-(5-(1-(3,5-Dichloropyridin-4-yl)ethoxy)-1H-indazol-3-yl)-4H-spiro[benzo[d][1,3]dioxine-2,4′-piperidine] TFA salt (0.10 g, 0.2 mmol, 1.0 equiv) in dichloromethane (2.0 mL) was treated with acetaldehyde (22 uL, 0.39 mmol, 2.0 equiv) and sodium triacetoxy borohydride (124.3 mg, 0.59 mmol, 3.0 equiv) at 0° C. After stirring at room temperature overnight, the reaction was concentrated onto Celite (1 g) under reduced pressure. The residue was purified on a Biotage automated chromatography system (RediSep Rf Gold 50 g HP C18 column), eluting with a gradient of 0 to 100% acetonitrile in water to give a white solid (11 mg, 11%). LCMS: m/z=539.2 (M+H). 1H NMR (400 MHZ, CDCl3) δ=13.02 (s, 1H), 8.63 (s, 2H), 7.55 (dd, J=1.8, 8.5 Hz, 1H), 7.46 (d, J=9.7 Hz, 1H), 7.44 (s, 1H), 7.12-7.06 (m, 2H), 6.98 (d, J=8.4 Hz, 1H), 6.08 (q, J=6.6 Hz, 1H), 5.01-4.91 (m, 2H), 2.61-2.52 (m, 2H), 2.48-2.36 (m, 2H), 1.91 (br s, 4H), 1.76 (d, J=6.6 Hz, 3H), 1.23 (s, 2H), 1.04 (br t, J=7.0 Hz, 3H).

### Example 157. (R)-1′-Cyclobutyl-6-(5-(1-(3,5-dichloropyridin-4-yl)ethoxy)-1H-indazol-3-yl)-4H-spiro[benzo[d][1,3]dioxine-2,4′-piperidine]

(R)-6-(5-(1-(3,5-Dichloropyridin-4-yl)ethoxy)-1H-indazol-3-yl)-4H-spiro[benzo[d][1,3]dioxine-2,4′-piperidine] TFA salt (160 mg, 0.26 mmol, 1.0 equiv) was treated with cyclobutanone (0.39 mL, 20.0 equiv, 5.3 mmol), sodium triacetoxy borohydride (668 mg, 12.0 equiv, 3.2 mmol) and N,N-diisopropylethylamine (0.55 mL, 12.0 equiv, 3.2 mmol) in anhydrous dichloromethane (2.0 mL) at 0° C. After stirring at room temperature for 6 hours, the reaction was diluted with dichloromethane (10 mL) and saturated sodium bicarbonate (10 mL). The mixture was stirred at room temperature for 1 hour. The organic layer was separated, dried over sodium sulfate, and filtered. The filtrate was concentrated onto Celite (2 g) under reduced pressure and purified on a Biotage automated chromatography system (RediSep Rf Gold R Reversed-phase C18), eluting with a gradient of 0 to 100% acetonitrile in water to give as an off-white solid (80 mg, 54% yield). LCMS (ESI): m/z=603.2; 1H NMR (400 MHZ, DMSO-d6) δ=13.12-12.93 (m, 1H), 8.64-8.55 (m, 2H), 7.55 (dd, J=2.1, 8.6 Hz, 1H), 7.49-7.41 (m, 2H), 7.12-7.06 (m, 2H), 6.97 (d, J=8.4 Hz, 1H), 6.08 (q, J=6.8 Hz, 1H), 5.04-4.86 (m, 2H), 2.85-2.70 (m, 1H), 2.37 (br s, 3H), 2.28 (dt, J=4.3, 7.3 Hz, 1H), 2.03-1.95 (m, 3H), 1.93-1.77 (m, 1H), 1.77-1.73 (m, 3H), 1.67-1.58 (m, 2H), 1.53-1.43 (m, 2H), 1.28 (br s, 4H).

### (R)-1-(3,5-dichloro-2-fluoro-4-pyridyl)ethanol and(S)-1-(3,5-dichloro-2-fluoro-4-pyridyl)ethanol

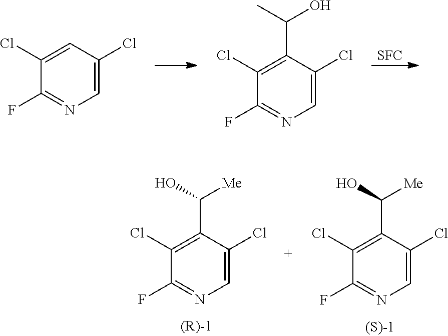

Step 1. 1-(3,5-Dichloro-2-fluoro-4-pyridyl)ethanol 3,5-Dichloro-2-fluoro-pyridine (50 g; 0.3 mol) and acetaldehyde (264 g 6 mol) in THF was added LDA (1.5 eq.) dropwise slowly at −78° C. After the addition was complete the solution was allowed to warm to rt. The reaction was quenched with aqueous ammonium chloride solution, extracted with ethyl acetate (200 mL×3), dried over Na₂SO₄ and concentrated. The residue was purified by silica gel column (PE/EA=30/1) to give (20 g) a light-yellow oil. LCMS m/z=211 (M+H). ¹H NMR (400 MHZ, CDCl3) δ 8.24 (s, 1H), 5.71 (d, 1H) 5.35 (q, 1H), 1.42 (d, 3H).

Step 2. (R)-1-(3,5-Dichloro-2-fluoro-4-pyridyl)ethanol and (S)-1-(3,5-Dichloro-2-fluoro-4-pyridyl)ethanol. Racemic product step 1 was separated using chiral SFC and Amylose Neo 50 column (3 mm×5 um), with MeOH 10-50%. (R)-1-(3,5-dichloro-2-methyl-4-pyridyl)ethanol and(S)-1-(3,5-dichloro-2-methyl-4-pyridyl)ethanol

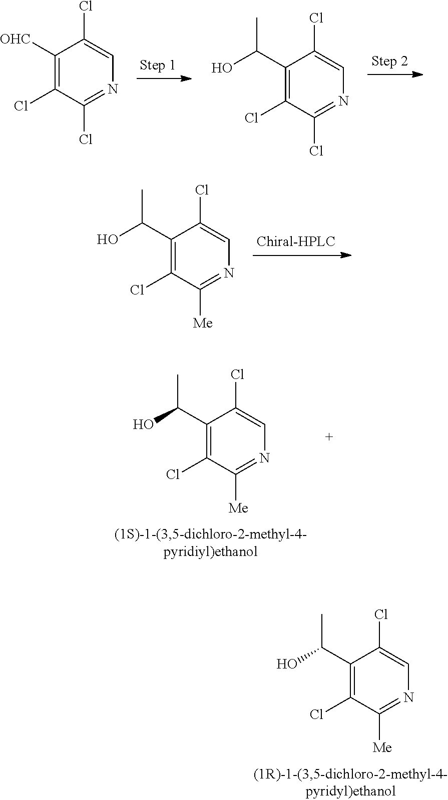

Step 1. (Trichloro-4-pyridyl)ethanol. A 500 mL three-neck round bottom flask was charged with 2,3,5-trichloropyridine-4-carbaldehyde (40 g, 0.19 mol) and THF (200 mL). MeMgBr (70 mL, 0.21 mol) was added in portions and the mixture was stirred at −70° C. for 1 h. The reaction was quenched with aqueous ammonium chloride solution, extracted with ethyl acetate (200 mL×3), dried over Na₂SO₄ and concentrated. The residue was purified by silica gel column (PE/EA=30/1) to give (33 g) a yellow liquid (33 g, 77%). LCMS: m/z=227 (M+H).

Step 2. 1-(3,5-Dichloro-2-methyl-4-pyridyl)ethanol. A mixture of product step 1 (33 g, 0.147 mmol), methylboronic acid (26.3 g, 0.429 mmol) K2CO3 (40 g, 0.290 mmol) and Pd(PPh3)2Cl2 (3 g) in dioxane (300 mL) was stirred at 110° C. overnight. The resulting mixture was filtered and the filtrate was concentrated in vacuo to give the crude product, which was further purified by silica gel column chromatography to give a yellow liquid (15 g, 50%). LCMS: m/z=206.1 (M+H). The product was separated by Prep-HPLC (Chiralpak ID 5×25 cm, hexanes/ethanol (80/20), 60 mL/min. 38C.°) to give (S)-1-(3,5-dichloro-2-methyl-4-pyridyl)ethanol (5 g) and (R)-1-(3,5-dichloro-2-methyl-4-pyridyl)ethanol R (5 g) as yellow liquid. Peak 1 5.5 min; (S)-1-(3,5-dichloro-2-methyl-4-pyridyl)ethanol. LCMS: m/z=206.1 (M+H). 1H NMR (400 MHZ, CDCl3) δ 8.36 (s, 1H), 5.57 (m, 1H), 2.93 (b, 1H), 2.64 (s, 3H), 1.65 (d, 3H). Peak 2 6.9 min; (R)-1-(3,5-dichloro-2-methyl-4-pyridyl)ethanol. LCMS: m/z=206.1 (M+H). 1H NMR (400 MHZ, CDCl3) δ 8.36 (s, 1H), 5.57 (m, 1H), 2.93 (b, 1H), 2.64 (s, 3H), 1.65 (d, 3H).

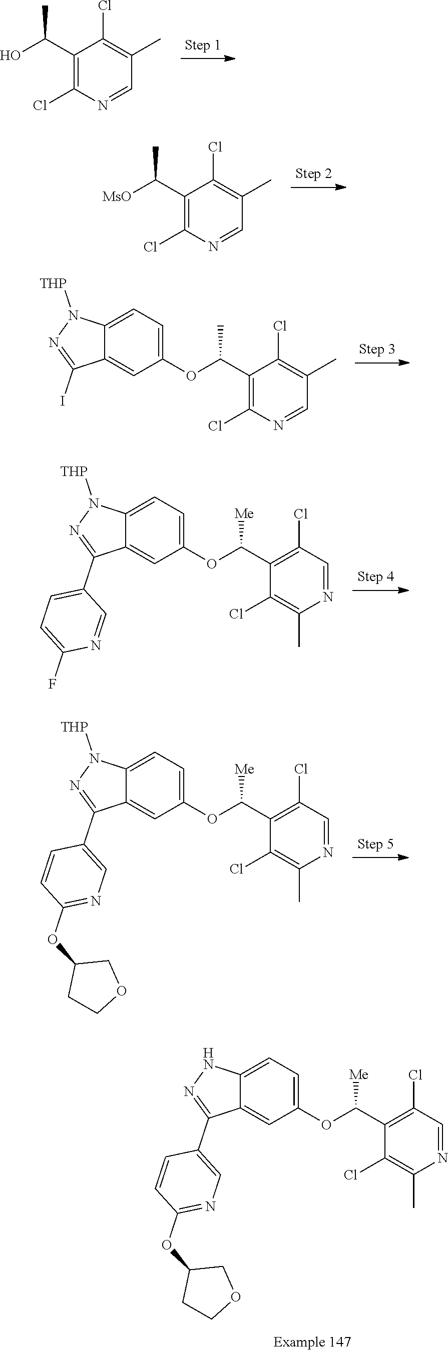

### Example 147. 5-((R)-1-(3,5-Dichloro-2-methylpyridin-4-yl)ethoxy)-3-(6-(((R)-tetrahydrofuran-3-yl)oxy)pyridin-3-yl)-1H-indazole

Step 1. (S)-1-(3,5-Dichloro-2-methylpyridin-4-yl)ethyl methanesulfonate. Methanesulfonyl chloride (1.35 mL, 17.5 mmol, 1.2 equiv) was added to a solution of compound (S)-263-1 (3 g, 14.6 mmol, 1 equiv) and triethylamine (4 mL, 29.1 mmol, 2 equiv) in anhydrous dichloromethane (30 mL) at −10° C. After two hours, the reaction was warmed to room temperature and diluted with deionized water (30 mL, 0° C.). The layers were separated and the aqueous layer was extracted with dichloromethane (3×15 mL). The combined organic layers were dried over sodium sulfate, filtered and concentrated under reduced pressure. The crude yellow oil was absorbed onto Celite (20 g) and was purified on an Interchim automated chromatography system (Sorbtech, 100 g silica gel column) eluting with a gradient of 0 to 100% methyl tert-butyl ether and heptanes to give compound a white solid (4.04 g, 96%). LCMS: m/z=284.1 (M+H).

Step 2. 5-((R)-1-(3,5-Dichloro-2-methylpyridin-4-yl)ethoxy)-3-iodo-1-(tetrahydro-2H-pyran-2-yl)-1H-indazole. A mixture of compound 3-iodo-1-tetrahydropyran-2-yl-indazol-5-ol (5.38 g, 15.6 mmol, 1.1 equiv), (S)-1-(3,5-dichloro-2-methylpyridin-4-yl)ethyl methanesulfonate (4.04 g, 14.2 mmol, 1 equiv) and cesium carbonate (9.27 g, 28.5 mmol, 2 equiv) in acetonitrile (80 mL) was heated at 80° C. overnight. The reaction was cooled to room temperature and was diluted with ethyl acetate (100 mL) and saturated brine solution (100 mL). The layers were separated and the aqueous layer was extracted with ethyl acetate (3×30 mL). The combined organic layers were dried over sodium sulfate, filtered and concentrated under reduced pressure. The residue was absorbed onto Celite (30 g) and was purified on an Interchim automated chromatography system (Sorbtech, 80 g silica gel column), eluting with a gradient of 0 to 100% ethyl acetate in heptanes to give compound 287-1 (6.5 g, 86%) as a white solid. LCMS: m/z=532.1 (M+H).

Step 3. 5-((R)-1-(3,5-Dichloro-2-methylpyridin-4-yl)ethoxy)-3-(6-fluoropyridin-3-yl)-1-(tetrahydro-2H-pyran-2-yl)-1H-indazole. A mixture of 5-((R)-1-(3,5-dichloro-2-methylpyridin-4-yl)ethoxy)-3-iodo-1-(tetrahydro-2H-pyran-2-yl)-1H-indazole (1 g, 1.87 mmol, 1 equiv), 2-fluoro-5-(4,4,5,5-tetramethyl-1,3,2-dioxaborolan-2-yl)pyridine (503 mg, 2.26 mmol, 1.2 equiv), [1,1′-bis(diphenylphosphino)ferrocene]-palladium(II) dichloride (137 mg, 0.187 mmol, 0.1 equiv) and potassium carbonate (774 mg, 5.61 mmol, 3 equiv) in a 20 to 1 mixture of 1,4-dioxane and water (9.4 mL) was sparged with nitrogen for 10 minutes. After heating at 90° C. overnight, the reaction as cooled to room temperature and was diluted with water (10 mL) and ethyl acetate (10 mL). The layers were separated and the aqueous layer was extracted with ethyl acetate (3×5 mL). The combined organic layers were dried over sodium sulfate, filtered and concentrated under reduced pressure. The residue was absorbed onto Celite (20 g) and was purified on an Interchim automated chromatography system (Sorbtech, 80 g silica gel column), eluting with a gradient of 0 to 100% ethyl acetate in heptanes to give a white solid (940, 99%). LCMS: m/z=501.1 (M+H).

Step 4. 5-((R)-1-(3,5-Dichloro-2-methylpyridin-4-yl)ethoxy)-1-(tetrahydro-2H-pyran-2-yl)-3-(6-(((R)-tetrahydrofuran-3-yl)oxy)pyridin-3-yl)-1H-indazole. Potassium t-butoxide (56 mg, 0.5 mmol, 1.25 equiv) was added to a solution of 5-((R)-1-(3,5-dichloro-2-methylpyridin-4-yl)ethoxy)-3-(6-fluoropyridin-3-yl)-1-(tetrahydro-2H-pyran-2-yl)-1H-indazole (44 mg, 0.5 mmol, 1.25 equiv) in 1-methyl-2-pyrrolidone (5 mL) at room temperature and the mixture was stirred for 10 minutes. (3R)-Tetrahydrofuran-3-ol (200.6 mg, 0.4 mmol, 1 equiv) was added and the mixture was stirred at room temperature for 18 hours. Additional (3R)-tetrahydrofuran-3-ol (17.6 mg, 0.2 mmol, 0.5 equiv) and potassium t-butoxide (22 mg, 0.2 mmol, 0.5 equiv) were added. The mixture was stirred at room temperature for 3 hours at which time LCMS analysis indicated that the reaction was complete. The mixture was diluted with ethyl acetate (40 mL) and water (20 mL). The layers were separated and the aqueous layer was extracted with ethyl acetate (3×20 mL). The combined organic layers were washed with saturated brine (40 mL), dried over sodium sulfate, filtered and concentrated under reduced pressure. The residue was purified on an Interchim automated chromatography system (40 g Sorbtech silica gel column), eluting with a gradient of 0) to 50% ethyl acetate in heptanes to give a colorless oil (150 mg, 65% yield). LCMS m/z=569 (M+H).

Step 5. 5-((R)-1-(3,5-Dichloro-2-methylpyridin-4-yl)ethoxy)-3-(6-(((R)-tetrahydrofuran-3-yl)oxy)pyridin-3-yl)-1H-indazole. Trifluoroacetic acid (1.5 mL) was added to a solution of 5-((R)-1-(3,5-dichloro-2-methylpyridin-4-yl)ethoxy)-1-(tetrahydro-2H-pyran-2-yl)-3-(6-(((R)-tetrahydrofuran-3-yl)oxy)pyridin-3-yl)-1H-indazole (150 mg, 0.263 mmol, 1 equiv) in dichloromethane (1.5 mL) at room temperature. After stirring for 16 hours, the mixture was concentrated under reduced pressure onto Celite (8 g) and purified on an InterChim automated chromatography system (RediSep Rf GOLD 50 g HP C18 column), eluting with a gradient of 0 to 100% acetonitrile in water, to give a white solid (110 mg, 86%). LCMS: m/z=485 (M+H); 1H NMR (400 MHZ, DMSO-d6) δ ppm 13.15 (s, 1H) 8.42-8.46 (m, 2H) 8.05 (dd, J=8.6, 2.4 Hz, 1H) 7.49 (d, J=9.0 Hz, 1H) 7.07-7.13 (m, 2H) 6.97 (dd, J=8.6, 0.6 Hz, 1H) 6.07-6.13 (m, 1H) 5.58 (ddt, J=6.5, 4.4, 2.0, 2.0 Hz, 1H) 3.99 (dd, J=10.3, 4.8 Hz, 1H) 3.78-3.95 (m, 3H) 2.58 (s, 3H) 2.24-2.35 (m, 1H) 2.02-2.12 (m, 1H) 1.76 (d, J=6.7 Hz, 3H).

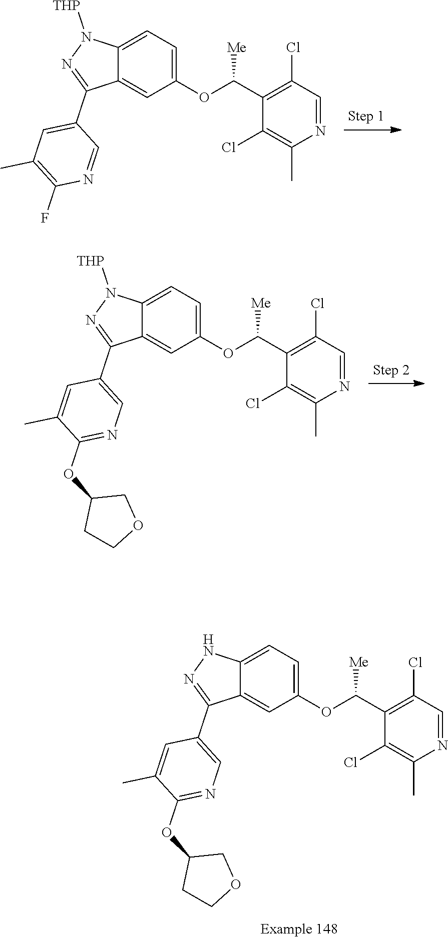

Example 148. 5-((R)-1-(3,5-Dichloro-2-methylpyridin-4-yl)ethoxy)-3-(5-methyl-6-(((R)-tetrahydrofuran-3-yl)oxy)pyridin-3-yl)-1H-indazole Step 1. 5-((R)-1-(3,5-Dichloro-2-methylpyridin-4-yl)ethoxy)-3-(5-methyl-6-(((R)-tetrahydrofuran-3-yl)oxy)pyridin-3-yl)-1-(tetrahydro-2H-pyran-2-yl)-1H-indazole. A mixture of 5-[(1R)-1-(3,5-dichloro-2-methyl-4-pyridyl)ethoxy]-3-(6-fluoro-5-methyl-3-pyridyl)-1-tetrahydropyran-2-yl-indazole (0.150 g, 0.29 mmol, 1 equiv), (3R)-tetrahydrofuran-3-ol (0.373 g, 4.2 mmol, 14.34 equiv) and cesium carbonate (0.295 g, 0.90 mmol, 3.13 equiv) in 1-methyl-2-pyrrolidone (2.5 mL) was heated at 120° C. for 16 hours. The reaction mixture was cooled to room temperature and diluted with water (80 mL). After stirring at room temperature for 1 hour, the resulting precipitate was filtered, washed with water (2×20 mL) and dried at 50° C. for 2 hours and at room temperature for 18 hours under vacuum to give an off-white solid (0.146 g, 86%). LCMS: m/z=583 (M+H).

Step 2. 5-((R)-1-(3,5-Dichloro-2-methylpyridin-4-yl)ethoxy)-3-(5-methyl-6-(((R)-tetrahydrofuran-3-yl)oxy)pyridin-3-yl)-1H-indazole: A solution of 5-((R)-1-(3,5-dichloro-2-methylpyridin-4-yl)ethoxy)-3-(5-methyl-6-(((R)-tetrahydrofuran-3-yl)oxy)pyridin-3-yl)-1-(tetrahydro-2H-pyran-2-yl)-1H-indazole (146 mg, 0.25 mmol, 1 equiv) in dichloromethane (2 mL) was treated with trifluoroacetic acid (2 mL, 25.9 mmol, 111.1 equiv) at room temperature. After 2.5 hours, the reaction mixture was concentrated under reduced pressure, diluted with saturated sodium carbonate (25 mL) and extracted with dichloromethane (2×15 mL). The combined organic layers were washed with saturated brine (15 mL), dried over magnesium sulfate, filtered and concentrated under reduced pressure. The residue was purified on a Biotage automated chromatography system (Biotage 25 g silica gel column, 20 μm), eluting with a gradient of 0 to 70% ethyl acetate in heptanes. Product fractions were concentrated and the residue was precipitated from a mixture of ethyl acetate (1 mL), methanol (0.2 mL), MTBE (1 mL) and heptanes (15 mL) to give an off-white solid (37 mg, 30%) after drying at 50° C. for 5 hours under vacuum. LCMS (ESI) m/z=499 (M+H); 1H NMR (400 MHZ, DMSO-d6) δ=13.11 (br s, 1H), 8.42 (s, 1H), 8.25 (d, J=2.1 Hz, 1H), 7.90 (dd, J=0.8, 2.3 Hz, 1H), 7.48 (d, J=9.0 Hz, 1H), 7.10 (dd, J=2.3, 9.0 Hz, 1H), 7.05 (d, J=2.1 Hz, 1H), 6.12-6.06 (m, 1H), 5.58 (tdd, J=2.1, 4.4, 6.5 Hz, 1H), 4.02-3.97 (m, 1H), 3.93-3.78 (m, 3H), 2.56 (s, 3H), 2.33-2.21 (m, 4H), 2.16-1.98 (m, 1H), 1.75 (d, J=6.6 Hz, 3H).

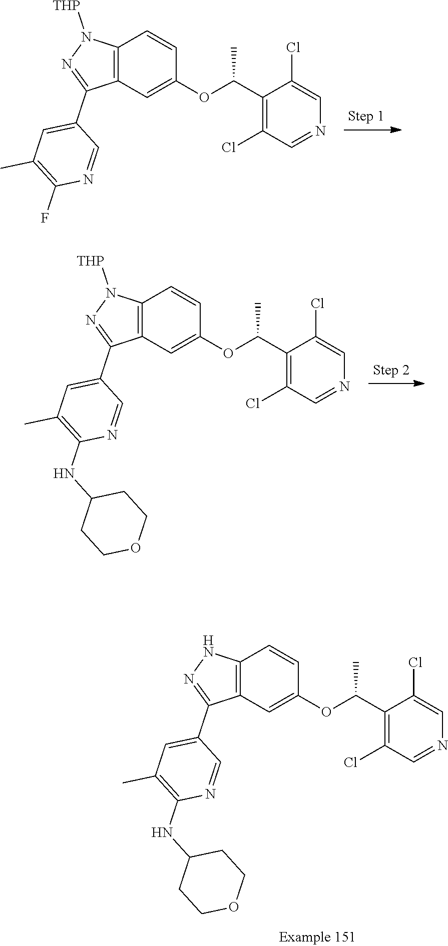

### Example 151. (R)-5-(5-(1-(3,5-Dichloropyridin-4-yl)ethoxy)-1H-indazol-3-yl)-3-methyl-N-(tetrahydro-2H-pyran-4-yl)pyridin-2-amine

Step 1. 5-[5-[(1R)-1-(3,5-Dichloro-4-pyridyl)ethoxy]-1-tetrahydropyran-2-yl-indazol-3-yl]-3-methyl-N-tetrahydropyran-4-yl-pyridin-2-amine. To a solution of 5-[(1R)-1-(3,5-dichloro-4-pyridyl)ethoxy]-3-(6-fluoro-5-methyl-3-pyridyl)-1-tetrahydropyran-2-yl-indazole (100 mg, 0.2 mmol, 1.0 eq) and tetrahydropyran-4-amine (101 mg, 1.0 mmol, 5.0 eq) in DMSO (2.5 mL) was added DIEA (258 mg, 2.0 mmol, 10.0 eq) at 25° C., the resulting mixture was stirred at 130° C. for 48 h. After cooled to room temperature, the reaction mixture was diluted with 10 ml water and extracted with EtOAc (10 mL×2). The combined organic layers were washed with water, brine, dried and concentrated. The crude product was purified by Prep-TLC (DCM/MeOH=15/1) to give a yellow solid (100 mg, 86%). LCMS m/z=582 (M+1).

Step 2: 5-[5-[(1R)-1-(3,5-dichloro-4-pyridyl)ethoxy]-1H-indazol-3-yl]-3-methyl-N-tetrahydropyran-4-yl-pyridin-2-amine. To a solution of 5-[5-[(1R)-1-(3,5-dichloro-4-pyridyl)ethoxy]-1-tetrahydropyran-2-yl-indazol-3-yl]-3-methyl-N-tetrahydropyran-4-yl-pyridin-2-amine (90 mg, 0.15 mmol, 1.0 eq) in DCM (3 mL) was added TFA (1 mL) at 0° C., the resulting mixture was stirred at r.t. for 3 h. After completion, the reaction mixture was concentrated. The crude product was treated with DCM/MeOH (V:V=5/1, 10 mL), K₂CO₃ (excess) was added to the solution and stirred for 20 minutes at rt, then DCM (20 mL) was added. The solid was filtered out and the filtrate was concentrated, the residue was purified by Prep-TLC (DCM/MeOH=10/1) to give a white solid (54 mg, 71% yield). LCMS m/z=498 (M+1). 1H-NMR (400 MHZ, DMSO-d6) δ 12.91 (s, 1H), 8.58 (s, 2H), 8.30 (s, 1H), 7.55 (s, 1H), 7.44 (d, J=9.2 Hz, 1H), 7.12 (s, 1H), 7.07 (d, J=8.8 Hz, 1H), 6.09 (q, J=6.4 Hz, 1H), 5.75 (d, J=8.0 Hz, 1H), 4.26-4.14 (m, 1H), 3.91 (d, J=10.8 Hz, 2H), 3.51-3.39 (m, 2H), 2.17 (s, 3H), 1.91 (d, J=12.0 Hz, 2H), 1.74 (d, J=6.4 Hz, 3H), 1.67-1.54 (m, 2H).

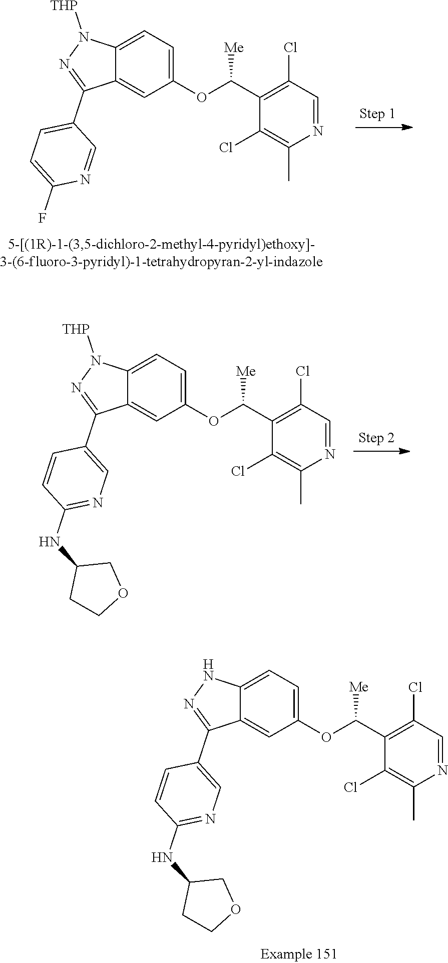

### Example 155. 5-(5-((R)-1-(3,5-Dichloro-2-methylpyridin-4-yl)ethoxy)-1H-indazol-3-yl)-N-((R)-tetrahydrofuran-3-yl)pyridin-2-amine

Step 1. 5-(5-((R)-1-(3,5-Dichloro-2-methylpyridin-4-yl)ethoxy)-1-(tetrahydro-2H-pyran-2-yl)-1H-indazol-3-yl)-N-((R)-tetrahydrofuran-3-yl)pyridin-2-amine (467-2): Potassium carbonate (250 mg, 1.4 mmol, 3.5 equiv) was added to a mixture of 5-((R)-1-(3,5-dichloro-2-methylpyridin-4-yl)ethoxy)-3-(6-fluoropyridin-3-yl)-1-(tetrahydro-2H-pyran-2-yl)-1H-indazole (201 mg, 0.4 mmol, 1 equiv) and (3R)-tetrahydrofuran-3-amine 4-methylbenzenesulfonic acid salt (207 mg, 0.8 mmol, 2 equiv) in 1-methyl-2-pyrrolidone (5 mL) at room temperature. After heating at 120° C. for 16 hours, LCMS analysis indicated that a 9 to 1 mixture of starting material and product was present. Additional (3R)-tetrahydrofuran-3-amine tosyl salt (207 mg, 0.8 mmol, 2 equiv) and potassium carbonate (220 mg, 1.6 mmol, 4 equiv) were added, and the mixture was stirred at 150° C. for 48 hours. After cooling to room temperature, the mixture was diluted with ethyl acetate (80 mL) and water (40 mL). The layers were separated and the aqueous layer was extracted with ethyl acetate (3×30 mL). The combined organic layers were washed with saturated brine (50 mL), dried over sodium sulfate, filtered, and concentrated under reduced pressure. The residue was purified on an Interchim automated chromatography system (Sorbtech, 40 g silica gel column), eluting with a gradient of 0 to 100% ethyl acetate in heptanes to give a white foam (120 mg, 53%). LCMS m/z=568 (M+H).

Step 2. 5-(5-((R)-1-(3,5-Dichloro-2-methylpyridin-4-yl)ethoxy)-1H-indazol-3-yl)-N-((R)-tetrahydrofuran-3-yl)pyridin-2-amine. Trifluoroacetic acid (1.2 mL) was added to a solution of product step 1 (120 mg, 0.211 mmol, 1 equiv) in dichloromethane (1.2 mL) at room temperature. After stirring at room temperature for 16 hours, the mixture was concentrated under reduced pressure onto Celite (8 g), then purified on an Interchim automated chromatography system (RediSep Rf GOLD 50 g HP C18 column), eluting with a gradient of 0 to 100% acetonitrile in water to give a white solid as the TFA salt (75 mg, 75%). LCMS m/z=484 (M+H); 1H NMR (400 MHZ, DMSO-d6) δ=12.91 (s, 1H), 8.42 (s, 1H), 8.28 (d, J=2.1 Hz, 1H), 7.72 (dd, J=2.3, 8.7 Hz, 1H), 7.44 (d, J=8.9 Hz, 1H), 7.07 (dd, J=2.3, 9.0 Hz, 1H), 7.02 (s, 1H), 6.97 (d, J=6.1 Hz, 1H), 6.63 (dd, J=0.7, 8.7 Hz, 1H), 6.08 (q, J=6.6 Hz, 1H), 4.44 (ttd, J=4.0, 5.9, 7.6 Hz, 1H), 3.94 (dd, J=5.9, 8.8 Hz, 1H), 3.90-3.84 (m, 1H), 3.75 (dt, J=5.6, 8.1 Hz, 1H), 3.59 (dd, J=3.9, 8.8 Hz, 1H), 2.60 (s, 3H), 2.22 (qd, J=7.4, 12.6 Hz, 1H), 1.89-1.81 (m, 1H), 1.75 (d, J=6.7 Hz, 3H).

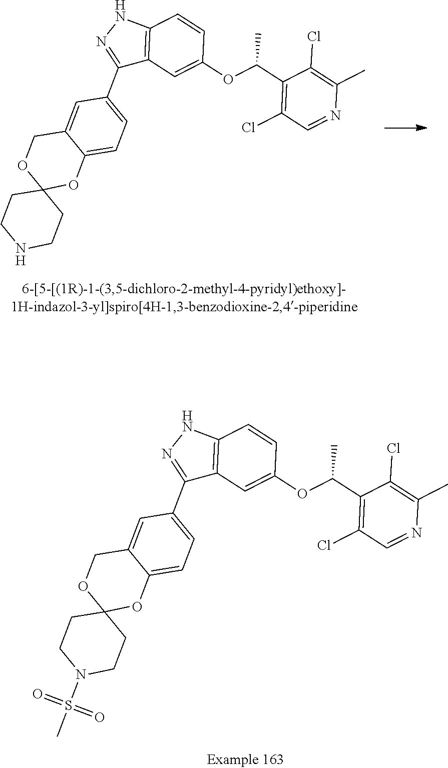 [SMILES: Cc1ncc(Cl)c([C@@H](C)Oc2ccc3[nH]nc(-c4ccc5c(c4)COC4(CCNCC4)O5)c3c2)c1Cl ; Cc1ncc(Cl)c([C@@H](C)Oc2ccc3[nH]nc(-c4ccc5c(c4)COC4(CCN(S(C)(=O)=O)CC4)O5)c3c2)c1Cl]

### Example 163. (R)-6-(5-(1-(3,5-Dichloro-2-methylpyridin-4-yl)ethoxy)-1H-indazol-3-yl)-1′-(methylsulfonyl)-4H-spiro[benzo[d][1,3]dioxine-2,4′-piperidine]

Triethylamine (30 uL, 1.0 equiv, 0.2 mmol) and methanesulfonyl chloride (17 uL, 1.0 equiv, 0.2 mmol) were sequentially added at 0° C. to a solution of 6-[5-[(1R)-1-(3,5-dichloro-2-methyl-4-pyridyl)ethoxy]-1H-indazol-3-yl]spiro[4H-1,3-benzodioxine-2,4′-piperidine] TFA salt (112 mg, 1.0 equiv, 0.2 mmol) in dichloromethane (3.0 mL). After stirring at room temperature for 1 hour, the reaction was diluted with saturated sodium bicarbonate (5 mL) and extracted with dichloromethane (3×5 mL). The combined organic layers were dried over sodium sulfate, filtered and concentrated under reduced pressure. The residue was purified on a Biotage automated chromatography system (Sorbtech, 12 g silica gel column), eluting with a gradient of 0 to 100% ethyl acetate in heptane to give an off-white solid (87 mg, 41%). LCMS m/z=603.1 (M+H); 1H NMR (400 MHZ, DMSO-d6) δ=13.03 (s, 1H), 8.49 (s, 1H), 7.57 (dd, J=2.1, 8.4 Hz, 1H), 7.52-7.44 (m, 2H), 7.15-7.04 (m, 2H), 7.00 (d, J=8.4 Hz, 1H), 6.11 (q, J=6.6 Hz, 1H), 5.05-4.95 (m, 2H), 3.29-3.25 (m, 1H), 3.00-2.94 (m, 4H), 2.53-2.51 (m, 3H), 2.11-1.99 (m, 5H), 1.75 (d, J=6.7 Hz, 4H).

### Example 166. (R)-6-(5-(1-(3,5-Dichloro-2-methylpyridin-4-yl)ethoxy)-1H-indazol-3-yl)-1′-(2,2-difluoroethyl)-4H-spiro[benzo[d][1,3]dioxine-2,4′-piperidine]

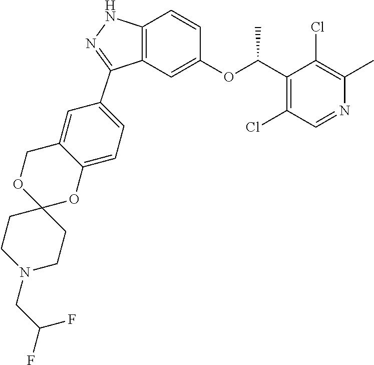 [SMILES: Cc1ncc(Cl)c([C@@H](C)Oc2ccc3[nH]nc(-c4ccc5c(c4)COC4(CCN(CC(F)F)CC4)O5)c3c2)c1Cl]

6-[5-[(1R)-1-(3,5-Dichloro-2-methyl-4-pyridyl)ethoxy]-1H-indazol-3-yl]spiro[4H-1,3-benzodioxine-2,4′-piperidine] TFA salt (136 mg, 0.26 mmol, 1.0 equiv), cesium carbonate (0.5 g, 1.6 mmol, 3.0 equiv) and 2,2-difluoroethyl trifluoromethanesulfonate (0.15 mL, 1.2 mmol, 4.5 equiv) in acetonitrile (2.5 mL) was heated at 50° C. overnight. The reaction was cooled to room temperature, diluted with ethyl acetate (40 mL) and washed with water (20 mL). The organic layer was dried over sodium sulfate, filtered and concentrated under reduced pressure. The residue was purified on a Biotage automated chromatography system (Sorbtech, 12 g silica gel column), eluting with a gradient of 0 to 100% ethyl acetate in heptane to give a white solid (49 mg, 33%). LCMS m/z=589.2 (M+H); 1H NMR (400 MHZ, DMSO-d6) δ=13.01 (s, 1H), 8.48 (s, 1H), 7.55 (dd, J=1.8, 8.5 Hz, 1H), 7.50-7.42 (m, 2H), 7.12-7.06 (m, 2H), 6.96 (d, J=8.4 Hz, 1H), 6.40-5.96 (m, 2H), 5.02-4.90 (m, 2H), 2.81 (dt, J=4.3, 15.7 Hz, 2H), 2.68 (br s, 4H), 2.51 (s, 3H), 1.95-1.86 (m, 4H), 1.75 (d, J=6.6 Hz, 3H).

Examples 88-169 were synthesized using procedures described in the previous examples.

| | | |
|---|---|---|
| Example 88 5-[5-[(1R)- 1-(3,5-dimethyl- pyridazin-4- yl)ethoxy]- 1H-indazol-3-yl]- 2-methoxy- pyridine-3- carbonitrile | 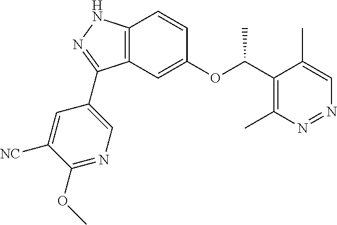 [SMILES: COc1ncc(-c2n[nH]c3ccc(O[C@H](C)c4c(C)cnnc4C)cc23)cc1C#N] | LCMS m/z = 401.3 (M + H); 1H NMR (400 MHz, DMSO-d6) δ 13.33 (s, 1H), 8.89 (d, J = 2.4 Hz, 1H), 8.82 (s, 1H), 8.58 (d, J = 2.4 Hz, 1H), 7.52 (d, J = 8.8 Hz, 1H), 7.16 (s, 1H), 7.10 (dd, J = 9.2 Hz, 1.2 Hz, 1H), 5.93 (q, J = 6.4 Hz, 1H), 4.08 (s, 3H), 2.80 (s, 3H), 2.46 (s, 3H), 1.68 (d, J = 6.4 Hz, 3H) |
| Example 89 (R)-2- (dimethyl- amino)-5-(5-(1- (3,5-dimethyl- pyridazin-4- yl)ethoxy)-1H- indazol-3- yl)nicotino- nitrile | 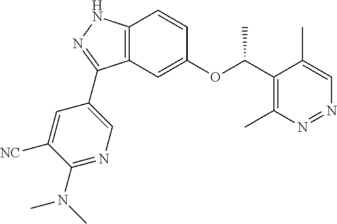 [SMILES: Cc1cnnc(C)c1[C@@H](C)Oc1ccc2[nH]nc(-c3cnc(N(C)C)c(C#N)c3)c2c1] | LCMS m/z = 414.3 (M + H); 1H NMR (400 MHz, DMSO-d6) δ 13.16 (s, 1H), 8.82 (s, 1H), 8.78 (d, J = 2.4 Hz, 1H), 8.20 (d, J = 2.4 Hz, 1H), 7.49 (d, J = 9.2 Hz, 1H), 7.09 (dd, J = 9.2 Hz, J = 2.4 Hz, 1H), 7.06 (d, J = 2.0 Hz, 1H), 5.92 (q, J = 6.8 Hz, 1H), 3.30 (s, 6H), 2.79 (s, 3H), 2.47 (s, 3H), 1.67 (d, J = 6.8 Hz, 3H) |
| Example 90 3-(5-((R)-1- (3,5-dimethyl- pyridazin-4- yl)ethoxy)-1H- indazol-3-yl)- 5-(tetrahydro- furan-3-yl) benzonitrile | 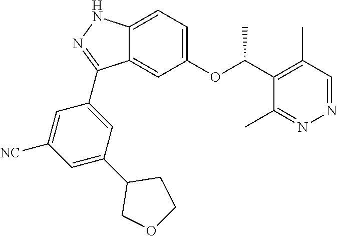 [SMILES: Cc1cnnc(C)c1[C@@H](C)Oc1ccc2[nH]nc(-c3cc(C#N)cc(C4CCOC4)c3)c2c1] | LCMS m/z = 440.3 (M + H); 1H NMR (400 MHz, DMSO-d6) δ 13.36 (s, 1H), 8.82 (s, 1H), 7.99-7.96 (m, 2H), 7.78 (d, J = 1.2 Hz, 1H), 7.55 (d, J = 9.2 Hz, 1H), 7.12 (dd, J = 9.2 Hz, 1.2 Hz, 1H), 7.04 (s, 1H), 5.94 (q, J = 6.4 Hz, 1H), 4.08-4.01 (m, 2H), 3.87-3.81 (m, 1H), 3.68-3.62 (m, 1H), 3.59-3.52 (m, 1H), 2.80 (s, 3H), 2.46 (s, 3H), 2.33-2.42 (m, 1H), 2.04-1.96 (m, 1H), 1.68 (d, J = 6.4 Hz, 3H). |
| Example 91 (S)-3-(5-(1-(3,5- dimethyl- pyridazin-4- yl)ethoxy)-1H- indazol-3-yl)- 5-(6-methyl- 2,6-diazaspiro [3.3]heptan-2- yl)benzonitrile | 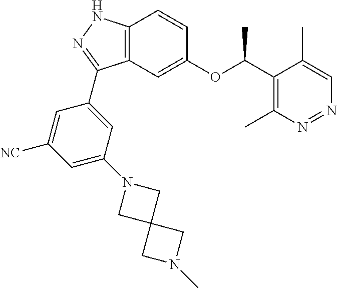 [SMILES: Cc1cnnc(C)c1[C@H](C)Oc1ccc2[nH]nc(-c3cc(C#N)cc(N4CC5(CN(C)C5)C4)c3)c2c1] | LCMS m/z = 480.4 (M + H); 1H NMR (400 MHz, DMSO-d6) δ 13.27 (s, 1H), 8.83 (s, 1H), 7.53 (d, J = 8.8 Hz, 1H), 7.31 (s, 1H), 7.12 (dd, J = 8.8 Hz, 2.0 Hz, 1H), 7.03 (s, 1H), 6.97 (d, J = 2.0 Hz, 1H), 6.87 (s, 1H), 5.91 (q, J = 6.4 Hz, 1H), 4.01 (s, 4H), 3.57 (s, 4H), 2.79 (s, 3H), 2.46 (s, 3H), 2.37 (s, 3H), 1.6 (d, J = 6.4 Hz, 3H) |
| Example 92 6-[5-[(1R)-1- (3,5-dichloro- 4-pyridyl) ethoxy]-1H- indazol-3- yl]spiro [chromane-2,4′- piperidine]-4- one | 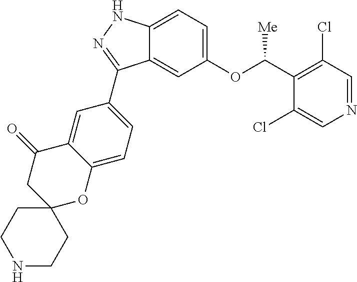 [SMILES: C[C@@H](Oc1ccc2[nH]nc(-c3ccc4c(c3)C(=O)CC3(CCNCC3)O4)c2c1)c1c(Cl)cncc1Cl] | LCMS m/z = 523.1. (M + H); 1H NMR (400 MHz, DMSO-d6) δ = 13.13 (s, 1H), 8.61 (s, 2H), 8.08 (d, J = 2.2 Hz, 1H), 7.94 (dd, J = 2.3, 8.6 Hz, 1H), 7.49 (d, J = 9.4 Hz, 1H), 7.21 (d, J = 8.6 Hz, 1H), 7.13 (s, 1H), 7.13-7.09 (m, 1H), 6.08 (q, J = 6.6 Hz, 1H), 2.91-2.81 (m, 4H), 2.76-2.69 (m, 2H), 1.86 (br d, J = 15.7 Hz, 2H), 1.76 (d, J = 6.6 Hz, 3H), 1.71-1.58 (m, 2H) |
| Example 93 (R)-5-(5-(1- (3,5-dimethyl- pyridazin-4-yl) ethoxy)-1H- indazol-3-yl)- 2-fluoro-3- methoxy- benzonitrile | 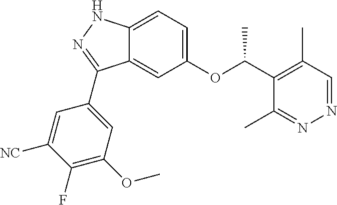 [SMILES: COc1cc(-c2n[nH]c3ccc(O[C@H](C)c4c(C)cnnc4C)cc23)cc(C#N)c1F] | LCMS m/z = 418.2 (M + H); 1H NMR (400 MHz, DMSO-d6) δ 13.35 (s, 1H), 8.82 (s, 1H), 7.83 (dd, J = 8.4 Hz, 1.2 Hz, 1H), 7.68 (dd, J = 9.2 Hz, 1.2 Hz, 1H), 7.53 (d, J = 8.8 Hz, 1H), 7.13-7.09 (m, 2H), 5.93 (q, J = 6.8 Hz, 1H), 3.98 (s, 3H), 2.79 (s, 3H), 2.47 (s, 3H), 1.68 (d, J = 6.4 Hz, 3H) |
| Example 94 6-[5-[(1R)-1- (3,5-dichloro- 4-pyridyl) ethoxy]-1H- indazol-3-yl] spiro[4H-1,3- benzodioxine- 2,4′- piperidine] | 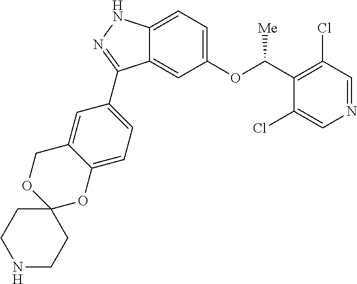 [SMILES: C[C@@H](Oc1ccc2[nH]nc(-c3ccc4c(c3)COC3(CCNCC3)O4)c2c1)c1c(Cl)cncc1Cl] | LCMS m/z = 511.1 (M + H); 1H NMR (400 MHz, CDCl3) δ = 13.05 (s, 1H), 8.80-8.22 (m, 4H), 7.59 (dd, J = 2.0, 8.4 Hz, 1H), 7.47 (dd, J = 4.0, 5.8 Hz, 2H), 7.12-7.06 (m, 2H), 7.03 (d, J = 8.4 Hz, 1H), 6.08 (q, J = 6.6 Hz, 1H), 5.10-4.96 (m, 2H), 3.26-3.17 (m, 4H), 2.17-2.05 (m, 4H), 1.76 (d, J = 6.6 Hz, 3H) |
| Example 95 6-[5-[(1R)-1- (3,5-dichloro-4- pyridyl)ethoxy]- 1H-indazol-3-yl]- 1′-ethylspiro [chromane-2,4′- piperidine]-4-one | 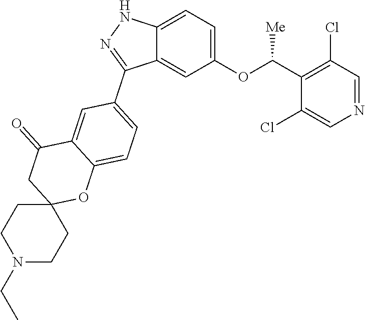 [SMILES: CCN1CCC2(CC1)CC(=O)c1cc(-c3n[nH]c4ccc(O[C@H](C)c5c(Cl)cncc5Cl)cc34)ccc1O2] | LCMS m/z = 551.2 (M + H); 1H NMR (400 MHz, DMSO-d6) δ = 13.13 (br s, 1H), 8.61 (s, 2H), 8.08 (d, J = 2.3 Hz, 1H), 7.94 (dd, J = 2.3, 8.6 Hz, 1H), 7.49 (d, J = 9.3 Hz, 1H), 7.20 (d, J = 8.6 Hz, 1H), 7.13 (s, 1H), 7.11 (d, J = 9.1 Hz, 1H), 6.08 (q, J = 6.6 Hz, 1H), 2.89 (s, 2H), 2.61 (br d, J = 11.7 Hz, 2H), 2.42-2.30 (m, 4H), 1.96 (br d, J = 13.7 Hz, 2H), 1.76 (d, J = 6.7 Hz, 5H), 1.02 (t, J = 7.2 Hz, 3H) |
| Example 96 6-[5-[(1R)-1- (3,5-dichloro-4- pyridyl)ethoxy]- 1H-indazol-3-yl]- 1′-ethylspiro [4H-1,3- benzodioxine- 2,4′-piperidine] | 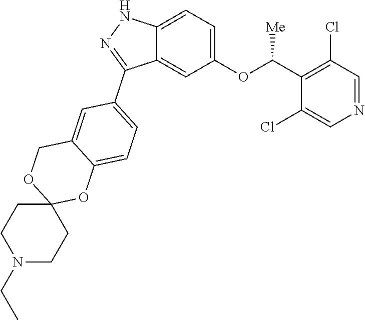 [SMILES: CCN1CCC2(CC1)OCc1cc(-c3n[nH]c4ccc(O[C@H](C)c5c(Cl)cncc5Cl)cc34)ccc1O2] | LCMS m/z = 539.2 (M + H); 1H NMR (400 MHz, CDCl3) δ = 13.02 (s, 1H), 8.63 (s, 2H), 7.55 (dd, J = 1.8, 8.5 Hz, 1H), 7.46 (d, J = 9.7 Hz, 1H), 7.44 (s, 1H), 7.12-7.06 (m, 2H), 6.98 (d, J = 8.4 Hz, 1H), 6.08 (q, J = 6.6 Hz, 1H), 5.01-4.91 (m, 2H), 2.61-2.52 (m, 2H), 2.48- 2.36 (m, 2H), 1.91 (br s, 4H), 1.76 (d, J = 6.6 Hz, 3H), 1.23 (s, 2H), 1.04 (br t, J = 7.0 Hz, 3H) |
| Example 97 (S)-3-(5-(1-(3,5- dimethyl- pyridazin-4-yl) ethoxy)-6- methoxy-1H- indazol-3-yl)-5- methoxy- benzonitrile | 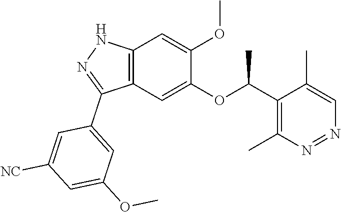 [SMILES: COc1cc(C#N)cc(-c2n[nH]c3cc(OC)c(O[C@@H](C)c4c(C)cnnc4C)cc23)c1] | LCMS m/z = 430.2 (M + H); 1H NMR (400 MHz, DMSO-d6) δ 13.14 (s, 1H), 8.83 (s, 1H), 7.66-7.64 (m, 1H), 7.55-7.54 (m, 1H), 7.46- 7.45 (m, 1H), 7.04 (s, 1H), 6.97 (s, 1H), 5.88 (q, J = 6.8 Hz, 1H), 3.90-3.89 (m, 6H), 2.77 (s, 3H), 2.46 (s, 3H), 1.68 (d, J = 6.4 Hz, 3H) |
| Example 98 (R)-4-(5-(1-(3,5- dimethyl- pyridazin-4-yl) ethoxy)-6- methoxy-1H- indazol-3-yl)-6- methoxy- picolinonitrile | 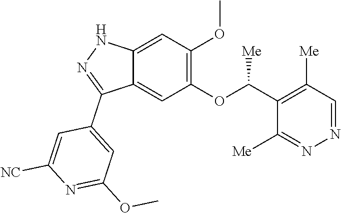 [SMILES: COc1cc(-c2n[nH]c3cc(OC)c(O[C@H](C)c4c(C)cnnc4C)cc23)cc(C#N)n1] | LCMS m/z = 431.1 (M + H); 1H NMR (400 MHz, DMSO-d6) δ 13.45 (brs, 1H), 8.83 (s, 1H), 7.96 (s, 1H), 7.37 (s, 1H), 7.09 (s, 1H), 7.08 (s, 1H), 5.92 (q, J = 6.8 Hz, 1H), 3.98 (s, 3H), 3.90 (s, 3H), 2.79 (s, 3H), 2.47 (s, 3H), 1.69 (d, J = 6.4 Hz, 3H) |
| Example 99 (R)-6-(5-(1-(3,5- dimethyl- pyridazin-4-yl) ethoxy)-1H- indazol-3-yl)-1′- ethylspiro [chroman-2,4′- piperidin]-4-one | 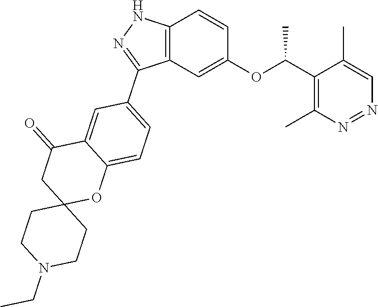 [SMILES: CCN1CCC2(CC1)CC(=O)c1cc(-c3n[nH]c4ccc(O[C@H](C)c5c(C)cnnc5C)cc34)ccc1O2] | LCMS m/z = 512.4 (M + H); 1H NMR (400 MHz, DMSO-d6) δ 13.13 (s, 1H), 8.87 (s, 1H), 8.05 (s, 1H), 7.93 (d, J = 8.4 Hz, 1H), 7.50 (d, J = 8.8 Hz, 1H), 7.18 (d, J = 8.8 Hz, 1H), 7.09 (d, J = 8.8 Hz, 1H), 6.97 (s, 1H), 5.84 (q, J = 7.2 Hz, 1H), 2.90 (s, 2H), 2.76 (s, 3H), 2.67- 2.57 (m, 4H), 2.45-2.33 (m, 5H), 2.00-1.97 (m, 2H), 1.77-1.69 (m, 2H), 1.68 (d, J = 6.4 Hz, 3H), 1.04 (t, J = 6.8 Hz, 3H) |
| Example 100 (R)-4-(5-(1-(3,5- dimethyl- pyridazin-4- yl)ethoxy)-1H- indazol-3-yl)-6- methoxy- picolinonitrile | 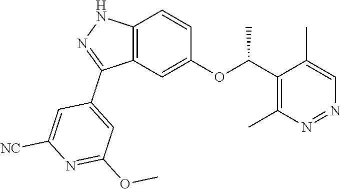 [SMILES: COc1cc(-c2n[nH]c3ccc(O[C@H](C)c4c(C)cnnc4C)cc23)cc(C#N)n1] | LCMS m/z = 401.2 (M + H); 1H NMR (400 MHz, DMSO-d6) δ 13.67 (brs, 1H), 8.83 (s, 1H), 8.00 (d, J = 2.4 Hz, 1H), 7.58 (d, J = 8.8 Hz, 1H), 7.41 (d, J = 2.4 Hz, 1H), 7.15-7.12 (m, 2H), 5.97 (q, J = 6.8 Hz, 1H), 3.99 (s, 3H), 2.80 (s, 3H), 2.48 (s, 3H), 1.70 (d, J = 6.8 Hz, 3H) |
| Example 101 (R)-3-(5-(1-(3,5- dimethyl- pyridazin-4-yl) ethoxy)-6- methoxy-1H- indazol-3-yl)-5- methoxy- benzonitrile | 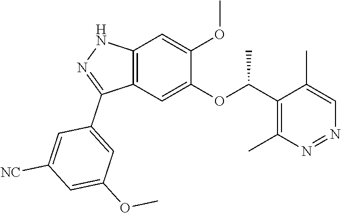 [SMILES: COc1cc(C#N)cc(-c2n[nH]c3cc(OC)c(O[C@H](C)c4c(C)cnnc4C)cc23)c1] | LCMS m/z = 430.2 (M + H); 1H NMR (400 MHz, DMSO-d6) δ 13.14 (s, 1H), 8.83 (s, 1H), 7.65-7.64 (m, 1H), 7.54 (d, J = 0.8 Hz, 1H), 7.45 (dd, J = 2.8 Hz, 1.6 Hz, 1H), 7.04 (s, 1H), 6.97 (s, 1H), 5.88 (q, J = 6.8 Hz, 1H), 3.90-3.89 (m, 6H), 2.77 (s, 3H), 3.46 (s, 3H), 1.68 (d, J = 6.8 Hz, 3H) |
| Example 102 (R)-5-(5-(1-(3,5- dimethyl- pyridazin-4-yl) ethoxy)-6- methoxy-1H- indazol-3-yl)- 2,3-dimethoxy- benzonitrile | 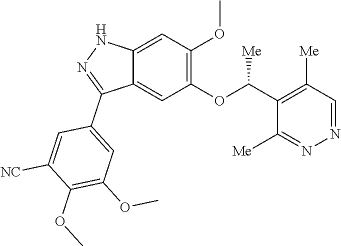 [SMILES: COc1cc2[nH]nc(-c3cc(C#N)c(OC)c(OC)c3)c2cc1O[C@H](C)c1c(C)cnnc1C] | LCMS m/z = 460.3 (M + H); 1H NMR (400 MHz, DMSO-d6) δ 13.13 (s, 1H), 8.86 (s, 1H), 7.47 (d, J = 8.8 Hz, 1H), 7.22 (d, J = 8.0 Hz, 1H), 7.05 (s, 1H), 6.80 (s, 1H), 5.69 (q, J = 6.8 Hz, 1H), 3.96 (s, 3H), 3.95 (s, 3H), 3.89 (s, 3H), 2.73 (s, 3H), 2.41 (s, 3H), 1.64 (d, J = 6.8 Hz, 3H) |
| Example 103 6-[5-[(1R)-1- (3,5-dichloro-4- pyridyl)ethoxy]- 1H-indazol-3-yl]- 1′-ethylspiro [chromane-2,4′- piperidine]-4-ol | 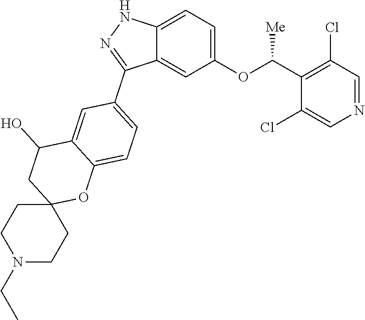 [SMILES: CCN1CCC2(CC1)CC(O)c1cc(-c3n[nH]c4ccc(O[C@H](C)c5c(Cl)cncc5Cl)cc34)ccc1O2] | LCMS m/z = 553.2 (M + H); 1H NMR (400 MHz, DMSO-d6) δ = 13.00 (br s, 1H), 9.56 (br s, 1H), 8.69-8.53 (m, 2H), 8.00-7.83 (m, 1H), 7.57-7.45 (m, 2H), 7.20-7.07 (m, 2H), 6.97 (br d, J = 6.7 Hz, 1H), 6.14-6.03 (m, 1H), 5.53 (br d, J = 15.8 Hz, 1H), 4.89-4.79 (m, 1H), 3.46-3.35 (m, 1H), 3.17 (br s, 5H), 2.30- 2.04 (m, 3H), 1.91 (br d, J = 5.1 Hz, 4H), 1.77 (dd, J = 2.0, 6.7 Hz, 3H), 1.24 (br s, 3H) |
| Example 104 6-[5-[(1R)-1- (3,5-dichloro-4- pyridyl)ethoxy]- 1H-indazol-3- yl]spiro [chromane-2,4′- piperidine] | 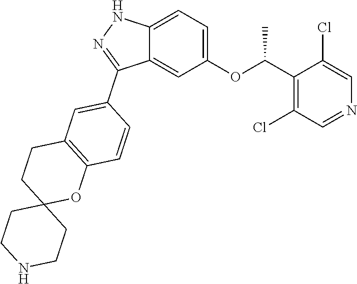 [SMILES: C[C@@H](Oc1ccc2[nH]nc(-c3ccc4c(c3)CCC3(CCNCC3)O4)c2c1)c1c(Cl)cncc1Cl] | LCMS m/z = 509.2 (M + H); 1H NMR (400 MHz, DMSO-d6) δ = 12.98 (s, 1H), 8.62 (s, 2H), 8.49 (br s, 2H), 7.51-7.44 (m, 3H), 7.12- 7.05 (m, 2H), 6.97 (d, J = 8.4 Hz, 1H), 6.07 (q, J = 6.6 Hz, 1H), 3.28-3.22 (m, 2H), 3.19- 3.11 (m, 3H), 2.97-2.82 (m, 2H), 1.99-1.90 (m, 4H), 1.87-1.78 (m, 2H), 1.76 (d, J = 6.6 Hz, 3H) |
| Example 105 5-[5-[(1R)-1- (3,5-dichloro-4- pyridyl)ethoxy]- 1H-indazol-3-yl]- 3-methoxy-2- (4-piperidyloxy) benzonitrile | 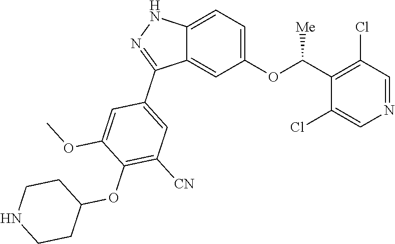 [SMILES: COc1cc(-c2n[nH]c3ccc(O[C@H](C)c4c(Cl)cncc4Cl)cc23)cc(C#N)c1OC1CCNCC1] | LCMS m/z = 538 (M + H); 1H NMR (400 MHz, DMSO-d6) δ = 13.30 (br s, 1H), 8.58 (s, 2H), 7.75 (d, J = 2.0 Hz, 1H), 7.54-7.51 (m, 1H), 7.48 (d, J = 2.0 Hz, 1H), 7.16-7.12 (m, 2H), 6.14 (q, J = 6.6 Hz, 1H), 4.55 (td, J = 4.3, 8.3 Hz, 1H), 3.95 (s, 3H), 3.19-3.13 (m, 2H), 2.80-2.71 (m, 2H), 1.98 (br d, J = 6.0 Hz, 2H), 1.79-1.73 (m, 5H) |
| Example 106 (R)-6-(5-(1-(3,5- dimethyl- pyridazin-4-yl) ethoxy)-1H- indazol-3-yl)-1′- ethyl-4H- spiro[benzo[d] [1,3]dioxine- 2,4′-piperidine] | 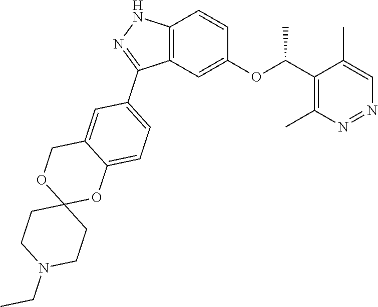 [SMILES: CCN1CCC2(CC1)OCc1cc(-c3n[nH]c4ccc(O[C@H](C)c5c(C)cnnc5C)cc34)ccc1O2] | LCMS m/z = 500.4 (M + H); 1H NMR (400 MHz, DMSO-d6) δ 12.99 (s, 1H), 8.86 (s, 1H), 7.50 (dd, J = 8.8 Hz, 2.0 Hz, 1H), 7.46 (d, J = 9.2 Hz, 1H), 7.34 (d, J = 1.2 Hz, 1H), 7.07 (dd, J = 8.8 Hz, 1.6 Hz, 1H), 6.95-6.93 (m, 2H), 5.82 (q, J = 6.8 Hz, 1H), 4.94-4.90 (m, 2H), 2.78 (s, 3H), 2.49-2.45 (m, 4H), 2.43 (s, 3H), 2.40 (q, J = 7.2 Hz, 2H), 1.93-1.87 (m, 4H), 1.68 (d, J = 6.8 Hz, 3H), 1.03 (t, J = 7.2 Hz, 3H) |
| Example 107 6-(5-((R)-1-(3,5- dimethyl- pyridazin-4-yl) ethoxy)-1H- indazol-3-yl)-1′- ethylspiro [chroman-2,4′- piperidin]-4-ol | 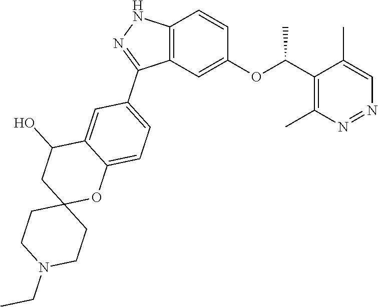 [SMILES: CCN1CCC2(CC1)CC(O)c1cc(-c3n[nH]c4ccc(O[C@H](C)c5c(C)cnnc5C)cc34)ccc1O2] | LCMS m/z = 514.3 (M + H); 1H NMR (400 MHz, DMSO-d6) δ 12.96 (s, 1H), 8.85 (d, J = 8.4 Hz, 1H), 7.88-7.83 (m, 1H), 7.47-7.45 (m, 2H), 7.07 (dd, J = 8.8 Hz, 2.4 Hz, 1H), 7.00 (s, 1H), 6.90 (d, J = 8.4 Hz, 1H), 5.83 (q, J = 6.8 Hz, 1H), 5.51 (s, 1H), 4.81-4.77 (m, 1H), 3.31- 2.78 (m, 2H), 2.77 (d, J = 5.2 Hz, 3H), 2.73- 2.56 (m, 4H), 2.44 (d, J = 4.8 Hz, 3H), 2.21- 2.14 (m, 2H), 1.91-1.73 (m, 4H), 1.68 (d, J = 6.8 Hz, 3H), 1.14-1.03 (m, 3H) |
| Example 108 (R)-5-(5-(1-(3,5- dichloropyridin- 4-yl)ethoxy)-1H- indazol-3-yl)-2- methoxy-3- methylbenzamide | 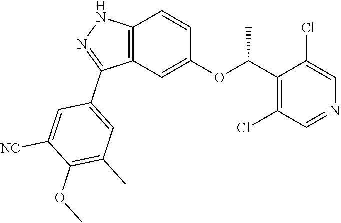 [SMILES: COc1c(C)cc(-c2n[nH]c3ccc(O[C@H](C)c4c(Cl)cncc4Cl)cc23)cc1C#N] | LCMS m/z = 453.1 (M + H); 1H NMR (400 MHz, DMSO-d6) δ 13.28 (s, 1H), 8.60 (s, 2H), 7.96-7.95 (m, 1H), 7.85 (d, J = 1.6 Hz, 1H), 7.52-7.50 (m, 1H), 7.14-7.11 (m, 2H), 6.14 (q, J = 6.8 Hz, 1H), 3.98 (s, 3H), 2.39 (s, 3H), 1.76 (d, J = 6.4 Hz, 3H) |
| Example 109 (R)-3-cyclo- propyl-5-(5-(1- (3,5-dichloro- pyridin-4-yl) ethoxy)-1H- indazol-3-yl) benzonitrile | 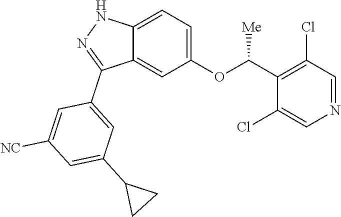 [SMILES: C[C@@H](Oc1ccc2[nH]nc(-c3cc(C#N)cc(C4CC4)c3)c2c1)c1c(Cl)cncc1Cl] | LCMS m/z = 449.1 (M + H); 1H-NMR (400 MHz, DMSO-d6) δ 13.34 (s, 1H), 8.59 (s, 2H), 7.88-7.79 (m, 2H), 7.56-7.50 (m, 2H), 7.15- 7.13 (m, 2H), 6.15 (q, J = 6.8 Hz, 1H), 2.15- 2.08 (m, 1H), 1.76 (d, J = 6.8 Hz, 3H), 1.10- 1.05 (m, 2H), 0.87-0.83 (m, 2H) |
| Example 110 (R)-5-(5-(1-(3,5- dimethyl- pyridazin-4-yl) ethoxy)-1H- indazol-3-yl)-2- ((1-isopropyl- piperidin-4-yl) oxy)-3-methyl- benzonitrile | 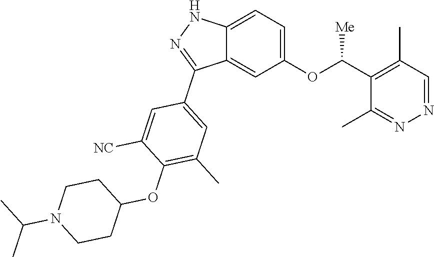 [SMILES: Cc1cc(-c2n[nH]c3ccc(O[C@H](C)c4c(C)cnnc4C)cc23)cc(C#N)c1OC1CCN(C(C)C)CC1] | LCMS m/z = 525.3 (M + H); 1H-NMR (400 MHz, DMSO-d6) δ 13.30 (s, 1H), 8.83 (s, 1H), 7.90 (s, 2H), 7.52 (d, J = 8.8 Hz, 1H), 7.12 (dd, J = 9.2, 2.4 Hz, 1H), 6.99 (d, J = 2.4 Hz, 1H), 5.94 (d, J = 6.4 Hz, 1H), 4.60-4.13 (m, 1H), 3.34-2.96 (m, 5H), 2.81 (s, 3H), 2.46 (s, 3H), 2.39 (s, 3H), 2.33-1.91 (m, 4H), 1.69 (d, J = 6.4 Hz, 3H), 1.35-1.13 (m, 6H) |
| Example 111 (R)-6-(5-(1-(3,5- dimethyl- pyridazin-4-yl) ethoxy)-1H- indazol-3-yl)-1′- ethylspiro [chroman-2,4′- piperidine] | 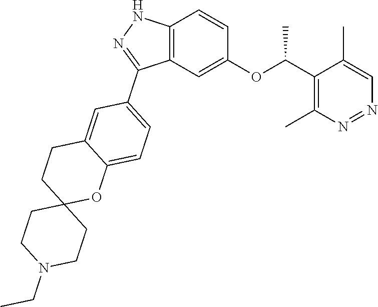 [SMILES: CCN1CCC2(CCc3cc(-c4n[nH]c5ccc(O[C@H](C)c6c(C)cnnc6C)cc45)ccc3O2)CC1] | LCMS m/z = 498.4 (M + H); 1H NMR (400 MHz, DMSO-d6) δ 12.95 (s, 1H), 8.85 (s, 1H), 7.47-7.41 (m, 2H), 7.38 (s, 1H), 7.08 (dd, J = 8.8 Hz, 2.4 Hz, 1H), 6.91-6.89 (m, 2H), 5.83 (q, J = 6.8 Hz, 1H), 3.26-3.23 (m, 2H), 3.13- 2.71 (m, 9H), 2.43 (s, 3H), 1.98-1.75 (m, 6H), 1.69 (d, J = 6.8 Hz, 3H), 1.26-1.10 (m, 3H) |
| Example 112 (R)-6-(5-(1-(3,5- dimethyl- pyridazin-4-yl) ethoxy)-1H- indazol-3-yl)-1′- isopropyl-4H- spiro[benzo[d] [1,3]dioxine- 2,4′-piperidine] | 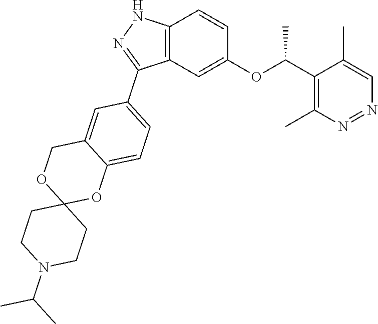 [SMILES: Cc1cnnc(C)c1[C@@H](C)Oc1ccc2[nH]nc(-c3ccc4c(c3)COC3(CCN(C(C)C)CC3)O4)c2c1] | LCMS mz = 514.4 (M + H); 1H NMR (400 MHz, DMSO-d6) δ 13.01 (s, 1H), 8.86 (s, 1H), 7.50 (dd, J = 8.8 Hz, 2.0 Hz, 1H), 7.46 (d, J = 8.8 Hz, 1H), 7.34 (d, J = 1.6 Hz, 1H), 7.07 (dd, J = 9.2 Hz, 2.4 Hz, 1H), 6.96-6.93 (m, 2H), 5.83 (q, J = 6.4 Hz, 1H), 5.83-5.82 (m, 2H), 2.78 (s, 3H), 2.70-2.49 (m, 5H), 2.43 (s, 3H), 1.91-1.89 (m, 4H), 1.68 (d, J = 6.4 Hz, 3H), 1.02 (d, J = 6.4 Hz, 6H) |
| Example 113 (R)-5-(5-(1-(3,5- dimethyl- pyridazin-4-yl) ethoxy)-1H- indazol-3-yl)-3- methyl-2-((1- (oxetan-3-yl) piperidin-4- yl)oxy) benzonitrile | 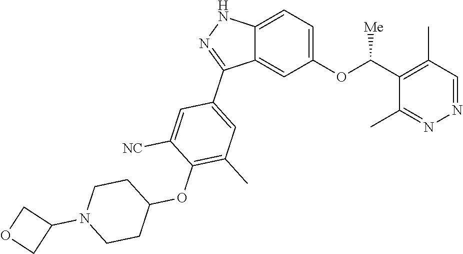 [SMILES: Cc1cc(-c2n[nH]c3ccc(O[C@H](C)c4c(C)cnnc4C)cc23)cc(C#N)c1OC1CCN(C2COC2)CC1] | LCMS m/z = 539.3 (M + H); 1H NMR (400 MHz, DMSO-d6) δ 13.27 (s, 1H), 8.83 (s, 1H), 7.88 (s, 2H), 7.52 (d, J = 8.8 Hz, 1H), 7.11 (dd, J = 9.2, 2.4 Hz, 1H), 6.99 (d, J = 2.0 Hz, 1H), 5.91 (q, J = 6.8 Hz, 1H), 4.68-4.56 (m, 4H), 4.36-4.19 (m, 1H), 3.54-3.43 (m, 1H), 2.80 (s, 3H), 2.70-2.64 (m, 2H), 2.46 (s, 3H), 2.38 (s, 3H), 2.08-1.85 (m, 6H), 1.69 (d, J = 6.8 Hz, 3H) |
| Example 114 (R)-5-(5-(1-(3,5- Dichloropyridin- 4-yl)ethoxy)-1H- indazol-3-yl)-2- ((1-ethyl- piperidin-4-yl) oxy)-3- methoxy- benzonitrile | 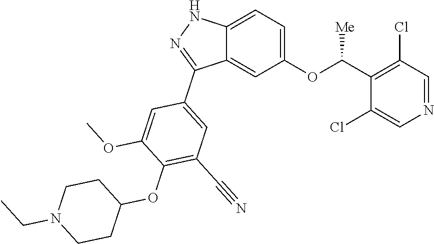 [SMILES: CCN1CCC(Oc2c(C#N)cc(-c3n[nH]c4ccc(O[C@H](C)c5c(Cl)cncc5Cl)cc34)cc2OC)CC1] | LCMS m/z = 566 (M + H); 1H NMR (400 MHz, DMSO-d6) δ = 13.28 (br s, 1H), 8.58 (s, 2H), 7.73 (d, J = 1.8 Hz, 1H), 7.52 (d, J = 8.9 Hz, 1H), 7.46 (d, J = 2.0 Hz, 1H), 7.18-7.09 (m, 2H), 6.14 (q, J = 6.7 Hz, 1H), 4.46 (td, J = 4.0, 8.2 Hz, 1H), 3.94 (s, 3H), 2.83-2.71 (m, 2H), 2.35 (q, J = 7.1 Hz, 2H), 2.15 (br t, J = 8.7 Hz, 2H), 1.90 (br s, 2H), 1.80-1.71 (m, 5H), 1.01 (t, J = 7.2 Hz, 3H) |
| Example 115 (R)-5-(5-(1-(3,5- dimethyl- pyridazin-4-yl) ethoxy)-1H- indazol-3-yl)-2- ((1-ethyl- piperidin-4-yl) oxy)-3-methyl- benzonitrile | 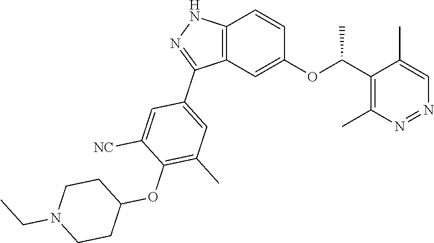 [SMILES: CCN1CCC(Oc2c(C)cc(-c3n[nH]c4ccc(O[C@H](C)c5c(C)cnnc5C)cc34)cc2C#N)CC1] | LCMS m/z = 511.3 (M + H); 1H NMR (400 MHz, DMSO-d6) δ 13.25 (s, 1H), 8.83 (s, 1H), 7.87 (s, 2H), 7.51 (d, J = 9.2 Hz, 1H), 7.11 (dd, J = 9.2 Hz, 2.0 Hz, 1H), 7.00 (s, 1H), 5.91 (q, J = 6.8 Hz, 1H), 4.30-4.24 (m, 1H), 2.85-2.79 (m, 5H), 2.46 (s, 3H), 2.37-2.30 (m, 5H), 2.11- 1.97 (m, 4H), 1.85-1.80 (m, 2H), 1.69 (d, J = 6.4 Hz, 3H), 1.02 (t, J = 6.8 Hz, 3H) |
| Example 116 (R)-5-(5-(1-(3,5- dimethyl- pyridazin-4-yl) ethoxy)-6- methoxy-1H- indazol-3-yl)-2- methoxy-3- methyl- benzonitrile | 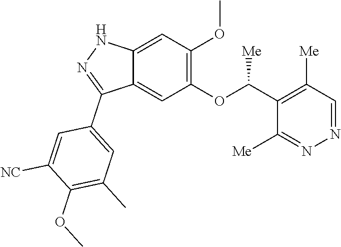 [SMILES: COc1cc2[nH]nc(-c3cc(C)c(OC)c(C#N)c3)c2cc1O[C@H](C)c1c(C)cnnc1C] | LCMS m/z = 444.1 (M + H); 1H NMR (400 MHz, DMSO-d6) δ 13.05 (s, 1H), 8.83 (s, 1H), 7.85 (s, 2H), 7.02 (s, 1H), 6.93 (s, 1H), 5.86 (q, J = 6.4 Hz, 1H), 3.97 (s, 3H), 3.90 (s, 3H), 2.78 (s, 3H), 2.45 (s, 3H), 2.37 (s, 3H), 1.68 (d, J = 6.4 Hz, 3H) |
| Example 117 Name (R)-3- cyclopropyl-5-(5- (1-(3,5-dimethyl- pyridazin-4-yl) ethoxy)-6- methoxy-1H- indazol-3-yl) benzonitrile | 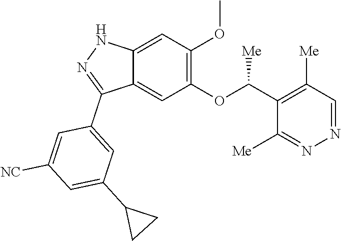 [SMILES: COc1cc2[nH]nc(-c3cc(C#N)cc(C4CC4)c3)c2cc1O[C@H](C)c1c(C)cnnc1C] | LCMS m/z = 440.2 (M + H); 1H NMR (400 MHz, DMSO-d6) δ 13.10 (s, 1H), 8.82 (s, 1H), 7.82-7.81 (m, 1H), 7.79-7.78 (m, 1H), 7.47 (t, J = 1.6 Hz, 1H), 7.04 (s, 1H), 6.96 (s, 1H), 5.87 (q, J = 6.4 Hz, 1H), 3.90 (s, 3H), 2.78 (s, 3H), 2.45 (s, 3H), 2.14-2.07 (m, 1H), 1.68 (d, J = 5.6 Hz, 3H), 1.09-1.06 (m, 2H), 0.85- 0.83 (m, 2H) |
| Example 118 (R)-5-(5-(1-(3,5- Dichloropyridin- 4-yl)ethoxy)-1H- indazol-3-yl)-2- (piperidin-4- yloxy) benzonitrile | 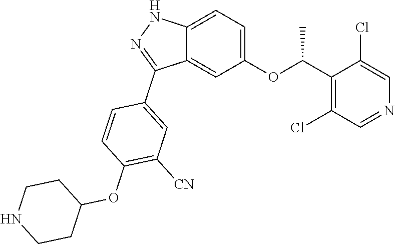 [SMILES: C[C@@H](Oc1ccc2[nH]nc(-c3ccc(OC4CCNCC4)c(C#N)c3)c2c1)c1c(Cl)cncc1Cl] | LCMS m/z = 508.2 (M + H); 1H NMR (400 MHz, CD3OD) δ = 8.51 (s, 2H), 8.05 (dd, J = 2.3, 8.9 Hz, 1H), 7.88 (d, J = 2.2 Hz, 1H), 7.47 (d, J = 9.0 Hz, 1H), 7.41 (d, J = 8.9 Hz, 1H), 7.17 (dd, J = 2.3, 9.0 Hz, 1H), 7.02 (d, J = 2.2 Hz, 1H), 6.10 (q, J = 6.7 Hz, 1H), 5.01 (tt, J = 3.0, 5.9 Hz, 1H), 3.47 (tdd, J = 3.3, 9.5, 12.8 Hz, 2H), 3.33 (br s, 1H), 3.30-3.27 (m, 1H), 2.31-2.13 (m, 4H), 1.82 (d, J = 6.6 Hz, 3H) |
| Example 119 5-[5-[(1R)-1- (3,5-dichloro-4- pyridyl)ethoxy]- 1H-indazol-3-yl]- 2-[(1-ethyl-4- piperidyl)oxy] piperidine-3- carbonitrile | 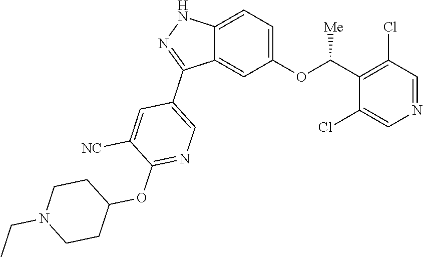 [SMILES: CCN1CCC(Oc2ncc(-c3n[nH]c4ccc(O[C@H](C)c5c(Cl)cncc5Cl)cc34)cc2C#N)CC1] | LCMS m/z = 537.2 (M + H); 1H NMR (400 MHz, DMSO-d6) δ = 13.37 (s, 1H), 9.54-9.25 (m, 1H), 8.90-8.83 (m, 1H), 8.57 (s, 2H), 8.55 (dd, J = 2.4, 4.6 Hz, 1H), 7.53 (d, J = 9.0 Hz, 1H), 7.23 (s, 1H), 7.17-7.09 (m, 1H), 6.15 (q, J = 6.6 Hz, 1H), 5.55-5.34 (m, 1H), 3.62 (br d, J = 11.9 Hz, 1H), 3.56-3.48 (m, 1H), 3.25- 3.07 (m, 4H), 2.46-2.40 (m, 1H), 2.35-2.23 (m, 1H), 2.22-2.07 (m, 1H), 1.98-1.87 (m, 1H), 1.77 (d, J = 6.6 Hz, 3H), 1.30-1.24 (m, 3H) |
| Example 120 (R)-2-((1- cyclobutyl- piperidin-4-yl) oxy)-5-(5-(1- (3,5-dimethyl- pyridazin-4-yl) ethoxy)-1H- indazol-3-yl)-3- methyl- benzonitrile | 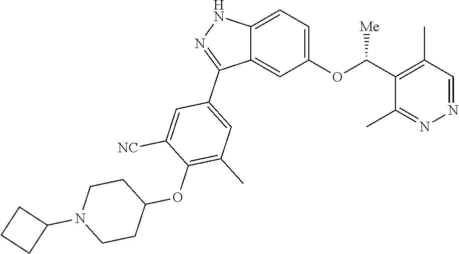 [SMILES: Cc1cc(-c2n[nH]c3ccc(O[C@H](C)c4c(C)cnnc4C)cc23)cc(C#N)c1OC1CCN(C2CCC2)CC1] | LCMS m/z = 537.4 (M + H); 1H NMR (400 MHz, DMSO-d6) δ 13.26 (s, 1H), 8.82 (s, 1H), 7.87 (s, 2H), 7.51 (d, J = 9.2 Hz, 1H), 7.11 (dd, J = 9.2 Hz, 2.0 Hz, 1H), 6.99 (d, J = 2.4 Hz, 1H), 5.91 (q, J = 6.0 Hz, 1H), 4.29-4.22 (m, 1H), 2.79 (s, 3H), 2.73-2.63 (m, 4H), 2.58-2.53 (m, 1H), 2.46 (s, 3H), 2.37 (s, 3H), 2.03-1.90 (m, 6H), 1.84-1.57 (m, 7H) |
| Example 121 6-[5-[(1R)-1- (3,5-dichloro- 4-pyridyl) ethoxy]-1H- indazol-3-yl]-1′- ethylspiro [chromane-2,4′- piperidine] | 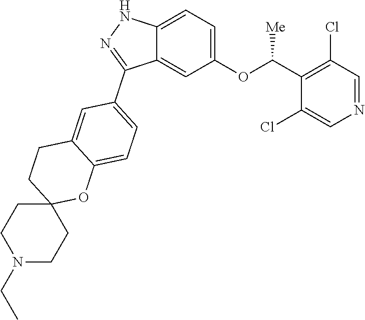 [SMILES: CCN1CCC2(CCc3cc(-c4n[nH]c5ccc(O[C@H](C)c6c(Cl)cncc6Cl)cc45)ccc3O2)CC1] | LCMS m/z = 537.2 (M + H); 1H NMR (400 MHz, DMSO-d6) δ = 12.99 (br s, 1H), 9.98- 9.68 (m, 1H), 8.62 (s, 2H), 7.52-7.45 (m, 1H), 7.12-7.06 (m, 2H), 6.98 (d, J = 8.4 Hz, 1H), 6.08 (q, J = 6.5 Hz, 1H), 3.44 (br d, J = 12.3 Hz, 3H), 3.24-3.09 (m, 6H), 2.95-2.85 (m, 2H), 2.07-1.96 (m, 3H), 1.95-1.88 (m, 3H), 1.79-1.73 (m, 4H), 1.32-1.21 (m, 6H) |
| Example 122 5-[5-[(1R)-1- (3,5-dichloro-4- pyridyl)ethoxy]- 1H-indazol-3-yl]- 2-(2-hydroxy-2- methyl-propoxy) pyridine-3- carbonitrile | 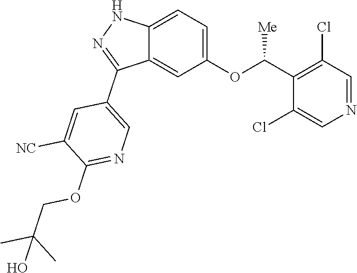 [SMILES: C[C@@H](Oc1ccc2[nH]nc(-c3cnc(OCC(C)(C)O)c(C#N)c3)c2c1)c1c(Cl)cncc1Cl] | LCMS m/z = 498.1 (M + H); 1H NMR (400 MHz, DMSO-d6) δ = 13.33 (br s, 1H), 8.85 (d, J = 2.4 Hz, 1H), 8.57 (s, 2H), 8.50 (d, J = 2.4 Hz, 1H), 7.52 (d, J = 9.0 Hz, 1H), 7.24 (d, J = 2.1 Hz, 1H), 7.12 (dd, J = 2.3, 9.0 Hz, 1H), 6.16 (q, J = 6.7 Hz, 1H), 4.74 (s, 1H), 4.27 (s, 2H), 1.77 (d, J = 6.6 Hz, 3H), 1.26 (s, 6H) |
| Example 123 (R)-6-(5-(1-(3,5- dichloro-2- fluoropyridin-4- yl)ethoxy)-1H- indazol-3-yl)-1′- isopropyl-8- methoxy-4H- spiro[benzo[d] [1,3]dioxine-2,4′- piperidine] | 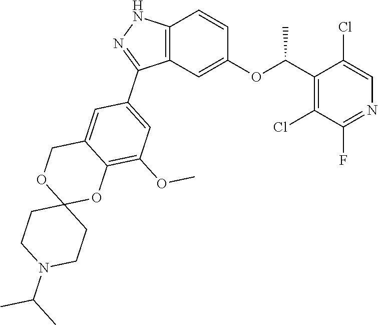 | LCMS m/z = 610.3 (M + H); 1H NMR (400 MHz, DMSO-d6) δ 13.04 (s, 1H), 8.36 (s, 1H), 7.48 (d, J = 9.6 Hz, 1H), 7.28 (d, J = 1.6 Hz, 1H), 7.12-7.09 (m, 2H), 6.98 (s, 1H), 6.10 (q, J = 6.8 Hz, 1H), 4.98-4.87 (m, 2H), 3.84 (s, 3H), 2.81-2.71 (m, 1H), 2.59-2.53 (m, 4H), 1.91-1.84 (m, 4H), 1.76 (d, J = 6.4 Hz, 3H), 1.00 (d, J = 6.8 Hz, 6H) |
| Example 124 5-[5-[(1R)-1- (3,5-dichloro-4- pyridyl)ethoxy]- 1H-indazol-3-yl]- 2-[(1-isopropyl- 4-piperidyl)oxy] benzonitrile | 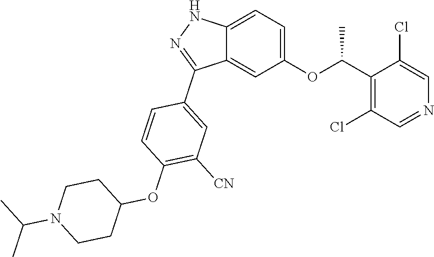 [SMILES: CC(C)N1CCC(Oc2ccc(-c3n[nH]c4ccc(O[C@H](C)c5c(Cl)cncc5Cl)cc34)cc2C#N)CC1] | LCMS m/z = 550.1 (M + H); 1H NMR (400 MHz, CD3OD) δ = 8.52 (s, 2H), 8.11-8.04 (m, 1H), 7.95-7.87 (m, 1H), 7.48 (d, J = 9.0 Hz, 1H), 7.47-7.42 (m, 1H), 7.19 (dd, J = 2.3, 9.0 Hz, 1H), 7.06 (d, J = 2.2 Hz, 1H), 6.13 (q, J = 6.6 Hz, 1H), 3.71-3.55 (m, 2H), 3.50-3.43 (m, 3H), 2.47-2.31 (m, 2H), 2.23 (br d, 15.8 Hz, 2H), 2.12-1.97 (m, 1H), 1.83 (d, J = 6.6 Hz, 3H), 1.47-1.40 (m, 6H) |
| Example 125 (R)-6-(5-(1-(3,5- dimethyl- pyridazin-4-yl) ethoxy)-1H- indazol-3-yl)-1′- isopropyl-8- methoxy-4H- spiro[benzo[d] [1,3]dioxine-2,4′- piperidine] | 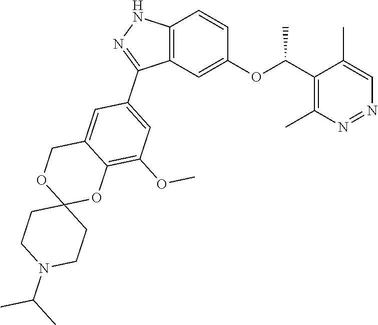 [SMILES: COc1cc(-c2n[nH]c3ccc(O[C@H](C)c4c(C)cnnc4C)cc23)cc2c1OC1(CCN(C(C)C)CC1)OC2] | LCMS m/z = 544.3 (M + H); 1H NMR (400 MHz, DMSO-d6) δ 13.00 (s, 1H), 8.84 (s, 1H), 7.47 (d, J = 9.2 Hz, 1H), 7.22 (d, J = 1.6 Hz, 1H), 7.08 (dd, J = 9.2 Hz, 1.6 Hz, 1H), 6.94 (s, 1H), 6.85 (s, 1H), 5.82 (q, J = 6.4 Hz, 1H), 4.96-4.83 (m, 2H), 3.82 (s, 3H), 2.88-2.81 (m, 1H), 2.78 (s, 3H), 2.61-2.58 (m, 4H), 2.42 (s, 3H), 1.91-1.84 (m, 4H), 1.67 (d, J = 6.8 Hz, 3H), 1.02 (d, J = 6.4 Hz, 6H) |
| Example 126 5-[5-[(1R)-1- (3,5-dichloro-4- pyridyl)ethoxy]- 1H-indazol-3-yl]- 2-[(1-isopropyl- 4-piperidyl)oxy]- 3-methoxy- benzonitrile | 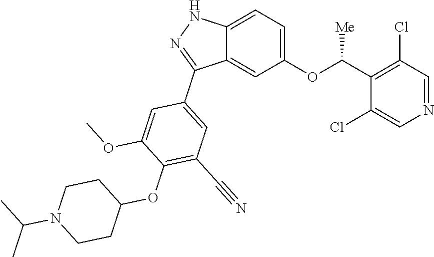 [SMILES: COc1cc(-c2n[nH]c3ccc(O[C@H](C)c4c(Cl)cncc4Cl)cc23)cc(C#N)c1OC1CCN(C(C)C)CC1] | LCMS m/z = 580 (M + H); 1H NMR (400 MHz, DMSO-d6) δ = 13.32 (br s, 1H), 9.43- 8.94 (m, 1H), 8.58 (s, 2H), 7.79 (d, J = 1.8 Hz, 1H), 7.55-7.50 (m, 2H), 7.17-7.12 (m, 2H), 6.51 (br s, 1H), 6.14 (q, J = 6.6 Hz, 1H), 4.59 (m, 1H), 3.97 (s, 3H), 3.58 (m, 1H), 3.23 (m, 2H), 3.18-2.90 (m, 2H), 2.32-2.23 (m, 1H), 2.15 (br s, 1H), 2.12-1.93 (m, 2H), 1.77 (d, J = 6.6 Hz, 3H), 1.35-1.21 (m, 6H) |
| Example 127 5-(5-((R)-1-(3,5- dimethyl- pyridazin-4-yl) ethoxy)-1H- indazol-3-yl)-2- (((S)-tetrahydro- furan-3-yl)oxy) nicotinonitrile | 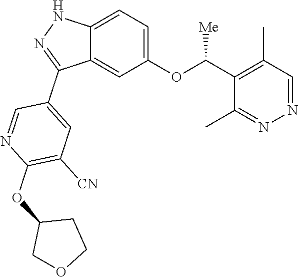 [SMILES: Cc1cnnc(C)c1[C@@H](C)Oc1ccc2[nH]nc(-c3cnc(O[C@H]4CCOC4)c(C#N)c3)c2c1] | LCMS m/z = 457.2 (M + H); 1H NMR (400 MHz, DMSO-d6) δ 13.34 (s, 1H), 8.86 (d, J = 2.4 Hz, 1H), 8.82 (s, 1H), 8.57 (d, J = 2.4 Hz, 1H), 7.52 (d, J = 9.2 Hz, 1H), 7.15 (d, J = 2.0 Hz, 1H), 7.10 (dd, J = 9.2 Hz, 2.4 Hz, 1H), 5.93 (q, J = 6.8 Hz, 1H), 5.72-5.69 (m, 1H), 4.01- 3.97 (m, 1H), 3.94-3.89 (m, 2H), 3.84-3.81 (m, 1H), 2.80 (s, 3H), 2.46 (s, 3H), 2.38-2.29 (m, 1H), 2.16-2.10 (m, 1H), 1.68 (d, J = 6.8 Hz, 3H) |
| Example 128 5-(5-((R)-1-(3,5- dimethyl- pyridazin-4-yl) ethoxy)-1H- indazol-3-yl)-2- (((R)-tetrahydro- furan-3-yl)oxy) nicotinonitrile | 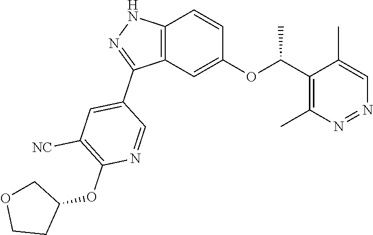 [SMILES: Cc1cnnc(C)c1[C@@H](C)Oc1ccc2[nH]nc(-c3cnc(O[C@@H]4CCOC4)c(C#N)c3)c2c1] | LCMS m/z = 457.2 (M + H); 1H NMR (400 MHz, DMSO-d6) δ 13.34 (s, 1H), 8.86 (d, J = 2.0 Hz, 1H), 8.82 (s, 1H), 8.57 (d, J = 2.4 Hz, 1H), 7.52 (d, J = 9.2 Hz, 1H), 7.15 (s, 1H), 7.11 (d, J = 8.8 Hz, 1H), 5.93 (q, J = 7.2 Hz, 1H), 5.71-5.69 (m, 1H), 4.00-3.90 (m, 3H), 3.84- 3.80 (m, 1H), 2.79 (s, 3H), 2.47 (s, 3H), 2.37- 2.32 (m, 1H), 2.14-2.05 (m, 1H), 1.68 (d, J = 6.4 Hz, 3H) |
| Example 129 5-(5-((R)-1-(3,5- dichloropyridin- 4-yl)ethoxy)-1H- indazol-3-yl)-2- (((S)-tetrahydro- furan-3-yl) amino) nicotinonitrile | 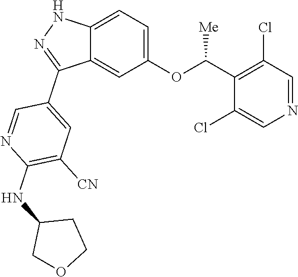 [SMILES: C[C@@H](Oc1ccc2[nH]nc(-c3cnc(N[C@H]4CCOC4)c(C#N)c3)c2c1)c1c(Cl)cncc1Cl] | LCMS m/z = 495.2 (M + H); 1H-NMR (400 MHz, DMSO-d6) δ 13.15 (s, 1H), 8.73 (d, J = 2.0 Hz, 1H), 8.57 (s, 2H), 8.14 (d, J = 2.0 Hz, 1H), 7.48 (d, J = 8.8 Hz, 1H), 7.35 (d, J = 6.0 Hz, 1H), 7.16-7.06 (m, 2H), 6.13 (q, J = 6.8 Hz, 1H), 4.67-4.57 (m, 1H), 3.98-3.85 (m, 2H), 3.80-3.71 (m, 1H), 3.67-3.61 (m, 1H), 2.29- 2.15 (m, 1H), 2.11-1.99 (m, 1H), 1.76 (d, J = 6.4 Hz, 3H) |
| Example 130 5-(5-((R)-1-(3,5- dichloropyridin- 4-yl)ethoxy)-1H- indazol-3-yl)-2- (((R)- tetrahydrofuran- 3-yl)amino) nicotinonitrile | 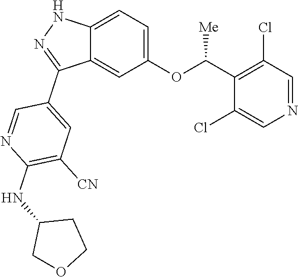 [SMILES: C[C@@H](Oc1ccc2[nH]nc(-c3cnc(N[C@@H]4CCOC4)c(C#N)c3)c2c1)c1c(Cl)cncc1Cl] | LCMS m/z = 495.1 (M + H); 1H NMR (400 MHz, DMSO-d6) δ 13.17 (s, 1H), 8.72 (d, J = 2.4 Hz, 1H), 8.58 (s, 2H), 8.15 (d, J = 2.4 Hz, 1H), 7.48 (d, J = 8.8 Hz, 1H), 7.35 (d, J = 6.0 Hz, 1H), 7.14 (d, J = 1.6 Hz, 1H), 7.10 (dd, J = 8.8 Hz, 2.0 Hz, 1H), 6.13 (q, J = 6.8 Hz, 1H), 4.66-4.59 (m, 1H), 3.97-3.87 (m, 2H), 3.75 (q, J = 6.0 Hz, 1H), 3.67-3.63 (m, 1H), 2.24-2.18 (m, 1H), 2.09-2.03 (m, 1H), 1.76 (d, J = 6.4 Hz, 3H) |
| Example 131 5-[5-[(1R)-1- (3,5-dichloro-4- pyridyl)ethoxy]- 6-methoxy-1H- indazol-3-yl]-2- (dimethylamino) pyridine-3- carbonitrile | 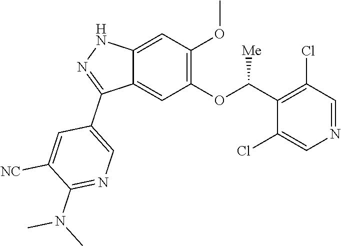 [SMILES: COc1cc2[nH]nc(-c3cnc(N(C)C)c(C#N)c3)c2cc1O[C@H](C)c1c(Cl)cncc1Cl] | LCMS m/z = 483.1 (M + H); 1H NMR (400 MHz, DMSO-d6) δ = 12.96 (s, 1H), 8.73 (d, J = 2.4 Hz, 1H), 8.59 (s, 2H), 8.08 (d, J = 2.4 Hz, 1H), 7.06-6.99 (m, 2H), 6.00 (q, J = 6.6 Hz, 1H), 3.86 (s, 3H), 3.29 (s, 6H), 1.76 (d, J = 6.7 Hz, 3H) |
| Example 132 (R)-5-(5-(1-(3,5- dichloropyridin- 4-yl)ethoxy)-1H- indazol-3-yl)-2- ((tetrahydro-2H- pyran-4- yl)amino) nicotinonitrile | 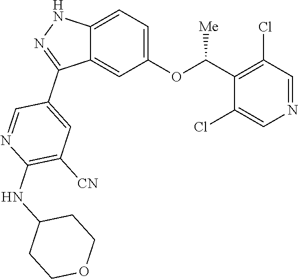 [SMILES: C[C@@H](Oc1ccc2[nH]nc(-c3cnc(NC4CCOCC4)c(C#N)c3)c2c1)c1c(Cl)cncc1Cl] | LCMS m/z = 509.2 (M + H); 1H-NMR (400 MHz, DMSO-d6) δ 13.14 (s, 1H), 8.73 (d, J = 2.4 Hz, 1H), 8.57 (s, 2H), 8.11 (d, J = 2.4 Hz, 1H), 7.48 (d, J = 8.8 Hz, 1H), 7.16-7.05 (m, 3H), 6.14 (q, J = 6.4 Hz, 1H), 4.28-4.25 (m, 1H), 3.93-3.89 (m, 2H), 3.45-3.39 (m, 2H), 1.85-1.81 (m, 2H), 1.76 (d, J = 6.4 Hz, 3H), 1.73-1.63 (m, 2H) |
| Example 133 5-(5-((R)-1-(3,5- dichloropyridin- 4-yl)ethoxy)-1H- indazol-3-yl)-2- (((S)-tetrahydro- furan-3-yl)oxy) nicotinonitrile | 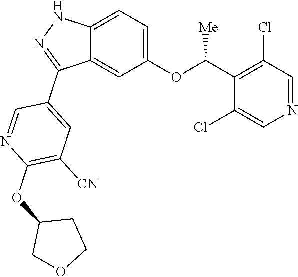 [SMILES: C[C@@H](Oc1ccc2[nH]nc(-c3cnc(O[C@H]4CCOC4)c(C#N)c3)c2c1)c1c(Cl)cncc1Cl] | LCMS m/z = 496.2 (M + H); 1H NMR (400 MHz, DMSO-d6) δ 13.36 (s, 1H), 8.85 (d, J = 2.4 Hz, 1H), 8.57 (s, 2H), 8.51 (d, J = 2.8 Hz, 1H), 7.52 (d, J = 8.8 Hz, 1H), 7.23 (d, J = 1.6 Hz, 1H), 7.12 (dd, J = 8.8 Hz, 2.0 Hz, 1H), 6.15 (q, J = 6.4 Hz, 1H), 5.72-5.69 (m, 1H), 4.01-3.96 (m, 1H), 3.94-3.90 (m, 2H), 3.84- 3.79 (m, 1 H), 2.37-2.30 (m, 1H), 2.16-2.07 (m, 1H), 1.76 (d, J = 6.8 Hz, 3H) |
| Example 134 5-(5-((R)-1-(3,5- dichloropyridin- 4-yl)ethoxy)-1H- indazol-3-yl)-2- (((R)- tetrahydrofuran- 3-yl)oxy) nicotinonitrile | 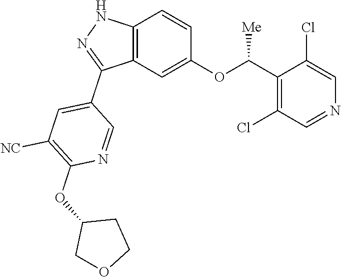 [SMILES: C[C@@H](Oc1ccc2[nH]nc(-c3cnc(O[C@@H]4CCOC4)c(C#N)c3)c2c1)c1c(Cl)cncc1Cl] | LCMS m/z = 496.3 (M + H); 1H NMR (400 MHz, DMSO-d6) δ 13.36 (s, 1H), 8.85 (d, J = 2.8 Hz, 1H), 8.58 (s, 2H), 8.52 (d, J = 2.4 Hz, 1H), 7.52 (d, J = 9.2 Hz, 1H), 7.23 (d, J = 2.4 Hz, 1H), 7.12 (dd, J = 9.0 Hz, 2.0 Hz, 1H), 6.16 (q, J = 6.4 Hz, 1H), 5.72-5.69 (m, 1H), 4.01-3.97 (m, 1H), 3.94-3.90 (m, 2H), 3.84- 3.79 (m, 1 H), 2.39-2.28 (m, 1H), 2.16-2.09 (m, 1H), 1.76 (d, J = 6.8 Hz, 3H) |
| Example 135 5-(5-((R)-1-(3,5- dichloropyridin-4- yl)ethoxy)-6- methyl-1H- indazol-3-yl)-2- (((R)- tetrahydrofuran- 3-yl)oxy) nicotinonitrile | 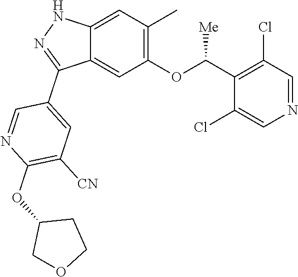 | LCMS m/z = 440.3 (M + H); 1H NMR (400 MHz, DMSO-d6) δ 13.20 (s, 1H), 8.78 (d, J = 2.4 Hz, 1H), 8.59 (s, 2H), 8.40 (d, J = 2.8 Hz, 1H), 7.41 (s, 1H), 6.90 (s, 1H), 6.09 (q, J = 6.8 Hz, 1H), 5.71-5.68 (m, 1H), 4.01-3.79 (m, 4H), 2.42 (s, 3H), 2.39-2.30 (m, 1H), 2.16-2.11 (m, 1H), 1.79 (d, J = 6.4 Hz, 3H) |
| Example 136 5-(5-((R)-1-(3,5- dichloropyridin- 4-yl)ethoxy)-6- methoxy-1H- indazol-3-yl)-2- (((R)- tetrahydrofuran- 3-yl)oxy) nicotinonitrile | 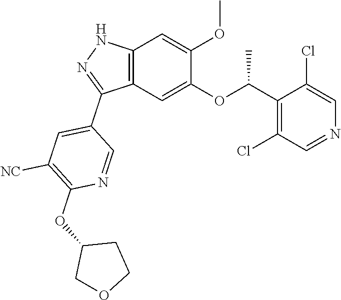 [SMILES: COc1cc2[nH]nc(-c3cnc(O[C@@H]4CCOC4)c(C#N)c3)c2cc1O[C@H](C)c1c(Cl)cncc1Cl] | LCMS m/z = 526.2 (M + H); 1H NMR (400 MHz, DMSO-d6) δ 13.15 (s, 1H), 8.80 (d, J = 2.4 Hz, 1H), 8.59 (s, 2H), 8.45 (d, J = 2.0 Hz, 1H), 7.10 (s, 1H), 7.03 (s, 1H), 6.00 (q, J = 6.4 Hz, 1H), 5.71-5.68 (m, 1H), 4.00-3.79 (m, 7H), 2.38-2.29 (m, 1H), 2.15-2.07 (m, 1H), 1.76 (d, J = 6.8 Hz, 3H). |
| Example 137 7-[5-[(1R)-1-(3,5- dichloro-4- pyridyl)ethoxy]- 1H-indazol-3-yl]- 1-methylsulfonyl- 2,3-dihydro- pyrido[2,3-b][1,4] oxazine | 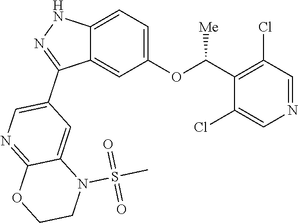 [SMILES: C[C@@H](Oc1ccc2[nH]nc(-c3cnc4c(c3)N(S(C)(=O)=O)CCO4)c2c1)c1c(Cl)cncc1Cl] | LCMS m/z = 520.1 (M + H); 1H NMR (400 MHz, DMSO-d6) δ = 13.16 (br s, 1H), 8.69 (d, J = 2.2 Hz, 1H), 8.43 (s, 1H), 8.18 (d, J = 2.3 Hz, 1H), 7.48 (d, J = 8.4 Hz, 1H), 7.10 (d, J = 8.3 Hz, 2H), 7.09 (s, 1H), 6.16-6.10 (m, 1H), 4.08-4.00 (m, 4H), 3.28-3.04 (m, 3H), 2.91 (s, 3H), 2.58-2.52 (m, 3H), 1.84-1.78 (m, 2H), 1.76 (d, J = 6.6 Hz, 3H), 1.63 (br s, 2H) |
| Example 138 5-(5-((R)-1-(3,5- dichloropyridin- 4-yl)ethoxy)-6- fluoro-1H- indazol-3-yl)-2- (((R)- tetrahydrofuran- 3-yl)oxy) nicotinonitrile | 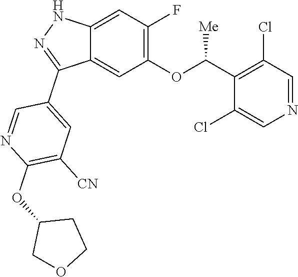 [SMILES: C[C@@H](Oc1cc2c(-c3cnc(O[C@@H]4CCOC4)c(C#N)c3)n[nH]c2cc1F)c1c(Cl)cncc1Cl] | LCMS m/z = 514.1 (M + H); 1H NMR (400 MHz, DMSO-d6) δ 13.42 (s, 1H), 8.84 (d, J = 2.4 Hz, 1H), 8.59 (s, 2H), 8.51 (d, J = 2.4 Hz, 1H), 7.51 (d, J = 10.8 Hz, 1H), 7.31 (d, J = 8.0 Hz, 1H), 6.19 (q, J = 7.2 Hz, 1H), 5.73-5.69 (m, 1H), 4.01-3.80 (m, 4H), 2.39-2.30 (m, 1H), 2.16-2.09 (m, 1H), 1.81 (d, J = 6.4 Hz, 3H). |
| Example 139 (R)-7-(5-(1-(3,5- dichloropyridin- 4-yl)ethoxy)-1H- indazol-3-yl)-1- (2,2,2-trifluoro- ethyl)-2,3- dihydro-1H- pyrido[2,3-b][1,4] oxazine 2,2,2- trifluoroacetate | 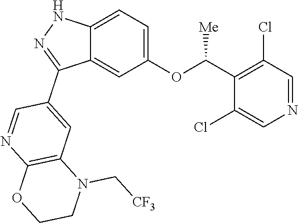 [SMILES: C[C@@H](Oc1ccc2[nH]nc(-c3cnc4c(c3)N(CC(F)(F)F)CCO4)c2c1)c1c(Cl)cncc1Cl] | LCMS m/z = 542.2 (M + H); 1H NMR (400 MHz, DMSO-d6) δ 13.59 (s, 1H), 8.55 (s, 2H), 8.31 (s, 1H), 8.03 (d, J = 1.2 Hz, 1H), 7.67 (s, 1H), 7.56 (d, J = 9.2 Hz, 1H), 7.49 (d, J = 2.0 Hz, 1H), 7.13 (dd, J = 9.2 Hz, 2.4 Hz, 1H), 6.16 (q, J = 6.8 Hz, 1H), 5.60-5.54 (m, 2H), 4.76- 4.75 (m, 2H), 3.60-3.59 (m, 2H), 1.78 (d, J = 6.8 Hz, 3H). |
| Example 140 7-[5-[(1R)-1-(3,5- dichloro-4- pyridyl)ethoxy]- 6-methoxy-1H- indazol-3-yl]-1- methylsulfonyl- 2,3-dihydro- pyrido[2,3-b][1,4] oxazine | 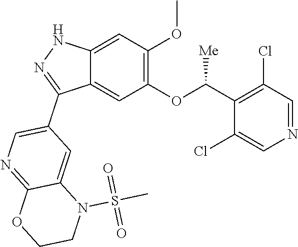 [SMILES: COc1cc2[nH]nc(-c3cnc4c(c3)N(S(C)(=O)=O)CCO4)c2cc1O[C@H](C)c1c(Cl)cncc1Cl] | LCMS m/z = 550.1 (M + H); 1H NMR (400 MHz, DMSO-d6) δ = 13.02 (br s, 1H), 8.58 (s, 2H), 8.44 (d, J = 2.1 Hz, 1H), 8.31 (d, J = 2.1 Hz, 1H), 7.17 (s, 1H), 7.05 (s, 1H), 6.01-5.94 (m, 1H), 4.51-4.43 (m, 2H), 3.92-3.86 (m, 5H), 3.26-3.17 (m, 3H), 1.76 (d, J = 6.7 Hz, 3H) |
| Example 141 (R)-7-(5-(1-(3,5- dichloropyridin- 4-yl)ethoxy)-1H- indazol-3-yl)-1- isopropyl-2,3- dihydro-1H- pyrido[2,3-b][1,4] oxazine | 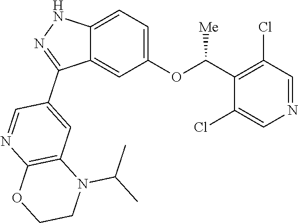 [SMILES: CC(C)N1CCOc2ncc(-c3n[nH]c4ccc(O[C@H](C)c5c(Cl)cncc5Cl)cc34)cc21] | LCMS m/z = 484 (M + H); 1H NMR (400 MHz, DMSO-d6) δ 13.07 (s, 1H), 8.57 (s, 2H), 7.74 (d, J = 2.0 Hz, 1H), 7.48 (d, J = 9.2 Hz, 1H), 7.43 (d, J = 2.0 Hz, 1H), 7.18 (d, J = 1.6 Hz, 1H), 7.12 (dd, J = 8.8 Hz, 2.4 Hz, 1H), 6.13 (q, J = 6.4 Hz, 1H), 4.35 (t, J = 4.4 Hz, 2H), 4.15-4.08 (m, 1H), 3.27-3.25 (m, 2H), 1.75 (d, J = 7.2 Hz, 3H), 1.18 (d, J = 2.0 Hz, 3H), 1.16 (d, J = 2.0 Hz, 3H) |
| Example 142 (R)-7-(5-(1-(3,5- dichloropyridin- 4-yl)ethoxy)-1H- indazol-3-yl)-1- (2,2-difluoro- ethyl)-2,3- dihydro-1H- pyrido[2,3-b][1,4] oxazine | 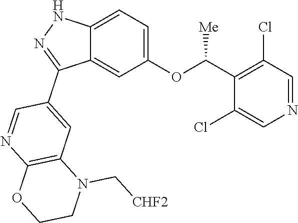 [SMILES: C[C@@H](Oc1ccc2[nH]nc(-c3cnc4c(c3)N(CC(F)F)CCO4)c2c1)c1c(Cl)cncc1Cl] | LCMS m/z = 506.2 (M + H); 1H NMR (400 MHz, DMSO-d6) δ 13.60 (brs, 1H), 8.56 (s, 2H), 8.25 (d, J = 1.6 Hz, 1H), 8.00 (d, J = 1.6 Hz, 1H), 7.62 (s, 1H), 7.56 (d, J = 9.2 Hz, 1H), 7.48 (d, J = 2.0 Hz, 1H), 7.13 (dd, J = 9.2 Hz, 2.4 Hz, 1H), 6.77-6.50 (m, 1H), 6.18 (q, J = 6.8 Hz, 1H), 5.07 (td, J = 14.4 Hz, 3.2 Hz, 2H), 4.75 (t, J = 4.4 Hz, 2H), 3.62-3.55 (m, 2H), 1.78 (d, J = 6.8 Hz, 3H) |
| Example 143 7-[6-chloro-5- [(1R)-1-(3,5- dichloro-2- methyl-4-pyridyl) ethoxy]-1H- indazol-3-yl]-1- methylsulfonyl- 2,3-dihydro- pyrido[2,3-b][1,4] oxazine | 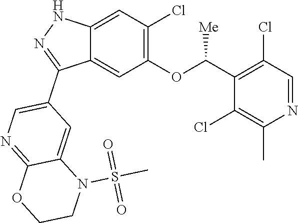 [SMILES: Cc1ncc(Cl)c([C@@H](C)Oc2cc3c(-c4cnc5c(c4)N(S(C)(=O)=O)CCO5)n[nH]c3cc2Cl)c1Cl] | LCMS m/z = 568.1 (M + H); 1H NMR (400 MHz, DMSO-d6) δ = 13.30 (s, 1H), 8.46 (s, 1H), 8.45 (d, J = 2.1 Hz, 1H), 8.18 (d, J = 2.1 Hz, 1H), 7.76 (s, 1H), 7.13 (s, 1H), 6.12 (q, J = 6.6 Hz, 1H), 4.53-4.43 (m, 2H), 3.94-3.86 (m, 2H), 3.22 (s, 3H), 2.59 (s, 3H), 1.81 (d, J = 6.6 Hz, 3H) |
| Example 144 7-[5-[(1R)-1-(3,5- dichloro-4- pyridyl)ethoxy]- 1H-indazol-3-yl]- 1-ethylsulfonyl]- 2,3-dihydro- pyrido[2,3-b][1,4] oxazine | 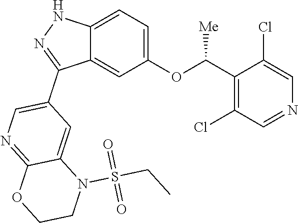 [SMILES: CCS(=O)(=O)N1CCOc2ncc(-c3n[nH]c4ccc(O[C@H](C)c5c(Cl)cncc5Cl)cc34)cc21] | LCMS m/z = 534.1 (M + H); 1H NMR (400 MHz, DMSO-d6) δ = 13.21 (s, 1H), 8.57 (s, 2H), 8.42 (d, J = 2.1 Hz, 1H), 8.35 (d, J = 2.1 Hz, 1H), 7.52 (d, J = 9.0 Hz, 1H), 7.25 (d, J = 2.1 Hz, 1H), 7.13 (dd, J = 2.3, 9.0 Hz, 1H), 6.13 (q, J = 6.6 Hz, 1H), 4.47 (t, J = 4.5 Hz, 2H), 3.96-3.86 (m, 2H), 3.42 (q, J = 7.3 Hz, 2H), 1.76 (d, J = 6.7 Hz, 3H), 1.26 (t, J = 7.3 Hz, 3H) |
| Example 145 7-[6-chloro-5- [(1R)-1-(3,5- dichloro-4- pyridyl)ethoxy]- 1H-indazol-3-yl]- 1-methylsulfonyl- 2,3-dihydro- pyrido[2,3-b][1,4] oxazine | 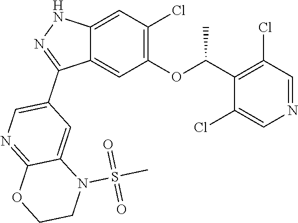 [SMILES: C[C@@H](Oc1cc2c(-c3cnc4c(c3)N(S(C)(=O)=O)CCO4)n[nH]c2cc1Cl)c1c(Cl)cncc1Cl] | LCMS m/z = 554 (M + H); 1H NMR (400 MHz, DMSO-d6) δ = 13.31 (s, 1H), 8.61 (s, 2H), 8.44 (d, J = 2.1 Hz, 1H), 8.31 (d, J = 2.1 Hz, 1H), 7.77 (s, 1H), 7.22 (s, 1H), 6.12 (q, J = 6.6 Hz, 1H), 4.48 (t, J = 4.6 Hz, 2H), 3.97- 3.85 (m, 2H), 3.22 (s, 3H), 1.81 (d, J = 6.6 Hz, 3H) |
| Example 146 R)-7-(5-(1-(3,5- Dichloropyridin- 4-yl)ethoxy)-6- methoxy-1H- indazol-3-yl)-2,3- dihydro-1H- pyrido[2,3-b][1,4] oxazine | 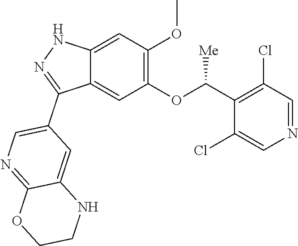 [SMILES: COc1cc2[nH]nc(-c3cnc4c(c3)NCCO4)c2cc1O[C@H](C)c1c(Cl)cncc1Cl] | LCMS m/z = 471.1 (M + H); 1H NMR (400 MHz, DMSO-d6) δ = 12.86 (s, 1H), 8.59 (s, 2H), 7.68 (d, J = 2.1 Hz, 1H), 7.31 (d, J = 2.0 Hz, 1H), 7.09 (s, 1H), 6.99 (s, 1H), 6.16 (s, 1H), 6.00-5.92 (m, 1H), 4.36-4.27 (m, 2H), 3.86 (s, 3H), 3.35-3.32 (m, 2H), 1.75 (d, J = 6.6 Hz, 3H) |
| Example 147 5-[(1R)-1-(3,5- dichloro-2- methyl-4-pyridyl) ethoxy]-3-[6- [(3R)-tetrahydro- furan-3-yl]oxy-3- pyridyl]-1H- indazole | 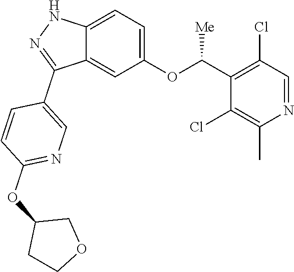 [SMILES: Cc1ncc(Cl)c([C@@H](C)Oc2ccc3[nH]nc(-c4ccc(O[C@@H]5CCOC5)nc4)c3c2)c1Cl] | LCMS m/z = 485.1 (M + H); 1H NMR (400 MHz, DMSO-d6) δ ppm 13.15 (s, 1 H) 8.42- 8.46 (m, 2 H) 8.05 (dd, J = 8.6, 2.4 Hz, 1 H) 7.49 (d, J = 9.0 Hz, 1 H) 7.07-7.13 (m, 2 H) 6.97 (dd, J = 8.6, 0.6 Hz, 1 H) 6.07-6.13 (m, 1 H) 5.58 (ddt, J = 6.5, 4.4, 2.0, 2.0 Hz, 1 H) 3.99 (dd, J = 10.3, 4.8 Hz, 1 H) 3.78-3.95 (m, 3 H) 2.58 (s, 3 H) 2.24-2.35 (m, 1 H) 2.02-2.12 (m, 1 H) 1.76 (d, J = 6.7 Hz, 3 H) |
| Example 148 5-[(1R)-1-(3,5- dichloro-2- methyl-4-pyridyl) ethoxy]-3-[5- methyl-6-[(3R)- tetrahydrofuran- 3-yl]oxy-3- pyridyl]-1H- indazole | 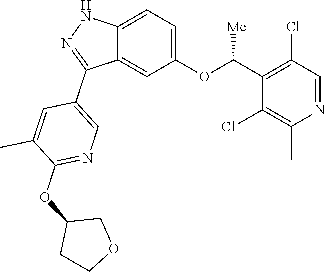 [SMILES: Cc1cc(-c2n[nH]c3ccc(O[C@H](C)c4c(Cl)cnc(C)c4Cl)cc23)cnc1O[C@@H]1CCOC1] | LCMS m/z = 499.1 (M + H); 1H NMR (400 MHz, DMSO-d6) δ ppm 13.12 (br s, 1 H) 8.43 (s, 1 H) 8.26 (d, J = 2.1 Hz, 1 H) 7.91 (dd, J = 2.3, 0.8 Hz, 1 H) 7.49 (d, J = 9.0 Hz, 1 H) 7.10 (dd, J = 9.0, 2.3 Hz, 1 H), 7.06 (d, J = 2.0 Hz, 1 H) 6.07-6.13 (m, 1 H) 5.59 (ddt, J = 6.5, 4.4, 2.1, 2.1 Hz, 1 H) 3.96-4.03 (m, 1 H) 3.87- 3.96 (m, 1 H) 3.78-3.87 (m, 2 H) 2.57 (s, 3 H) 2.25-2.34 (m, 1 H) 2.25 (s, 3 H) 2.00-2.15 (m, 1 H) 1.76 (d, J = 6.7 Hz, 3 H). |
| Example 149 5-[5-[(1R)-1-(3,5- dichloro-2- methyl-4-pyridyl) ethoxy]-6- methoxy-1H- indazol-3-yl]-N, N-dimethyl- pyridin-2-amine | 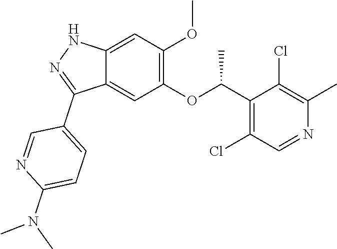 | LCMS m/z = 472 (M + H); 1H NMR (400 MHz, DMSO-d6) δ = 12.74 (s, 1 H) 8.45 (s, 1 H) 8.45 (s, 1 H) 8.34 (d, J = 2.1 Hz, 1 H) 7.80 (dd, J = 8.9, 2.4 Hz, 1 H) 6.98 (s, 1 H) 6.95 (s, 1 H) 6.74 (d, J = 8.9 Hz, 1 H) 5.93- 6.00 (m, 1 H) 3.87 (s, 3 H) 3.09 (s, 6 H) 2.61 (s, 3 H) 1.76 (d, J = 6.7 Hz, 3 H) |
| Example 150 5-[(1R)-1-(3,5- dichloro-2- methyl-4-pyridyl) ethoxy]-3-[6- [(3S)-tetrahydro- furan-3-yl]oxy-3- pyridyl]-1H- indazole | 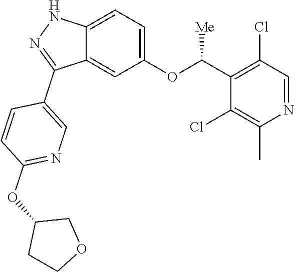 [SMILES: Cc1ncc(Cl)c([C@@H](C)Oc2ccc3[nH]nc(-c4ccc(O[C@H]5CCOC5)nc4)c3c2)c1Cl] | LCMS m/z = 485.1 (M + H); 1H NMR (400 MHz, DMSO-d6) δ ppm 13.15 (s, 1 H) 8.42- 8.47 (m, 2 H) 8.05 (dd, J = 8.6, 2.5 Hz, 1 H) 7.49 (d, J = 9.3 Hz, 1 H) 7.11 (d, J = 8.8 Hz, 1 H) 7.08 (d, J = 1.8 Hz, 1 H) 6.97 (dd, J = 8.7, 0.6 Hz, 1 H) 6.10 (q, J = 6.6 Hz, 1 H) 5.58 (ddt, J = 6.5, 4.4, 2.1, 2.1 Hz, 1 H) 3.95-4.01 (m, 1 H) 3.78-3.95 (m, 3 H) 2.58 (s, 3 H) 2.25- 2.35 (m, 1 H) 2.04-2.12 (m, 1 H) 1.76 (d, J = 6.7 Hz, 3 H) |
| Example 151 (R)-5-(5-(1-(3,5- dichloropyridin- 4-yl)ethoxy)-1H- indazol-3-yl)-3- methyl-N- (tetrahydro-2H- pyran-4-yl) pyridin-2-amine | 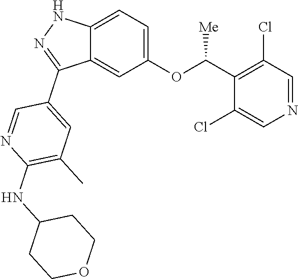 [SMILES: Cc1cc(-c2n[nH]c3ccc(O[C@H](C)c4c(Cl)cncc4Cl)cc23)cnc1NC1CCOCC1] | LCMS m/z = 498.2 (M + H); 1H NMR (400 MHz, DMSO-d6) δ 12.91 (s, 1H), 8.58 (s, 2H), 8.30 (s, 1H), 7.55 (s, 1H), 7.44 (d, J = 9.2 Hz, 1H), 7.12 (s, 1H), 7.07 (d, J = 8.8 Hz, 1H), 6.09 (q, J = 6.4 Hz, 1H), 5.75 (d, J = 8.0 Hz, 1H), 4.26-4.14 (m, 1H), 3.91 (d, J = 10.8 Hz, 2H), 3.51-3.39 (m, 2H), 2.17 (s, 3H), 1.91 (d, J = 12.0 Hz, 2H), 1.74 (d, J = 6.4 Hz, 3H), 1.67-1.54 (m, 2H) |
| Example 152 (R)-5-(1-(3,5- dichloropyridin- 4-yl)ethoxy)-3-(6- ((2-(methyl- sulfonyl)-2- azaspiro[3.3] heptan-6-yl)oxy) pyridin-3-yl)-1H- indazole | 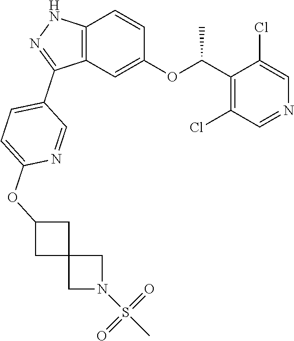 [SMILES: C[C@@H](Oc1ccc2[nH]nc(-c3ccc(OC4CC5(C4)CN(S(C)(=O)=O)C5)nc3)c2c1)c1c(Cl)cncc1Cl] | LCMS m/z = 574.2 (M + H); 1H NMR (400 MHz, DMSO-d6) δ 13.16 (s, 1H), 8.59 (s, 2H), 8.52 (d, J = 2.8 Hz, 1H), 8.02 (dd, J = 8.8 Hz, 2.4 Hz, 1H), 7.49 (d, J = 9.2 Hz, 1H), 7.16 (d, J = 2.0 Hz, 1H), 7.10 (dd, J = 8.8 Hz, 2.0 Hz, 1H), 6.94 (d, J = 8.4 Hz, 1H), 6.10 (q, J = 6.8 Hz, 1H), 5.15-5.11 (m, 1H), 3.99 (s, 2H), 3.90 (s, 2H), 2.99 (s, 3H), 2.81-2.76 (m, 2H), 2.35- 2.30 (m, 2H), 1.75 (d, J = 6.8 Hz, 3H) |
| Example 153 5-(5-((R)-1-(3,5- dichloropyridin- 4-yl)ethoxy)-1H- indazol-3-yl)-3- methyl-N-((S)- tetrahydrofuran- 3-yl)pyridin-2- amine | 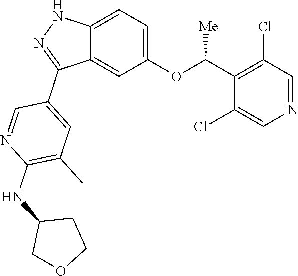 [SMILES: Cc1cc(-c2n[nH]c3ccc(O[C@H](C)c4c(Cl)cncc4Cl)cc23)cnc1N[C@H]1CCOC1] | LCMS m/z = 484.2 (M + H); 1H NMR (400 MHz, DMSO-d6) δ 12.93 (s, 1H), 8.59 (s, 2H), 8.32 (d, J = 2.0 Hz, 1H), 7.58 (d, J = 1.2 Hz, 1H), 7.45 (d, J = 9.2 Hz, 1H), 7.15-7.05 (m, 2H), 6.10 (q, J = 6.8 Hz, 1H), 6.00 (d, J = 6.0 Hz, 1H), 4.65-4.52 (m, 1H), 3.99-3.97 (m, 1H), 3.90 (q, J = 6.8 Hz, 1H), 3.75 (q, J = 6.0 Hz, 1H), 3.63-3.61 (m, 1H), 2.29-2.21 (m, 1H), 2.20 (s, 3H), 2.05-1.94 (m, 1H), 1.75 (d, J = 6.8 Hz, 3H). |
| Example 154 5-[(1R)-1-(3,5- dichloro-2- methyl-4-pyridyl) ethoxy]-3-[5- methyl-6-[(3S)- tetrahydrofuran-3- yl]oxy-3- pyridyl]-1H- indazole | 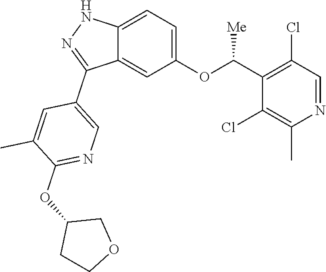 [SMILES: Cc1cc(-c2n[nH]c3ccc(O[C@H](C)c4c(Cl)cnc(C)c4Cl)cc23)cnc1O[C@H]1CCOC1] | LCMS m/z = 499.1 (M + H); 1H NMR (400 MHz, DMSO-d6) δ = 13.12 (s, 1H), 8.43 (s, 1H), 8.26 (t, J = 2.2 Hz, 1H), 7.91 (d, J = 1.3 Hz, 1H), 7.48 (d, J = 8.9 Hz, 1H), 7.10 (dd, J = 2.3, 9.0 Hz, 1H), 7.06 (s, 1H), 6.10 (q, J = 6.6 Hz, 1H), 5.59 (tdd, J = 2.1, 4.4, 6.5 Hz, 1H), 4.00 (ddd, J = 2.6, 4.8, 10.1 Hz, 1H), 3.94- 3.87 (m, 1H), 3.86-3.78 (m, 2H), 2.57 (s, 3H), 2.34-2.26 (m, 1H), 2.24 (s, 3H), 2.12-2.03 (m, 1H), 1.75 (d, J = 6.7 Hz, 3H). |
| Example 155 5-[5-[(1R)-1-(3,5- dichloro-2- methyl-4-pyridyl) ethoxy]-1H- indazol-3-yl]-N- [(3R)- tetrahydrofuran- 3-yl]pyridin-2- amine | 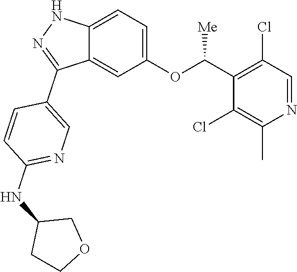 [SMILES: Cc1ncc(Cl)c([C@@H](C)Oc2ccc3[nH]nc(-c4ccc(N[C@@H]5CCOC5)nc4)c3c2)c1Cl] | LCMS m/z = 484.1 (M + H); 1H NMR (400 MHz, DMSO-d6) δ = 12.91 (s, 1H), 8.42 (s, 1H), 8.28 (d, J = 2.1 Hz, 1H), 7.72 (dd, J = 2.3, 8.7 Hz, 1H), 7.44 (d, J = 8.9 Hz, 1H), 7.07 (dd, J = 2.3, 9.0 Hz, 1H), 7.02 (s, 1H), 6.97 (d, J = 6.1 Hz, 1H), 6.63 (dd, J = 0.7, 8.7 Hz, 1H), 6.08 (q, J = 6.6 Hz, 1H), 4.44 (ttd, J = 4.0, 5.9, 7.6 Hz, 1H), 3.94 (dd, J = 5.9, 8.8 Hz, 1H), 3.90-3.84 (m, 1H), 3.75 (dt, J = 5.6, 8.1 Hz, 1H), 3.59 (dd, J = 3.9, 8.8 Hz, 1H), 2.60 (s, 3H), 2.22 (qd, J = 7.4, 12.6 Hz, 1H), 1.89- 1.81 (m, 1H), 1.75 (d, J = 6.7 Hz, 3H) |
| Example 156 5-[5-[(1R)-1-(3,5- dichloro-2- methyl-4-pyridyl) ethoxy]-1H- indazol-3-yl]-N- tetrahydrofuran- 4-yl-pyridin-2- amine | 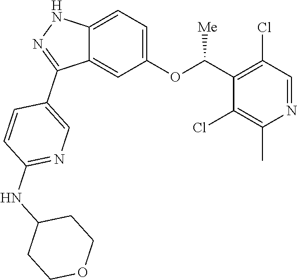 [SMILES: Cc1ncc(Cl)c([C@@H](C)Oc2ccc3[nH]nc(-c4ccc(NC5CCOCC5)nc4)c3c2)c1Cl] | LCMS m/z = 498.1 (M + H); 1H NMR (400 MHz, DMSO-d6) δ = 8.41 (s, 1H), 8.25 (d, J = 2.0 Hz, 1H), 7.70 (dd, J = 2.4, 8.7 Hz, 1H), 7.44 (d, J = 8.9 Hz, 1H), 7.07 (dd, J = 2.3, 9.0 Hz, 1H), 7.01 (d, J = 2.1 Hz, 1H), 6.71 (br d, J = 5.7 Hz, 1H), 6.60 (dd, J = 0.6, 8.7 Hz, 1H), 6.07 (q, J = 6.6 Hz, 1H), 3.99 (br s, 2H), 3.91 (br d, J = 11.5 Hz, 2H), 3.44 (dt, J = 1.8, 11.5 Hz, 2H), 2.61 (s, 3H), 1.98-1.91 (m, 2H), 1.75 (d, J = 6.7 Hz, 3H), 1.53-1.42 (m, 2H) |
| Example 157 1′-cyclobutyl-6- [5-[(1R)-1-(3,5- dichloro-4- pyridyl)ethoxy]- 1H-indazol-3-yl] spiro[4H-1,3- benzodioxine- 2,4′-piperidine] | 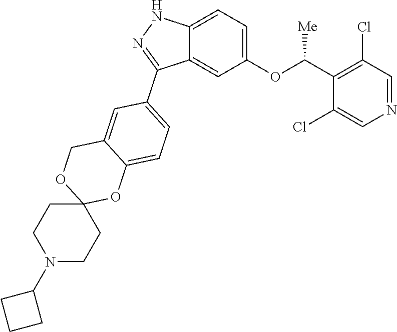 [SMILES: C[C@@H](Oc1ccc2[nH]nc(-c3ccc4c(c3)COC3(CCN(C5CCC5)CC3)O4)c2c1)c1c(Cl)cncc1Cl] | LCMS m/z = 565.2 (M + H); 1H NMR (400 MHz, DMSO-d6) δ = 13.12-12.93 (m, 1H), 8.64-8.55 (m, 2H), 7.55 (dd, J = 2.1, 8.6 Hz, 1H), 7.49-7.41 (m, 2H), 7.12-7.06 (m, 2H), 6.97 (d, J = 8.4 Hz, 1H), 6.08 (q, J = 6.8 Hz, 1H), 5.04-4.86 (m, 2H), 2.85-2.70 (m, 1H), 2.37 (br s, 3H), 2.28 (dt, J = 4.3, 7.3 Hz, 1H), 2.03-1.95 (m, 3H), 1.93-1.77 (m, 1H), 1.77- 1.73 (m, 3H), 1.67-1.58 (m, 2H), 1.53-1.43 (m, 2H), 1.28 (br s, 4H) |
| Example 158 5-(5-((R)-1-(3,5- dichloropyridin- 4-yl)ethoxy)-1H- indazol-3-yl)-3- methyl-N-((R)- tetrahydrofuran- 3-yl)pyridin-2- amine | 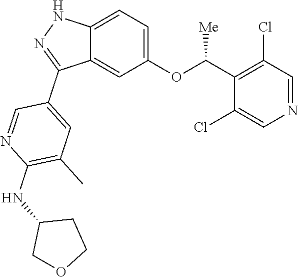 [SMILES: Cc1cc(-c2n[nH]c3ccc(O[C@H](C)c4c(Cl)cncc4Cl)cc23)cnc1N[C@@H]1CCOC1] | LCMS m/z = 484.2 (M + H); 1H-NMR (400 MHz, DMSO-d6) δ 12.94 (s, 1H), 8.59 (s, 2H), 8.29 (d, J = 2.0 Hz, 1H), 7.58 (d, J = 1.6 Hz, 1H), 7.44 (d, J = 8.8 Hz, 1H), 7.13-7.05 (m, 2H), 6.10 (q, J = 6.8 Hz, 1H), 6.00 (d, J = 6.0 Hz, 1H), 4.63-4.50 (m, 1H), 3.99 (dd, J = 8.8 Hz, 6.0 Hz, 1H), 3.90-3.87 (m, 1H), 3.80-3.70 (m, 1H), 3.61 (dd, J = 8.8 Hz, 4.8 Hz, 1H), 2.30-2.19 (m, 4H), 2.03-1.94 (m, 1H), 1.75 (d, J = 6.8 Hz, 3H). |
| Example 159 (R)-N-(5-(5-(1- (3,5-dichloro- pyridin-4-yl) ethoxy)-1H- indazol-3-yl) pyridin-2-yl)-2- (methylsulfonyl)- 2-azaspiro[3.3] heptan-6-amine | 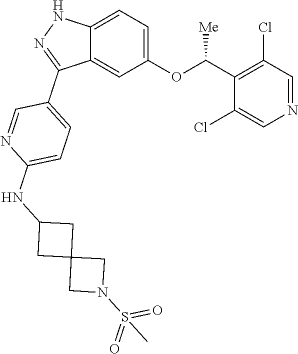 [SMILES: C[C@@H](Oc1ccc2[nH]nc(-c3ccc(NC4CC5(C4)CN(S(C)(=O)=O)C5)nc3)c2c1)c1c(Cl)cncc1Cl] | LCMS m/z = 573.2 (M + H); 1H-NMR (400 MHz, DMSO-d6) δ 12.92 (s, 1H), 8.58 (s, 2H), 8.39 (d, J = 2.4 Hz, 1H), 7.73 (dd, J = 8.8 Hz, 2.8 Hz, 1H), 7.44 (d, J = 8.8 Hz, 1H), 7.14 (d, J = 2.0 Hz, 1H), 7.07 (dd, J = 8.8 Hz, 2.0 Hz, 1H), 6.99 (d, J = 6.8 Hz, 1H), 6.53 (d, J = 8.8 Hz, 1H), 6.11 (q, J = 6.8 Hz, 1H), 4.26-4.14 (m, 1H), 3.97 (s, 2H), 3.85 (s, 2H), 2.98 (s, 3H), 2.69-2.60 (m, 2H), 2.12 (t, J = 10.0 Hz, 2H), 1.76 (d, J = 6.4 Hz, 3H) |
| Example 160 5-[5-[(1R)-1-(3,5- dichloro-2- methyl-4- pyridyl)ethoxy]- 1H-indazol-3-yl]- N-[(3S)- tetrahydrofuran- 3-yl]pyridin-2- amine | 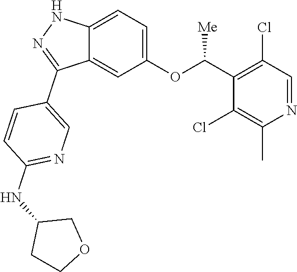 [SMILES: Cc1ncc(Cl)c([C@@H](C)Oc2ccc3[nH]nc(-c4ccc(N[C@H]5CCOC5)nc4)c3c2)c1Cl] | LCMS m/z = 484.1 (M + H); 1H NMR (400 MHz, DMSO-d6) δ = 12.91 (s, 1H), 8.41 (s, 1H), 8.29 (d, J = 2.1 Hz, 1H), 7.72 (dd, J = 2.3, 8.7 Hz, 1H), 7.44 (d, J = 9.0 Hz, 1H), 7.07 (dd, J = 2.3, 8.9 Hz, 1H), 7.02 (d, J = 2.0 Hz, 1H), 6.97 (d, J = 6.1 Hz, 1H), 6.63 (d, J = 8.7 Hz, 1H), 6.08 (q, J = 6.6 Hz, 1H), 4.44 (ttd, J = 4.1, 5.9, 7.6 Hz, 1H), 3.93 (dd, J = 5.9, 8.7 Hz, 1H), 3.90-3.84 (m, 1H), 3.75 (dt, J = 5.6, 8.1 Hz, 1H), 3.58 (dd, J = 3.9, 8.8 Hz, 1H), 2.60 (s, 3H), 2.22 (qd, J = 7.5, 12.6 Hz, 1H), 1.90- 1.81 (m, 1H), 1.75 (d, J = 6.7 Hz, 3H) |
| Example 161 (R)-3-(6-(2- oxaspiro[3.3] heptan-6-yloxy) pyridin-3-yl)-5- (1-(3,5- dichloropyridin- 4-yl)ethoxy)-1H- indazole | 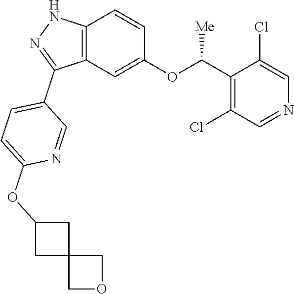 [SMILES: C[C@@H](Oc1ccc2[nH]nc(-c3ccc(OC4CC5(COC5)C4)nc3)c2c1)c1c(Cl)cncc1Cl] | LCMS m/z = 497.2 (M + H); 1H NMR (400 MHz, DMSO-d6) δ 13.15 (s, 1H), 8.59 (s, 2H), 8.52 (d, J = 2.4 Hz, 1H), 8.01 (dd, J = 8.4 Hz, 2.4 Hz, 1H), 7.48 (d, J = 9.2 Hz, 1H), 7.16 (s, 1H), 7.10 (dd, J = 9.2 Hz, 2.4 Hz, 1H), 6.93 (d, J = 8.4 Hz, 1H), 6.11 (q, J = 6.8 Hz, 1H), 5.08- 5.05 (m, 1H), 4.67 (s, 2H), 4.57 (s, 2H), 2.83- 2.78 (m, 2H), 2.32-2.27 (m, 2H), 1.76 (d, J = 6.8 Hz, 3H) |
| Example 162 5-((R)-1-(3,5- dichloropyridin- 4-yl)ethoxy)-3-(6- ((3-(methyl- sulfonyl)-3- azabicyclo[3.1.0] hexan-6-yl)oxy) pyridin-3-yl)-1H- indazole | 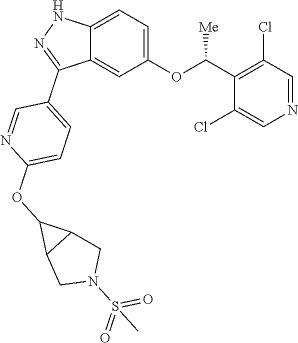 [SMILES: C[C@@H](Oc1ccc2[nH]nc(-c3ccc(OC4C5CN(S(C)(=O)=O)CC54)nc3)c2c1)c1c(Cl)cncc1Cl] | LCMS m/z = 560.2 (M + H); 1H-NMR (400 MHz, DMSO-d6) δ 13.21 (s, 1H), 8.65 (d, J = 2.0 Hz, 1H), 8.61 (s, 2H), 8.11 (dd, J = 8.8 Hz, 2.4 Hz, 1H), 7.52 (d, J = 8.8 Hz, 1H), 7.24 (d, J = 2.0 Hz, 1H), 7.13 (dd, J = 8.8 Hz, 2.4 Hz, 1H), 7.09 (d, J = 8.4 Hz, 1H), 6.15 (q, J = 6.4 Hz, 1H), 4.18 (t, J = 2.0 Hz, 1H), 3.60 (d, J = 9.2 Hz, 2H), 3.50 (d, J = 9.2 Hz, 2H), 2.99 (s, 3H), 2.12-2.01 (m, 2H), 1.79 (d, J = 6.4 Hz, 3H) |
| Example 163 6-[5-[(1R)-1-(3,5- dichloro-2- methyl-4-pyridyl) ethoxy]-1H- indazol-3-yl]-1′- methylsulfonyl- spiro[4H-1,3- benzodioxine- 2,4′-piperidine] | 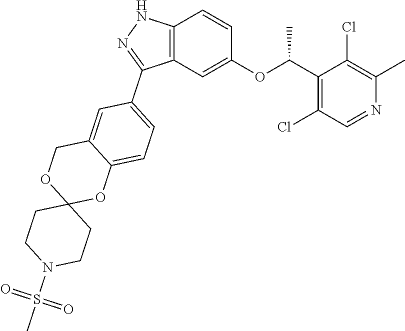 [SMILES: Cc1ncc(Cl)c([C@@H](C)Oc2ccc3[nH]nc(-c4ccc5c(c4)COC4(CCN(S(C)(=O)=O)CC4)O5)c3c2)c1Cl] | LCMS m/z = 603.1 (M + H); 1H NMR (400 MHz, DMSO-d6) δ = 13.03 (s, 1H), 8.49 (s, 1H), 7.57 (dd, J = 2.1, 8.4 Hz, 1H), 7.52-7.44 (m, 2H), 7.15-7.04 (m, 2H), 7.00 (d, J = 8.4 Hz, 1H), 6.11 (q, J = 6.6 Hz, 1H), 5.05-4.95 (m, 2H), 3.29-3.25 (m, 1H), 3.00-2.94 (m, 4H), 2.53-2.51 (m, 3H), 2.11-1.99 (m, 5H), 1.75 (d, J = 6.7 Hz, 4H) |
| Example 164 5-[5-[(1R)-1-(3,5- dichloro-2- methyl-4-pyridyl) ethoxy]-1H- indazol-3-yl]-3- methyl-N-[(3R)- tetrahydrofuran- 3-yl]pyridin-2- amine | 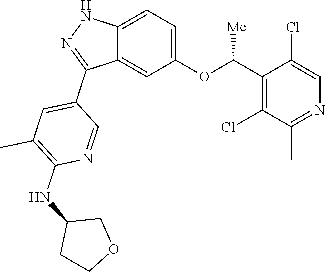 [SMILES: Cc1cc(-c2n[nH]c3ccc(O[C@H](C)c4c(Cl)cnc(C)c4Cl)cc23)cnc1N[C@@H]1CCOC1] | LCMS m/z = 498.2 (M + H); 1H NMR (400 MHz, DMSO-d6) δ = 12.91 (s, 1H), 8.43 (s, 1H), 8.17 (d, J = 1.8 Hz, 1H), 7.62 (s, 1H), 7.44 (d, J = 9.0 Hz, 1H), 7.08 (dd, J = 2.3, 9.0 Hz, 1H), 7.02 (s, 1H), 6.08 (q, J = 6.6 Hz, 1H), 5.98 (d, J = 5.9 Hz, 1H), 4.62-4.53 (m, 1H), 4.00 (dd, J = 6.3, 8.7 Hz, 1H), 3.96-3.85 (m, 1H), 3.75 (dt, J = 6.2, 8.0 Hz, 1H), 3.63 (dd, J = 4.7 8.7 Hz, 1H), 2.60 (s, 3H), 2.40-2.19 (m, 3H), 2.19 (s, 3H), 2.10-1.87 (m, 2H) |
| Example 165 6-[5-[(1R)-1-(3,5- dichloro-4- pyridyl)ethoxy]- 1H-indazol-3-yl]- 1′-methyl- sulfonyl-spiro [4H-1,3-benzo- dioxine-2,4′- piperidine] | 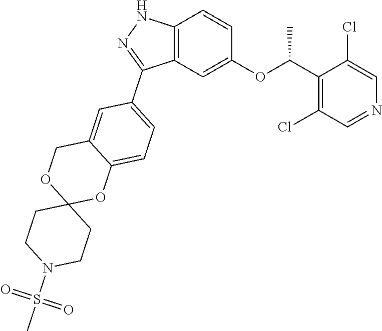 [SMILES: C[C@@H](Oc1ccc2[nH]nc(-c3ccc4c(c3)COC3(CCN(S(C)(=O)=O)CC3)O4)c2c1)c1c(Cl)cncc1Cl] | LCMS m/z = 589.1 (M + H); 1H NMR (400 NHz, CDCl3) δ = 9.92 (br s, 1H), 8.44 (s, 2H), 7.63 (dd, J = 2.1, 8.5 Hz, 1H), 7.44 (d, J = 2.0 Hz, 1H), 7.37 (dd, J = 0.7, 8.8 Hz, 1H), 7.17- 7.12 (m, 2H), 7.00 (d, J = 8.4 Hz, 1H), 6.04 (q, J = 6.6 Hz, 1H), 5.01-4.90 (m, 2H), 3.52- 3.44 (m, 2H), 3.44-3.34 (m, 2H), 2.87 (s, 3H), 2.18-2.05 (m, 4H), 1.82 (d, J = 6.7 Hz, 3H) |
| Example 166 6-[5-[(1R)-1-(3,5- dichloro-2- methyl-4-pyridyl) ethoxy]-1H- indazol-3-yl]-1′- (2,2-difluoro- ethyl)spiro[4H- 1,3-benzodioxine- 2,4′-piperidine] | 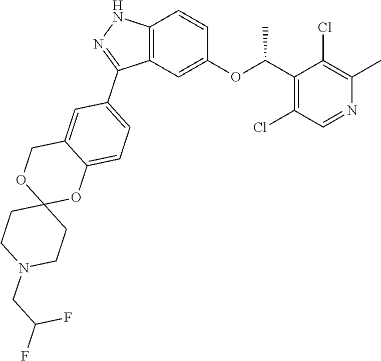 [SMILES: Cc1ncc(Cl)c([C@@H](C)Oc2ccc3[nH]nc(-c4ccc5c(c4)COC4(CCN(CC(F)F)CC4)O5)c3c2)c1Cl] | LCMS m/z = 589.2 (M + H); 1H NMR (400 MHz, DMSO-d6) δ = 13.01 (s, 1H), 8.48 (s, 1H), 7.55 (dd, J = 1.8, 8.5 Hz, 1H), 7.50-7.42 (m, 2H), 7.12-7.06 (m, 2H), 6.96 (d, J = 8.4 Hz, 1H), 6.40-5.96 (m, 2H), 5.02-4.90 (m, 2H), 2.81 (dt, J = 4.3, 15.7 Hz, 2H), 2.68 (br s, 4H), 2.51 (s, 3H), 1.95-1.86 (m, 4H), 1.75 (d, J = 6.6 Hz, 3H) |
| Example 167 6-[5-[(1R)-1-(3,5- dichloro-2- methyl-4-pyridyl) ethoxy]-1H- indazol-3-yl]-1′- (2,2,2-trifluoro- ethyl)spiro[4H- 1,3-benzodioxine- 2,4′-piperidine] | 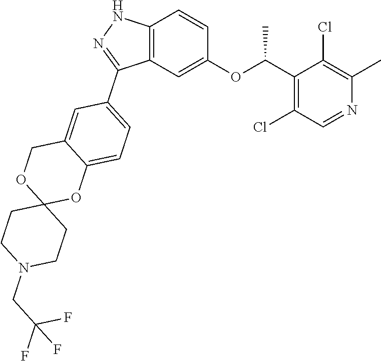 [SMILES: Cc1ncc(Cl)c([C@@H](C)Oc2ccc3[nH]nc(-c4ccc5c(c4)COC4(CCN(CC(F)(F)F)CC4)O5)c3c2)c1Cl] | LCMS m/z = 607.2 (M + H); 1H NMR (400 MHz, DMSO-d6) δ = 13.01 (s, 1H), 8.48 (s, 1H), 7.57-7.44 (m, 3H), 7.13-7.06 (m, 2H), 6.97 (d, J = 8.4 Hz, 1H), 6.50 (s, 1H), 6.10 (q, J = 6.7 Hz, 1H), 5.02-4.92 (m, 2H), 3.30-3.21 (m, 2H), 2.78 (br t, J = 5.1 Hz, 4H), 2.54 (s, 2H), 2.03-1.83 (m, 4H), 1.75 (d, J = 6.6 Hz, 3H) |
| Example 168 6-[5-[(1R)-1-(3,5- dichloro-4- pyridyl)ethoxy]- 1H-indazol-3-yl]- 1′-(2,2-difluoro- ethyl)spiro[4H- 1,3-benzodioxine- 2,4′-piperidine] | 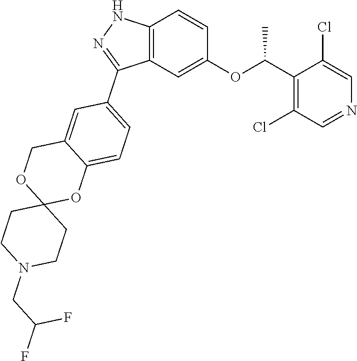 [SMILES: C[C@@H](Oc1ccc2[nH]nc(-c3ccc4c(c3)COC3(CCN(CC(F)F)CC3)O4)c2c1)c1c(Cl)cncc1Cl] | LCMS m/z = 575.2 (M + H); 1H NMR (400 MHz, CDCl3) δ = 9.86 (br s, 1H), 8.43 (s, 2H), 7.61 (dd, J = 2.1, 8.4 Hz, 1H), 7.42 (d, J = 2.0 Hz, 1H), 7.37 (d, J = 8.9 Hz, 1H), 7.13 (s, 1H), 7.15 (d, J = 8.8 Hz, 2H), 6.99 (d, J = 8.4 Hz, 1H), 6.10-5.74 (m, 2H), 5.00-4.90 (m, 2H), 2.83 (dt, J = 4.3, 15.0 Hz, 2H), 2.75 (t, J = 5.6 Hz, 3H), 2.11-1.93 (m, 4H), 1.82 (d, J = 6.6 Hz, 3H) |
| Example 169 6-[5-[(1R)-1-(3,5- dichloro-4- pyridyl)ethoxy]- 1H-indazol-3-yl]- 1′-(2,2,2- trifluoroethyl) spiro[4H-1,3- benzodioxine- 2,4′-piperidine] | 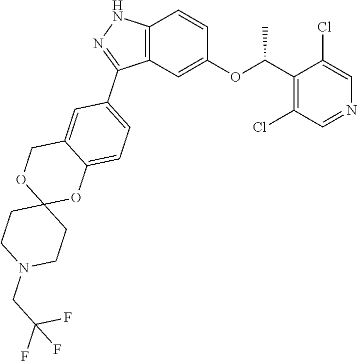 [SMILES: C[C@@H](Oc1ccc2[nH]nc(-c3ccc4c(c3)COC3(CCN(CC(F)(F)F)CC3)O4)c2c1)c1c(Cl)cncc1Cl] | LCMS m/z = 593.1 (M + H); 1H NMR (400 MHz, CDCl3) δ = 8.43 (s, 2H), 7.61 (dd, J = 1.8, 8.6 Hz, 1H), 7.42 (s, 1H), 7.37-7.30 (m, 1H), 7.17-7.10 (m, 2H), 6.99 (d, J = 8.4 Hz, 1H), 6.04 (q, J = 6.7 Hz, 1H), 5.00-4.88 (m, 2H), 3.07 (q, J = 9.7 Hz, 2H), 2.84 (t, J = 5.6 Hz, 4H), 2.22-1.89 (m, 5H), 1.82 (d, J = 6.7 Hz, 3H) |

### Kinase Assays

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. Streptavidin-coated magnetic beads were treated with biotinylated small molecule ligands for 30 minutes at room temperature to generate affinity resins for kinase assays. The liganded beads were blocked with excess biotin and washed with blocking buffer SeaBlock (Pierce), 1% BSA, 0.05% Tween 20, 1 mM DTT to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT).

Test compounds were prepared as 111X stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### Binding Constants (Kds)

Binding constants were calculated with a standard dose-response curve using the Hill equation:

$$Response = Background + \frac{Signal - Background}{1 + ( \frac{{Kd}^{Hill Slope}}{{Dose}^{Hill Slope}} )}$$

The Hill Slope was set to −1. Curves were fitted using a non-linear least square fit with the Levenberg-Marquardt algorithm.

TABLE 1

| | FGFR1 | FGFR2 | FGFR3 | FGFR4 |
|---|---|---|---|---|
| Example | (K_{d} nM) | (K_{d} nM) | (K_{d} nM) | (K_{d} nM) |
| 1. | B | B | A | A |
| 2. | D | D | D | A |
| 3. | D | D | D | C |
| 4. | D | D | C | C |
| 5. | D | B | C | A |
| 6. | D | D | D | C |
| 7. | D | D | B | A |
| 8. | A | A | A | A |
| 9. | A | A | A | A |
| 10. | A | A | A | A |
| 11. | A | B | A | A |
| 12. | A | A | A | A |
| 13. | A | A | A | A |
| 14. | A | A | A | A |
| 15. | D | D | B | A |
| 16. | A | A | A | A |
| 17. | A | A | A | A |
| 18. | C | C | B | A |
| 19. | A | A | A | A |
| 20. | B | B | A | A |
| 21. | D | D | C | A |
| 22. | A | A | A | A |
| 23. | D | D | C | A |
| 24. | A | A | A | A |
| 25. | D | D | D | A |
| 26. | D | D | C | A |
| 27. | B | C | A | A |
| 28. | C | D | C | A |
| 29. | A | A | A | A |
| 30. | A | D | A | A |
| 31. | C | D | B | B |
| 32. | D | D | D | A |
| 33. | D | D | C | A |
| 34. | C | C | A | A |
| 35. | A | A | A | A |
| 36. | A | A | A | A |
| 37. | D | D | D | A |
| 38. | A | A | A | A |
| 39. | D | D | C | A |
| 40. | C | C | A | A |
| 41. | A | B | A | A |
| 42. | D | D | B | A |
| 43. | C | C | A | A |
| 44. | D | D | C | A |
| 45. | C | D | A | A |
| 46. | D | D | D | A |
| 47. | B | B | A | C |
| 48. | D | D | A | C |
| 49. | D | D | C | B |
| 50. | D | D | D | A |
| 51. | D | D | C | B |
| 52. | D | C | B | A |
| 53. | C | A | A | A |
| 54. | C | C | A | A |
| 55. | D | D | D | C |
| 56. | C | C | B | A |
| 57. | C | D | C | A |
| 58. | A | A | A | A |
| 59. | D | D | D | D |
| 60. | D | D | D | D |
| 61. | D | D | D | C |
| 62. | D | D | D | B |
| 63. | D | D | C | A |
| 64. | D | D | D | B |
| 65. | D | D | D | D |
| 66. | D | D | D | C |
| 67. | B | C | B | A |
| 68. | D | D | D | A |
| 69. | B | A | A | A |
| 70. | D | D | D | D |
| 71. | C | D | C | A |
| 72. | A | A | A | A |
| 73. | D | C | B | A |
| 74. | C | C | A | A |
| 75. | B | B | A | A |
| 76. | C | D | B | A |
| 77. | C | B | A | A |
| 78. | D | D | C | A |
| 79. | A | A | A | A |
| 80. | B | A | A | A |
| 81. | D | D | D | A |
| 82. | B | B | A | A |
| 83. | C | D | C | A |
| 84. | C | C | A | A |
| 85. | D | D | C | A |
| 86. | D | D | D | C |
| 87. | D | C | C | C |
| 88. | C | C | B | A |
| 89. | B | B | A | A |
| 90. | C | A | A | A |
| 91. | D | D | D | D |
| 92. | A | A | A | A |
| 93. | D | D | D | B |
| 94. | A | A | A | A |
| 95. | A | A | A | A |
| 96. | A | A | A | A |
| 97. | D | D | D | C |
| 98. | B | B | B | A |
| 99. | A | A | A | A |
| 100. | C | C | B | A |
| 101. | A | A | A | v |
| 102. | B | B | A | A |
| 103. | 45 | B | A | A |
| 104. | A | A | A | A |
| 105. | D | D | C | A |
| 106. | A | A | A | A |
| 107. | A | A | A | A |
| 108. | C | C | A | A |
| 109. | D | D | C | A |
| 110. | A | A | A | A |
| 111. | A | A | A | A |
| 112. | A | A | A | A |
| 113. | A | A | A | A |
| 114. | | | | |
| 115. | A | A | A | A |
| 116. | A | A | A | A |
| 117. | B | C | B | A |
| 118. | | | | |
| 119. | A | A | A | A |
| 120. | A | A | A | A |
| 121. | A | A | A | A |
| 122. | A | A | A | A |
| 123. | C | C | A | A |
| 124. | A | A | A | A |
| 125. | A | A | A | A |
| 126. | C | D | C | A |
| 127. | B | A | A | A |
| 128. | B | B | A | A |
| 129. | A | A | A | A |
| 130. | A | A | A | A |
| 131. | A | A | A | A |
| 132. | A | A | A | A |
| 133. | B | A | A | A |
| 134. | B | A | A | A |
| 135. | A | A | A | A |
| 136. | A | A | A | A |
| 137. | C | B | A | A |
| 138. | B | C | A | A |
| 139. | D | D | D | D |
| 140. | C | C | B | A |
| 141. | D | D | D | A |
| 142. | D | D | D | C |
| 143. | D | D | B | B |
| 144. | D | D | D | A |
| 145. | D | D | D | A |
| 146. | A | A | A | A |
| 147. | A | A | A | A |
| 148. | C | C | A | A |
| 149. | A | A | A | A |
| 150. | A | A | A | A |
| 151. | B | B | A | A |
| 152. | A | A | A | A |
| 153. | A | A | A | A |
| 154. | C | C | A | A |
| 155. | A | A | A | A |
| 156. | A | A | A | A |
| 157. | A | A | A | A |
| 158. | A | B | A | A |
| 159. | A | A | A | A |
| 160. | A | A | A | A |
| 161. | A | A | A | A |
| 162. | A | A | A | A |
| 163. | B | A | A | B |
| 164. | C | A | A | A |
| 165. | A | A | A | A |
| 166. | B | B | A | A |
| 167. | B | B | A | A |
| 168. | A | A | A | A |
| 169. | B | B | A | A |

A = 0.1-100 nM
B = >100-250 nM
C = >250-1000 nM
D = >1000 nM

### Cell Viability Assays

### Cell Lines Used for Cell Viability Assays

| Cell line | Mutation or Fusion | Source |
|---|---|---|
| Ba/F3 | FGFR4-fusion | Advanced Cellular |
| BCR-FGFR4 | | Dynamics, (Seattle, WA) |
| Ba/F3 | FGFR3-fusion | Advanced Cellular |
| FGFR3-BAIAP2L1 | | Dynamics, (Seattle, WA) |
| Ba/F3 | FGFR1-fusion | Advanced Cellular |
| BCR-FGFR1 | | Dynamics, (Seattle, WA) |

### Ba/F3 Cell Viability Assays

Experimental Purpose: Recombinant kinase fusions are transduced into parental Ba/F3, which becomes dependent upon this constitutive kinase activity for IL3-independent survival. Inhibition of kinase activity leads to cell death, which is monitored using CellTiter-Glo® 2.0 (Promega) which measures intracellular ATP concentration that in turn serves as a marker for viability. BCR-FGFR4 Ba/F3, BCR-FGFR1 Ba/F3 and FGFR3-BAIAP2L1 Ba/F3 were obtained from Advanced Cellular Dynamics (Seattle, WA)

Cell Viability Assay Procedure: Cell Titer-Glo® 2.0 Luminescent cell viability assay reagent was purchased from Promega (Madison, WI). BCR-FGFR4 Ba/F3, BCR-FGFR1 Ba/F3 and FGFR3-BAIAP2L1 Ba/F3 cells were cultured in RPMI1640 media supplemented with 10% fetal bovine serum. Cultures were maintained at 37° C. in a humidified atmosphere of 5% CO₂ and 95% air.

Cells were plated in 96-well clear bottom/white plates (Corning #3903) at 10,000 cells/well in 100 μl of media, incubated overnight. The next day, test compound DMSO stock solutions were made at 10 mM and 2 μM final concentration. Compounds were then added to cells in a 9-dose, 10-fold dilution series starting at 30 μM with an HP 300e Digital Dispenser (each dose was applied in triplicate). DMSO was backfilled to each well up to 301 nL total volume of test compound+DMSO, and a total of 301 nL DMSO was added to a control/no test compound well in triplicate. The cells in cell culture plates were incubated with the compounds at 37° C. and 5% CO₂ for 48 hours. Then 50 μl of Cell Titer Glo 2.0 reagent was added to each well of the cell culture plates. The contents were covered from light and mixed on an orbital shaker at room temperature for 10 min. Luminescence was recorded by a Synergy H1 Microplate Reader (Biotek, Winooski, VT). Cells were assessed as a percentage of DMSO only treated control cells. Curves were plotted and IC₅₀ values were calculated using the GraphPad Prism 8 program based on a sigmoidal dose-response equation (4 parameter).

TABLE 2
Ba/F3 cell data for examples

| | FGFR4 | FGFR1 | FGFR3 |
|---|---|---|---|
| Example | (IC50 nM) | (IC50 nM) | (IC50 nM) |
| 8 | | A | A |
| 9 | | B | A |
| 13 | | A | A |
| 16 | | A | A |
| 17 | C | | |
| 20 | C | | |
| 25 | A | D | C |
| 26 | A | | |
| 28 | C | D | C |
| 30 | C | | |
| 34 | C | | |
| 40 | C | | |
| 41 | C | | |
| 42 | D | | |
| 45 | C | | |
| 49 | C | | |
| 53 | C | | |
| 72 | B | C | A |
| 79 | C | C | A |
| 82 | | | B |
| 92 | | C | A |
| 95 | | B | A |
| 96 | | B | A |
| 106 | | A | A |
| 111 | | A | A |
| 112 | | A | A |
| 119 | | C | A |
| 137 | | C | A |
| 147 | | D | B |
| 148 | | C | B |
| 150 | | C | A |
| 151 | | C | A |
| 152 | | B | A |
| 153 | | C | A |
| 155 | | B | A |
| 156 | | B | A |
| 157 | | C | A |
| 158 | | C | A |
| 159 | | A | A |
| 160 | | B | A |
| 162 | | B | A |
| 163 | | C | A |
| 164 | | D | A |
| 165 | | C | A |
| 166 | | D | B |
| 169 | | D | A |

A = 0.1-100 nM
B = >100-250 nM
C = >250-1000 nM
D = >1000 nM

1. A compound of formula (I) 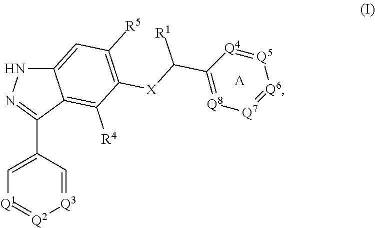
or a pharmaceutically acceptable salt thereof,
R¹ is C₁-C₆alkyl;
X=—O—, —S—, —NH—, or —N(C₁-C₃alkyl)-;
Q¹, Q², and Q³, are each independently N or C—R², wherein at least one of Q¹, Q², and Q³ is C—R²;
or, when Q¹ and Q² are both C—R², said R² groups together with the atoms to which they are attached, form a substituted or unsubstituted 5-7 membered cycloalkyl ring or a substituted or unsubstituted 5-7 membered heterocycloalkyl ring;
each R² is independently H, halogen, substituted or unsubstituted —C₁-C₆alkyl, —OR^{2a}; —NR^{2b}R^{2c}, —C(O)NR^{2b}R^{2c}, —SO₂C₁-C₆alkyl, —CN, —CF₃, —CF₂, —NR^{2b}COR^{2a}, or —NR^{2b}CONR^{2a}R^{2b},
or, when Q¹, Q², and Q³ are each C—R², each R² is independently H, halogen, substituted or unsubstituted —C₁-C₆alkyl, —OR^{2a}, —NR^{2b}R^{2c}, —C(O)NR^{2b}R^{2c}, —SO₂C₁-C₆alkyl, —CN, —CF₃, —CF₂, —NR²⁶COR^{2a}, —NR²⁶CONR^{2a}R^{2b} substituted or unsubstituted —C₃-C₆cycloalkyl, substituted or unsubstituted 3-6-membered heterocycloalkyl, substituted or unsubstituted bridged heterocycloalkyl, or a substituted or unsubstituted spiroheterocycloalkyl;
each R^{2a} is independently H, substituted or unsubstituted —C₁-C₆alkyl, —C₁-C₆haloalkyl, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, substituted or unsubstituted spiroheterocycloalkyl, substituted or unsubstituted —C₁-C₆alk-C₃-C₆cycloalkyl, or substituted or unsubstituted —C₁-C₆alk-O—C₁-C₆alkyl,
each R^{2b} is independently H, —C(O)-(substituted or unsubstituted 5- to 10-membered heteroaryl), —C(O)-(substituted or unsubstituted 5- to 6-membered heterocycloalkyl), substituted or unsubstituted —C₁-C₆alkyl, substituted or unsubstituted —C₁-C₆alk-O—C₁-C₆alkyl, —C(O)-(substituted or unsubstituted —C₃-C₆cycloalkyl), —C(O)N (substituted or unsubstituted C₁-C₆alkyl)₂, —C(O)C₁-C₃alkN (substituted or unsubstituted C₁-C₆alkyl)₂, substituted or unsubstituted —C(O)—OC₁-C₆alkyl, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, substituted or unsubstituted spiroheterocycloalkyl, or —C(O)CH—CH₂;
each R^{2c} is independently H, —C(O)-(substituted or unsubstituted 5- to 10-membered heteroaryl), —C(O)-(substituted or unsubstituted 5- to 6-membered heterocycloalkyl), substituted or unsubstituted —C₁-C₆alkyl, substituted or unsubstituted —C₁-C₆alk-O—C₁-C₆alkyl, —C(O)— (substituted or unsubstituted —C₃-C₆cycloalkyl), —C(O)N (substituted or unsubstituted C₁-C₆alkyl)₂, —C(O)C₁-C₃alkN (substituted or unsubstituted C₁-C₆alkyl)₂, substituted or unsubstituted —C(O)—OC₁-C₆alkyl, substituted or unsubstituted —C₃-C₆cycloalkyl, substituted or unsubstituted 3-6-membered heterocycloalkyl, substituted or unsubstituted bridged heterocycloalkyl, substituted or unsubstituted bridged cycloalkyl, substituted or unsubstituted spiroheterocycloalkyl, or —C(O)CH═CH₂;
Q⁴, Q⁵, Q⁶, Q⁷, and Q⁸, are each independently N or C—R³, wherein one or two of Q⁴, Q⁵, Q⁶, Q⁷, and Q³ is N and the remainder are C—R³, and
each R³ is independently H, halogen, C₁-C₆alkyl, C₁-C₆alkoxyl, or cycloalkyl;
R⁴ is H, halogen, —C₁-C₆alkyl, —C₁-C₆alkoxyl, or -cycloalkyl;
R⁵ is H, halogen, —C₁-C₆alkyl, —C₁-C₆alkoxyl, or -cycloalkyl;
wherein when ring A is 3,5-dichloropyridin-4-yl, and two of Q¹, Q², or Q³ are C—H, then the other of Q¹, Q², or Q³ is C—R² wherein R² is halogen, substituted or unsubstituted —C₁-C₆alkyl, —OR^{2a}, —NR^{2b}R^{2c}, —SO₂C₁-C₆alkyl, or —CN;
wherein the compound of formula (I) is not
N-(5-(5-(1-(3,5-dichloropyridin-4-yl)ethoxy)-1H-indazol-3-yl)pyridin-2-yl)-2-morpholinoacetamide,
(R)-3-(2-chloropyrimidin-5-yl)-5-(1-(3,5-dichloropyridin-4-yl)ethoxy)-1H-indazole,
(R)-5-(1-(3,5-dichloropyridin-4-yl)ethoxy)-3-(6-fluoropyridin-3-yl)-1H-indazole, or
(R)-3-(6-chloropyridin-3-yl)-5-(1-(3,5-dichloropyridin-4-yl)ethoxy)-1H-indazole.

2. The compound according to claim 1, wherein R¹ is —CH₃.

3. The compound according to claim 1, wherein X is O.

4. The compound according to claim 1, wherein Q⁶ is N.

5. The compound according to claim 1, wherein Q⁴, Q⁵, Q⁷, and Q⁸ are each C—R³.

6. The compound according to claim 5, wherein each R³ is independently H or —Cl.

7. The compound according to claim 1, wherein Q⁶ is N and Q⁷ is N.

8. The compound according to claim 7, wherein Q⁴, Q⁵, and Q⁸ are each C—R³.

9. The compound according to claim 8, wherein each R³ is independently H or —CH₃.

10. The compound according to claim 1, wherein Q³ is N; and Q¹ and Q² are each independently C—R².

11. The compound according to claim 1, wherein Q¹, Q², and Q³ are each independently C—R².

12. The compound according to claim 11, wherein Q² is C—H.

13. The compound according to claim 1, wherein the compound of formula (I) is a compound of formula (IA): 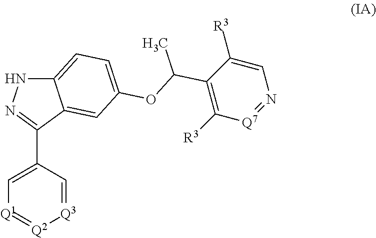
wherein Q⁷ is N or C—R³; and
each R³ is independently H, halogen, or C₁-C₃alkyl.

14.-62. (canceled)

63. The compound according to claim 1, wherein the compound of formula (I) is a compound of formula (IB): 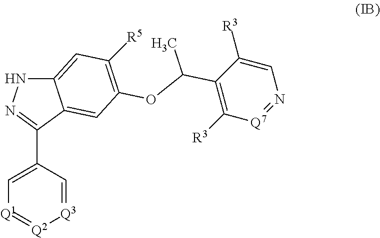
Q⁷ is N or C—R³;
each R³ is independently H, halogen, or C₁-C₃alkyl; and
R⁵ is halogen, —C₁-C₆alkyl, or —C₁-C₆ alkoxyl.

64.-76. (canceled)

77. A pharmaceutical composition comprising a compound of claim 1, or a pharmaceutically acceptable salt thereof, and a pharmaceutically acceptable excipient.

78. A method of treating cancer in a subject in need thereof comprising administering to the subject a compound of claim 1, or a pharmaceutically acceptable salt thereof.

79. The method of claim 78, wherein the cancer is urothelial carcinoma, breast carcinoma, endometrial adenocarcinoma, ovarian carcinoma, primary glioma, cholangiocarcinoma, gastric adenocarcinoma, non-small cell lung carcinoma, pancreatic exocrine carcinoma, oral, prostate, bladder, colorectal carcinoma, renal cell carcinoma, neuroendocrine carcinoma, myeloproliferative neoplasms, head and neck (squamous), melanoma, leiomyosarcoma, and/or sarcomas.

80. The method of claim 79, wherein the cancer is an intrahepatic cholangiocarcinoma.

81. The method of claim 78, wherein the cancer is an FGFR-mutant cancer.

82. The compound according to claim 1, wherein the compound is:
5-[5-[(1R)-1-(3,5-dichloro-4-pyridyl)ethoxy]-1H-indazol-3-yl]-2-methoxy-pyridin-3-amine;
N-(5-(5-((R)-1-(3,5-Dichloropyridin-4-yl)ethoxy)-1-(tetrahydro-2H-pyran-2-yl)-1H-indazol-3-yl)-2-methoxypyridin-3-yl)picolinamide;
(R)—N-(5-(5-(1-(3,5-Dichloropyridin-4-yl)ethoxy)-1H-indazol-3-yl)-2-methoxypyridin-3-yl)quinoline-8-carboxamide;
(R)-5-(5-(1-(3,5-Dichloropyridin-4-yl)ethoxy)-1H-indazol-3-yl)-2-methoxy-N-(pyridin-2-ylmethyl)pyridin-3-amine;
(R)—N-(5-(5-(1-(3,5-Dichloropyridin-4-yl)ethoxy)-1H-indazol-3-yl)-2-methoxypyridin-3-yl)morpholine-4-carboxamide;
(R)-5-(5-(1-(3,5-Dichloropyridin-4-yl)ethoxy)-1H-indazol-3-yl)-2-methoxy-N-((2-methylpyridin-4-yl)methyl)pyridin-3-amine;
(R)—N-(5-(5-(1-(3,5-Dichloropyridin-4-yl)ethoxy)-1H-indazol-3-yl)-2-methoxypyridin-3-yl)-4-methylpiperazine-1-carboxamide;
(R)-5-(5-(1-(3,5-Dichloropyridin-4-yl)ethoxy)-1H-indazol-3-yl)pyridin-2-amine;
(R)-5-(5-(1-(3,5-Dichloropyridin-4-yl)ethoxy)-1H-indazol-3-yl)-N-methylpicolinamide;
(R)-5-(1-(3,5-Dichloropyridin-4-yl)ethoxy)-3-(6-(methylsulfonyl)pyridin-3-yl)-1H-indazole;
(R)—N-(5-(5-(1-(3,5-Dichloropyridin-4-yl)ethoxy)-1H-indazol-3-yl)pyridin-2-yl)cyclopropanecarboxamide;
5-[5-[(1R)-1-(3,5-dichloro-4-pyridyl)ethoxy]-1H-indazol-3-yl]pyridine-2-carboxamide;
N-[5-[5-[(1R)-1-(3,5-dichloro-4-pyridyl)ethoxy]-1H-indazol-3-yl]-2-pyridyl]-2-(dimethylamino)-acetamide;
3-[5-[5-[(1R)-1-(3,5-dichloro-4-pyridyl)ethoxy]-1H-indazol-3-yl]-2-pyridyl]-1,1-dimethyl-urea;
N-[5-[5-[(1R)-1-(3,5-dichloro-4-pyridyl)ethoxy]-1H-indazol-3-yl]-2-methoxy-3-pyridyl]-2-(dimethylamino)acetamide;
N-[5-[5-[(1R)-1-(3,5-dichloro-4-pyridyl)ethoxy]-1H-indazol-3-yl]-2-pyridyl]morpholine-4-carboxamide;
7-[5-[(1R)-1-(3,5-dichloro-4-pyridyl)ethoxy]-1H-indazol-3-yl]-2,3-dihydro-1H-pyrido[2,3-b][1,4]oxazine;
[7-[5-[(1R)-1-(3,5-dichloro-4-pyridyl)ethoxy]-1H-indazol-3-yl]-2,3-dihydropyrido[2,3-b][1,4]oxazin-1-yl]-(2-pyridyl)methanone;
5-[5-[(1R)-1-(3,5-Dichloro-4-pyridyl)ethoxy]-1H-indazol-3-yl]-3-ethoxy-pyridin-2-amine;
[7-[5-[(1R)-1-(3,5-dichloro-4-pyridyl)ethoxy]-1H-indazol-3-yl]-2,3-dihydropyrido[2,3-b][1,4]oxazin-1-yl]-morpholino-methanone;
N-[5-[5-[(1R)-1-(3,5-dichloro-4-pyridyl)ethoxy]-1H-indazol-3-yl]-2-pyridyl]-4-methyl-piperazine-1-carboxamide;
[7-[5-[(1R)-1-(3,5-dichloro-4-pyridyl)ethoxy]-1H-indazol-3-yl]-2,3-dihydropyrido[2,3-b][1,4]oxazin-1-yl]-(2-pyridyl)methanone;
1-[7-[5-[(1R)-1-(3,5-dichloro-4-pyridyl)ethoxy]-1H-indazol-3-yl]-2,3-dihydropyrido[2,3-b][1,4]oxazin-1-yl]-2-(dimethylamino)ethanone;
N-[5-[5-[(1R)-1-(3,5-dichloro-4-pyridyl)ethoxy]-1H-indazol-3-yl]-3-fluoro-2-pyridyl]-2-(dimethylamino)acetamide;
[7-[5-[(1R)-1-(3,5-dichloro-4-pyridyl)ethoxy]-1H-indazol-3-yl]-2,3-dihydropyrido[2,3-b][1,4]oxazin-1-yl]-(2-methyl-4-pyridyl)methanone;
N-[5-[5-[(1R)-1-(3,5-dichloro-4-pyridyl)ethoxy]-1H-indazol-3-yl]-2-pyridyl]-3-hydroxy-pyrrolidine-1-carboxamide;
1-[7-[5-[(1R)-1-(3,5-dichloro-4-pyridyl)ethoxy]-1H-indazol-3-yl]-2,3-dihydropyrido[2,3-b][1,4]oxazin-1-yl]prop-2-en-1-one;
1-[7-[5-[(1R)-1-(3,5-dichloro-4-pyridyl)ethoxy]-1H-indazol-3-yl]-2,3-dihydropyrido[2,3-b][1,4]oxazin-1-yl]-3-(dimethylamino)propan-1-one;
isopropyl N-[5-[5-[(1R)-1-(3,5-dichloro-4-pyridyl)ethoxy]-1H-indazol-3-yl]-2-pyridyl]carbamate;
5-[5-[(1R)-1-(3,5-dichloro-4-pyridyl)ethoxy]-1H-indazol-3-yl]-3-(difluoromethoxy)pyridin-2-amine;
4-[5-[(1R)-1-(3,5-dichloro-4-pyridyl)ethoxy]-1H-indazol-3-yl]pyridin-2-amine;
2-amino-5-[5-[(1R)-1-(3,5-dichloro-4-pyridyl)ethoxy]-1H-indazol-3-yl]pyridine-3-carbonitrile;
N-[5-[5-[(1R)-1-(3,5-dichloro-4-pyridyl)ethoxy]-1H-indazol-3-yl]-2-methoxy-3-pyridyl]-3-(dimethylamino)propanamide;
5-[5-[(1R)-1-(3,5-dichloro-4-pyridyl)ethoxy]-1H-indazol-3-yl]-3-(trifluoromethoxy)pyridin-2-amine;
5-[5-[(1R)-1-(3,5-dichloro-4-pyridyl)ethoxy]-1H-indazol-3-yl]-3-ethoxy-pyridin-2-amine;
5-[5-[(1R)-1-(3,5-dichloro-4-pyridyl)ethoxy]-1H-indazol-3-yl]-3-methoxy-pyridin-2-amine;
(R)-4-(5-(1-(3,5-Dichloropyridin-4-yl)ethoxy)-1H-indazol-3-yl)-N-(2-methoxyethyl)pyridin-2-amine;
2-[[4-[5-[(1R)-1-(3,5-Dichloro-4-pyridyl)ethoxy]-1H-indazol-3-yl]-2-pyridyl]amino]ethanol;
5-[5-[(1R)-1-(3,5-Dichloro-4-pyridyl)ethoxy]-1H-indazol-3-yl]-3-(2,2,2-trifluoroethoxy)pyridin-2-amine;
7-[5-[(1R)-1-(3,5-dichloro-4-pyridyl)ethoxy]-1H-indazol-3-yl]-4H-pyrido[3,2-b][1,4]oxazin-3-one;
[7-[5-[(1R)-1-(3,5-dichloro-4-pyridyl)ethoxy]-1H-indazol-3-yl]-2,3-dihydropyrido[2,3-b][1,4]oxazin-1-yl]-(6-methyl-2-pyridyl)methanone;
[7-[5-[(1R)-1-(3,5-dichloro-4-pyridyl)ethoxy]-1H-indazol-3-yl]-2,3-dihydropyrido[2,3-b][1,4]oxazin-1-yl]-(2-methylpyrimidin-4-yl)methanone;
[7-[5-[(1R)-1-(3,5-dichloro-4-pyridyl)ethoxy]-1H-indazol-3-yl]-2,3-dihydropyrido[2,3-b][1,4]oxazin-1-yl]-(6-methyl-3-pyridyl)methanone;
[7-[5-[(1R)-1-(3,5-dichloro-4-pyridyl)ethoxy]-1H-indazol-3-yl]-2,3-dihydropyrido[2,3-b][1,4]oxazin-1-yl]-(2-methyl-3-pyridyl)methanone;
3-(dimethylamino)propyl 7-[5-[(1R)-1-(3,5-dichloro-4-pyridyl)ethoxy]-1H-indazol-3-yl]-2,3-dihydropyrido[2,3-b][1,4]oxazine-1-carboxylate;
3-[5-[(1R)-1-(3,5-dichloro-4-pyridyl)ethoxy]-1H-indazol-3-yl]-5-methoxy-aniline;
N-[3-[5-[(1R)-1-(3,5-dichloro-4-pyridyl)ethoxy]-1H-indazol-3-yl]-5-methoxy-phenyl]pyridine-2-carboxamide;
3-[5-[(1R)-1-(3,5-dichloro-4-pyridyl)ethoxy]-1H-indazol-3-yl]-5-methoxy-N-(2-pyridylmethyl)aniline;
3-[5-[(1R)-1-(3,5-dichloro-4-pyridyl)ethoxy]-1H-indazol-3-yl]-5-methoxy-N-(3-pyridylmethyl)aniline;
7-[5-[(1R)-1-(3,5-dichloro-4-pyridyl)ethoxy]-1H-indazol-3-yl]-1-(2-pyridylmethyl)-2,3-dihydropyrido[2,3-b][1,4]oxazine;
2-[7-[5-[(1R)-1-(3,5-dichloro-4-pyridyl)ethoxy]-1H-indazol-3-yl]-2,3-dihydropyrido[2,3-b][1,4]oxazin-1-yl]ethanol;
7-[5-[(1R)-1-(3,5-dichloro-4-pyridyl)ethoxy]-1H-indazol-3-yl]-1-(2-morpholinoethyl)-2,3-dihydropyrido[2,3-b][1,4]oxazine;
2-[7-[5-[(1R)-1-(3,5-dichloro-4-pyridyl)ethoxy]-1H-indazol-3-yl]-2,3-dihydropyrido[2,3-b][1,4]oxazin-1-yl]-N,N-dimethyl-ethanamine;
3-[5-[(1R)-1-(3,5-dimethylpyridazin-4-yl)ethoxy]-1H-indazol-3-yl]-5-methoxy-benzonitrile;
3-[5-[(1S)-1-(3,5-dimethylpyridazin-4-yl)ethoxy]-1H-indazol-3-yl]-5-methoxy-benzonitrile;
3-[5-[(1R)-1-(3,5-Dimethylpyridazin-4-yl)ethoxy]-1H-indazol-3-yl]-5-morpholino-benzonitrile;
3-[5-[(1S)-1-(3,5-Dimethylpyridazin-4-yl)ethoxy]-1H-indazol-3-yl]-5-morpholino-benzonitrile;
4-[5-[(1S)-1-(3,5-Dimethylpyridazin-4-yl)ethoxy]-1H-indazol-3-yl]-6-methyl-pyridine-2-carbonitrile;
5-[(1R)-1-(3,5-dimethylpyridazin-4-yl)ethoxy]-3-(3-ethoxy-5-methoxy-phenyl)-1H-indazole;
3-[5-[(1R)-1-(3,5-dimethylpyridazin-4-yl)ethoxy]-1H-indazol-3-yl]-5-methoxy-benzonitrile;
3-(3,5-dimethoxyphenyl)-5-[(1R)-1-(3,5-dimethylpyridazin-4-yl)ethoxy]-1H-indazole;
3-[5-[(1S)-1-(3,5-dimethylpyridazin-4-yl)ethoxy]-1H-indazol-3-yl]-5-methoxy-benzonitrile;
3-[5-[(1R)-1-(3,5-dimethylpyridazin-4-yl)ethoxy]-1H-indazol-3-yl]-5-ethoxy-benzonitrile;
3-[5-[(1R)-1-(3,5-dimethylpyridazin-4-yl)ethoxy]-1H-indazol-3-yl]-5-isopropoxy-benzonitrile;
3-(3,5-dimethoxyphenyl)-5-[(1S)-1-(3,5-dimethylpyridazin-4-yl)ethoxy]-1H-indazole;
5-[(1S)-1-(3,5-dimethylpyridazin-4-yl)ethoxy]-3-(3-ethoxy-5-methoxy-phenyl)-1H-indazole;
5-[(1R)-1-(3,5-dimethylpyridazin-4-yl)ethoxy]-3-(3-isopropoxy-5-methoxy-phenyl)-1H-indazole;
3-[5-[(1R)-1-(3,5-dimethylpyridazin-4-yl)ethoxy]-1H-indazol-3-yl]-5-(trifluoromethoxy)-benzonitrile;
3-[5-[(1R)-1-(3,5-dimethylpyridazin-4-yl)ethoxy]-1H-indazol-3-yl]-5-(4-methylpiperazin-1-yl)benzonitrile;
5-[(1R)-1-(3,5-dimethylpyridazin-4-yl)ethoxy]-3-[3-methoxy-5-(trifluoromethoxy)phenyl]-1H-indazole;
3-[5-[(1R)-1-(3,5-dimethylpyridazin-4-yl)ethoxy]-1H-indazol-3-yl]-5-morpholino-benzonitrile;
3-[5-[(1S)-1-(3,5-dimethylpyridazin-4-yl)ethoxy]-1H-indazol-3-yl]-5-morpholino-benzonitrile;
3-[5-[(1S)-1-(3,5-dimethylpyridazin-4-yl)ethoxy]-1H-indazol-3-yl]-5-(4-methylpiperazin-1-yl)benzonitrile;
3-[5-[(1R)-1-(3,5-dimethylpyridazin-4-yl)ethoxy]-1H-indazol-3-yl]-5-tetrahydrofuran-3-yloxy-benzonitrile;
3-[3-(cyclopropylmethoxy)-5-methoxy-phenyl]-5-[(1R)-1-(3,5-dimethylpyridazin-4-yl)ethoxy]-1H-indazole;
4-[5-[(1R)-1-(3,5-dimethylpyridazin-4-yl)ethoxy]-1H-indazol-3-yl]-6-methyl-pyridine-2-carbonitrile;
4-[5-[(1S)-1-(3,5-dimethylpyridazin-4-yl)ethoxy]-1H-indazol-3-yl]-6-methyl-pyridine-2-carbonitrile;
3-[5-[(1R)-1-(3,5-dimethylpyridazin-4-yl)ethoxy]-1H-indazol-3-yl]-5-tetrahydropyran-4-yloxy-benzonitrile;
3-[5-[(1R)-1-(3,5-dimethylpyridazin-4-yl)ethoxy]-1H-indazol-3-yl]-5-(2-hydroxyethoxy)benzonitrile;
3-[5-[(1R)-1-(3,5-dimethylpyridazin-4-yl)ethoxy]-1H-indazol-3-yl]-5-(2-ethoxyethoxy)-benzonitrile;
5-[5-[(1R)-1-(3,5-dichloro-4-pyridyl)ethoxy]-1H-indazol-3-yl]-2-methoxy-pyridine-3-carbonitrile;
3-[5-[(1R)-1-(3,5-dimethylpyridazin-4-yl)ethoxy]-1H-indazol-3-yl]-5-(2-methoxyethoxy)-benzonitrile;
5-[5-[(1R)-1-(3,5-dimethylpyridazin-4-yl)ethoxy]-1H-indazol-3-yl]-2,3-dimethoxy-benzonitrile;
5-[5-[(1R)-1-(3,5-dimethylpyridazin-4-yl)ethoxy]-1H-indazol-3-yl]-2-fluoro-3-methyl-benzonitrile;
3-(cyclopropylmethoxy)-5-[5-[(1R)-1-(3,5-dimethylpyridazin-4-yl)ethoxy]-1H-indazol-3-yl]benzonitrile;
3-[5-[(1R)-1-(3,5-dimethylpyridazin-4-yl)ethoxy]-1H-indazol-3-yl]-5-methyl-benzonitrile;
5-[5-[(1R)-1-(3,5-dimethylpyridazin-4-yl)ethoxy]-1H-indazol-3-yl]-2-methoxy-3-methyl-benzonitrile;
5-[5-[(1R)-1-(3,5-dichloro-4-pyridyl)ethoxy]-1H-indazol-3-yl]-2,3-dimethoxy-benzonitrile;
5-[5-[(1R)-1-(3,5-dimethylpyridazin-4-yl)ethoxy]-1H-indazol-3-yl]-3-fluoro-2-methoxy-benzonitrile;
5-[5-[(1R)-1-(3,5-dimethylpyridazin-4-yl)ethoxy]-1H-indazol-3-yl]benzene-1,3-dicarbonitrile;
3-cyclopropyl-5-[5-[(1R)-1-(3,5-dimethylpyridazin-4-yl)ethoxy]-1H-indazol-3-yl]benzonitrile;
5-[5-[(1R)-1-(3,5-dimethylpyridazin-4-yl)ethoxy]-1H-indazol-3-yl]-2-methoxy-benzene-1,3-dicarbonitrile;
3-cyclopentyl-5-[5-[(1R)-1-(3,5-dimethylpyridazin-4-yl)ethoxy]-1H-indazol-3-yl]benzonitrile;
3-[5-[(1R)-1-(3,5-dimethylpyridazin-4-yl)ethoxy]-1H-indazol-3-yl]-5-(6-methyl-2,6-diazaspiro[3.3]heptan-2-yl)benzonitrile;
(R)-6-(5-(1-(3,5-Dichloropyridin-4-yl)ethoxy)-1H-indazol-3-yl)spiro[chromane-2,4′-piperidin]-4-one;
(R)-6-(5-(1-(3,5-Dichloropyridin-4-yl)ethoxy)-1H-indazol-3-yl)-1′-ethylspiro[chromane-2,4′-piperidin]-4-one;
6-[5-[(1R)-1-(3,5-Dichloro-4-pyridyl)ethoxy]-1H-indazol-3-yl]-1′-ethyl-spiro[chromane-2,4′-piperidine]-4-ol;
(R)-6-(5-(1-(3,5-Dichloropyridin-4-yl)ethoxy)-1H-indazol-3-yl)-4H-spiro[benzo[d][1,3]dioxine-2,4′-piperidine] TFA salt;
(R)-6-(5-(1-(3,5-Dichloropyridin-4-yl)ethoxy)-1H-indazol-3-yl)-1′-ethyl-4H-spiro[benzo[d][1,3]dioxine-2,4′-piperidine];
(R)-1′-Cyclobutyl-6-(5-(1-(3,5-dichloropyridin-4-yl)ethoxy)-1H-indazol-3-yl)-4H-spiro[benzo[d][1,3]dioxine-2,4′-piperidine];
5-((R)-1-(3,5-Dichloro-2-methylpyridin-4-yl)ethoxy)-3-(6-(((R)-tetrahydrofuran-3-yl)oxy)pyridin-3-yl)-1H-indazole;
5-((R)-1-(3,5-Dichloro-2-methylpyridin-4-yl)ethoxy)-3-(5-methyl-6-(((R)-tetrahydrofuran-3-yl)oxy)pyridin-3-yl)-1H-indazole;
(R)-5-(5-(1-(3,5-Dichloropyridin-4-yl)ethoxy)-1H-indazol-3-yl)-3-methyl-N-(tetrahydro-2H-pyran-4-yl)pyridin-2-amine;
5-(5-((R)-1-(3,5-Dichloro-2-methylpyridin-4-yl)ethoxy)-1H-indazol-3-yl)-N-((R)-tetrahydrofuran-3-yl)pyridin-2-amine;
(R)-6-(5-(1-(3,5-Dichloro-2-methylpyridin-4-yl)ethoxy)-1H-indazol-3-yl)-1′-(methylsulfonyl)-4H-spiro[benzo[d][1,3]dioxine-2,4′-piperidine];
(R)-6-(5-(1-(3,5-Dichloro-2-methylpyridin-4-yl)ethoxy)-1H-indazol-3-yl)-1′-(2,2-difluoroethyl)-4H-spiro[benzo[d][1,3]dioxine-2,4′-piperidine];
5-[5-[(1R)-1-(3,5-dimethylpyridazin-4-yl)ethoxy]-1H-indazol-3-yl]-2-methoxy-pyridine-3-carbonitrile;
(R)-2-(dimethylamino)-5-(5-(1-(3,5-dimethylpyridazin-4-yl)ethoxy)-1H-indazol-3-yl)nicotinonitrile;
3-(5-((R)-1-(3,5-dimethylpyridazin-4-yl)ethoxy)-1H-indazol-3-yl)-5-(tetrahydrofuran-3-yl)benzonitrile;
(S)-3-(5-(1-(3,5-dimethylpyridazin-4-yl)ethoxy)-1H-indazol-3-yl)-5-(6-methyl-2,6-diazaspiro[3.3]heptan-2-yl)benzonitrile;
6-[5-[(1R)-1-(3,5-dichloro-4-pyridyl)ethoxy]-1H-indazol-3-yl]spiro[chromane-2,4′-piperidine]-4-one;
(R)-5-(5-(1-(3,5-dimethylpyridazin-4-yl)ethoxy)-1H-indazol-3-yl)-2-fluoro-3-methoxybenzonitrile;
6-[5-[(1R)-1-(3,5-dichloro-4-pyridyl)ethoxy]-1H-indazol-3-yl]spiro[4H-1,3-benzodioxine-2,4′-piperidine];
6-[5-[(1R)-1-(3,5-dichloro-4-pyridyl)ethoxy]-1H-indazol-3-yl]-1′-ethyl-spiro[chromane-2,4′-piperidine]-4-one;
6-[5-[(1R)-1-(3,5-dichloro-4-pyridyl)ethoxy]-1H-indazol-3-yl]-1′-ethyl-spiro[4H-1,3-benzodioxine-2,4′-piperidine];
(S)-3-(5-(1-(3,5-dimethylpyridazin-4-yl)ethoxy)-6-methoxy-1H-indazol-3-yl)-5-methoxybenzonitrile;
(R)-4-(5-(1-(3,5-dimethylpyridazin-4-yl)ethoxy)-6-methoxy-1H-indazol-3-yl)-6-methoxypicolinonitrile;
(R)-6-(5-(1-(3,5-dimethylpyridazin-4-yl)ethoxy)-1H-indazol-3-yl)-1′-ethylspiro[chroman-2,4′-piperidin]-4-one;
(R)-4-(5-(1-(3,5-dimethylpyridazin-4-yl)ethoxy)-1H-indazol-3-yl)-6-methoxypicolinonitrile;
(R)-3-(5-(1-(3,5-dimethylpyridazin-4-yl)ethoxy)-6-methoxy-1H-indazol-3-yl)-5-methoxybenzonitrile;
(R)-5-(5-(1-(3,5-dimethylpyridazin-4-yl)ethoxy)-6-methoxy-1H-indazol-3-yl)-2,3-dimethoxybenzonitrile;
6-[5-[(1R)-1-(3,5-dichloro-4-pyridyl)ethoxy]-1H-indazol-3-yl]-1′-ethyl-spiro[chromane-2,4′-piperidine]-4-ol;
6-[5-[(1R)-1-(3,5-dichloro-4-pyridyl)ethoxy]-1H-indazol-3-yl]spiro[chromane-2,4′-piperidine];
5-[5-[(1R)-1-(3,5-dichloro-4-pyridyl)ethoxy]-1H-indazol-3-yl]-3-methoxy-2-(4-piperidyloxy)benzonitrile;
(R)-6-(5-(1-(3,5-dimethylpyridazin-4-yl)ethoxy)-1H-indazol-3-yl)-1′-ethyl-4H-spiro[benzo[d][1,3]dioxine-2,4′-piperidine];
6-(5-((R)-1-(3,5-dimethylpyridazin-4-yl)ethoxy)-1H-indazol-3-yl)-1′-ethylspiro[chroman-2,4′-piperidin]-4-ol;
(R)-5-(5-(1-(3,5-dichloropyridin-4-yl)ethoxy)-1H-indazol-3-yl)-2-methoxy-3-methylbenzonitrile;
(R)-3-cyclopropyl-5-(5-(1-(3,5-dichloropyridin-4-yl)ethoxy)-1H-indazol-3-yl)benzonitrile;
(R)-5-(5-(1-(3,5-dimethylpyridazin-4-yl)ethoxy)-1H-indazol-3-yl)-2-((1-isopropylpiperidin-4-yl)oxy)-3-methylbenzonitrile;
(R)-6-(5-(1-(3,5-dimethylpyridazin-4-yl)ethoxy)-1H-indazol-3-yl)-1′-ethylspiro[chroman-2,4′-piperidine];
(R)-6-(5-(1-(3,5-dimethylpyridazin-4-yl)ethoxy)-1H-indazol-3-yl)-1′-isopropyl-4H-spiro[benzo[d][1,3]dioxine-2,4′-piperidine];
(R)-5-(5-(1-(3,5-dimethylpyridazin-4-yl)ethoxy)-1H-indazol-3-yl)-3-methyl-2-((1-(oxetan-3-yl)piperidin-4-yl)oxy)benzonitrile;
(R)-5-(5-(1-(3,5-Dichloropyridin-4-yl)ethoxy)-1H-indazol-3-yl)-2-((1-ethylpiperidin-4-yl)oxy)-3-methoxybenzonitrile;
(R)-5-(5-(1-(3,5-dimethylpyridazin-4-yl)ethoxy)-1H-indazol-3-yl)-2-((1-ethylpiperidin-4-yl)oxy)-3-methylbenzonitrile;
(R)-5-(5-(1-(3,5-dimethylpyridazin-4-yl)ethoxy)-6-methoxy-1H-indazol-3-yl)-2-methoxy-3-methylbenzonitrile;
Name (R)-3-cyclopropyl-5-(5-(1-(3,5-dimethylpyridazin-4-yl)ethoxy)-6-methoxy-1H-indazol-3-yl)benzonitrile;
(R)-5-(5-(1-(3,5-Dichloropyridin-4-yl)ethoxy)-1H-indazol-3-yl)-2-(piperidin-4-yloxy)benzonitrile;
5-[5-[(1R)-1-(3,5-dichloro-4-pyridyl)ethoxy]-1H-indazol-3-yl]-2-[(1-ethyl-4-piperidyl)oxy]pyridine-3-carbonitrile;
(R)-2-((1-cyclobutylpiperidin-4-yl)oxy)-5-(5-(1-(3,5-dimethylpyridazin-4-yl)ethoxy)-1H-indazol-3-yl)-3-methylbenzonitrile;
6-[5-[(1R)-1-(3,5-dichloro-4-pyridyl)ethoxy]-1H-indazol-3-yl]-1′-ethyl-spiro[chromane-2,4′-piperidine];
5-[5-[(1R)-1-(3,5-dichloro-4-pyridyl)ethoxy]-1H-indazol-3-yl]-2-(2-hydroxy-2-methyl-propoxy)pyridine-3-carbonitrile;
(R)-6-(5-(1-(3,5-dichloro-2-fluoropyridin-4-yl)ethoxy)-1H-indazol-3-yl)-1′-isopropyl-8-methoxy-4H-spiro[benzo[d][1,3]dioxine-2,4′-piperidine];
5-[5-[(1R)-1-(3,5-dichloro-4-pyridyl)ethoxy]-1H-indazol-3-yl]-2-[(1-isopropyl-4-piperidyl)oxy]benzonitrile;
(R)-6-(5-(1-(3,5-dimethylpyridazin-4-yl)ethoxy)-1H-indazol-3-yl)-1′-isopropyl-8-methoxy-4H-spiro[benzo[d][1,3]dioxine-2,4′-piperidine];
5-[5-[(1R)-1-(3,5-dichloro-4-pyridyl)ethoxy]-1H-indazol-3-yl]-2-[(1-isopropyl-4-piperidyl)oxy]-3-methoxy-benzonitrile;
5-(5-((R)-1-(3,5-dimethylpyridazin-4-yl)ethoxy)-1H-indazol-3-yl)-2-(((S)-tetrahydrofuran-3-yl)oxy)nicotinonitrile;
5-(5-((R)-1-(3,5-dimethylpyridazin-4-yl)ethoxy)-1H-indazol-3-yl)-2-(((R)-tetrahydrofuran-3-yl)oxy)nicotinonitrile;
5-(5-((R)-1-(3,5-dichloropyridin-4-yl)ethoxy)-1H-indazol-3-yl)-2-(((S)-tetrahydrofuran-3-yl)amino)nicotinonitrile;
5-(5-((R)-1-(3,5-dichloropyridin-4-yl)ethoxy)-1H-indazol-3-yl)-2-(((R)-tetrahydrofuran-3-yl)amino)nicotinonitrile;
5-[5-[(1R)-1-(3,5-dichloro-4-pyridyl)ethoxy]-6-methoxy-1H-indazol-3-yl]-2-(dimethylamino)pyridine-3-carbonitrile;
(R)-5-(5-(1-(3,5-dichloropyridin-4-yl)ethoxy)-1H-indazol-3-yl)-2-((tetrahydro-2H-pyran-4-yl)amino)nicotinonitrile;
5-(5-((R)-1-(3,5-dichloropyridin-4-yl)ethoxy)-1H-indazol-3-yl)-2-(((S)-tetrahydrofuran-3-yl)oxy)nicotinonitrile;
5-(5-((R)-1-(3,5-dichloropyridin-4-yl)ethoxy)-1H-indazol-3-yl)-2-(((R)-tetrahydrofuran-3-yl)oxy)nicotinonitrile;
5-(5-((R)-1-(3,5-dichloropyridin-4-yl)ethoxy)-6-methyl-1H-indazol-3-yl)-2-(((R)-tetrahydrofuran-3-yl)oxy)nicotinonitrile;
5-(5-((R)-1-(3,5-dichloropyridin-4-yl)ethoxy)-6-methoxy-1H-indazol-3-yl)-2-(((R)-tetrahydrofuran-3-yl)oxy)nicotinonitrile;
7-[5-[(1R)-1-(3,5-dichloro-4-pyridyl)ethoxy]-1H-indazol-3-yl]-1-methylsulfonyl-2,3-dihydropyrido[2,3-b][1,4]oxazine;
5-(5-((R)-1-(3,5-dichloropyridin-4-yl)ethoxy)-6-fluoro-1H-indazol-3-yl)-2-(((R)-tetrahydrofuran-3-yl)oxy)nicotinonitrile;
(R)-7-(5-(1-(3,5-dichloropyridin-4-yl)ethoxy)-1H-indazol-3-yl)-1-(2,2,2-trifluoroethyl)-2,3-dihydro-1H-pyrido[2,3-b][1,4]oxazine 2,2,2-trifluoroacetate;
7-[5-[(1R)-1-(3,5-dichloro-4-pyridyl)ethoxy]-6-methoxy-1H-indazol-3-yl]-1-methylsulfonyl-2,3-dihydropyrido[2,3-b][1,4]oxazine;
(R)-7-(5-(1-(3,5-dichloropyridin-4-yl)ethoxy)-1H-indazol-3-yl)-1-isopropyl-2,3-dihydro-1H-pyrido[2,3-b][1,4]oxazine;
(R)-7-(5-(1-(3,5-dichloropyridin-4-yl)ethoxy)-1H-indazol-3-yl)-1-(2,2-difluoroethyl)-2,3-dihydro-1H-pyrido[2,3-b][1,4]oxazine;
7-[6-chloro-5-[(1R)-1-(3,5-dichloro-2-methyl-4-pyridyl)ethoxy]-1H-indazol-3-yl]-1-methylsulfonyl-2,3-dihydropyrido[2,3-b][1,4]oxazine;
7-[5-[(1R)-1-(3,5-dichloro-4-pyridyl)ethoxy]-1H-indazol-3-yl]-1-ethylsulfonyl-2,3-dihydropyrido[2,3-b][1,4]oxazine;
7-[6-chloro-5-[(1R)-1-(3,5-dichloro-4-pyridyl)ethoxy]-1H-indazol-3-yl]-1-methylsulfonyl-2,3-dihydropyrido[2,3-b][1,4]oxazine;
R)-7-(5-(1-(3,5-Dichloropyridin-4-yl)ethoxy)-6-methoxy-1H-indazol-3-yl)-2,3-dihydro-1H-pyrido[2,3-b][1,4]oxazine;
5-[(1R)-1-(3,5-dichloro-2-methyl-4-pyridyl)ethoxy]-3-[6-[(3R)-tetrahydrofuran-3-yl]oxy-3-pyridyl]-1H-indazole;
5-[(1R)-1-(3,5-dichloro-2-methyl-4-pyridyl)ethoxy]-3-[5-methyl-6-[(3R)-tetrahydrofuran-3-yl]oxy-3-pyridyl]-1H-indazole;
5-[5-[(1R)-1-(3,5-dichloro-2-methyl-4-pyridyl)ethoxy]-6-methoxy-1H-indazol-3-yl]-N,N-dimethyl-pyridin-2-amine;
5-[(1R)-1-(3,5-dichloro-2-methyl-4-pyridyl)ethoxy]-3-[6-[(3S)-tetrahydrofuran-3-yl]oxy-3-pyridyl]-1H-indazole;
(R)-5-(5-(1-(3,5-dichloropyridin-4-yl)ethoxy)-1H-indazol-3-yl)-3-methyl-N-(tetrahydro-2H-pyran-4-yl)pyridin-2-amine;
(R)-5-(1-(3,5-dichloropyridin-4-yl)ethoxy)-3-(6-((2-(methylsulfonyl)-2-azaspiro[3.3]heptan-6-yl)oxy)pyridin-3-yl)-1H-indazole;
5-(5-((R)-1-(3,5-dichloropyridin-4-yl)ethoxy)-1H-indazol-3-yl)-3-methyl-N—((S)-tetrahydrofuran-3-yl)pyridin-2-amine;
5-[(1R)-1-(3,5-dichloro-2-methyl-4-pyridyl)ethoxy]-3-[5-methyl-6-[(3S)-tetrahydrofuran-3-yl]oxy-3-pyridyl]-1H-indazole;
5-[5-[(1R)-1-(3,5-dichloro-2-methyl-4-pyridyl)ethoxy]-1H-indazol-3-yl]-N-[(3R)-tetrahydrofuran-3-yl]pyridin-2-amine;
5-[5-[(1R)-1-(3,5-dichloro-2-methyl-4-pyridyl)ethoxy]-1H-indazol-3-yl]-N-tetrahydropyran-4-yl-pyridin-2-amine;
l′-cyclobutyl-6-[5-[(1R)-1-(3,5-dichloro-4-pyridyl)ethoxy]-1H-indazol-3-yl]spiro[4H-1,3-benzodioxine-2,4′-piperidine];
5-(5-((R)-1-(3,5-dichloropyridin-4-yl)ethoxy)-1H-indazol-3-yl)-3-methyl-N-((R)-tetrahydrofuran-3-yl)pyridin-2-amine;
(R)—N-(5-(5-(1-(3,5-dichloropyridin-4-yl)ethoxy)-1H-indazol-3-yl)pyridin-2-yl)-2-(methylsulfonyl)-2-azaspiro[3.3]heptan-6-amine;
5-[5-[(1R)-1-(3,5-dichloro-2-methyl-4-pyridyl)ethoxy]-1H-indazol-3-yl]-N-[(3S)-tetrahydrofuran-3-yl]pyridin-2-amine;
(R)-3-(6-(2-oxaspiro[3.3]heptan-6-yloxy)pyridin-3-yl)-5-(1-(3,5-dichloropyridin-4-yl)ethoxy)-1H-indazole;
5-((R)-1-(3,5-dichloropyridin-4-yl)ethoxy)-3-(6-((3-(methylsulfonyl)-3-azabicyclo[3.1.0]hexan-6-yl)oxy)pyridin-3-yl)-1H-indazole;
6-[5-[(1R)-1-(3,5-dichloro-2-methyl-4-pyridyl)ethoxy]-1H-indazol-3-yl]-1′-methylsulfonyl-spiro[4H-1,3-benzodioxine-2,4′-piperidine];
5-[5-[(1R)-1-(3,5-dichloro-2-methyl-4-pyridyl)ethoxy]-1H-indazol-3-yl]-3-methyl-N-[(3R)-tetrahydrofuran-3-yl]pyridin-2-amine;
6-[5-[(1R)-1-(3,5-dichloro-4-pyridyl)ethoxy]-1H-indazol-3-yl]-1′-methylsulfonyl-spiro[4H-1,3-benzodioxine-2,4′-piperidine];
6-[5-[(1R)-1-(3,5-dichloro-2-methyl-4-pyridyl)ethoxy]-1H-indazol-3-yl]-1′-(2,2-difluoroethyl)spiro[4H-1,3-benzodioxine-2,4′-piperidine];
6-[5-[(1R)-1-(3,5-dichloro-2-methyl-4-pyridyl)ethoxy]-1H-indazol-3-yl]-1′-(2,2,2-trifluoroethyl)spiro[4H-1,3-benzodioxine-2,4′-piperidine];
6-[5-[(1R)-1-(3,5-dichloro-4-pyridyl)ethoxy]-1H-indazol-3-yl]-1′-(2,2-difluoroethyl)spiro[4H-1,3-benzodioxine-2,4′-piperidine]; or
6-[5-[(1R)-1-(3,5-dichloro-4-pyridyl)ethoxy]-1H-indazol-3-yl]-1′-(2,2,2-trifluoroethyl)spiro[4H-1,3-benzodioxine-2,4′-piperidine].
